(19) United States
(12) Patent Application Publication
(10) Pub. No.: US 2025/0388529 A1
(43) Pub. Date: Dec. 25, 2025

(54) N-Substituted Phenylalkylamines and Their Use as Therapeutic Agents

(71) Applicant: Alexander Shulgin Research Institute, Inc., Lafayette, CA (US)

(72) Inventor: Nicholas V. Cozzi, Hartford, WI (US)

(21) Appl. No.: 18/878,887

(22) PCT Filed: Jun. 26, 2023
(86) PCT No.: PCT/US2023/026258

§ 371 Date: Dec. 25, 2024

(51) Int. Cl. C07C 217/60; A61K 31/137; A61K 45/06

(52) U.S. Cl. CPC C07C 217/60; A61K 31/137; A61K 45/06

(57) ABSTRACT

Provided are N-substituted phenylalkylamines, methods of making the same, and methods of using such compounds, for example, as receptor probes, as modulators of neurotransmission, and as therapeutic agents, for example as CNS agents. Also provided are pharmaceutical compositions and methods of their use to treat certain disorders, such as disorders related to serotonergic neurotransmission, mental health disorders, and ion-channel mediated conditions, such as seizure disorders.

### CROSS-REFERENCE

Priority is claimed under PCT Art. 8 (1) and Rule 4.10 to U.S. Appl. No. 63/355,632, filed Jun. 26, 2022, which is incorporated by reference for all purposes as if fully set forth herein.

### FIELD OF THE INVENTION

This disclosure relates in some aspects to N-substituted phenylalkylamine compounds. In some aspects, it further relates to methods of synthesizing the compounds, compositions containing the compounds, and methods of using such compounds, including their administration to subjects. In some aspects, useful features of the compounds include neuromodulatory activity.

### BACKGROUND OF THE INVENTION

Tryptamine-class psychedelic compounds such as psilocybin and DMT have re-emerged as promising therapeutic candidates for treating numerous psychiatric conditions including depressive disorders, substance use disorders, and anxiety-related disorders (Vollenweider and Preller, Nat Rev Neurosci, 2020; 21 (11): 611-624; D'Souza et al., Neuropsychopharmacol., 2022; 47 (10): 1854-1862). Aside from their remarkable subjective effects on consciousness, there is increasing interest in their physiological effects and their potential application in the treatment of physical and neurological disorders. Further, while much progress has been made in recent years towards understanding the structure-activity relationships underlying the effects of classical psychedelics, many open questions remain concerning the efficacy and potential risks of these compounds for the treatment of complex diseases that may lack effective treatments, such as mood disorders (e.g., depressive disorders, bipolar disorder) and psychotic disorders (e.g., schizophrenia). As such, there is an ongoing unmet need for novel alternative treatments, especially those which minimize side effects, increase access, optimize efficacy, and even improve upon existing classical psychedelics by invoking new therapeutic mechanisms.

Provided herein are compounds, compositions, methods, uses, and pharmaceutical kits to meet these needs and others, and having such advantages and improvements as will become readily apparent through the disclosure below.

### INCORPORATION BY REFERENCE

Each patent, publication, and non-patent literature cited in the application is hereby incorporated by reference in its entirety, as if each was incorporated by reference individually, and as if each is fully set forth herein. However, where such reference is made, it is for the general purpose of providing context for discussing disclosed features, and unless specifically stated otherwise, should not be construed as an admission that the document or underlying information, in any jurisdiction, is prior art or part of the common general knowledge in the art.

### BRIEF SUMMARY OF THE INVENTION

The following presents a simplified summary of some embodiments of the invention in order to provide a basic understanding of the invention. This summary is not an extensive overview of the invention. It is not intended to identify key or critical elements of the invention or to delineate the scope of the invention. Its sole purpose is to present some embodiments of the invention in a simplified form as a prelude to the more detailed description that is presented later.

In a first aspect, provided is a compound of Formula (II):

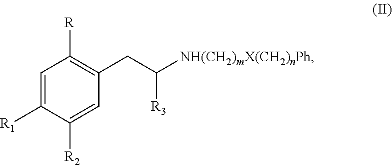

wherein:
m and n are each independently an integer from 1 to 13, provided that the sum of m+n is from 6 to 14;
X is O, S, or NH;
R and R2 are each independently C1-C8 alkoxy, wherein each C1-C8 alkoxy is optionally substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate;
R1 is selected from the group consisting of hydrogen, halogen, C1-C8 alkyl, C1-C8 alkoxy, C1-C8 thioalkyl, C2-C8 alkenyl, C2-C8 alkynyl, C3-C8 cycloalkyl, C3-C8 cycloalkenyl, aryl or heterocyclyl, each of which is optionally substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate;
R3 is hydrogen or C1-C8 alkyl; and
Ph is phenyl optionally substituted by halogen, azido, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate; or a pharmaceutically acceptable salt thereof.

In some embodiments, R and R2 are both —OCH₃.

In some embodiments, R3 is hydrogen, —CH₃, or —CH₂CH₃. In some embodiments, R3 is hydrogen or —CH₃. In some embodiments, R3 is —CH₃ or —CH₂CH₃. In some embodiments, R3 is hydrogen. In some embodiments, R3 is —CH₃. In some embodiments, R3 is —CH₂CH₃.

In some embodiments, R1 is F, Cl, Br, or I. In some embodiments, R1 is Br. In some embodiments, R1 is C1-C8 alkyl. In some embodiments, R1 is —CH₃ or —CH₂CH₃. In some embodiments, R1 is C1-C8 thioalkyl. In some embodiments, R1 is —SCH₃. In some embodiments, R1 is unsubstituted phenyl. In some embodiments, R1 is phenyl substituted by azido or C1-C8 alkoxy.

In some embodiments, X is O. In embodiments, X is S. In embodiments, X is NH.

In some embodiments, the sum of m+n is from 8 to 12. In some embodiments, the sum of m+n is from 9 to 11. In some embodiments, the sum of m+n is 10. In some embodiments, m is 6 and n is 4.

In another aspect, provided is a compound selected from Table 4, or a pharmaceutically acceptable salt thereof.

In some embodiments, the compound is

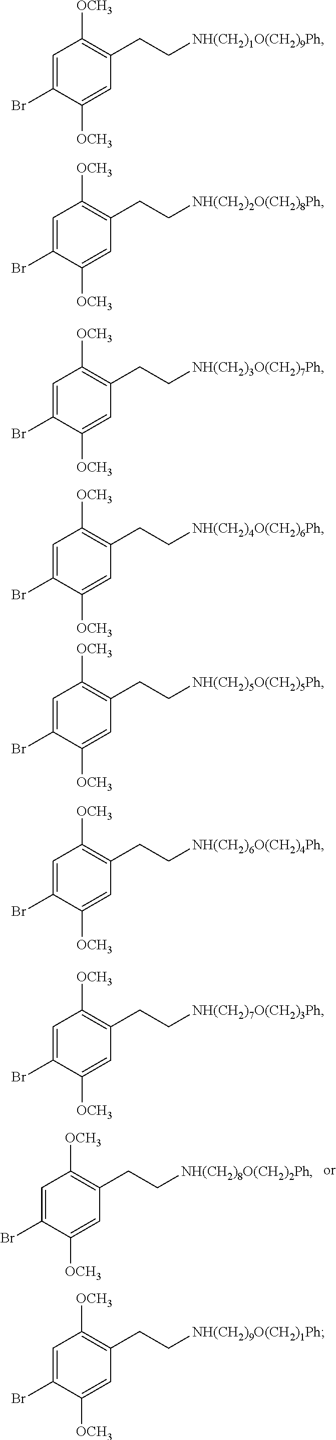

or a pharmaceutically acceptable salt thereof.

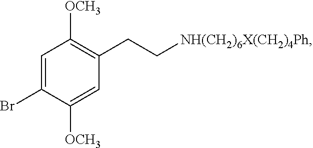

In some embodiments, the compound is or a pharmaceutically acceptable salt thereof.

Also provided is a pharmaceutical composition comprising a therapeutically effective amount of the compound of the preceding embodiments, or a pharmaceutically acceptable salt thereof, and a pharmaceutically acceptable carrier, diluent, or excipient.

In some embodiments, the composition is suitable for oral, buccal, sublingual, intranasal, injectable, subcutaneous, intravenous, or transdermal administration.

In some embodiments, the composition is in unit dosage form. In some embodiments, the unit dosage form comprises the compound, or a pharmaceutically acceptable salt thereof, in a total amount of between about 1 and about 500 mg, between about 2.5 and about 250 mg, or between about 5 and about 125 mg. In some embodiments, the composition is an immediate release, controlled release, sustained release, extended release, or modified release formulation.

In some embodiments, the composition further comprises a therapeutically effective amount of an additional active compound, or a pharmaceutically acceptable salt thereof. In some embodiments, the additional active compound is selected from the group consisting of amino acids, antioxidants, anti-inflammatory agents, analgesics, antineuropathic and antinociceptive agents, antimigraine agents, anxiolytics, antidepressants, antipsychotics, anti-PTSD agents, dissociatives, cannabinoids, immunostimulants, anti-cancer agents, antiemetics, orexigenics, antiulcer agents, antihistamines, antihypertensives, anticonvulsants, antiepileptics, bronchodilators, neuroprotectants, nootropics, empathogens, psychedelics, monoamine oxidase inhibitors, tryptamines, terpenes, phenethylamines, sedatives, stimulants, and vitamins; or a pharmaceutically acceptable salt thereof.

In some embodiments, the additional active compound, or a pharmaceutically acceptable salt thereof, acts to increase a therapeutic effect, provide an additional therapeutic effect, decrease an unwanted effect, increase stability or shelf-life, improve bioavailability, induce synergy, or alter pharmacokinetics or pharmacodynamics. In some embodiments, the additional therapeutic effect is an antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, nootropic, empathogenic, psychedelic, sedative, or stimulant effect.

In another aspect, provided is the compound of any of the preceding embodiments, or a pharmaceutically acceptable salt thereof, for use in the treatment of a medical condition.

In another aspect, provided is the compound of any of the preceding embodiments, or a pharmaceutically acceptable salt thereof, for the manufacture of a medicament for the treatment of a medical condition.

In another aspect, provided is a method for modulating neurotransmission in a mammal, comprising administering to the mammal a therapeutically effective amount of the compound of any the preceding embodiments, or a pharmaceutically acceptable salt thereof.

Also provided is a method of treating a medical condition in a mammal in need of such treatment, the method comprising administering the compound of any of the preceding embodiments, or a pharmaceutically acceptable salt thereof.

In some embodiments, the medical condition is a disorder linked to dysregulation or inadequate functioning of neurotransmission. In some embodiments, the disorder linked to dysregulation or inadequate functioning of neurotransmission is that of monoaminergic neurotransmission. In some embodiments, the disorder linked to dysregulation or inadequate functioning of neurotransmission is that of serotonergic neurotransmission.

In some embodiments, the medical condition is a mental health disorder. In some embodiments, the mental health disorder is selected from the group consisting of schizophrenia, schizoaffective disorder, schizotypal disorder, acute and transient psychotic disorder, delusional disorder, a substance-induced psychotic disorder, bipolar disorder, bipolar type I disorder, bipolar type II disorder, cyclothymic disorder, post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders.

In some embodiments, the medical condition is a seizure disorder. In some embodiments, the seizure disorder is epilepsy.

In some embodiments, the medical condition is a disorder linked to dysregulation or inadequate functioning of a voltage-gated ion channel. In some embodiments, the voltage-gated ion channel is a voltage-gated sodium channel. In some embodiments, the compound inhibits the activity of the voltage-gated sodium channel.

In some embodiments, the mammal has a genetic variation associated with drug metabolism, including a genetic variation relating to CYP2D6 or CYP3A4 enzymes; or associated with a mental health disorder, trauma or stressor related disorder, depression, or anxiety, and including a genetic variation in mGluR5 or FKBP5; or relating to a membrane transporter, such as SERT, DAT, NET, or VMAT.

In some embodiments, the mammal has altered epigenetic regulation of a gene the expression of which is associated with a mental health condition or susceptibility to a mental health treatment, such as the SIGMAR1 gene for the non-opioid sigma-1 receptor.

In some embodiments, the mammal is a human.

Also provided is a method of reducing the symptoms of a mental health disorder in a human, the method comprising identifying a human in need of said reducing, and administering to the human the compound of any of the preceding embodiments, or a pharmaceutically acceptable salt thereof, or the pharmaceutical composition of any of the preceding embodiments.

Also provided is a method of improving mental health or functioning in a human, the method comprising identifying a human in need of said improving, and administering to the human the compound of any of the preceding embodiments, or a pharmaceutically acceptable salt thereof, or the pharmaceutical composition of any of the preceding embodiments.

The foregoing has outlined broadly and in summary certain pertinent features of the disclosure so that the detailed description of the invention that follows may be better understood, and so that the present contribution to the art can be more fully appreciated. Hence, this summary is to be considered as a brief and general synopsis of only some of the objects and embodiments disclosed herein, is provided solely for the benefit and convenience of the reader, and is not intended to limit in any manner the scope, or range of equivalents, to which the claims are lawfully entitled. Additional features of the invention are described hereinafter. It should be appreciated by those in the art that all disclosed specific compositions and methods are only exemplary, and may be readily utilized as a basis for modifying or designing other compositions and methods for carrying out the same purposes. Such equivalent compositions and methods will be appreciated to be also within the scope and spirit of the invention as set forth in the claims.

The headings within this document are being utilized only to expedite its review by a reader. They should not be construed as limiting the invention in any manner.

### BRIEF SUMMARY OF THE DRAWINGS

To further clarify various aspects of the invention, a more particular description thereof will be rendered by reference to certain exemplary embodiments thereof which are illustrated in the figures. It will be understood and appreciated that the figures depict only illustrated embodiments of the invention and are not to be considered limiting of its scope. They are simply provided as exemplary illustrations of some embodiments of the invention. Certain aspects of the invention are therefore further described and explained with additional specificity and detail, but still by way of example only, with reference to the following accompanying figures.

FIG. 1 is a visual representation of the receptor affinities and functional activities of 2C-B and N-(4-bromo-2,5-dimethoxyphenethyl)-6-(4-phenylbutoxy) hexan-1-amine (XOB).

FIG. 2 shows the ¹H NMR spectrum of XOB in CDCl₃. Chemical shifts were referenced with respect to the TMS peak at 0 ppm. 2D COSY was performed to unambiguously assign resonances and 1H integrals were normalized relative to the triplet resonance at 2.64 ppm, which was assigned a value of 2.00 protons (for the two benzylic protons).

FIG. 3A shows the total ion chromatogram and FIG. 3B shows the electron impact mass spectrum of XOB. XOB eluted as a single peak in the GC at 11.068 min.

FIG. 4A shows the absorbance chromatogram of XOB, and FIG. 4B shows the corresponding data in tabular form. Liquid chromatography was performed using a Waters Acquity I-Class UPLC system equipped with a Waters HSS T3 column. Absorbance was monitored at a wavelength of 269 nm using a photodiode array detector. XOB (8) eluted as a single peak with a retention time of 1.53 min and represented 96.76% of the total peak area.

FIG. 5A and FIG. 5B show the high resolution mass spectra of XOB. The mass spectra were acquired using a Waters Xevo G2-XS quadrupole time-of-flight (QTof) mass spectrometer operating in ESI-positive mode.

FIG. 6 shows sequence alignment between TMD4 of the human β2 adrenergic receptor and TMD4 of the human 5-HT_{2A} receptor.

FIG. 7 shows sequence alignment between TMD6 of the human β2 adrenergic receptor and TMD6 of the human 5-HT_{2A} receptor.

FIG. 8 shows sequence alignment between TMD7 of the human β2 adrenergic receptor and TMD7 of the human 5-HT_{2A} receptor.

FIG. 9 shows sequence alignment between ECL2 of the human β2 adrenergic receptor and ECL2 of the human 5-HT_{2A} receptor.

FIG. 10 shows sequence alignment between ECL3 of the human β2 adrenergic receptor and ECL3 of the human 5-HT_{2A} receptor.

FIG. 11A shows the chemical structure of the clozapine tracer. FIG. 11B shows the chemical structure of the NAN-190 tracer. FIG. 11C and FIG. 11D show saturation binding of clozapine tracer to transiently expressed HiBiT-5-HT_{2A}. FIG. 11E and FIG. 11F show saturation binding of clozapine tracer to transiently expressed HiBiT-5-HT_{2C}. FIG. 11G and FIG. 11H show saturation binding of NAN-190 tracer to transiently expressed HiBiT-5-HT_{1A}. FIG. 11I shows the equilibrium dissociation constants (K_{D}) for these tracers.

FIGS. 12A-12D show binding characteristics of XOB and serotonin for 5-HT receptors by NanoBRET. Competitive displacement of fluorescent tracers by increasing concentrations of unmodified XOB and serotonin for transiently expressed HiBiT-5-HT_{2A} (FIG. 12A), HiBiT-5-HT_{2C} (FIG. 12B), and HiBiT-5-HT_{1A} (FIG. 12C). FIG. 12D shows the equilibrium binding constants (Kᵢ).

FIG. 13 shows binding characteristics of clozapine tracer for HiBiT-5-HT_{2A} in equilibrium and real time. (Top) Kinetic binding analysis; (Bottom) Binding characteristics in equilibrium and kinetic modes.

FIG. 14 shows binding characteristics of serotonin and XOB for HiBiT-5-HT_{2A} in equilibrium and real time. Kinetic binding analysis for (Top) Serotonin and (Bottom) XOB. The table shows binding characteristics in equilibrium and kinetic modes.

FIGS. 15A-15C show Gq-mediated calcium flux agonist and antagonist activity of XOB at 5-HT_{2A}, 5-HT_{2B}, and 5-HT_{2C} receptors. Gq-mediated calcium flux responses in agonist mode (top) and antagonist mode (bottom) comparing XOB to positive control (5-HT for agonist mode and clozapine for antagonist mode) at 5-HT_{2A} (FIG. 15A), 5-HT_{2B} (FIG. 15B), and 5-HT_{2C} (FIG. 15C) receptors stably expressed in Flp-In 293 T-Rex inducible cell lines. 5-HT (3.2 nM) concentration was used for antagonist mode. All data represent mean and S.E.M from three independent experiments measured in triplicate.

FIGS. 16A-16D show acute effects of XOB on hNav1.1 using manual patch clamp. FIG. 16A shows representative I_{Na} current traces from the same HEK cell at baseline (top), 4 min post 10 μM XOB perfusion (middle), and 10 minutes post washout (bottom). FIG. 16B shows combined current-voltage (I-V) relationship of cells at baseline, 10 μM XOB, and after washout. FIG. 16C shows representative peak I_{Na} traces showing the baseline initial sweep (black) and 3.9 min post-perfusion of 1, 5 and 10 μM XOB (gray). HEK cells used in A-B were stably expressing human Naᵥ1.1, while those used in C-D expressed both Naᵥ1.1 and Naᵥβ1 subunits. FIG. 16D shows diary plots of peak I_{Na} evoked using a voltage step from −120 mV to 0 mV for 250 ms every 5.21 s.

FIGS. 17A-17D show that XOB shows a concentration-dependent inhibition of peak I_{Na} using automated patch clamp (SyncroPatch). FIG. 17A shows the concentration response curve of XOB on peak I_{Na} (IC₅₀: 4.29 μM, 95% CI: 3.24 μM to 5.55 μM; Hill slope: 1.45, 95% CI: 0.99 to 2.19). FIG. 17B shows a SyncroPatch 384-well array showing heat map of peak I_{Na} inhibition by XOB perfusion. Vehicle and 1 μM TTX conditions are displayed in the two far left and far right columns, respectively. FIG. 17C shows representative single well peak I_{Na} traces from the SyncroPatch for vehicle (grey), 10 μM XOB (black1), and 1 μM TTX (black) conditions. FIG. 17D shows the time course of normalized peak I_{Na} inhibition shows the mean peak I_{Na} value for each concentration group of cells at all sweeps under baseline (<120 s) or drug condition (>120 s). The dotted line denotes the start of drug perfusion. The experiment was performed with HEK cells stably co-expressing human Naᵥ1.1 and Naᵥβ1.

FIGS. 18A-18C show the concentration response of XOB on I-V relationship and voltage-dependent properties of hNaᵥ1.1+h1 using automated patch clamp (SyncroPatch). FIG. 18A shows the effects of increasing concentrations of XOB on I-V curves. FIG. 18B shows peak I_{Na} density elicited using a voltage step from −120 mV to −20 mV taken from I-V data in FIG. 18A. FIG. 18C shows a comparison of voltage-dependence of conductance (G/G_{Max}) and voltage-dependence of availability (I/I_{Max}) between vehicle (open dots) and 3 μM XOB (solid dots) conditions. The experiment was performed with HEK cells stably co-expressing human Naᵥ1.1 and Naᵥβ1.

FIGS. 19A-19J show the effects of 10 μM XOB on wildtype mouse prefrontal cortex layer V pyramidal neuron membrane properties and action potential firing. FIG. 19A shows input resistance, FIG. 19B shows resting membrane potential, FIG. 19C shows 1^{st} AP threshold, FIG. 19D shows capacitance, FIG. 19E shows spike half-width, FIG. 19F shows 1^{st} AP peak amplitude, FIG. 19G shows maximum dV/dt, FIG. 19H shows minimum dV/dt, and FIG. 19I shows AP firing frequency at 400 pA current injection; and FIG. 19J shows frequency-current relationship of PFC layer V pyramidal neurons at baseline (solid dots) and after perfusion of 10 μM XOB (open dots).

FIG. 20 shows representative action potential traces of a wildtype mouse prefrontal cortex layer V pyramidal neuron at 0, 100, 200, 300, and 400 pA current injections in baseline (left column) and 10 μM XOB conditions (right column).

### DETAILED DESCRIPTION OF THE INVENTION

While various aspects and features of certain embodiments are summarized above, the following detailed description illustrates several exemplary embodiments in further detail to enable one of skill to practice such embodiments, and to make and use the full scope of the invention claimed. The described examples are provided for illustrative purposes and are not intended to limit the scope of the invention or its applications, which includes all embodiments and formulations thereof, not only those expressly described. It will be understood that many modifications, substitutions, changes, and variations in the described examples, embodiments, applications, and details of the invention illustrated herein can be made by those in the art without departing from the spirit of the invention, or the scope of the invention as claimed.

### A. GENERAL DEFINITIONS AND TERMS

As used in this specification and the appended claims, the singular forms “a,” “an,” and “the” include plural referents unless the context clearly dictates otherwise. Thus, for example, reference to “an active agent” includes reference to a combination of two or more active agents, and reference to “an excipient” includes reference to a combination of two or more excipients. While the term “one or more” may be used, its absence (or its replacement by the singular) does not signify the singular only, but simply underscores the possibility of multiple agents or ingredients in particular embodiments.

The terms “comprising,” “including,” “such as,” and “having” are intended to be inclusive and not exclusive (i.e., there may be other elements in addition to the recited elements). Thus, the term “including” as used herein means, and is used interchangeably with, the phrase “including but not limited to.” The term “or” is used herein to mean, and is used interchangeably with, the term “and/or,” unless context clearly indicates otherwise.

Unless otherwise indicated, all numbers expressing quantities of ingredients, properties such as concentration, reaction conditions, and so forth, used to describe and claim certain embodiments of the invention are to be understood as being modified in some instances by the term “about.” Accordingly, in embodiments, the numerical parameters set forth in the written description and attached claims are approximations that can vary depending upon the desired properties sought to be obtained by a particular embodiment. In embodiments, “about” refers to plus or minus five percent (±5%) of the recited unit of measure. In embodiments, the term “from,” when used in the context of a numerical range, is inclusive of both boundary numbers in said range. For example, the phrase “from 1 to 10” is inclusive of the numbers 1 and 10. The term “substantially,” where it is applied to modify a feature or limitation herein, will be read in the context of the invention and in light of the knowledge in the art to provide the appropriate certainty, e.g., by using a standard that is recognized in the art for measuring the meaning of “substantially” as a term of degree, or by ascertaining the scope as would one of skill in the art.

In some embodiments (equivalently and as shorthand, “in embodiments”), the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments may contain certain errors necessarily resulting from the standard deviation found in their respective testing measurements.

A comprehensive list of the abbreviations utilized by organic chemists of ordinary skill in the art appears in the first issue of each volume of the Journal of Organic Chemistry; this list is typically presented in a table entitled Standard List of Abbreviations; the current list as of the date of this filing is hereby incorporated by reference as if fully set forth herein.

Unless defined otherwise, all technical and scientific terms herein have the meaning as commonly understood by one having ordinary skill in the art to which this invention belongs, who as a shorthand may be referred to simply as “one of skill.” Further definitions that may assist the reader in understanding the disclosed embodiments are as follows; however, it will be appreciated that such definitions are not intended to limit the scope of the invention, which shall be properly interpreted and understood by reference to the full specification (as well as any plain meaning known to one of skill in the relevant art) in view of the language used in the appended claims. The terminology used herein is for the purpose of describing particular embodiments only and is not intended to be limiting.

Generally, the nomenclature used and procedures performed herein are those known in fields relating to one or more aspects of the invention, such as biology, pharmacology, neuroscience, organic chemistry, synthetic chemistry, and/or medicinal chemistry, and are those that will be well known and commonly employed in such fields. Standard techniques and procedures will be those generally performed according to conventional methods in the art.

“Alkyl” will be understood to include straight or branched radicals having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” can also be used. Preferably, an alkyl group comprises from 1 to 10 carbon atoms, more preferably from 1 to 6 carbon atoms, more preferably from 1 to 4 carbon atoms, and most preferably from 1 to 3 carbon atoms. For any alkyl, the alkyl may be optionally substituted at one or more positions by deuterium, halogen, alkyl, alkyl ester, hydroxy, alkoxy, carboxy, formyl, aryl, cycloalkyl, heterocycloalkyl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, —OP(O)(OH)₂, —OC(O)H, —OSO₂OH, —OC(O)NH₂, and —SONH₂.

“Alkanyl” refers to saturated branched, straight-chain, or cyclic alkyl radicals derived by the removal of one hydrogen atom from a single carbon atom of a parent alkane. Typical alkanyl groups include methanyl; ethanyl; propanyls such as propan-1-yl, propan-2-yl (isopropyl), and cyclopropan-1-yl; butanyls such as butan-1-yl, butan-2-yl (sec-butyl), 2-methyl-propan-1-yl (isobutyl), 2-methyl-propan-2-yl (t-butyl), and cyclobutan-1-yl; etc.

“Alkenyl” refers to an unsaturated branched, straight-chain, or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include ethenyl; propenyls such as prop-1-en-1-yl, prop-1-en-2-yl, prop-2-en-1-yl (allyl), prop-2-en-2-yl, cycloprop-1-en-1-yl, and cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2yl, buta-1,3-dien-1-yl, buta-1,3-dien-2-yl, cyclobut-1-en-1-yl, cyclobut-1-en-3-yl, and cyclobuta-1,3-dien-1-yl; and the like.

“Alkynyl” refers to an unsaturated branched, straight-chain, or cyclic alkyl radical having at least one carbon-carbon triple bond derived by the removal of one hydrogen atom from a single carbon atom of a parent alkyne. Typical alkynyl groups include ethynyl; propynyls such as prop-1-yn-1-yl, and prop-2-yn-1-yl; butynyls such as but-1-yn-1-yl, but-1-yn-3-yl, and but-3-yn-1-yl; and the like.

“Aryl” refers to a monovalent aromatic hydrocarbon radical derived by the removal of one hydrogen atom from a single carbon atom of a parent aromatic ring system. Typical aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. Preferably, an aryl group comprises from 6 to 20 or more preferably from 6 to 12 carbon atoms.

“Cycloalkyl” refers to a saturated monocyclic, bicyclic, fused bicyclic or bridged polycyclic ring assembly containing from 3 to 12 ring atoms, or the number of atoms indicated. Cycloalkyl can include any number of carbons, such as 3 to 6 carbon atoms, 4 to 6 carbon atoms, 5 to 6 carbon atoms, 3 to 8 carbon atoms, 4 to 8 carbon atoms, 5 to 8 carbon atoms, 6 to 8 carbon atoms, 7 to 8 carbon atoms, 3 to 9 carbon atoms, 4 to 9 carbon atoms, 5 to 9 carbon atoms, 6 to 9 carbon atoms, 7 to 9 carbon atoms, 8 to 9 carbon atoms, 3 to 10 carbon atoms, 4 to 10 carbon atoms, 5 to 10 carbon atoms, 6 to 10 carbon atoms, 7 to 10 carbon atoms, 8 to 10 carbon atoms, 9 to 10 carbon atoms, 3 to 11 carbon atoms, 4 to 11 carbon atoms, 5 to 11 carbon atoms, 6 to 11 carbon atoms, 7 to 11 carbon atoms, 8 to 11 carbon atoms, 9 to 11 carbon atoms, 10 to 11 carbon atoms, 3 to 12 carbon atoms, 4 to 12 carbon atoms, 5 to 12 carbon atoms, 6 to 12 carbon atoms, 7 to 12 carbon atoms, 8 to 12 carbon atoms, 9 to 12 carbon atoms, 10 to 12 carbon atoms, and 11 to 12 carbon atoms. Monocyclic cycloalkyl rings include, e.g., cyclopropyl, cyclobutyl, cyclopentyl, cyclohexyl, and cyclooctyl. Bicyclic compounds include spirocyclic compounds, fused bicyclic compounds and bridged bicyclic compounds. Bicyclic and polycyclic cycloalkyl rings include, e.g., norbornane, bicyclooctane, decahydronaphthalene and adamantane. When cycloalkyl is a monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Cycloalkenyl” refers to a mono- or multi-cyclic hydrocarbon ring system that contains one or more double bonds in at least one ring. However, if there is more than one double bond, the double bonds cannot form a fully delocalized pi-electron system throughout all the rings (otherwise the group would be “aryl,” as defined herein). When composed of two or more rings, the rings may be connected together in a fused fashion. Cycloalkenyl can include any number of carbons, such as 3 to 6 carbon atoms, 4 to 6 carbon atoms, 5 to 6 carbon atoms, 3 to 8 carbon atoms, 4 to 8 carbon atoms, 5 to 8 carbon atoms, 6 to 8 carbon atoms, 7 to 8 carbon atoms, 3 to 9 carbon atoms, 4 to 9 carbon atoms, 5 to 9 carbon atoms, 6 to 9 carbon atoms, 7 to 9 carbon atoms, 8 to 9 carbon atoms, 3 to 10 carbon atoms, 4 to 10 carbon atoms, 5 to 10 carbon atoms, 6 to 10 carbon atoms, 7 to 10 carbon atoms, 8 to 10 carbon atoms, 9 to 10 carbon atoms, 3 to 11 carbon atoms, 4 to 11 carbon atoms, 5 to 11 carbon atoms, 6 to 11 carbon atoms, 7 to 11 carbon atoms, 8 to 11 carbon atoms, 9 to 11 carbon atoms, 10 to 11 carbon atoms, 3 to 12 carbon atoms, 4 to 12 carbon atoms, 5 to 12 carbon atoms, 6 to 12 carbon atoms, 7 to 12 carbon atoms, 8 to 12 carbon atoms, 9 to 12 carbon atoms, 10 to 12 carbon atoms, and 11 to 12 carbon atoms. Representative cycloalkenyl groups include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. A cycloalkenyl group may be unsubstituted or substituted.

“Halogen” refers to fluorine, chlorine, bromine, and iodine.

“Heterocycloalkyl” or “heterocyclyl” refers to a cycloalkyl as defined above, having from 3 to 12 ring members and from 1 to 4 heteroatoms of N, O and S. Heterocycloalkyl includes bicyclic compounds which include a heteroatom. Bicyclic compounds includes spirocyclic compounds, fused bicyclic compounds, and bridged bicyclic compounds The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C1-6 alkyl or oxo (═O), among many others.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C0-6, C1-2, C1-3, C1-4, C1-5, C1-6, C2-3, C2-4, C2-5, C2-6, C3-4, C3-5, C3-6, C4-5, C4-6 and C5-6. In some instances, the alkyl component can be absent. The heterocycloalkyl component is as defined above. Alkyl-heterocycloalkyl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C0-6, C1-2, C1-3, C1-4, C1-5, C1-6, C2-3, C2-4, C2-5, C2-6, C3-4, C3-5, C3-6, C4-5, C4-6 and C5-6. In some instances, the alkyl component can be absent. The heteroaryl component is as defined within. Alkyl-heteroaryl groups can be substituted or unsubstituted.

“Alkoxy” refers to the formula-OR, wherein R is an alkyl, an alkenyl, an alkynyl, a cycloalkyl, a cycloalkenyl, aryl, or heterocyclyl, as defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

“Acyl” refers to a hydrogen, an alkyl, an alkenyl, an alkynyl, a cycloalkyl, a cycloalkenyl, aryl, or heterocyclyl, connected via a carbonyl group as a substituent. Examples include formyl, acetyl, propanoyl, benzoyl, and acryl. An acyl may be substituted or unsubstituted.

“Aryloxy” refers to an aryl moiety, as defined herein, attached to an oxygen atom, wherein the oxygen atom serves as the attaching point to the remainder of the molecule. An aryloxy may be substituted or unsubstituted. Exemplary aryloxy groups include phenoxy, tolyloxy (including p-tolyloxy, m-toyloxy, and o-tolyloxy), ethylphenyloxy (including p-ethylphenyloxy, m-ethylphenyloxy, and o-ethylphenyloxy), naphthyloxy, and the like.

“Alkylamino” refers to gropus such as N-alkylamino (i.e., R—NHR′) and N,N-dialkylamino (i.e., R—NR′R″), wherein the amino groups are independently substituted with one alkyl radical (i.e., R′) or with two alkyl radicals (i.e., R′ and R″), respectively; and wherein R represents an alkyl as defined herein. Non-limiting examples of alkylamino radicals include mono or dialkylamino such as N-methylamino, N-ethylamino, N,N-dimethylamino, N,N-diethylamino, and the like. An alkylamino can be unsubstituted or substituted.

“Alkylthio” or “thioalkyl” refer to the formula —SR, wherein R is an alkyl, an alkenyl, an alkynyl, a cycloalkyl, a cycloalkenyl, aryl, or heterocyclyl, as defined herein. In some embodiments, wherein R is aryl, the —SR moiety is termed “thioaryl.” A non-limiting list of alkylthio or thioalkyl includes methylthio, ethylthio, n-propylthio, isopropylthio, n-butylthio, iso-butylthio, sec-butylthio, phenylthio, and benzylthio. An alkylthio or thioalkyl may be substituted or unsubstituted.

“Arylamido” refers to an aryl moiety, as defined herein, attached to an amide moiety, wherein the amide moiety serves as the attaching point to the remainder of the molecule. In some embodiments, an arylamido has the formula Ar—C(═O)NH—* or Ar—NH—C(═O)—*, wherein Ar is aryl as defined herein, and * represents the point of connection to the remainder of the molecule. An arylamido can be substituted or unsubstituted.

“Alkylamido” refers to an alkyl moiety, as defined herein, attached to an amide moiety, wherein the amide moiety serves as the attaching point to the remainder of the molecule. In some embodiments, an alkylamido has the formula Ak-C(—O)NH—* or Ak-NH—C(═O)—*, wherein Ak is alkyl as defined herein, and * represents the point of connection to the remainder of the molecule. An alkylamido can be substituted or unsubstituted.

“Haloalkyl” will be understood to include any alkyl group as defined above, wherein one or more hydrogen atoms are replaced by a halogen (e.g., a fluorine, a chlorine, a bromine, or an iodine). Where an alkyl radical is substituted by more than one halogen, it may be referred to using a prefix corresponding to the number of halogen substitutions. For example, dihaloalkyl refers to an alkyl substituted by two halo groups, which may be, but are not necessarily, the same halogen. Examples of haloalkyl groups include difluoromethyl (—CHF₂), bromofluoromethyl (—CHBrF), trifluoromethyl (—CF₃), and 2-fluoroethyl (—CH₂CH₂F). Additional examples of haloalkyl groups include —CHF₂, —CH₂F, —CH₂CF₃, —CH₂CHF₂, —CH₂CH₂F, —CH(CH₃)(CF₃), —CH(CH₃)(CHF₂), and —CH(CH₃)(CH₂F).

“Hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl and 2,2-dihydroxyethyl. A hydroxyalkyl may be substituted or unsubstituted.

“Haloalkoxy” refers to an —O-alkyl group in which one or more of the hydrogen atoms are replaced by a halogen (e.g., mono-haloalkoxy, di-haloalkoxy and tri-haloalkoxy). The halogens may be the same or different in each instance. Such groups include but are not limited to, chloromethoxy, fluoromethoxy, difluoromethoxy, trifluoromethoxy, 1-chloro-2-fluoro-methoxy and 2-fluoroisobutoxy. A haloalkoxy may be substituted or unsubstituted.

“Sulfenyl” refers to an —SR group in which R can be hydrogen, alkyl, alkenyl, alkynyl, cycloalkyl, cycloalkenyl, aryl, or heterocyclyl, as defined herein. A sulfenyl may be substituted or unsubstituted.

“Sulfinyl” refers to an —S(═O)—R group in which R can be the same as defined with respect to sulfenyl. A sulfinyl may be substituted or unsubstituted.

“Sulfonyl” refers to an —SO₂R group in which R can be the same as defined with respect to sulfenyl. “Alkylsulfonyl” specifically refers to an —SO₂R group in which R is alkyl, as defined herein. A sulfonyl (or alkylsulfonyl) may be substituted or unsubstituted.

“Carboxy” refers to a —RC(═O)O— group in which R can be hydrogen, alkyl, alkenyl, alkynyl, cycloalkyl, cycloalkenyl, aryl, or heterocyclyl, as defined herein. A carboxy may be substituted or unsubstituted.

“Ester” and “C-carboxy” refer to a —C(═O) OR group in which R can be the same as defined with respect to O-carboxy. “Alkyl ester” refers to a —C(═O) OR group in which R is alkyl, as defined herein. Ester and C-carboxy groups may be substituted or unsubstituted.

“Thiocarbonyl” refers to a —C(═S) R group in which R can be the same as defined with respect to O-carboxy. A thiocarbonyl may be substituted or unsubstituted.

“Trihalomethanesulfonyl” refers to an X₃CSO₂— group wherein each X is a halogen.

“Trihalomethanesulfonamido” refers to an X₃CS(O)₂N(R_{A})— group wherein each X is a halogen, and R_{A} is hydrogen, alkyl, alkenyl, alkynyl, cycloalkyl, cycloalkenyl, aryl, or heterocyclyl, as defined herein.

“S-sulfonamido” refers to a —SO₂N(R_{A}R_{B}) group in which R_{A} and R_{B} can be independently hydrogen, alkyl, alkenyl, alkynyl, cycloalkyl, cycloalkenyl, aryl, or heterocyclyl, as defined herein. An S-sulfonamido may be substituted or unsubstituted.

“N-sulfonamido” refers to a RSO₂N(R_{A})— group in which R and R_{A} can be independently hydrogen, alkyl, alkenyl, alkynyl, cycloalkyl, cycloalkenyl, aryl, or heterocyclyl, as defined herein. An N-sulfonamido may be substituted or unsubstituted.

“Carbamoyl” includes O-carbamoyl and N-carbamoyl groups. “O-carbamoyl” refers to a —OC(—O)N(R_{A}R_{B}) group in which R_{A} and R_{B} can be independently hydrogen, alkyl, alkenyl, alkynyl, cycloalkyl, cycloalkenyl, aryl, or heterocyclyl, as defined herein. An O-carbamoyl may be substituted or unsubstituted. “N-carbamoyl” refers to an ROC(═O)N(R_{A})— group in which R and R_{A} can be independently hydrogen, alkyl, alkenyl, alkynyl, cycloalkyl, cycloalkenyl, aryl, or heterocyclyl, as defined herein. An N-carbamoyl may be substituted or unsubstituted.

“O-thiocarbamyl” refers to a —OC(═S)—N(R_{A}R_{B}) group in which R_{A} and R_{B} can be independently hydrogen, alkyl, alkenyl, alkynyl, cycloalkyl, cycloalkenyl, aryl, or heterocyclyl, as defined herein. An O-thiocarbamyl may be substituted or unsubstituted.

“N-thiocarbamyl” refers to an ROC(═S)N(R_{A})— group in which R and R_{A} can be independently hydrogen, alkyl, alkenyl, alkynyl, cycloalkyl, cycloalkenyl, aryl, or heterocyclyl, as defined herein. An N-thiocarbamyl may be substituted or unsubstituted.

“C-amido” group refers to a —C(═O)N(R_{A}R_{B}) group in which R_{A} and R_{B} can be independently hydrogen, alkyl, alkenyl, alkynyl, cycloalkyl, cycloalkenyl, aryl, or heterocyclyl, as defined herein. A C-amido may be substituted or unsubstituted.

“N-amido” refers to a RC(═O)N(R_{A})— group in which R and R_{A} can be independently hydrogen, alkyl, alkenyl, alkynyl, cycloalkyl, cycloalkenyl, aryl, or heterocyclyl, as defined herein. An N-amido may be substituted or unsubstituted.

“Optionally substituted” unless otherwise specified means that a group may be unsubstituted, or substituted by one or more of the substituents listed for that group. Likewise, when a group is described as being “unsubstituted or substituted” if substituted, the substituent(s) may be selected from one or more of the indicated substituents. When there are more than one substituents, the substituents may be the same or different. In one embodiment, an optionally substituted group has one substituent. In another embodiment, an optionally substituted group has two substituents. In another embodiment, an optionally substituted group has three substituents. In another embodiment, an optionally substituted group has four substituents. If no substituents are indicated for an “optionally substituted” or “substituted” group, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), heteroaryl(alkyl), (heterocyclyl)alkyl, hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, azido, silyl, sulfenyl, sulfinyl, sulfonyl, haloalkyl, haloalkoxy, trihalomethanesulfonyl, trihalomethanesulfonamido, an amino, a mono-substituted amino group, a di-substituted amino group, and a tri-substituted amino group.

Still additional definitions and abbreviations are provided elsewhere herein.

### B. COMPOUNDS

Psychedelics are a unique class of psychoactive drugs defined by their ability to alter thought, feeling, and perception (Masters and Houston, The Varieties of Psychedelic Experience., Dell Publishing Co., Inc., 1966; Nichols, Pharmacol Rev., 2016; 68 (2): 264-355). Major chemotypes of the psychedelic class include phenylalkylamines, tryptamines, and lysergamides (Nichols, Pharmacol Rev., 2016; 68 (2): 264-355). The typical psychoactive effects on consciousness of psychedelic phenylalkylamines such as 2-(4-bromo-2,5-dimethoxyphenyl) ethanamine (2C-B) (Shulgin and Carter, Psychopharmacol Commun 1., 1975; 93-98) are primarily mediated via activation of the 5-HT_{2A} subtype of serotonin (5-HT) receptors (Glennon et al., Life Sci., 1984; 35 (25): 2505-2511; Titeler et al., Psychopharmacology (Berl), 1988; 94 (2): 213-216; Vollenweider et al., Neuroreport, 1998; 9 (17): 3897-3902). It is believed that activation of the 5-HT_{2A} receptor is also important for the psychoactivity of lysergamides such as lysergic acid diethylamide (LSD), and tryptamines such as N,N-dimethyltryptamine (DMT) and psilocin.

The 5-HT_{2A} receptor, like the β₂ adrenergic receptor, belongs to the superfamily of G protein-coupled receptors containing seven transmembrane domains (TMDs). Addition of a phenethyloxyhexyl side-chain to the β₂ agonist salbutamol resulted in salmeterol, a β₂ agonist with improved potency and duration of action in the treatment of bronchoconstriction associated with asthma and chronic obstructive pulmonary disease (COPD) (Johnson, Med Res Rev., 1995; 15 (3): 225-257). The extended linear dimension of the salmeterol molecule (25 Å) compared to the salbutamol molecule (11 Å) allows the side-chain of salmeterol to bind to an accessory binding region within the β₂ receptor, distinct from the agonist binding domain, termed the exosite (Johnson, Med Res Rev., 1995; 15 (3): 225-257; Masureel et al., Nat Chem Biol., 2018; 14 (11): 1059-1066).

Evidence from site-directed mutagenesis, chimeric β₁/β₂ receptors, photoaffinity labelling, x-ray diffraction crystallography, and computer modeling of the β₂ receptor localizes the exosite within TMDs 4, 6, and 7 and extracellular loops (ECL) 2 and 3 of the β₂ receptor. (Green et al., J Biol Chem., 1996; 271 (39): 24029-24035; Isogaya et al., Mol Pharmacol., 1998; 54 (4): 616-622; Johnson, Med Res Rev., 1995; 15 (3): 225-257; Masureel et al., Nat Chem Biol., 2018; 14 (11): 1059-1066; Rong 1999). The X-ray crystal structure of salmeterol bound to the β₂ receptor shows that the aryloxyalkyl tail occupies a cleft formed by residues from extracellular loop ECL2, ECL3, and the extracellular ends of TMD6 and TMD7 (Masureel et al., Nat Chem Biol., 2018; 14 (11): 1059-1066).

Following an amino acid sequence alignment between the human β2 receptor and the human 5-HT_{2A} receptor using the UniProt Align alignment routine (https://www.uniprot.org/), it was observed that the amino acid sequences in three TMDs of the 5 HT_{2A} receptor show high sequence homology (70-94%) with the three TMDs comprising the β₂ receptor exosite, while ECL 2 and 3 exhibited lower homology (<50% conserved). Based on these sequence homologies, it was hypothesized that the 5-HT_{2A} receptor, like the β₂ receptor, contains an exosite that might engage an extended N-linked side-chain of a modified 5-HT_{2A} agonist.

In a first aspect, provided herein are substituted phenylalkylamine compounds bearing an N-linked side chain. Phenylalkylamines are stimulant, entactogenic/empathogenic, and/or hallucinogenic/psychedelic substances that share similar chemical structures with amphetamine, catecholamines, synthetic cathinones, and other substances (Nelson et al., Emerg Med Clin North Am., 2014; 32 (1): 1-28). Phenylalkylamines comprise both natural and synthetic substances, including classical phenethylamines, for example, MDMA, MDEA, and MBDB, and psychoactive phenethylamines that include mescaline-derived compounds, for example, TMA, DOM, DOET, DOI, and DOC. More recently described phenylalkylamines include bromodragonfly, benzofuran, N-benzyl substituted phenylalkylamine substances, and the “2C-series,” e.g., 2C-I, 2C-E, 2C-B, and other “2C-X” compounds (Schifano et al., World Psychiatry, 2015; 14 (1): 15-26; Shulgin & Shulgin, PIHKAL: A Chemical Love Story, Transform Press, Berkeley, CA, 1991).

In some embodiments, the term “phenylalkylamine” refers to a phenylalkylamine having the structure of Formula (A), wherein R^{N1}, R^{N2}, R^{α}, R^{β}, and each of R²-R⁶ are as defined herein and as understood in the art:

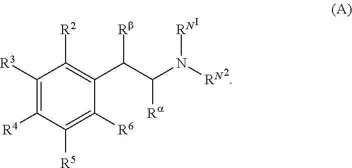

In some embodiments of Formula (A), R^{N1}, R^{N2}, R^{α}, R^{β}, and each of R²-R⁶ are independently hydrogen, halogen, C1-C5 alkyl, C2-C8 alkenyl, C2-C8 alkynyl, C3-C8 cycloalkyl (independently or ring closed with the nitrogen, when RN), C3-C8 cycloalkenyl (independently or ring closed with the nitrogen, when RN), aryl, or heterocyclyl; including where R3 and R4 may be joined together to form a dioxole (as with MDMA), a furan, a tetrahydrofuran, a thiophene, a pyrrole, a pyridine, a pyrrolidine, an ethylene oxide, an ethylenimine, a trimethylene oxide, a pyran, a piperidine, an imidazole, a thiazole, a dioxane, a morpholine, a pyrimidine, or otherwise so as to create a benzene heterocycle; and any of which are optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate, —OP(O)(OH)₂, —OC(O)H, —OSO₂OH, —OC(O)NH₂, and —SONH.

In some embodiments of Formula (A), R^{N1}, R^{N2}, R^{α}, R^{β}, and each of R²⁻⁶ are independently hydrogen, deuterium, halogen, optionally substituted alkyl, optionally substituted alkenyl, optionally substituted alkynyl, optionally substituted cycloalkyl, optionally substituted cycloalkenyl, optionally substituted aryl, or optionally substituted heterocyclyl. In some embodiments, R³ and R⁴ are joined together to form an optionally substituted heterocyclyl, such as a dioxole (as with MDMA), a furan, a tetrahydrofuran, a thiophene, a pyrrole, a pyridine, a pyrrolidine, an ethylene oxide, an ethylenimine, a trimethylene oxide, a pyran, a piperidine, an imidazole, a thiazole, a dioxane, a morpholine, or a pyrimidine. In some embodiments, R³ and R⁴ are joined together to form an optionally substituted aryl, such as a phenyl. In some embodiments, the phenethylamine comprises a quaternary ammonium cation wherein each of R^{N1}, R^{N2}, and an additional R^{N3} are independently an alkyl group or an aryl group, and with all other substituents as above. In embodiments, the phenethylamine is a quaternary salt, in which an additional R^{N3} is connected to the nitrogen to which R^{N1} and R^{N2} are bound; wherein R^{N3} is optionally substituted alkyl, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl.

In some embodiments, a substituted phenylalkylamine compound of the disclosure comprises a phenylalkylamine of Formula (A) bearing an N-linked side chain. In some embodiments, a substituted phenylalkylamine compound of the disclosure comprises a phenylalkylamine of Formula (A), wherein one of RN1 and RN2 is a side chain as described in embodiments herein, and the other of RN1 and RN2 is as defined above.

In some embodiments, the term “phenylalkylamine” refers to a 2C phenethylamine. The 2C series of phenethylamines, known and referred to herein as the “2C” or “2C-X” compounds, are ring-substituted phenethylamines containing methoxy groups on the 2 and 5 positions of the benzene ring, along with often lipophilic substituents at the 4 position. In some embodiments, the 2C phenethylamine is a phenylalkylamine having the structure of Formula (B),

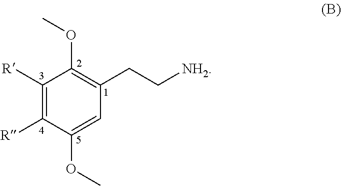

In some embodiments of Formula (B), R′ and R″ are selected independently from hydrogen, N, NO₂, Cl, Br, F, I, CH₃, CH₃O—, CH₃CH₂O—, CH₃CH₂CH₂O—, CF₃O—, CF₃CH₂O—, CF₃CH₂CH₂O—, CH₃CH₂—, CH₃CH₂CH₂—, CH₃S—, CH₃CH₂S—, (CH₃)₂CHS—, CH₃CH₂CH₂S—, CH₃CH₂CH₂CH₂S—, (CH₃)₃CS—, CF₃CH₂—, CF₃CH₂CH₂—, CF₃S—, CF₃CH₂S—, CF₃CH₂CH₂S—, FCH₂CH₂S—, or (CH₂)₁₋₆(CH₂)₀₋₅, where (CH₂)₁₋₆ is any of a fused (when CH₂=0) or unfused 3-, 4-, 5-, or 6-membered ring system, which may include a bridged or aromatic ring system. In some embodiments, R′ is hydrogen and R″ is as defined above. In some embodiments, both R′ and R″ are hydrogen.

In some embodiments, a substituted phenylalkylamine compound of the disclosure comprises a phenylalkylamine of Formula (B) bearing an N-linked side chain. In some embodiments, a substituted phenylalkylamine compound of the disclosure comprises a phenylalkylamine of Formula (B), wherein the primary amine is substituted by a side chain as described in embodiments herein.

Modification of the 2C aromatic ring in different positions produces distinct compounds with altered neurochemical actions. Many 2C compounds show affinity for different subtypes of serotonin 5-HT₂ receptor, some interfere with the reuptake of dopamine, serotonin, and noradrenaline, while 2C-B acts as al-adrenergic receptor agonist (Nichols, Pharmacology & Therapeutics. 2004; 101 (2): 131-181; Villalobos et al, British Journal of Pharmacology. 2004; 141 (7): 1167-1174). In some embodiments, a substituted phenylalkylamine compound of the disclosure comprises a 2C-X bearing an N-linked side chain, wherein the 2C-X is any of 2C-B, 2C-B-AN, 2C-B-Butterfly, 2C-B-Fly-NBOMe, 2C-B-Fly-NB2EtO5Cl, 2C-Bn, 2C-Bu, 2C-B-5-Hemifly, 2C-C, 2C-C-3, 2C-CN, 2C-CP, 2C-D, 2C-E, 2C-μF, 2C-F, 2C-G, 2C-G-1, 2C-G-2, 2C-G-3, 2C-G-4, 2C-G-5, 2C-G-6, 2C-G-N, 2C-H, 2C-I, 2CB-Ind, 2C-iP, 2C-N, 2C-NH2, 2C-PYR, 2C-PIP, 2C-O, 2C-O-4, 2C-MOM, 2C-P, 2C-Ph, 2C-Se, 2C-T, 2C-T-2, 2C-T-3, 2C-T-4, 2C-T-5, 2C-T-6, 2C-T-7, 2C-T-8, 2C-T-9, 2C-T-10, 2C-T-11, 2C-T-12, 2C-T-13, 2C-T-14, 2C-T-15, 2C-T-16, 2C-T-17, 2C-T-18, 2C-T-19, 2C-T-21, 2C-T-21.5, 2C-T-22, 2C-T-23, 2C-T-24, 2C-T-25, 2C-T-27, 2C-T-28, 2C-T-30, 2C-T-31, 2C-T-32, 2C-T-33, 2C-DFM, 2C-TFM, 2C-TFE, 2C-YN, 2C-V, and 2C-AL, as such compounds will be understood in the art, and where such compounds are amine-substituted with a side chain as defined herein.

As used herein, in the context of the substituted phenylalkylamine compounds of the disclosure, the term “side chain” refers to an optionally substituted alkyl chain (e.g., optionally substituted n-decyl). In some embodiments, one or more methylene units of the alkyl chain may be replaced by a heteroatom (e.g., an oxygen; thereby introducing an ether linkage into the side chain). In some embodiments, the alkyl chain is substituted. For example, in some embodiments, the side chain is aryl-substituted (e.g., substituted by an optionally substituted phenyl ring).

In some embodiments, the side chain has the structure of —(CH₂)ₘX(CH₂)ₙPh, wherein X is a methylene (CH₂) or a heteroatom linker (e.g., O, S, NH, etc.); Ph is an optionally substituted aryl, heteroaryl, cycloalkyl, or heterocycloalkyl; and m and n are each independently an integer from 1 to 20. In some embodiments, X is CH₂. In some embodiments, X is O. In some embodiments, X is S. In some embodiments, X is NH. In some embodiments, the side chain is an aralkyloxyalkyl (e.g., a side chain having the formula of —(CH₂)ₘO(CH₂)ₙPh), and the disclosed compound is N-aralkyloxyalkyl-substituted phenylalkylamine.

In some embodiments, Ph is an optionally substituted aryl, heteroaryl, cycloalkyl, or heterocycloalkyl. In embodiments, Ph is an optionally substituted 3-membered, 4-membered, 5-membered, 6-membered, 7-membered, 8-membered, 9-membered or 10-membered aryl, heteroaryl, cycloalkyl, or heterocycloalkyl. In embodiments, Ph is an optionally substituted aryl. In embodiments, Ph is an optionally substituted phenyl. In embodiments, Ph is an unsubstituted phenyl. In embodiments, Ph is a phenyl substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate.

In some embodiments, m is an integer from 1 to 20. In embodiments, m is 1. In embodiments, m is 2. In embodiments, m is 3. In embodiments, m is 4. In embodiments, m is 5. In embodiments, m is 6. In embodiments, m is 7. In embodiments, m is 8. In embodiments, m is 9. In embodiments, m is 10. In embodiments, m is 11. In embodiments, m is 12. In embodiments, m is 13. In embodiments, m is 14. In embodiments, m is 15. In embodiments, m is 16. In embodiments, m is 17. In embodiments, m is 18. In embodiments, m is 19. In embodiments, m is 20. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, n is 14. In embodiments, n is 15. In embodiments, n is 16. In embodiments, n is 17. In embodiments, n is 18. In embodiments, n is 19. In embodiments, n is 20. In embodiments, the sum of m+n is from 4 to 16. In embodiments, the sum of m+n is from 6 to 14. In embodiments, the sum of m+n is from 7 to 13. In embodiments, the sum of m+n is from 8 to 12. In embodiments, the sum of m+n is from 9 to 11. In embodiments, the sum of m+n is 6. In embodiments, the sum of m+n is 7. In embodiments, the sum of m+n is 8. In embodiments, the sum of m+n is 9. In embodiments, the sum of m+n is 10. In embodiments, the sum of m+n is 11. In embodiments, the sum of m+n is 12. In embodiments, the sum of m+n is 13. In embodiments, the sum of m+n is 14. In embodiments, m and n are selected from the following pair series: 1 and 9, 2 and 8, 3 and 7, 4 and 6, 5 and 5, 6 and 4, 7 and 3, 8 and 2, 9 and 1.

In some embodiments, the compound has the structure of Formula (I),

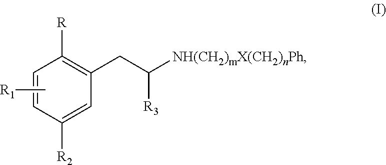

wherein:
m and n are selected from the following pair series: 1 and 9, 2 and 8, 3 and 7, 4 and 6, 5 and 5, 6 and 4, 7 and 3, 8 and 2, 9 and 1; R and R2 are, independently, hydrogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, any of which are optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate; R1 represents 1-3 substituents independently selected from the group consisting of hydrogen, halogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, and nitrate; R3 is hydrogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R3 is selected from the group comprising halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, and nitrate; and Ph is optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, and nitrate.

In embodiments, m and n are selected from the following pair series: 1 and 9, 2 and 8, 3 and 7, 4 and 6, 5 and 5, 6 and 4, 7 and 3, 8 and 2, 9 and 1. In embodiments, m is 1 and n is 9. In embodiments, m is 2 and n is 8. In embodiments, m is 3 and n is 7. In embodiments, m is 4 and n is 6. In embodiments, m is 5 and n is 5. In embodiments, m is 6 and n is 4. In embodiments, m is 7 and n is 3. In embodiments, m is 8 and n is 2. In embodiments, m is 9 and n is 1.

In some embodiments, R and R2 are, independently, hydrogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, any of which are optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is hydrogen. In embodiments, R is C1-C8 alkyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is unsubstituted C1-C8 alkyl. In embodiments, R is substituted C1-C8 alkyl. In embodiments, R is C1-C8 alkoxy optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is unsubstituted C1-C8 alkoxy. In embodiments, R is substituted C1-C8 alkoxy. In embodiments, R is C1-C8 alkenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is unsubstituted C1-C8 alkenyl. In embodiments, R is substituted C1-C8 alkenyl. In embodiments, R is C1-C8 alkynyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is unsubstituted C1-C8 alkynyl. In embodiments, R is substituted C1-C8 alkynyl. In embodiments, R is C3-C8 cycloalkyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is unsubstituted C3-C8 cycloalkyl. In embodiments, R is substituted C3-C8 cycloalkyl. In embodiments, R is C3-C8 cycloalkenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is unsubstituted C3-C8 cycloalkenyl. In embodiments, R is substituted C3-C8 cycloalkenyl. In embodiments, R is aryl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is unsubstituted aryl. In embodiments, R is substituted aryl. In embodiments, R is heterocyclyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is unsubstituted heterocyclyl. In embodiments, R is substituted heterocyclyl.

In some embodiments, R2 is hydrogen. In embodiments, R2 is C1-C8 alkyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is unsubstituted C1-C8 alkyl. In embodiments, R2 is substituted C1-C8 alkyl. In embodiments, R2 is C1-C8 alkoxy optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is unsubstituted C1-C8 alkoxy. In embodiments, R2 is substituted C1-C8 alkoxy. In embodiments, R2 is C1-C8 alkenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is unsubstituted C1-C8 alkenyl. In embodiments, R2 is substituted C1-C8 alkenyl. In embodiments, R2 is C1-C8 alkynyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is unsubstituted C1-C8 alkynyl. In embodiments, R2 is substituted C1-C8 alkynyl. In embodiments, R2 is C3-C8 cycloalkyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is unsubstituted C3-C8 cycloalkyl. In embodiments, R2 is substituted C3-C8 cycloalkyl. In embodiments, R2 is C3-C8 cycloalkenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is unsubstituted C3-C8 cycloalkenyl. In embodiments, R2 is substituted C3-C8 cycloalkenyl. In embodiments, R2 is aryl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is unsubstituted aryl. In embodiments, R2 is substituted aryl. In embodiments, R2 is heterocyclyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is unsubstituted heterocyclyl. In embodiments, R2 is substituted heterocyclyl.

In some embodiments, R1 represents 1-3 substituents independently selected from the group consisting of hydrogen, halogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, and nitrate. In embodiments, R1 represents 1 substituent selected from the group consisting of hydrogen, halogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, and nitrate. In embodiments, R1 represents 2 substituents independently selected from the group consisting of hydrogen, halogen, C1-C8 alkyl, C1-C8 alkoxy, C2-C8 alkenyl, C2-C8 alkynyl, C₃-C₈ cycloalkyl, C3-C8 cycloalkenyl, aryl or heterocyclyl, optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, and nitrate. In embodiments, R1 represents 3 substituents independently selected from the group consisting of hydrogen, halogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, and nitrate.

In some embodiments, R1 is hydrogen. In embodiments, R1 is halogen. In embodiments, R₁ is F, Cl, Br, or I. In embodiments, R1 is C1-C8 alkyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is unsubstituted C1-C8 alkyl. In embodiments, R1 is substituted C1-C8 alkyl. In embodiments, R1 is C1-C8 alkoxy optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is unsubstituted C1-C8 alkoxy. In embodiments, R1 is substituted C1-C8 alkoxy. In embodiments, R1 is C1-C8 alkenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is unsubstituted C1-C8 alkenyl. In embodiments, R1 is substituted C1-C8 alkenyl. In embodiments, R1 is C1-C8 alkynyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R₁ is unsubstituted C1-C8 alkynyl. In embodiments, R1 is substituted C1-C8 alkynyl. In embodiments, R1 is C3-C8 cycloalkyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is unsubstituted C3-C8 cycloalkyl. In embodiments, R1 is substituted C3-C8 cycloalkyl. In embodiments, R1 is C3-C8 cycloalkenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is unsubstituted C3-C8 cycloalkenyl. In embodiments, R1 is substituted C3-C8 cycloalkenyl. In embodiments, R1 is aryl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is unsubstituted aryl. In embodiments, R1 is substituted aryl. In embodiments, R1 is heterocyclyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is unsubstituted heterocyclyl. In embodiments, R1 is substituted heterocyclyl.

In some embodiments, R3 is hydrogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate; or R3 is selected from the group comprising halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, and nitrate.

In some embodiments, R3 is hydrogen, C1-C8 alkyl, C1-C8 alkoxy, C2-C8 alkenyl, C2-C8 alkynyl, C3-C8 cycloalkyl, C3-C8 cycloalkenyl, aryl or heterocyclyl, optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, nitrate. In embodiments, R3 is hydrogen. In embodiments, R3 is C1-C8 alkyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R3 is unsubstituted C1-C8 alkyl. In embodiments, R3 is substituted C1-C8 alkyl. In embodiments, R3 is C1-C8 alkoxy optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R3 is unsubstituted C1-C8 alkoxy. In embodiments, R3 is substituted C1-C8 alkoxy. In embodiments, R3 is C1-C8 alkenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R3 is unsubstituted C1-C8 alkenyl. In embodiments, R3 is substituted C1-C8 alkenyl. In embodiments, R3 is C1-C8 alkynyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R3 is unsubstituted C1-C8 alkynyl. In embodiments, R3 is substituted C1-C8 alkynyl. In embodiments, R3 is C3-C8 cycloalkyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R3 is unsubstituted C3-C8 cycloalkyl. In embodiments, R3 is substituted C3-C8 cycloalkyl. In embodiments, R3 is C3-C8 cycloalkenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R3 is unsubstituted C3-C8 cycloalkenyl. In embodiments, R3 is substituted C3-C8 cycloalkenyl. In embodiments, R3 is aryl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R3 is unsubstituted aryl. In embodiments, R3 is substituted aryl. In embodiments, R3 is heterocyclyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R3 is unsubstituted heterocyclyl. In embodiments, R3 is substituted heterocyclyl.

In some embodiments, R3 is selected from the group comprising halogen, alkyl ester, hydroxy, alkoxy, carboxy, formyl, aryloxy, amino, alkylamino, arylamido, alkylamido, thiol, thioalkyl, thioaryl, alkylsulfonyl, alkylcarbamoyl, arylcarbamoyl, nitro, cyano, and nitrate. In embodiments, R3 is halogen. In embodiments, R3 is alkyl. In embodiments, R3 is alkyl ester. In embodiments, R3 is hydroxy. In embodiments, R3 is alkoxy. In embodiments, R3 is carboxy. In embodiments, R3 is formyl. In embodiments, R3 is aryloxy. In embodiments, R3 is amino. In embodiments, R3 is alkylamino. In embodiments, R3 is arylamido. In embodiments, R3 is alkylamido. In embodiments, R3 is thiol. In embodiments, R3 is thioalkyl. In embodiments, R3 is thioaryl. In embodiments, R3 is alkylsulfonyl. In embodiments, R3 is alkylcarbamoyl. In embodiments, R3 is arylcarbamoyl. In embodiments, R3 is nitro. In embodiments, R3 is cyano. In embodiments, R3 is nitrate.

In some embodiments, Ph is phenyl optionally substituted at one or more positions by a halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, and nitrate. In embodiments, Ph is unsubstituted phenyl. In embodiments, Ph is substituted phenyl.

Exemplary side chain variants of Formula (I) are shown in Table 1, which may according to embodiments herein be claimed, for example, as individual compounds, as part of compositions comprising an individual compound, as part of compositions comprising mixtures of two (or more) compounds, and as such compounds and/or compositions for use in preparing medicaments for treatment, or for use (as such compounds and/or compositions) in methods for modulating neurotransmission, methods of treating a medical condition or improving the symptoms thereof, and/or methods of improving mental health or functioning.

TABLE 1
Exemplary Side Chain Variants of Formula (I)

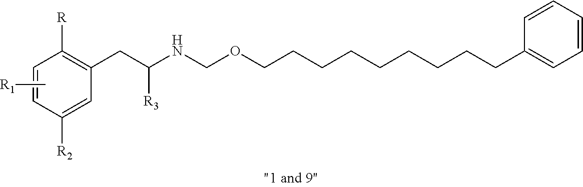

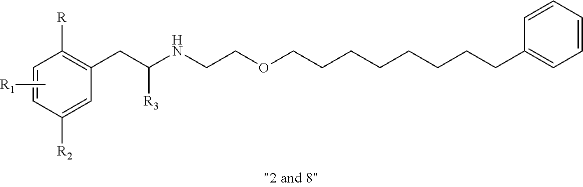

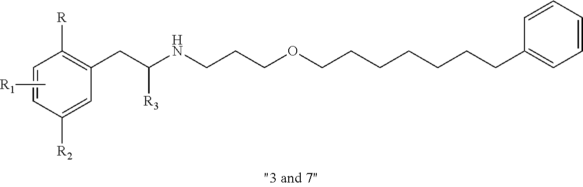

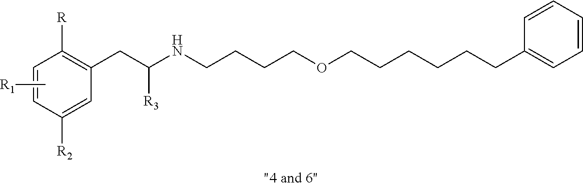

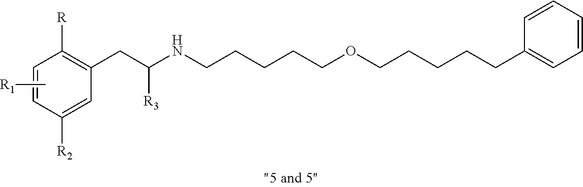

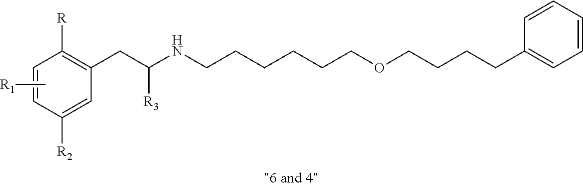

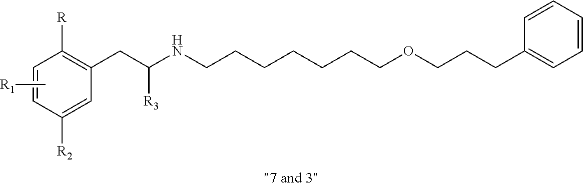

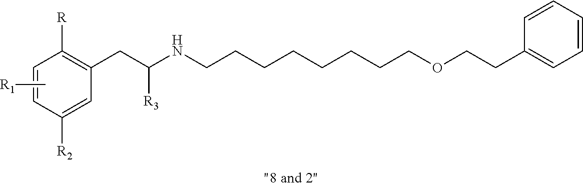

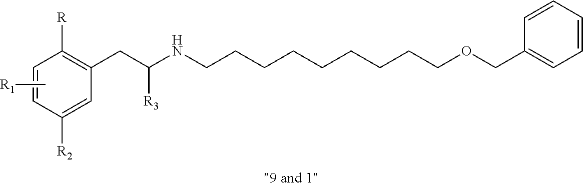

In some embodiments, the compound has the structure of Formula (II),

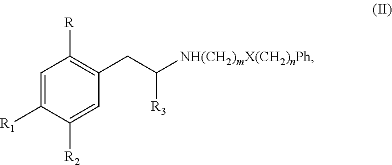

or a pharmaceutically acceptable salt thereof, wherein m and n are each independently an integer from 1 to 13, provided that the sum of m+n is from 6 to 14; X is O, S, or NH; R and R2 are each independently C1-C8 alkoxy, wherein each C1-C8 alkoxy is optionally substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate; R1 is selected from the group consisting of hydrogen, halogen, C1-C8 alkyl, C1-C8 alkoxy, C1-C8 thioalkyl, C2-C8 alkenyl, C2-C8 alkynyl, C3-C8 cycloalkyl, C3-C8 cycloalkenyl, aryl or heterocyclyl, each of which is optionally substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate; R3 is hydrogen or C1-C8 alkyl; and Ph is phenyl optionally substituted by halogen, azido, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate.

In some embodiments, m and n are each independently an integer from 1 to 13, provided that the sum of m+n is from 6 to 14. In embodiments, m is 1. In embodiments, m is 2. In embodiments, m is 3. In embodiments, m is 4. In embodiments, m is 5. In embodiments, m is 6. In embodiments, m is 7. In embodiments, m is 8. In embodiments, m is 9. In embodiments, m is 10. In embodiments, m is 11. In embodiments, m is 12. In embodiments, m is 13. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10. In embodiments, n is 11. In embodiments, n is 12. In embodiments, n is 13. In embodiments, the sum of m+n is from 6 to 14. In embodiments, the sum of m+n is from 7 to 13. In embodiments, the sum of m+n is from 8 to 12. In embodiments, the sum of m+n is from 9 to 11. In embodiments, the sum of m+n is 6. In embodiments, the sum of m+n is 7. In embodiments, the sum of m+n is 8. In embodiments, the sum of m+n is 9. In embodiments, the sum of m+n is 10. In embodiments, the sum of m+n is 11. In embodiments, the sum of m+n is 12. In embodiments, the sum of m+n is 13. In embodiments, the sum of m+n is 14.

In some embodiments, X is O, S, or NH. In embodiments, X is O. In embodiments, X is S. In embodiments, X is NH.

In some embodiments, R and R2 are each independently C1-C8 alkoxy, wherein each C1-C8 alkoxy is optionally substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R is C1-C8 alkoxy. In embodiments, R is methoxy (—OCH₃). In embodiments, R is unsubstituted C1-C8 alkoxy. In embodiments, R is C1-C8 alkoxy, wherein the C1-C8 alkoxy is substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R2 is C1-C8 alkoxy. In embodiments, R2 is methoxy (—OCH₃). In embodiments, R2 is unsubstituted C1-C8 alkoxy. In embodiments, R2 is C1-C8 alkoxy, wherein the C1-C8 alkoxy is substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate.

In some embodiments, R1 is selected from the group consisting of hydrogen, halogen, C1-C8 alkyl, C1-C8 alkoxy, C1-C8 thioalkyl, C2-C8 alkenyl, C2-C8 alkynyl, C3-C8 cycloalkyl, C3-C8 cycloalkenyl, aryl or heterocyclyl, each of which is optionally substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is hydrogen. In embodiments, R1 is halogen. In embodiments, R1 is F, Cl, Br, or I. In embodiments, R1 is F. In embodiments, R1 is Cl. In embodiments, R1 is Br. In embodiments, R1 is I. In embodiments, R1 is C1-C8 alkyl. In embodiments, R1 is methyl (—CH₃) or ethyl (—CH₂CH₃). In embodiments, R1 is methyl. In embodiments, R1 is ethyl. In embodiments, R1 is unsubstituted C1-C8 alkyl. In embodiments, R1 is C1-C8 alkyl substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is C1-C8 alkoxy. In embodiments, R1 is methoxy (—OCH₃). In embodiments, R1 is ethoxy (—OCH₂CH₃). In embodiments, R1 is unsubstituted C1-C8 alkoxy. In embodiments, R1 is C1-C8 alkoxy substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is C1-C8 thioalkyl. In embodiments, R1 is methylthio (—SCH₃). In embodiments, R1 is ethylthio (—SCH₂CH₃). In embodiments, R1 is unsubstituted C1-C8 thioalkyl. In embodiments, R1 is C1-C8 thioalkyl substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is C2-C8 alkenyl. In embodiments, R1 is unsubstituted C1-C8 alkenyl. In embodiments, R1 is C1-C8 alkenyl substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is C2-C8 alkynyl. In embodiments, R1 is unsubstituted C1-C8 alkynyl. In embodiments, R1 is C1-C8 alkynyl substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is C3-C8 cycloalkyl. In embodiments, R1 is unsubstituted C3-C8 cycloalkyl. In embodiments, R1 is C3-C8 cycloalkyl substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is C3-C8 cycloalkenyl. In embodiments, R1 is aryl. In embodiments, R1 is unsubstituted aryl. In embodiments, R1 is aryl substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, R1 is heterocyclyl. In embodiments, R1 is unsubstituted heterocyclyl. In embodiments, R1 is heterocyclyl substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate.

In some embodiments, R3 is hydrogen or C1-C8 alkyl. In embodiments, R3 is hydrogen. In embodiments, R3 is C1-C8 alkyl. In embodiments, R3 is methyl (—CH₃) or ethyl (—CH₂CH₃). In embodiments, R3 is methyl. In embodiments, R3 is ethyl.

In some embodiments, Ph is phenyl optionally substituted by halogen, azido, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, Ph is phenyl. In embodiments, Ph is unsubstituted phenyl. In embodiments, Ph is phenyl substituted by halogen, azido, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate. In embodiments, Ph is phenyl substituted by halogen. In embodiments, Ph is phenyl substituted by F, Cl, Br or I. In embodiments, Ph is phenyl substituted by azido. In embodiments, Ph is phenyl substituted by alkyl. In embodiments, Ph is phenyl substituted by alkyl ester. In embodiments, Ph is phenyl substituted by hydroxy. In embodiments, Ph is phenyl substituted by alkoxy. In embodiments, Ph is phenyl substituted by methoxy. In embodiments, Ph is phenyl substituted by carboxy. In embodiments, Ph is phenyl substituted by formyl. In embodiments, Ph is phenyl substituted by aryl. In embodiments, Ph is phenyl substituted by heterocyclyl. In embodiments, Ph is phenyl substituted by amino. In embodiments, Ph is phenyl substituted by alkylamino. In embodiments, Ph is phenyl substituted by arylamido. In embodiments, Ph is phenyl substituted by alkylamido. In embodiments, Ph is phenyl substituted by thiol. In embodiments, Ph is phenyl substituted by thioalkyl. In embodiments, Ph is phenyl substituted by thioaryl. In embodiments, Ph is phenyl substituted by alkylsulfonyl. In embodiments, Ph is phenyl substituted by alkylcarbamoyl. In embodiments, Ph is phenyl substituted by arylcarbamoyl. In embodiments, Ph is phenyl substituted by nitro. In embodiments, Ph is phenyl substituted by cyano. In embodiments, Ph is phenyl substituted by nitrate.

In some embodiments, the compound has the structure of Formulae (IIA), (IIB), or (IIC), as shown below in Table 2, wherein m, n, X, and Ph are as defined above for Formula (II).

TABLE 2
Formulae (IIA), (IIB), and (IIC)

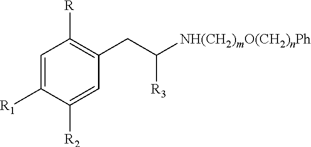

(IIA)

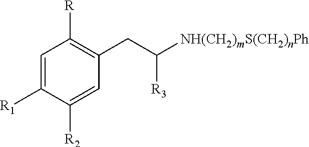

(IIB)

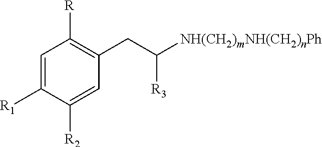

(IIC)

In some embodiments, the compound has the structure of any of Formulae (III)-(XXVI), as shown below in Table 3.

TABLE 3
Formulae (III)-(XXVI)

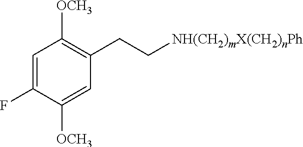

(III)

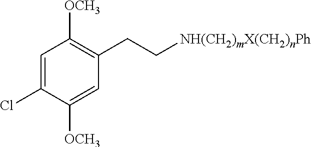

(IV)

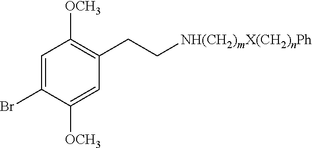

(V)

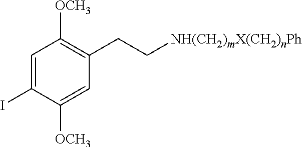

(VI)

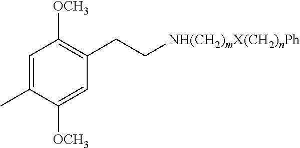

(VII)

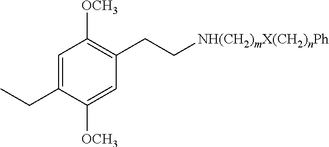

(VIII)

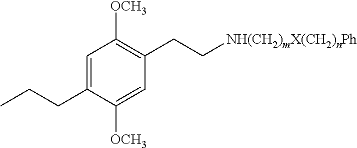

(IX)

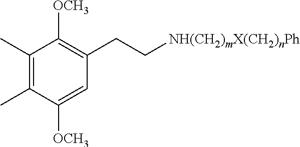

(X)

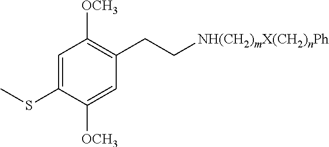

(XI)

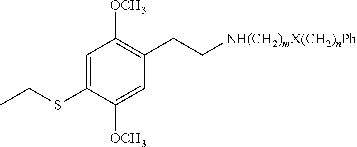

(XII)

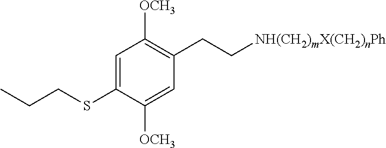

(XIII)

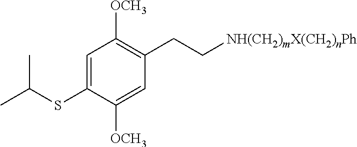

(XIV)

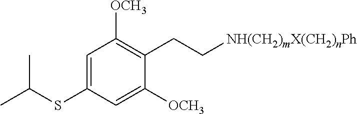

(XV)

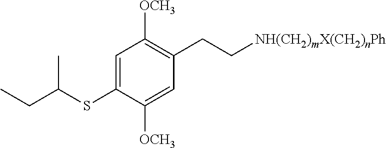

(XVI)

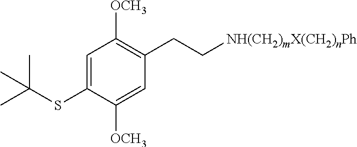

(XVII)

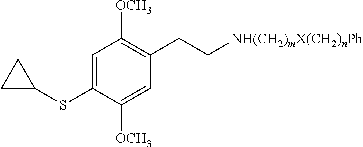

(XVIII)

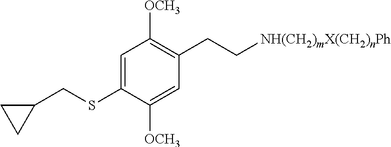

(XIX)

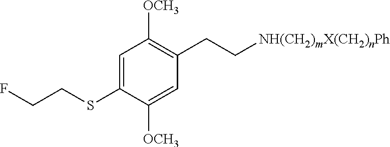

(XX)

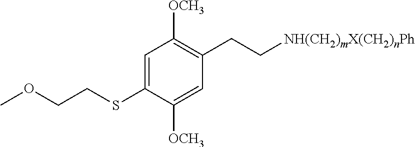

(XXI)

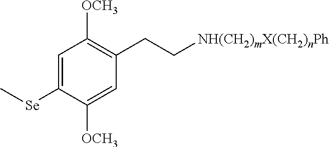

(XXII)

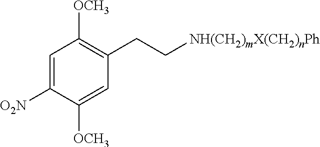

(XXIII)

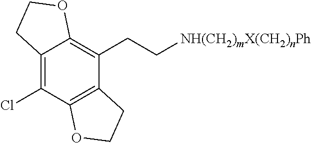

(XXIV)

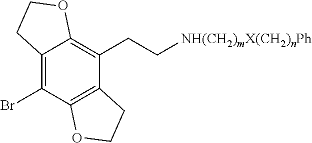

(XXV)

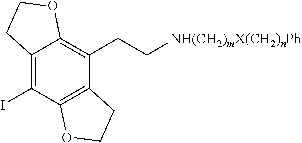

(XXVI)

In some embodiments, the compound is selected from Table 4, wherein the compound has the structure below, with R, R₁, R₂, R₃, m, X, n, and Ph as defined in the Table:

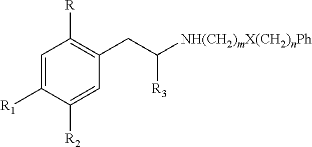

TABLE 4
Exemplary N-Substituted Phenylalkylamines

| No. | R | R1 | R2 | R3 | m | X | n | Ph |
|---|---|---|---|---|---|---|---|---|
| 1 | OCH₃ | Br | OCH₃ | H | 1 | O | 9 | 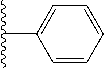 [SMILES: *c1ccccc1] |
| 2 | OCH₃ | Br | OCH₃ | H | 1 | O | 10 | 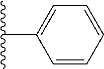 [SMILES: *c1ccccc1] |
| 3 | OCH₃ | Br | OCH₃ | H | 2 | O | 8 | 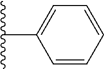 [SMILES: *c1ccccc1] |
| 4 | OCH₃ | Br | OCH₃ | H | 3 | O | 7 | 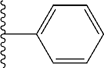 [SMILES: *c1ccccc1] |
| 5 | OCH₃ | Br | OCH₃ | H | 4 | O | 6 | 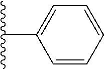 [SMILES: *c1ccccc1] |
| 6 | OCH₃ | Br | OCH₃ | H | 5 | O | 5 | 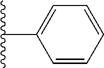 [SMILES: *c1ccccc1] |
| 7 | OCH₃ | Br | OCH₃ | H | 6 | O | 4 | 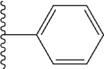 [SMILES: *c1ccccc1] |
| 8 | OCH₃ | Br | OCH₃ | H | 7 | O | 3 | 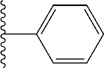 [SMILES: *c1ccccc1] |
| 9 | OCH₃ | Br | OCH₃ | H | 8 | O | 2 | 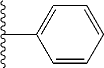 [SMILES: *c1ccccc1] |
| 10 | OCH₃ | Br | OCH₃ | H | 9 | O | 1 | 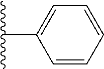 [SMILES: *c1ccccc1] |
| 11 | OCH₃ | Br | OCH₃ | H | 1 | S | 9 | 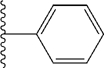 [SMILES: *c1ccccc1] |
| 12 | OCH₃ | Br | OCH₃ | H | 1 | S | 10 | 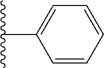 [SMILES: *c1ccccc1] |
| 13 | OCH₃ | Br | OCH₃ | H | 2 | S | 8 | 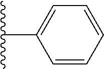 [SMILES: *c1ccccc1] |
| 14 | OCH₃ | Br | OCH₃ | H | 3 | S | 7 | 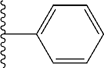 [SMILES: *c1ccccc1] |
| 15 | OCH₃ | Br | OCH₃ | H | 4 | S | 6 | 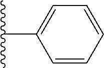 [SMILES: *c1ccccc1] |
| 16 | OCH₃ | Br | OCH₃ | H | 5 | S | 5 | 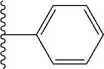 [SMILES: *c1ccccc1] |
| 17 | OCH₃ | Br | OCH₃ | H | 6 | S | 4 | 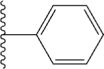 [SMILES: *c1ccccc1] |
| 18 | OCH₃ | Br | OCH₃ | H | 7 | S | 3 | 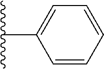 [SMILES: *c1ccccc1] |
| 19 | OCH₃ | Br | OCH₃ | H | 8 | S | 2 | 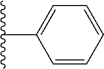 [SMILES: *c1ccccc1] |
| 20 | OCH₃ | Br | OCH₃ | H | 9 | S | 1 | 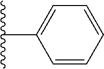 [SMILES: *c1ccccc1] |
| 21 | OCH₃ | Br | OCH₃ | H | 1 | NH | 9 | 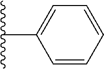 [SMILES: *c1ccccc1] |
| 22 | OCH₃ | Br | OCH₃ | H | 1 | NH | 10 | 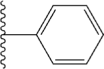 [SMILES: *c1ccccc1] |
| 23 | OCH₃ | Br | OCH₃ | H | 2 | NH | 8 | 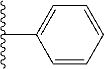 [SMILES: *c1ccccc1] |
| 24 | OCH₃ | Br | OCH₃ | H | 3 | NH | 7 | 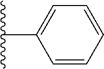 [SMILES: *c1ccccc1] |
| 25 | OCH₃ | Br | OCH₃ | H | 4 | NH | 6 | 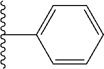 [SMILES: *c1ccccc1] |
| 26 | OCH₃ | Br | OCH₃ | H | 5 | NH | 5 | 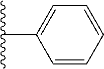 [SMILES: *c1ccccc1] |
| 27 | OCH₃ | Br | OCH₃ | H | 6 | NH | 4 | 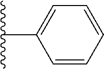 [SMILES: *c1ccccc1] |
| 28 | OCH₃ | Br | OCH₃ | H | 7 | NH | 3 | 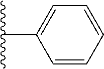 [SMILES: *c1ccccc1] |
| 29 | OCH₃ | Br | OCH₃ | H | 8 | NH | 2 | 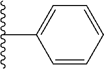 [SMILES: *c1ccccc1] |
| 30 | OCH₃ | Br | OCH₃ | H | 9 | NH | 1 | 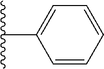 [SMILES: *c1ccccc1] |
| 31 | OCH₃ | Br | OCH₃ | H | 1 | O | 9 | 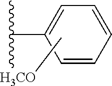 |
| 32 | OCH₃ | Br | OCH₃ | H | 1 | O | 10 | 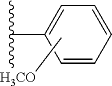 |
| 33 | OCH₃ | Br | OCH₃ | H | 2 | O | 8 | 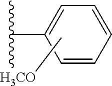 |
| 34 | OCH₃ | Br | OCH₃ | H | 3 | O | 7 | 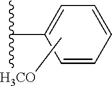 |
| 35 | OCH₃ | Br | OCH₃ | H | 4 | O | 6 | 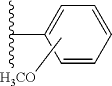 |
| 36 | OCH₃ | Br | OCH₃ | H | 5 | O | 5 | 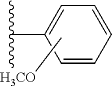 |
| 37 | OCH₃ | Br | OCH₃ | H | 6 | O | 4 | 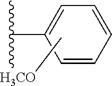 |
| 38 | OCH₃ | Br | OCH₃ | H | 7 | O | 3 | 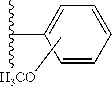 |
| 39 | OCH₃ | Br | OCH₃ | H | 8 | O | 2 | 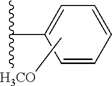 |
| 40 | OCH₃ | Br | OCH₃ | H | 9 | O | 1 | 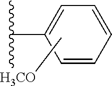 |
| 41 | OCH₃ | Br | OCH₃ | H | 1 | S | 9 | 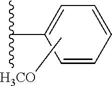 |
| 42 | OCH₃ | Br | OCH₃ | H | 1 | S | 10 | 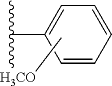 |
| 43 | OCH₃ | Br | OCH₃ | H | 2 | S | 8 | 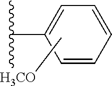 |
| 44 | OCH₃ | Br | OCH₃ | H | 3 | S | 7 | 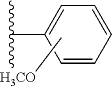 |
| 45 | OCH₃ | Br | OCH₃ | H | 4 | S | 6 | 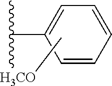 |
| 46 | OCH₃ | Br | OCH₃ | H | 5 | S | 5 | 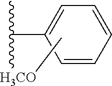 |
| 47 | OCH₃ | Br | OCH₃ | H | 6 | S | 4 | 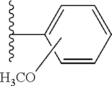 |
| 48 | OCH₃ | Br | OCH₃ | H | 7 | S | 3 | 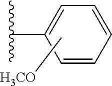 |
| 49 | OCH₃ | Br | OCH₃ | H | 8 | S | 2 | 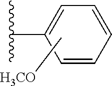 |
| 50 | OCH₃ | Br | OCH₃ | H | 9 | S | 1 | 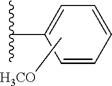 |
| 51 | OCH₃ | Br | OCH₃ | H | 1 | NH | 9 | 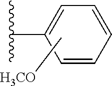 |
| 52 | OCH₃ | Br | OCH₃ | H | 1 | NH | 10 | 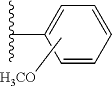 |
| 53 | OCH₃ | Br | OCH₃ | H | 2 | NH | 8 | 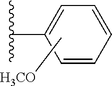 |
| 54 | OCH₃ | Br | OCH₃ | H | 3 | NH | 7 | 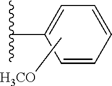 |
| 55 | OCH₃ | Br | OCH₃ | H | 4 | NH | 6 | 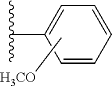 |
| 56 | OCH₃ | Br | OCH₃ | H | 5 | NH | 5 | 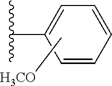 |
| 57 | OCH₃ | Br | OCH₃ | H | 6 | NH | 4 | 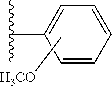 |
| 58 | OCH₃ | Br | OCH₃ | H | 7 | NH | 3 | 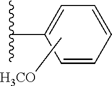 |
| 59 | OCH₃ | Br | OCH₃ | H | 8 | NH | 2 | 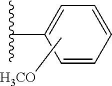 |
| 60 | OCH₃ | Br | OCH₃ | H | 9 | NH | 1 | 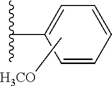 |
| 61 | OCH₃ | Br | OCH₃ | H | 1 | O | 9 | 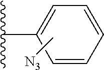 |
| 62 | OCH₃ | Br | OCH₃ | H | 1 | O | 10 | 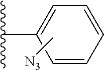 |
| 63 | OCH₃ | Br | OCH₃ | H | 2 | O | 8 | 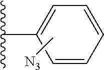 |
| 64 | OCH₃ | Br | OCH₃ | H | 3 | O | 7 | 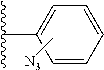 |
| 65 | OCH₃ | Br | OCH₃ | H | 4 | O | 6 | 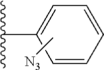 |
| 66 | OCH₃ | Br | OCH₃ | H | 5 | O | 5 | 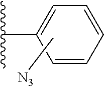 |
| 67 | OCH₃ | Br | OCH₃ | H | 6 | O | 4 | 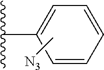 |
| 68 | OCH₃ | Br | OCH₃ | H | 7 | O | 3 | 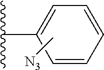 |
| 69 | OCH₃ | Br | OCH₃ | H | 8 | O | 2 | 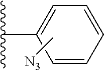 |
| 70 | OCH₃ | Br | OCH₃ | H | 9 | O | 1 | 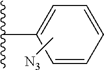 |
| 71 | OCH₃ | Br | OCH₃ | H | 1 | S | 9 | 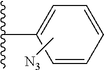 |
| 72 | OCH₃ | Br | OCH₃ | H | 1 | S | 10 | 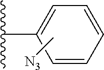 |
| 73 | OCH₃ | Br | OCH₃ | H | 2 | S | 8 | 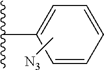 |
| 74 | OCH₃ | Br | OCH₃ | H | 3 | S | 7 | 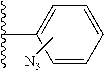 |
| 75 | OCH₃ | Br | OCH₃ | H | 4 | S | 6 | 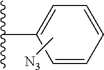 |
| 76 | OCH₃ | Br | OCH₃ | H | 5 | S | 5 | 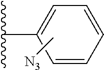 |
| 77 | OCH₃ | Br | OCH₃ | H | 6 | S | 4 | 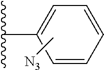 |
| 78 | OCH₃ | Br | OCH₃ | H | 7 | S | 3 | 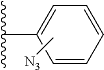 |
| 79 | OCH₃ | Br | OCH₃ | H | 8 | S | 2 | 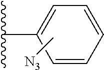 |
| 80 | OCH₃ | Br | OCH₃ | H | 9 | S | 1 | 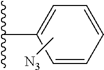 |
| 81 | OCH₃ | Br | OCH₃ | H | 1 | NH | 9 | 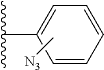 |
| 82 | OCH₃ | Br | OCH₃ | H | 1 | NH | 10 | 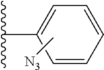 |
| 83 | OCH₃ | Br | OCH₃ | H | 2 | NH | 8 | 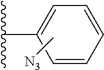 |
| 84 | OCH₃ | Br | OCH₃ | H | 3 | NH | 7 | 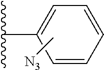 |
| 85 | OCH₃ | Br | OCH₃ | H | 4 | NH | 6 | 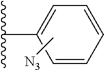 |
| 86 | OCH₃ | Br | OCH₃ | H | 5 | NH | 5 | 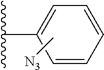 |
| 87 | OCH₃ | Br | OCH₃ | H | 6 | NH | 4 | 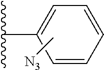 |
| 88 | OCH₃ | Br | OCH₃ | H | 7 | NH | 3 | 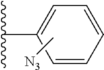 |
| 89 | OCH₃ | Br | OCH₃ | H | 8 | NH | 2 | 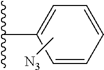 |
| 90 | OCH₃ | Br | OCH₃ | H | 9 | NH | 1 | 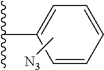 |
| 91 | OCH₃ | Br | OCH₂CH₃ | H | 1 | O | 9 | 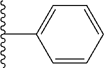 [SMILES: *c1ccccc1] |
| 92 | OCH₃ | Br | OCH₂CH₃ | H | 2 | O | 8 | 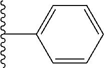 [SMILES: *c1ccccc1] |
| 93 | OCH₃ | Br | OCH₂CH₃ | H | 3 | O | 7 | 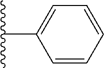 [SMILES: *c1ccccc1] |
| 94 | OCH₃ | Br | OCH₂CH₃ | H | 4 | O | 6 | 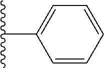 [SMILES: *c1ccccc1] |
| 95 | OCH₃ | Br | OCH₂CH₃ | H | 5 | O | 5 | 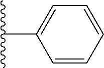 [SMILES: *c1ccccc1] |
| 96 | OCH₃ | Br | OCH₂CH₃ | H | 6 | O | 4 | 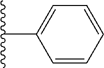 [SMILES: *c1ccccc1] |
| 97 | OCH₃ | Br | OCH₂CH₃ | H | 7 | O | 3 | 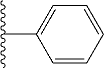 [SMILES: *c1ccccc1] |
| 98 | OCH₃ | Br | OCH₂CH₃ | H | 8 | O | 2 | 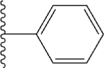 [SMILES: *c1ccccc1] |
| 99 | OCH₃ | Br | OCH₂CH₃ | H | 9 | O | 1 | 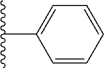 [SMILES: *c1ccccc1] |
| 100 | OCH₃ | Br | OCH₂CH₂CH₃ | H | 1 | O | 9 | 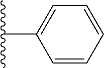 [SMILES: *c1ccccc1] |
| 101 | OCH₃ | Br | OCH₂CH₂CH₃ | H | 2 | O | 8 | 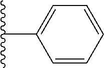 [SMILES: *c1ccccc1] |
| 102 | OCH₃ | Br | OCH₂CH₂CH₃ | H | 3 | O | 7 | 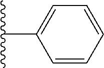 [SMILES: *c1ccccc1] |
| 103 | OCH₃ | Br | OCH₂CH₂CH₃ | H | 4 | O | 6 | 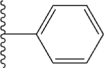 [SMILES: *c1ccccc1] |
| 104 | OCH₃ | Br | OCH₂CH₂CH₃ | H | 5 | O | 5 | 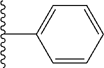 [SMILES: *c1ccccc1] |
| 105 | OCH₃ | Br | OCH₂CH₂CH₃ | H | 6 | O | 4 | 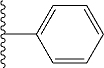 [SMILES: *c1ccccc1] |
| 106 | OCH₃ | Br | OCH₂CH₂CH₃ | H | 7 | O | 3 | 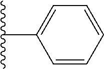 [SMILES: *c1ccccc1] |
| 107 | OCH₃ | Br | OCH₂CH₂CH₃ | H | 8 | O | 2 | 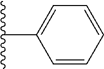 [SMILES: *c1ccccc1] |
| 108 | OCH₃ | Br | OCH₂CH₂CH₃ | H | 9 | O | 1 | 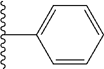 [SMILES: *c1ccccc1] |
| 109 | OCH₂CH₃ | Br | OCH₃ | H | 1 | O | 9 | 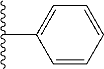 [SMILES: *c1ccccc1] |
| 110 | OCH₂CH₃ | Br | OCH₃ | H | 2 | O | 8 | 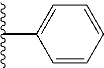 [SMILES: *c1ccccc1] |
| 111 | OCH₂CH₃ | Br | OCH₃ | H | 3 | O | 7 | 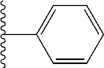 [SMILES: *c1ccccc1] |
| 112 | OCH₂CH₃ | Br | OCH₃ | H | 4 | O | 6 | 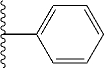 [SMILES: *c1ccccc1] |
| 113 | OCH₂CH₃ | Br | OCH₃ | H | 5 | O | 5 | 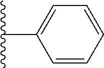 [SMILES: *c1ccccc1] |
| 114 | OCH₂CH₃ | Br | OCH₃ | H | 6 | O | 4 | 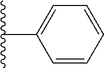 [SMILES: *c1ccccc1] |
| 115 | OCH₂CH₃ | Br | OCH₃ | H | 7 | O | 3 | 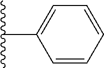 [SMILES: *c1ccccc1] |
| 116 | OCH₂CH₃ | Br | OCH₃ | H | 8 | O | 2 | 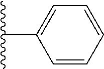 [SMILES: *c1ccccc1] |
| 117 | OCH₂CH₃ | Br | OCH₃ | H | 9 | O | 1 | 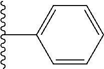 [SMILES: *c1ccccc1] |
| 118 | OCH₂CH₂CH₃ | Br | OCH₃ | H | 1 | O | 9 | 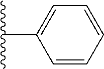 [SMILES: *c1ccccc1] |
| 119 | OCH₂CH₂CH₃ | Br | OCH₃ | H | 2 | O | 8 | 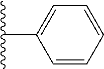 [SMILES: *c1ccccc1] |
| 120 | OCH₂CH₂CH₃ | Br | OCH₃ | H | 3 | O | 7 | 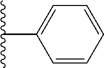 [SMILES: *c1ccccc1] |
| 121 | OCH₂CH₂CH₃ | Br | OCH₃ | H | 4 | O | 6 | 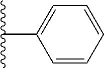 [SMILES: *c1ccccc1] |
| 122 | OCH₂CH₂CH₃ | Br | OCH₃ | H | 5 | O | 5 | 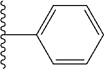 [SMILES: *c1ccccc1] |
| 123 | OCH₂CH₂CH₃ | Br | OCH₃ | H | 6 | O | 4 | 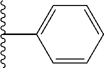 [SMILES: *c1ccccc1] |
| 124 | OCH₂CH₂CH₃ | Br | OCH₃ | H | 7 | O | 3 | 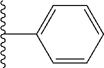 [SMILES: *c1ccccc1] |
| 125 | OCH₂CH₂CH₃ | Br | OCH₃ | H | 8 | O | 2 | 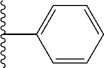 [SMILES: *c1ccccc1] |
| 126 | OCH₂CH₂CH₃ | Br | OCH₃ | H | 9 | O | 1 | 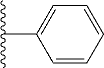 [SMILES: *c1ccccc1] |
| 127 | OCH₃ | I | OCH₃ | H | 1 | O | 9 | 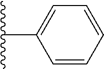 [SMILES: *c1ccccc1] |
| 128 | OCH₃ | I | OCH₃ | H | 1 | O | 10 | 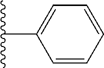 [SMILES: *c1ccccc1] |
| 129 | OCH₃ | I | OCH₃ | H | 2 | O | 8 | 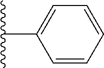 [SMILES: *c1ccccc1] |
| 130 | OCH₃ | I | OCH₃ | H | 3 | O | 7 | 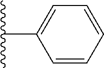 [SMILES: *c1ccccc1] |
| 131 | OCH₃ | I | OCH₃ | H | 4 | O | 6 | 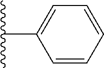 [SMILES: *c1ccccc1] |
| 132 | OCH₃ | I | OCH₃ | H | 5 | O | 5 | 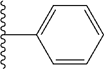 [SMILES: *c1ccccc1] |
| 133 | OCH₃ | I | OCH₃ | H | 6 | O | 4 | 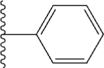 [SMILES: *c1ccccc1] |
| 134 | OCH₃ | I | OCH₃ | H | 7 | O | 3 | 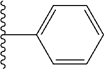 [SMILES: *c1ccccc1] |
| 135 | OCH₃ | I | OCH₃ | H | 8 | O | 2 | 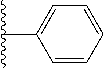 [SMILES: *c1ccccc1] |
| 136 | OCH₃ | I | OCH₃ | H | 9 | O | 1 | 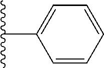 [SMILES: *c1ccccc1] |
| 137 | OCH₃ | I | OCH₃ | H | 1 | S | 9 | 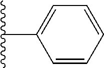 [SMILES: *c1ccccc1] |
| 138 | OCH₃ | I | OCH₃ | H | 1 | S | 10 | 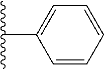 [SMILES: *c1ccccc1] |
| 139 | OCH₃ | I | OCH₃ | H | 2 | S | 8 | 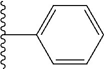 [SMILES: *c1ccccc1] |
| 140 | OCH₃ | I | OCH₃ | H | 3 | S | 7 | 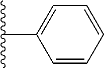 [SMILES: *c1ccccc1] |
| 141 | OCH₃ | I | OCH₃ | H | 4 | S | 6 | 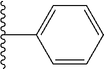 [SMILES: *c1ccccc1] |
| 142 | OCH₃ | I | OCH₃ | H | 5 | S | 5 | 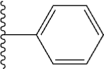 [SMILES: *c1ccccc1] |
| 143 | OCH₃ | I | OCH₃ | H | 6 | S | 4 | 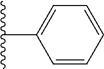 [SMILES: *c1ccccc1] |
| 144 | OCH₃ | I | OCH₃ | H | 7 | S | 3 | 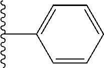 [SMILES: *c1ccccc1] |
| 145 | OCH₃ | I | OCH₃ | H | 8 | S | 2 | 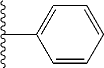 [SMILES: *c1ccccc1] |
| 146 | OCH₃ | I | OCH₃ | H | 9 | S | 1 | 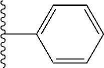 [SMILES: *c1ccccc1] |
| 147 | OCH₃ | I | OCH₃ | H | 1 | NH | 9 | 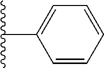 [SMILES: *c1ccccc1] |
| 148 | OCH₃ | I | OCH₃ | H | 1 | NH | 10 | 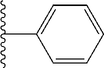 [SMILES: *c1ccccc1] |
| 149 | OCH₃ | I | OCH₃ | H | 2 | NH | 8 | 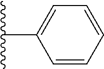 [SMILES: *c1ccccc1] |
| 150 | OCH₃ | I | OCH₃ | H | 3 | NH | 7 | 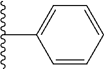 [SMILES: *c1ccccc1] |
| 151 | OCH₃ | I | OCH₃ | H | 4 | NH | 6 | 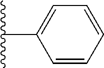 [SMILES: *c1ccccc1] |
| 152 | OCH₃ | I | OCH₃ | H | 5 | NH | 5 | 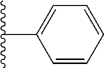 [SMILES: *c1ccccc1] |
| 153 | OCH₃ | I | OCH₃ | H | 6 | NH | 4 | 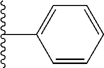 [SMILES: *c1ccccc1] |
| 154 | OCH₃ | I | OCH₃ | H | 7 | NH | 3 | 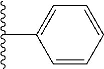 [SMILES: *c1ccccc1] |
| 155 | OCH₃ | I | OCH₃ | H | 8 | NH | 2 | 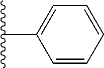 [SMILES: *c1ccccc1] |
| 156 | OCH₃ | I | OCH₃ | H | 9 | NH | 1 | 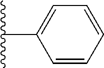 [SMILES: *c1ccccc1] |
| 157 | OCH₃ | I | OCH₃ | H | 1 | O | 9 | 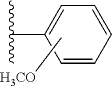 |
| 158 | OCH₃ | I | OCH₃ | H | 1 | O | 10 | 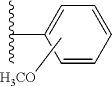 |
| 159 | OCH₃ | I | OCH₃ | H | 2 | O | 8 | 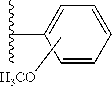 |
| 160 | OCH₃ | I | OCH₃ | H | 3 | O | 7 | 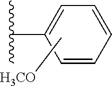 |
| 161 | OCH₃ | I | OCH₃ | H | 4 | O | 6 | 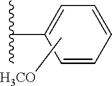 |
| 162 | OCH₃ | I | OCH₃ | H | 5 | O | 5 | 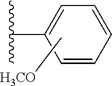 |
| 163 | OCH₃ | I | OCH₃ | H | 6 | O | 4 | 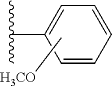 |
| 164 | OCH₃ | I | OCH₃ | H | 7 | O | 3 | 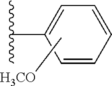 |
| 165 | OCH₃ | I | OCH₃ | H | 8 | O | 2 | 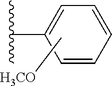 |
| 166 | OCH₃ | I | OCH₃ | H | 9 | O | 1 | 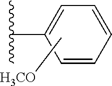 |
| 167 | OCH₃ | I | OCH₃ | H | 1 | S | 9 | 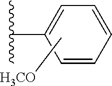 |
| 168 | OCH₃ | I | OCH₃ | H | 1 | S | 10 | 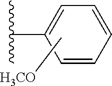 |
| 169 | OCH₃ | I | OCH₃ | H | 2 | S | 8 | 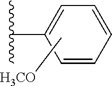 |
| 170 | OCH₃ | I | OCH₃ | H | 3 | S | 7 | 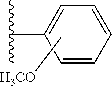 |
| 171 | OCH₃ | I | OCH₃ | H | 4 | S | 6 | 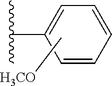 |
| 172 | OCH₃ | I | OCH₃ | H | 5 | S | 5 | 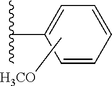 |
| 173 | OCH₃ | I | OCH₃ | H | 6 | S | 4 | 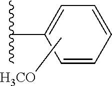 |
| 174 | OCH₃ | I | OCH₃ | H | 7 | S | 3 | 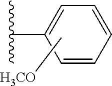 |
| 175 | OCH₃ | I | OCH₃ | H | 8 | S | 2 | 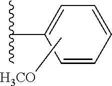 |
| 176 | OCH₃ | I | OCH₃ | H | 9 | S | 1 | 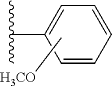 |
| 177 | OCH₃ | I | OCH₃ | H | 1 | NH | 9 | 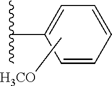 |
| 178 | OCH₃ | I | OCH₃ | H | 1 | NH | 10 | 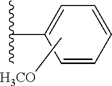 |
| 179 | OCH₃ | I | OCH₃ | H | 2 | NH | 8 | 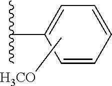 |
| 180 | OCH₃ | I | OCH₃ | H | 3 | NH | 7 | 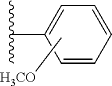 |
| 181 | OCH₃ | I | OCH₃ | H | 4 | NH | 6 | 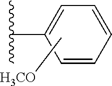 |
| 182 | OCH₃ | I | OCH₃ | H | 5 | NH | 5 | 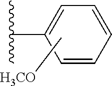 |
| 183 | OCH₃ | I | OCH₃ | H | 6 | NH | 4 | 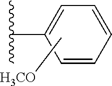 |
| 184 | OCH₃ | I | OCH₃ | H | 7 | NH | 3 | 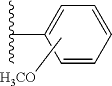 |
| 185 | OCH₃ | I | OCH₃ | H | 8 | NH | 2 | 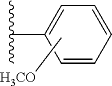 |
| 186 | OCH₃ | I | OCH₃ | H | 9 | NH | 1 | 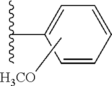 |
| 187 | OCH₃ | I | OCH₃ | H | 1 | O | 9 | 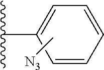 |
| 188 | OCH₃ | I | OCH₃ | H | 1 | O | 10 | 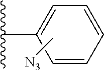 |
| 189 | OCH₃ | I | OCH₃ | H | 2 | O | 8 | 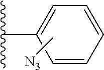 |
| 190 | OCH₃ | I | OCH₃ | H | 3 | O | 7 | 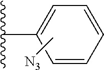 |
| 191 | OCH₃ | I | OCH₃ | H | 4 | O | 6 | 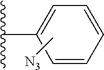 |
| 192 | OCH₃ | I | OCH₃ | H | 5 | O | 5 | 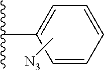 |
| 193 | OCH₃ | I | OCH₃ | H | 6 | O | 4 | 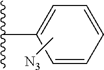 |
| 194 | OCH₃ | I | OCH₃ | H | 7 | O | 3 | 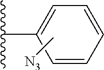 |
| 195 | OCH₃ | I | OCH₃ | H | 8 | O | 2 | 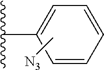 |
| 196 | OCH₃ | I | OCH₃ | H | 9 | O | 1 | 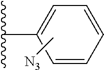 |
| 197 | OCH₃ | I | OCH₃ | H | 1 | S | 9 | 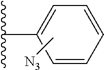 |
| 198 | OCH₃ | I | OCH₃ | H | 1 | S | 10 | 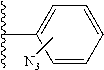 |
| 199 | OCH₃ | I | OCH₃ | H | 2 | S | 8 | 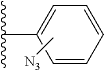 |
| 200 | OCH₃ | I | OCH₃ | H | 3 | S | 7 | 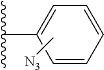 |
| 201 | OCH₃ | I | OCH₃ | H | 4 | S | 6 | 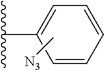 |
| 202 | OCH₃ | I | OCH₃ | H | 5 | S | 5 | 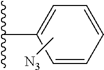 |
| 203 | OCH₃ | I | OCH₃ | H | 6 | S | 4 | 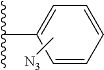 |
| 204 | OCH₃ | I | OCH₃ | H | 7 | S | 3 | 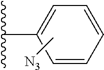 |
| 205 | OCH₃ | I | OCH₃ | H | 8 | S | 2 | 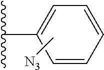 |
| 206 | OCH₃ | I | OCH₃ | H | 9 | S | 1 | 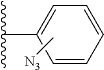 |
| 207 | OCH₃ | I | OCH₃ | H | 1 | NH | 9 | 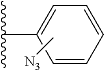 |
| 208 | OCH₃ | I | OCH₃ | H | 1 | NH | 10 | 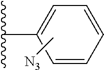 |
| 209 | OCH₃ | I | OCH₃ | H | 2 | NH | 8 | 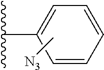 |
| 210 | OCH₃ | I | OCH₃ | H | 3 | NH | 7 | 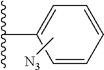 |
| 211 | OCH₃ | I | OCH₃ | H | 4 | NH | 6 | 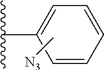 |
| 212 | OCH₃ | I | OCH₃ | H | 5 | NH | 5 | 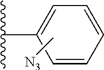 |
| 213 | OCH₃ | I | OCH₃ | H | 6 | NH | 4 | 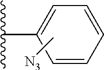 |
| 214 | OCH₃ | I | OCH₃ | H | 7 | NH | 3 | 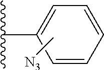 |
| 215 | OCH₃ | I | OCH₃ | H | 8 | NH | 2 | 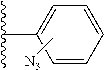 |
| 216 | OCH₃ | I | OCH₃ | H | 9 | NH | 1 | 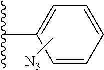 |
| 217 | OCH₃ | I | OCH₂CH₃ | H | 1 | O | 9 | 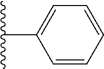 [SMILES: *c1ccccc1] |
| 218 | OCH₃ | I | OCH₂CH₃ | H | 2 | O | 8 | 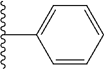 [SMILES: *c1ccccc1] |
| 219 | OCH₃ | I | OCH₂CH₃ | H | 3 | O | 7 | 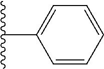 [SMILES: *c1ccccc1] |
| 220 | OCH₃ | I | OCH₂CH₃ | H | 4 | O | 6 | 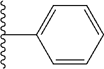 [SMILES: *c1ccccc1] |
| 221 | OCH₃ | I | OCH₂CH₃ | H | 5 | O | 5 | 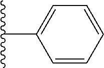 [SMILES: *c1ccccc1] |
| 222 | OCH₃ | I | OCH₂CH₃ | H | 6 | O | 4 | 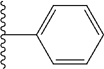 [SMILES: *c1ccccc1] |
| 223 | OCH₃ | I | OCH₂CH₃ | H | 7 | O | 3 | 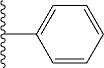 [SMILES: *c1ccccc1] |
| 224 | OCH₃ | I | OCH₂CH₃ | H | 8 | O | 2 | 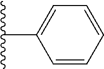 [SMILES: *c1ccccc1] |
| 225 | OCH₃ | I | OCH₂CH₃ | H | 9 | O | 1 | 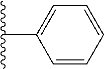 [SMILES: *c1ccccc1] |
| 226 | OCH₃ | I | OCH₂CH₂CH₃ | H | 1 | O | 9 | 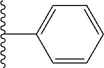 [SMILES: *c1ccccc1] |
| 227 | OCH₃ | I | OCH₂CH₂CH₃ | H | 2 | O | 8 | 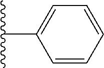 [SMILES: *c1ccccc1] |
| 228 | OCH₃ | I | OCH₂CH₂CH₃ | H | 3 | O | 7 | 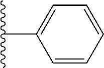 [SMILES: *c1ccccc1] |
| 229 | OCH₃ | I | OCH₂CH₂CH₃ | H | 4 | O | 6 | 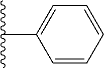 [SMILES: *c1ccccc1] |
| 230 | OCH₃ | I | OCH₂CH₂CH₃ | H | 5 | O | 5 | 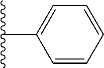 [SMILES: *c1ccccc1] |
| 231 | OCH₃ | I | OCH₂CH₂CH₃ | H | 6 | O | 4 | 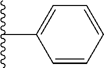 [SMILES: *c1ccccc1] |
| 232 | OCH₃ | I | OCH₂CH₂CH₃ | H | 7 | O | 3 | 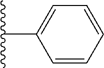 [SMILES: *c1ccccc1] |
| 233 | OCH₃ | I | OCH₂CH₂CH₃ | H | 8 | O | 2 | 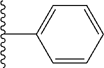 [SMILES: *c1ccccc1] |
| 234 | OCH₃ | I | OCH₂CH₂CH₃ | H | 9 | O | 1 | 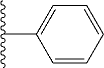 [SMILES: *c1ccccc1] |
| 235 | OCH₂CH₃ | I | OCH₃ | H | 1 | O | 9 | 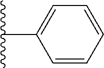 [SMILES: *c1ccccc1] |
| 236 | OCH₂CH₃ | I | OCH₃ | H | 2 | O | 8 | 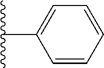 [SMILES: *c1ccccc1] |
| 237 | OCH₂CH₃ | I | OCH₃ | H | 3 | O | 7 | 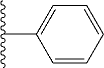 [SMILES: *c1ccccc1] |
| 238 | OCH₂CH₃ | I | OCH₃ | H | 4 | O | 6 | 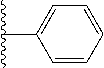 [SMILES: *c1ccccc1] |
| 239 | OCH₂CH₃ | I | OCH₃ | H | 5 | O | 5 | 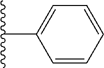 [SMILES: *c1ccccc1] |
| 240 | OCH₂CH₃ | I | OCH₃ | H | 6 | O | 4 | 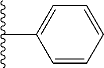 [SMILES: *c1ccccc1] |
| 241 | OCH₂CH₃ | I | OCH₃ | H | 7 | O | 3 | 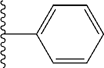 [SMILES: *c1ccccc1] |
| 242 | OCH₂CH₃ | I | OCH₃ | H | 8 | O | 2 | 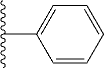 [SMILES: *c1ccccc1] |
| 243 | OCH₂CH₃ | I | OCH₃ | H | 9 | O | 1 | 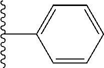 [SMILES: *c1ccccc1] |
| 244 | OCH₂CH₂CH₃ | I | OCH₃ | H | 1 | O | 9 | 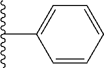 [SMILES: *c1ccccc1] |
| 245 | OCH₂CH₂CH₃ | I | OCH₃ | H | 2 | O | 8 | 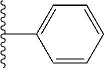 [SMILES: *c1ccccc1] |
| 246 | OCH₂CH₂CH₃ | I | OCH₃ | H | 3 | O | 7 | 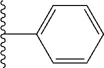 [SMILES: *c1ccccc1] |
| 247 | OCH₂CH₂CH₃ | I | OCH₃ | H | 4 | O | 6 | 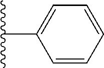 [SMILES: *c1ccccc1] |
| 248 | OCH₂CH₂CH₃ | I | OCH₃ | H | 5 | O | 5 | 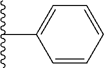 [SMILES: *c1ccccc1] |
| 249 | OCH₂CH₂CH₃ | I | OCH₃ | H | 6 | O | 4 | 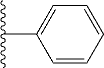 [SMILES: *c1ccccc1] |
| 250 | OCH₂CH₂CH₃ | I | OCH₃ | H | 7 | O | 3 | 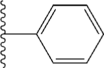 [SMILES: *c1ccccc1] |
| 251 | OCH₂CH₂CH₃ | I | OCH₃ | H | 8 | O | 2 | 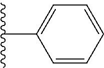 [SMILES: *c1ccccc1] |
| 252 | OCH₂CH₂CH₃ | I | OCH₃ | H | 9 | O | 1 | 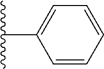 [SMILES: *c1ccccc1] |
| 253 | OCH₃ | CH₃ | OCH₃ | H | 1 | O | 9 | 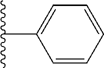 [SMILES: *c1ccccc1] |
| 254 | OCH₃ | CH₃ | OCH₃ | H | 1 | O | 10 | 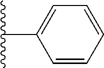 [SMILES: *c1ccccc1] |
| 255 | OCH₃ | CH₃ | OCH₃ | H | 2 | O | 8 | 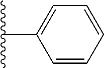 [SMILES: *c1ccccc1] |
| 256 | OCH₃ | CH₃ | OCH₃ | H | 3 | O | 7 | 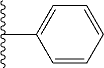 [SMILES: *c1ccccc1] |
| 257 | OCH₃ | CH₃ | OCH₃ | H | 4 | O | 6 | 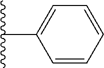 [SMILES: *c1ccccc1] |
| 258 | OCH₃ | CH₃ | OCH₃ | H | 5 | O | 5 | 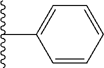 [SMILES: *c1ccccc1] |
| 259 | OCH₃ | CH₃ | OCH₃ | H | 6 | O | 4 | 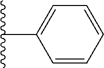 [SMILES: *c1ccccc1] |
| 260 | OCH₃ | CH₃ | OCH₃ | H | 7 | O | 3 | 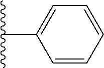 [SMILES: *c1ccccc1] |
| 261 | OCH₃ | CH₃ | OCH₃ | H | 8 | O | 2 | 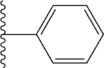 [SMILES: *c1ccccc1] |
| 262 | OCH₃ | CH₃ | OCH₃ | H | 9 | O | 1 | 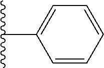 [SMILES: *c1ccccc1] |
| 263 | OCH₃ | CH₃ | OCH₃ | H | 1 | S | 9 | 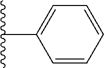 [SMILES: *c1ccccc1] |
| 264 | OCH₃ | CH₃ | OCH₃ | H | 1 | S | 10 | 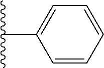 [SMILES: *c1ccccc1] |
| 265 | OCH₃ | CH₃ | OCH₃ | H | 2 | U | 8 | 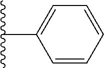 [SMILES: *c1ccccc1] |
| 266 | OCH₃ | CH₃ | OCH₃ | H | 3 | S | 7 | 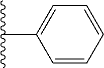 [SMILES: *c1ccccc1] |
| 267 | OCH₃ | CH₃ | OCH₃ | H | 4 | S | 6 | 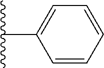 [SMILES: *c1ccccc1] |
| 268 | OCH₃ | CH₃ | OCH₃ | H | 5 | S | 5 | 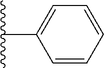 [SMILES: *c1ccccc1] |
| 269 | OCH₃ | CH₃ | OCH₃ | H | 6 | S | 4 | 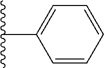 [SMILES: *c1ccccc1] |
| 270 | OCH₃ | CH₃ | OCH₃ | H | 7 | S | 3 | 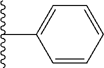 [SMILES: *c1ccccc1] |
| 271 | OCH₃ | CH₃ | OCH₃ | H | 8 | S | 2 | 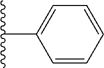 [SMILES: *c1ccccc1] |
| 272 | OCH₃ | CH₃ | OCH₃ | H | 9 | S | 1 | 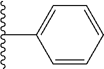 [SMILES: *c1ccccc1] |
| 273 | OCH₃ | CH₃ | OCH₃ | H | 1 | NH | 9 | 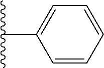 [SMILES: *c1ccccc1] |
| 274 | OCH₃ | CH₃ | OCH₃ | H | 1 | NH | 10 | 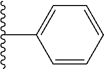 [SMILES: *c1ccccc1] |
| 275 | OCH₃ | CH₃ | OCH₃ | H | 2 | NH | 8 | 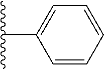 [SMILES: *c1ccccc1] |
| 276 | OCH₃ | CH₃ | OCH₃ | H | 3 | NH | 7 | 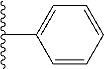 [SMILES: *c1ccccc1] |
| 277 | OCH₃ | CH₃ | OCH₃ | H | 4 | NH | 6 | 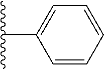 [SMILES: *c1ccccc1] |
| 278 | OCH₃ | CH₃ | OCH₃ | H | 5 | NH | 5 | 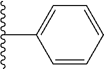 [SMILES: *c1ccccc1] |
| 279 | OCH₃ | CH₃ | OCH₃ | H | 6 | NH | 4 | 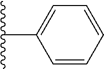 [SMILES: *c1ccccc1] |
| 280 | OCH₃ | CH₃ | OCH₃ | H | 7 | NH | 3 | 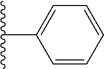 [SMILES: *c1ccccc1] |
| 281 | OCH₃ | CH₃ | OCH₃ | H | 8 | NH | 2 | 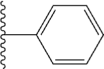 [SMILES: *c1ccccc1] |
| 282 | OCH₃ | CH₃ | OCH₃ | H | 9 | NH | 1 | 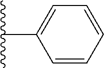 [SMILES: *c1ccccc1] |
| 283 | OCH₃ | CH₃ | OCH₃ | H | 1 | O | 9 | 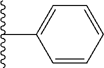 [SMILES: *c1ccccc1] |
| 284 | OCH₃ | CH₃ | OCH₃ | H | 1 | O | 10 | 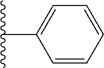 [SMILES: *c1ccccc1] |
| 285 | OCH₃ | CH₃ | OCH₃ | H | 2 | O | 8 | 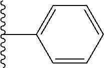 [SMILES: *c1ccccc1] |
| 286 | OCH₃ | CH₃ | OCH₃ | H | 3 | O | 7 | 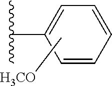 |
| 287 | OCH₃ | CH₃ | OCH₃ | H | 4 | O | 6 | 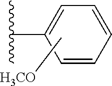 |
| 288 | OCH₃ | CH₃ | OCH₃ | H | 5 | O | 5 | 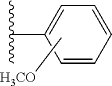 |
| 289 | OCH₃ | CH₃ | OCH₃ | H | 6 | O | 4 | 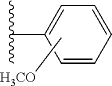 |
| 290 | OCH₃ | CH₃ | OCH₃ | H | 7 | O | 3 | 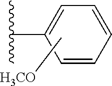 |
| 291 | OCH₃ | CH₃ | OCH₃ | H | 8 | O | 2 | 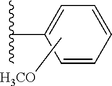 |
| 292 | OCH₃ | CH₃ | OCH₃ | H | 9 | O | 1 | 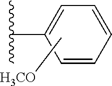 |
| 293 | OCH₃ | CH₃ | OCH₃ | H | 1 | S | 9 | 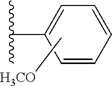 |
| 294 | OCH₃ | CH₃ | OCH₃ | H | 1 | S | 10 | 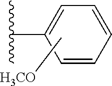 |
| 295 | OCH₃ | CH₃ | OCH₃ | H | 2 | S | 8 | 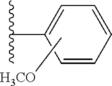 |
| 296 | OCH₃ | CH₃ | OCH₃ | H | 3 | S | 7 | 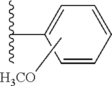 |
| 297 | OCH₃ | CH₃ | OCH₃ | H | 4 | S | 6 | 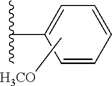 |
| 298 | OCH₃ | CH₃ | OCH₃ | H | 5 | S | 5 | 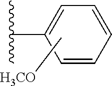 |
| 299 | OCH₃ | CH₃ | OCH₃ | H | 6 | S | 4 | 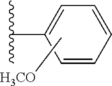 |
| 300 | OCH₃ | CH₃ | OCH₃ | H | 7 | S | 3 | 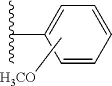 |
| 301 | OCH₃ | CH₃ | OCH₃ | H | 8 | S | 2 | 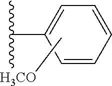 |
| 302 | OCH₃ | CH₃ | OCH₃ | H | 9 | S | 1 | 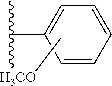 |
| 303 | OCH₃ | CH₃ | OCH₃ | H | 1 | NH | 9 | 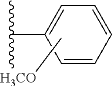 |
| 304 | OCH₃ | CH₃ | OCH₃ | H | 1 | NH | 10 | 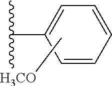 |
| 305 | OCH₃ | CH₃ | OCH₃ | H | 2 | NH | 8 | 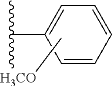 |
| 306 | OCH₃ | CH₃ | OCH₃ | H | 3 | NH | 7 | 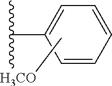 |
| 307 | OCH₃ | CH₃ | OCH₃ | H | 4 | NH | 6 | 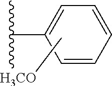 |
| 308 | OCH₃ | CH₃ | OCH₃ | H | 5 | NH | 5 | 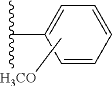 |
| 309 | OCH₃ | CH₃ | OCH₃ | H | 6 | NH | 4 | 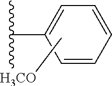 |
| 310 | OCH₃ | CH₃ | OCH₃ | H | 7 | NH | 3 | 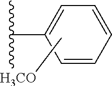 |
| 311 | OCH₃ | CH₃ | OCH₃ | H | 8 | NH | 2 | 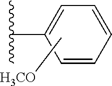 |
| 312 | OCH₃ | CH₃ | OCH₃ | H | 9 | NH | 1 | 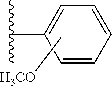 |
| 313 | OCH₃ | CH₃ | OCH₃ | H | 1 | O | 9 | 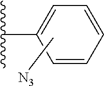 |
| 314 | OCH₃ | CH₃ | OCH₃ | H | 1 | O | 10 | 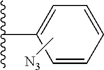 |
| 315 | OCH₃ | CH₃ | OCH₃ | H | 2 | O | 8 | 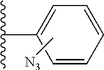 |
| 316 | OCH₃ | CH₃ | OCH₃ | H | 3 | O | 7 | 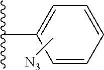 |
| 317 | OCH₃ | CH₃ | OCH₃ | H | 4 | O | 6 | 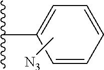 |
| 318 | OCH₃ | CH₃ | OCH₃ | H | 5 | O | 5 | 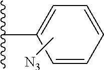 |
| 319 | OCH₃ | CH₃ | OCH₃ | H | 6 | O | 4 | 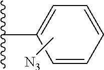 |
| 320 | OCH₃ | CH₃ | OCH₃ | H | 7 | O | 3 | 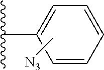 |
| 321 | OCH₃ | CH₃ | OCH₃ | H | 8 | O | 2 | 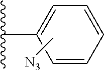 |
| 322 | OCH₃ | CH₃ | OCH₃ | H | 9 | O | 1 | 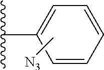 |
| 323 | OCH₃ | CH₃ | OCH₃ | H | 1 | S | 9 | 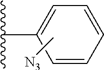 |
| 324 | OCH₃ | CH₃ | OCH₃ | H | 1 | S | 10 | 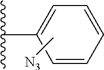 |
| 325 | OCH₃ | CH₃ | OCH₃ | H | 2 | S | 8 | 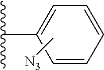 |
| 326 | OCH₃ | CH₃ | OCH₃ | H | 3 | S | 7 | 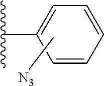 |
| 327 | OCH₃ | CH₃ | OCH₃ | H | 4 | S | 6 | 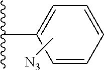 |
| 328 | OCH₃ | CH₃ | OCH₃ | H | 5 | S | 5 | 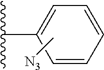 |
| 329 | OCH₃ | CH₃ | OCH₃ | H | 6 | S | 4 | 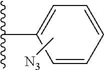 |
| 330 | OCH₃ | CH₃ | OCH₃ | H | 7 | S | 3 | 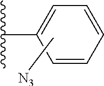 |
| 331 | OCH₃ | CH₃ | OCH₃ | H | 8 | S | 2 | 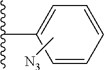 |
| 332 | OCH₃ | CH₃ | OCH₃ | H | 9 | S | 1 | 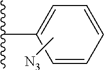 |
| 333 | OCH₃ | CH₃ | OCH₃ | H | 1 | NH | 9 | 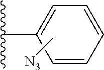 |
| 334 | OCH₃ | CH₃ | OCH₃ | H | 1 | NH | 10 | 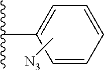 |
| 335 | OCH₃ | CH₃ | OCH₃ | H | 2 | NH | 8 | 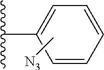 |
| 336 | OCH₃ | CH₃ | OCH₃ | H | 3 | NH | 7 | 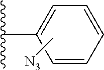 |
| 337 | OCH₃ | CH₃ | OCH₃ | H | 4 | NH | 6 | 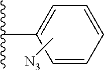 |
| 338 | OCH₃ | CH₃ | OCH₃ | H | 5 | NH | 5 | 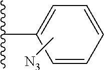 |
| 339 | OCH₃ | CH₃ | OCH₃ | H | 6 | NH | 4 | 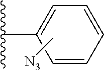 |
| 340 | OCH₃ | CH₃ | OCH₃ | H | 7 | NH | 3 | 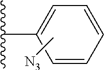 |
| 341 | OCH₃ | CH₃ | OCH₃ | H | 8 | NH | 2 | 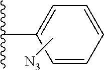 |
| 342 | OCH₃ | CH₃ | OCH₃ | H | 9 | NH | 1 | 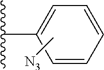 |
| 343 | OCH₃ | CH₃ | OCH₂CH₃ | H | 1 | O | 9 | 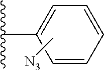 |
| 344 | OCH₃ | CH₃ | OCH₂CH₃ | H | 2 | O | 8 | 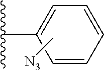 |
| 345 | OCH₃ | CH₃ | OCH₂CH₃ | H | 3 | O | 7 | 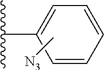 |
| 346 | OCH₃ | CH₃ | OCH₂CH₃ | H | 4 | O | 6 | 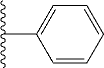 [SMILES: *c1ccccc1] |
| 347 | OCH₃ | CH₃ | OCH₂CH₃ | H | 5 | O | 5 | 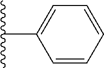 [SMILES: *c1ccccc1] |
| 348 | OCH₃ | CH₃ | OCH₂CH₃ | H | 6 | O | 4 | 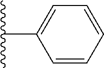 [SMILES: *c1ccccc1] |
| 349 | OCH₃ | CH₃ | OCH₂CH₃ | H | 7 | O | 3 | 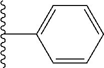 [SMILES: *c1ccccc1] |
| 350 | OCH₃ | CH₃ | OCH₂CH₃ | H | 8 | O | 2 | 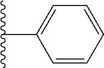 [SMILES: *c1ccccc1] |
| 351 | OCH₃ | CH₃ | OCH₂CH₃ | H | 9 | O | 1 | 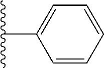 [SMILES: *c1ccccc1] |
| 352 | OCH₃ | CH₃ | OCH₂CH₂CH₃ | H | 1 | O | 9 | 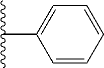 [SMILES: *c1ccccc1] |
| 353 | OCH₃ | CH₃ | OCH₂CH₂CH₃ | H | 2 | O | 8 | 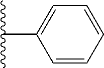 [SMILES: *c1ccccc1] |
| 354 | OCH₃ | CH₃ | OCH₂CH₂CH₃ | H | 3 | O | 7 | 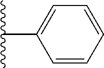 [SMILES: *c1ccccc1] |
| 355 | OCH₃ | CH₃ | OCH₂CH₂CH₃ | H | 4 | O | 6 | 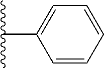 [SMILES: *c1ccccc1] |
| 356 | OCH₃ | CH₃ | OCH₂CH₂CH₃ | H | 5 | O | 5 | 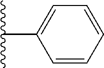 [SMILES: *c1ccccc1] |
| 357 | OCH₃ | CH₃ | OCH₂CH₂CH₃ | H | 6 | O | 4 | 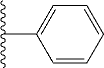 [SMILES: *c1ccccc1] |
| 358 | OCH₃ | CH₃ | OCH₂CH₂CH₃ | H | 7 | O | 3 | 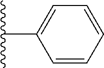 [SMILES: *c1ccccc1] |
| 359 | OCH₃ | CH₃ | OCH₂CH₂CH₃ | H | 8 | O | 2 | 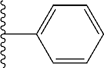 [SMILES: *c1ccccc1] |
| 360 | OCH₃ | CH₃ | OCH₂CH₂CH₃ | H | 9 | O | 1 | 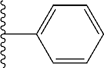 [SMILES: *c1ccccc1] |
| 361 | OCH₂CH₃ | CH₃ | OCH₃ | H | 1 | O | 9 | 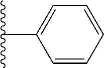 [SMILES: *c1ccccc1] |
| 362 | OCH₂CH₃ | CH₃ | OCH₃ | H | 2 | O | 8 | 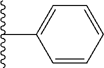 [SMILES: *c1ccccc1] |
| 363 | OCH₂CH₃ | CH₃ | OCH₃ | H | 3 | O | 7 | 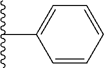 [SMILES: *c1ccccc1] |
| 364 | OCH₂CH₃ | CH₃ | OCH₃ | H | 4 | O | 6 | 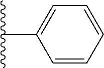 [SMILES: *c1ccccc1] |
| 365 | OCH₂CH₃ | CH₃ | OCH₃ | H | 5 | O | 5 | 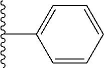 [SMILES: *c1ccccc1] |
| 366 | OCH₂CH₃ | CH₃ | OCH₃ | H | 6 | O | 4 | 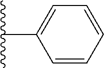 [SMILES: *c1ccccc1] |
| 367 | OCH₂CH₃ | CH₃ | OCH₃ | H | 7 | O | 3 | 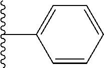 [SMILES: *c1ccccc1] |
| 368 | OCH₂CH₃ | CH₃ | OCH₃ | H | 8 | O | 2 | 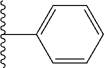 [SMILES: *c1ccccc1] |
| 369 | OCH₂CH₃ | CH₃ | OCH₃ | H | 9 | O | 1 | 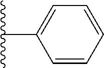 [SMILES: *c1ccccc1] |
| 370 | OCH₂CH₂CH₃ | CH₃ | OCH₃ | H | 1 | O | 9 | 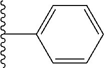 [SMILES: *c1ccccc1] |
| 371 | OCH₂CH₂CH₃ | CH₃ | OCH₃ | H | 2 | O | 8 | 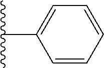 [SMILES: *c1ccccc1] |
| 372 | OCH₂CH₂CH₃ | CH₃ | OCH₃ | H | 3 | O | 7 | 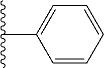 [SMILES: *c1ccccc1] |
| 373 | OCH₂CH₂CH₃ | CH₃ | OCH₃ | H | 4 | O | 6 | 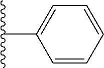 [SMILES: *c1ccccc1] |
| 374 | OCH₂CH₂CH₃ | CH₃ | OCH₃ | H | 5 | O | 5 | 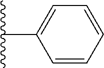 [SMILES: *c1ccccc1] |
| 375 | OCH₂CH₂CH₃ | CH₃ | OCH₃ | H | 6 | O | 4 | 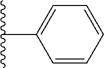 [SMILES: *c1ccccc1] |
| 376 | OCH₂CH₂CH₃ | CH₃ | OCH₃ | H | 7 | O | 3 | 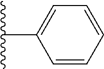 [SMILES: *c1ccccc1] |
| 377 | OCH₂CH₂CH₃ | CH₃ | OCH₃ | H | 8 | O | 2 | 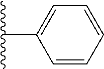 [SMILES: *c1ccccc1] |
| 378 | OCH₂CH₂CH₃ | CH₃ | OCH₃ | H | 9 | O | 1 | 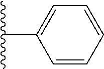 [SMILES: *c1ccccc1] |
| 379 | OCH₃ | SCH₃ | OCH₃ | H | 1 | O | 9 | 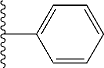 [SMILES: *c1ccccc1] |
| 380 | OCH₃ | SCH₃ | OCH₃ | H | 1 | O | 10 | 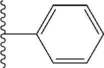 [SMILES: *c1ccccc1] |
| 381 | OCH₃ | SCH₃ | OCH₃ | H | 2 | O | 8 | 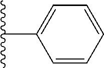 [SMILES: *c1ccccc1] |
| 382 | OCH₃ | SCH₃ | OCH₃ | H | 3 | O | 7 | 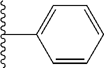 [SMILES: *c1ccccc1] |
| 383 | OCH₃ | SCH₃ | OCH₃ | H | 4 | O | 6 | 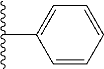 [SMILES: *c1ccccc1] |
| 384 | OCH₃ | SCH₃ | OCH₃ | H | 5 | O | 5 | 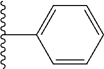 [SMILES: *c1ccccc1] |
| 385 | OCH₃ | SCH₃ | OCH₃ | H | 6 | O | 4 | 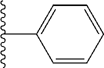 [SMILES: *c1ccccc1] |
| 386 | OCH₃ | SCH₃ | OCH₃ | H | 7 | O | 3 | 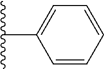 [SMILES: *c1ccccc1] |
| 387 | OCH₃ | SCH₃ | OCH₃ | H | 8 | O | 2 | 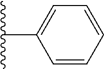 [SMILES: *c1ccccc1] |
| 388 | OCH₃ | SCH₃ | OCH₃ | H | 9 | O | 1 | 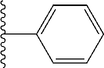 [SMILES: *c1ccccc1] |
| 389 | OCH₃ | SCH₃ | OCH₃ | H | 1 | S | 9 | 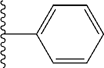 [SMILES: *c1ccccc1] |
| 390 | OCH₃ | SCH₃ | OCH₃ | H | 1 | S | 10 | 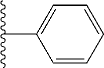 [SMILES: *c1ccccc1] |
| 391 | OCH₃ | SCH₃ | OCH₃ | H | 2 | S | 8 | 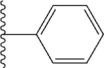 [SMILES: *c1ccccc1] |
| 392 | OCH₃ | SCH₃ | OCH₃ | H | 3 | S | 7 | 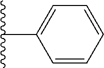 [SMILES: *c1ccccc1] |
| 393 | OCH₃ | SCH₃ | OCH₃ | H | 4 | S | 6 | 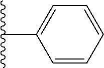 [SMILES: *c1ccccc1] |
| 394 | OCH₃ | SCH₃ | OCH₃ | H | 5 | S | 5 | 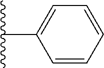 [SMILES: *c1ccccc1] |
| 395 | OCH₃ | SCH₃ | OCH₃ | H | 6 | S | 4 | 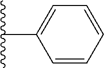 [SMILES: *c1ccccc1] |
| 396 | OCH₃ | SCH₃ | OCH₃ | H | 7 | S | 3 | 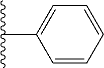 [SMILES: *c1ccccc1] |
| 397 | OCH₃ | SCH₃ | OCH₃ | H | 8 | S | 2 | 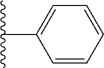 [SMILES: *c1ccccc1] |
| 398 | OCH₃ | SCH₃ | OCH₃ | H | 9 | S | 1 | 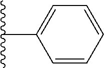 [SMILES: *c1ccccc1] |
| 399 | OCH₃ | SCH₃ | OCH₃ | H | 1 | NH | 9 | 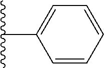 [SMILES: *c1ccccc1] |
| 400 | OCH₃ | SCH₃ | OCH₃ | H | 1 | NH | 10 | 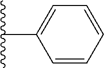 [SMILES: *c1ccccc1] |
| 401 | OCH₃ | SCH₃ | OCH₃ | H | 2 | NH | 8 | 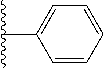 [SMILES: *c1ccccc1] |
| 402 | OCH₃ | SCH₃ | OCH₃ | H | 3 | NH | 7 | 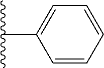 [SMILES: *c1ccccc1] |
| 403 | OCH₃ | SCH₃ | OCH₃ | H | 4 | NH | 6 | 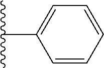 [SMILES: *c1ccccc1] |
| 404 | OCH₃ | SCH₃ | OCH₃ | H | 5 | NH | 5 | 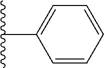 [SMILES: *c1ccccc1] |
| 405 | OCH₃ | SCH₃ | OCH₃ | H | 6 | NH | 4 | 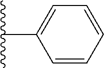 [SMILES: *c1ccccc1] |
| 406 | OCH₃ | SCH₃ | OCH₃ | H | 7 | NH | 3 | 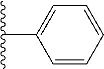 [SMILES: *c1ccccc1] |
| 407 | OCH₃ | SCH₃ | OCH₃ | H | 8 | NH | 2 | 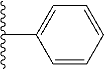 [SMILES: *c1ccccc1] |
| 408 | OCH₃ | SCH₃ | OCH₃ | H | 9 | NH | 1 | 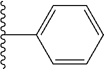 [SMILES: *c1ccccc1] |
| 409 | OCH₃ | SCH₃ | OCH₃ | H | 1 | O | 9 | 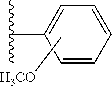 |
| 410 | OCH₃ | SCH₃ | OCH₃ | H | 1 | O | 10 | 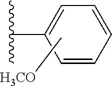 |
| 411 | OCH₃ | SCH₃ | OCH₃ | H | 2 | O | 8 | 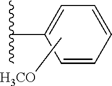 |
| 412 | OCH₃ | SCH₃ | OCH₃ | H | 3 | O | 7 | 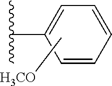 |
| 413 | OCH₃ | SCH₃ | OCH₃ | H | 4 | O | 6 | 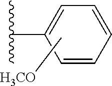 |
| 414 | OCH₃ | SCH₃ | OCH₃ | H | 5 | O | 5 | 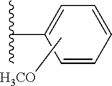 |
| 415 | OCH₃ | SCH₃ | OCH₃ | H | 6 | O | 4 | 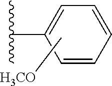 |
| 416 | OCH₃ | SCH₃ | OCH₃ | H | 7 | O | 3 | 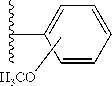 |
| 417 | OCH₃ | SCH₃ | OCH₃ | H | 8 | O | 2 | 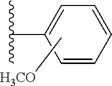 |
| 418 | OCH₃ | SCH₃ | OCH₃ | H | 9 | O | 1 | 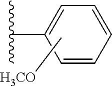 |
| 419 | OCH₃ | SCH₃ | OCH₃ | H | 1 | S | 9 | 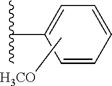 |
| 420 | OCH₃ | SCH₃ | OCH₃ | H | 1 | S | 10 | 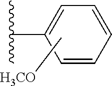 |
| 421 | OCH₃ | SCH₃ | OCH₃ | H | 2 | S | 8 | 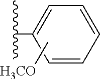 |
| 422 | OCH₃ | SCH₃ | OCH₃ | H | 3 | S | 7 | 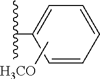 |
| 423 | OCH₃ | SCH₃ | OCH₃ | H | 4 | S | 6 | 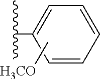 |
| 424 | OCH₃ | SCH₃ | OCH₃ | H | 5 | S | 5 | 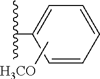 |
| 425 | OCH₃ | SCH₃ | OCH₃ | H | 6 | S | 4 | 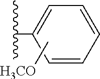 |
| 426 | OCH₃ | SCH₃ | OCH₃ | H | 7 | S | 3 | 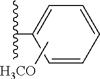 |
| 427 | OCH₃ | SCH₃ | OCH₃ | H | 8 | S | 2 | 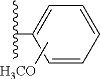 |
| 428 | OCH₃ | SCH₃ | OCH₃ | H | 9 | S | 1 | 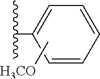 |
| 429 | OCH₃ | SCH₃ | OCH₃ | H | 1 | NH | 9 | 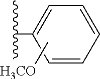 |
| 430 | OCH₃ | SCH₃ | OCH₃ | H | 1 | NH | 10 | 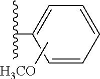 |
| 431 | OCH₃ | SCH₃ | OCH₃ | H | 2 | NH | 8 | 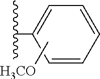 |
| 432 | OCH₃ | SCH₃ | OCH₃ | H | 3 | NH | 7 | 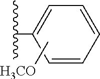 |
| 433 | OCH₃ | SCH₃ | OCH₃ | H | 4 | NH | 6 | 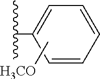 |
| 434 | OCH₃ | SCH₃ | OCH₃ | H | 5 | NH | 5 | 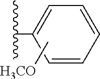 |
| 435 | OCH₃ | SCH₃ | OCH₃ | H | 6 | NH | 4 | 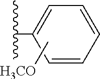 |
| 436 | OCH₃ | SCH₃ | OCH₃ | H | 7 | NH | 3 | 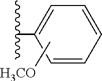 |
| 437 | OCH₃ | SCH₃ | OCH₃ | H | 8 | NH | 2 | 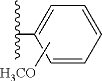 |
| 438 | OCH₃ | SCH₃ | OCH₃ | H | 9 | NH | 1 | 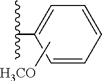 |
| 439 | OCH₃ | SCH₃ | OCH₃ | H | 1 | O | 9 | 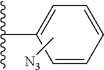 |
| 440 | OCH₃ | SCH₃ | OCH₃ | H | 1 | O | 10 | 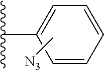 |
| 441 | OCH₃ | SCH₃ | OCH₃ | H | 2 | O | 8 | 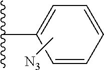 |
| 442 | OCH₃ | SCH₃ | OCH₃ | H | 3 | O | 7 | 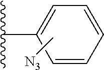 |
| 443 | OCH₃ | SCH₃ | OCH₃ | H | 4 | O | 6 | 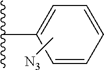 |
| 444 | OCH₃ | SCH₃ | OCH₃ | H | 5 | O | 5 | 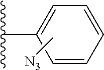 |
| 445 | OCH₃ | SCH₃ | OCH₃ | H | 6 | O | 4 | 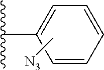 |
| 446 | OCH₃ | SCH₃ | OCH₃ | H | 7 | O | 3 | 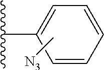 |
| 447 | OCH₃ | SCH₃ | OCH₃ | H | 8 | O | 2 | 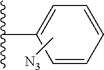 |
| 448 | OCH₃ | SCH₃ | OCH₃ | H | 9 | O | 1 | 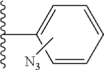 |
| 449 | OCH₃ | SCH₃ | OCH₃ | H | 1 | S | 9 | 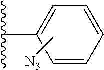 |
| 450 | OCH₃ | SCH₃ | OCH₃ | H | 1 | S | 10 | 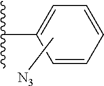 |
| 451 | OCH₃ | SCH₃ | OCH₃ | H | 2 | S | 8 | 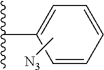 |
| 452 | OCH₃ | SCH₃ | OCH₃ | H | 3 | S | 7 | 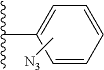 |
| 453 | OCH₃ | SCH₃ | OCH₃ | H | 4 | S | 6 | 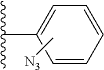 |
| 454 | OCH₃ | SCH₃ | OCH₃ | H | 5 | S | 5 | 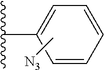 |
| 455 | OCH₃ | SCH₃ | OCH₃ | H | 6 | S | 4 | 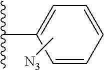 |
| 456 | OCH₃ | SCH₃ | OCH₃ | H | 7 | S | 3 | 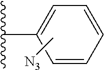 |
| 457 | OCH₃ | SCH₃ | OCH₃ | H | 8 | S | 2 | 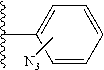 |
| 458 | OCH₃ | SCH₃ | OCH₃ | H | 9 | S | 1 | 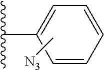 |
| 459 | OCH₃ | SCH₃ | OCH₃ | H | 1 | NH | 9 | 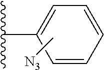 |
| 460 | OCH₃ | SCH₃ | OCH₃ | H | 1 | NH | 10 | 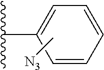 |
| 461 | OCH₃ | SCH₃ | OCH₃ | H | 2 | NH | 8 | 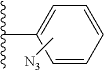 |
| 462 | OCH₃ | SCH₃ | OCH₃ | H | 3 | NH | 7 | 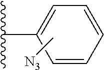 |
| 463 | OCH₃ | SCH₃ | OCH₃ | H | 4 | NH | 6 | 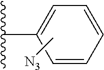 |
| 464 | OCH₃ | SCH₃ | OCH₃ | H | 5 | NH | 5 | 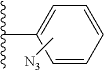 |
| 465 | OCH₃ | SCH₃ | OCH₃ | H | 6 | NH | 4 | 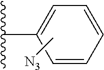 |
| 466 | OCH₃ | SCH₃ | OCH₃ | H | 7 | NH | 3 | 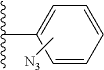 |
| 467 | OCH₃ | SCH₃ | OCH₃ | H | 8 | NH | 2 | 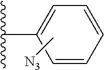 |
| 468 | OCH₃ | SCH₃ | OCH₃ | H | 9 | NH | 1 | 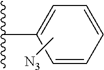 |
| 469 | OCH₃ | SCH₃ | OCH₂CH₃ | H | 1 | O | 9 | 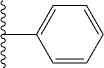 [SMILES: *c1ccccc1] |
| 470 | OCH₃ | SCH₃ | OCH₂CH₃ | H | 2 | O | 8 | 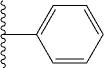 [SMILES: *c1ccccc1] |
| 471 | OCH₃ | SCH₃ | OCH₂CH₃ | H | 3 | O | 7 | 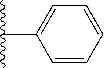 [SMILES: *c1ccccc1] |
| 472 | OCH₃ | SCH₃ | OCH₂CH₃ | H | 4 | O | 6 | 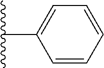 [SMILES: *c1ccccc1] |
| 473 | OCH₃ | SCH₃ | OCH₂CH₃ | H | 5 | O | 5 | 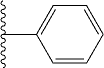 [SMILES: *c1ccccc1] |
| 474 | OCH₃ | SCH₃ | OCH₂CH₃ | H | 6 | O | 4 | 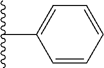 [SMILES: *c1ccccc1] |
| 475 | OCH₃ | SCH₃ | OCH₂CH₃ | H | 7 | O | 3 | 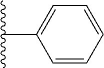 [SMILES: *c1ccccc1] |
| 476 | OCH₃ | SCH₃ | OCH₂CH₃ | H | 8 | O | 2 | 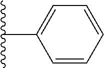 [SMILES: *c1ccccc1] |
| 477 | OCH₃ | SCH₃ | OCH₂CH₃ | H | 9 | O | 1 | 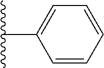 [SMILES: *c1ccccc1] |
| 478 | OCH₃ | SCH₃ | OCH₂CH₂CH₃ | H | 1 | O | 9 | 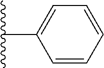 [SMILES: *c1ccccc1] |
| 479 | OCH₃ | SCH₃ | OCH₂CH₂CH₃ | H | 2 | O | 8 | 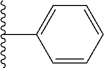 [SMILES: *c1ccccc1] |
| 480 | OCH₃ | SCH₃ | OCH₂CH₂CH₃ | H | 3 | O | 7 | 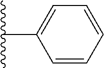 [SMILES: *c1ccccc1] |
| 481 | OCH₃ | SCH₃ | OCH₂CH₂CH₃ | H | 4 | O | 6 | 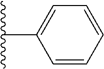 [SMILES: *c1ccccc1] |
| 482 | OCH₃ | SCH₃ | OCH₂CH₂CH₃ | H | 5 | O | 5 | 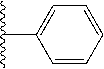 [SMILES: *c1ccccc1] |
| 483 | OCH₃ | SCH₃ | OCH₂CH₂CH₃ | H | 6 | O | 4 | 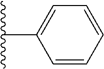 [SMILES: *c1ccccc1] |
| 484 | OCH₃ | SCH₃ | OCH₂CH₂CH₃ | H | 7 | O | 3 | 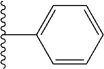 [SMILES: *c1ccccc1] |
| 485 | OCH₃ | SCH₃ | OCH₂CH₂CH₃ | H | 8 | O | 2 | 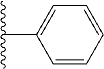 [SMILES: *c1ccccc1] |
| 486 | OCH₃ | SCH₃ | OCH₂CH₂CH₃ | H | 9 | O | 1 | 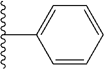 [SMILES: *c1ccccc1] |
| 487 | OCH₂CH₃ | SCH₃ | OCH₃ | H | 1 | O | 9 | 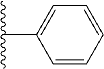 [SMILES: *c1ccccc1] |
| 488 | OCH₂CH₃ | SCH₃ | OCH₃ | H | 2 | O | 8 | 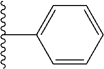 [SMILES: *c1ccccc1] |
| 489 | OCH₂CH₃ | SCH₃ | OCH₃ | H | 3 | O | 7 | 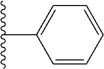 [SMILES: *c1ccccc1] |
| 490 | OCH₂CH₃ | SCH₃ | OCH₃ | H | 4 | O | 6 | 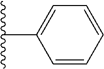 [SMILES: *c1ccccc1] |
| 491 | OCH₂CH₃ | SCH₃ | OCH₃ | H | 5 | O | 5 | 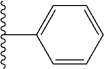 [SMILES: *c1ccccc1] |
| 492 | OCH₂CH₃ | SCH₃ | OCH₃ | H | 6 | O | 4 | 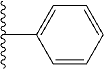 [SMILES: *c1ccccc1] |
| 493 | OCH₂CH₃ | SCH₃ | OCH₃ | H | 7 | O | 3 | 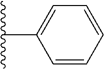 [SMILES: *c1ccccc1] |
| 494 | OCH₂CH₃ | SCH₃ | OCH₃ | H | 8 | O | 2 | 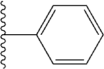 [SMILES: *c1ccccc1] |
| 495 | OCH₂CH₃ | SCH₃ | OCH₃ | H | 9 | O | 1 | 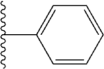 [SMILES: *c1ccccc1] |
| 496 | OCH₂CH₂CH₃ | SCH₃ | OCH₃ | H | 1 | O | 9 | 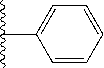 [SMILES: *c1ccccc1] |
| 497 | OCH₂CH₂CH₃ | SCH₃ | OCH₃ | H | 2 | O | 8 | 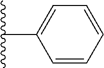 [SMILES: *c1ccccc1] |
| 498 | OCH₂CH₂CH₃ | SCH₃ | OCH₃ | H | 3 | O | 7 | 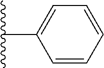 [SMILES: *c1ccccc1] |
| 499 | OCH₂CH₂CH₃ | SCH₃ | OCH₃ | H | 4 | O | 6 | 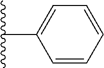 [SMILES: *c1ccccc1] |
| 500 | OCH₂CH₂CH₃ | SCH₃ | OCH₃ | H | 5 | O | 5 | 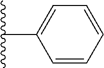 [SMILES: *c1ccccc1] |
| 501 | OCH₂CH₂CH₃ | SCH₃ | OCH₃ | H | 6 | O | 4 | 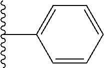 [SMILES: *c1ccccc1] |
| 502 | OCH₂CH₂CH₃ | SCH₃ | OCH₃ | H | 7 | O | 3 | 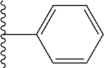 [SMILES: *c1ccccc1] |
| 503 | OCH₂CH₂CH₃ | SCH₃ | OCH₃ | H | 8 | O | 2 | 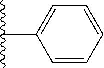 [SMILES: *c1ccccc1] |
| 504 | OCH₂CH₂CH₃ | SCH₃ | OCH₃ | H | 9 | O | 1 | 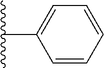 [SMILES: *c1ccccc1] |
| 505 | OCH₃ | I | OCH₃ | CH₃ | 1 | O | 9 | 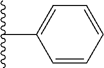 [SMILES: *c1ccccc1] |
| 506 | OCH₃ | I | OCH₃ | CH₃ | 1 | O | 10 | 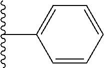 [SMILES: *c1ccccc1] |
| 507 | OCH₃ | I | OCH₃ | CH₃ | 2 | O | 8 | 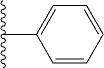 [SMILES: *c1ccccc1] |
| 508 | OCH₃ | I | OCH₃ | CH₃ | 3 | O | 7 | 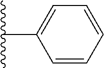 [SMILES: *c1ccccc1] |
| 509 | OCH₃ | I | OCH₃ | CH₃ | 4 | O | 6 | 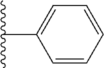 [SMILES: *c1ccccc1] |
| 510 | OCH₃ | I | OCH₃ | CH₃ | 5 | O | 5 | 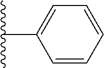 [SMILES: *c1ccccc1] |
| 511 | OCH₃ | I | OCH₃ | CH₃ | 6 | O | 4 | 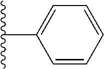 [SMILES: *c1ccccc1] |
| 512 | OCH₃ | I | OCH₃ | CH₃ | 7 | O | 3 | 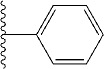 [SMILES: *c1ccccc1] |
| 513 | OCH₃ | I | OCH₃ | CH₃ | 8 | O | 2 | 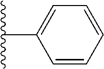 [SMILES: *c1ccccc1] |
| 514 | OCH₃ | I | OCH₃ | CH₃ | 9 | O | 1 | 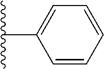 [SMILES: *c1ccccc1] |
| 515 | OCH₃ | I | OCH₃ | CH₃ | 1 | S | 9 | 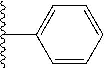 [SMILES: *c1ccccc1] |
| 516 | OCH₃ | I | OCH₃ | CH₃ | 1 | S | 10 | 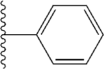 [SMILES: *c1ccccc1] |
| 517 | OCH₃ | I | OCH₃ | CH₃ | 2 | S | 8 | 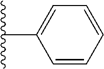 [SMILES: *c1ccccc1] |
| 518 | OCH₃ | I | OCH₃ | CH₃ | 3 | S | 7 | 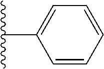 [SMILES: *c1ccccc1] |
| 519 | OCH₃ | I | OCH₃ | CH₃ | 4 | S | 6 | 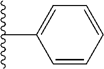 [SMILES: *c1ccccc1] |
| 520 | OCH₃ | I | OCH₃ | CH₃ | 5 | S | 5 | 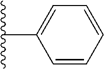 [SMILES: *c1ccccc1] |
| 521 | OCH₃ | I | OCH₃ | CH₃ | 6 | S | 4 | 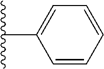 [SMILES: *c1ccccc1] |
| 522 | OCH₃ | I | OCH₃ | CH₃ | 7 | S | 3 | 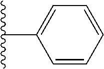 [SMILES: *c1ccccc1] |
| 523 | OCH₃ | I | OCH₃ | CH₃ | 8 | S | 2 | 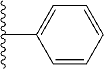 [SMILES: *c1ccccc1] |
| 524 | OCH₃ | I | OCH₃ | CH₃ | 9 | S | 1 | 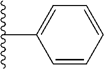 [SMILES: *c1ccccc1] |
| 525 | OCH₃ | I | OCH₃ | CH₃ | 1 | NH | 9 | 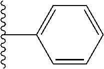 [SMILES: *c1ccccc1] |
| 526 | OCH₃ | I | OCH₃ | CH₃ | 1 | NH | 10 | 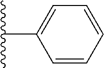 [SMILES: *c1ccccc1] |
| 527 | OCH₃ | I | OCH₃ | CH₃ | 2 | NH | 8 | 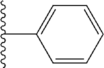 [SMILES: *c1ccccc1] |
| 528 | OCH₃ | I | OCH₃ | CH₃ | 3 | NH | 7 | 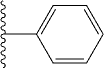 [SMILES: *c1ccccc1] |
| 529 | OCH₃ | I | OCH₃ | CH₃ | 4 | NH | 6 | 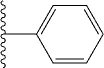 [SMILES: *c1ccccc1] |
| 530 | OCH₃ | I | OCH₃ | CH₃ | 5 | NH | 5 | 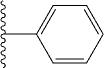 [SMILES: *c1ccccc1] |
| 531 | OCH₃ | I | OCH₃ | CH₃ | 6 | NH | 4 | 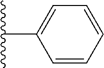 [SMILES: *c1ccccc1] |
| 532 | OCH₃ | I | OCH₃ | CH₃ | 7 | NH | 3 | 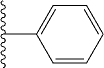 [SMILES: *c1ccccc1] |
| 533 | OCH₃ | I | OCH₃ | CH₃ | 8 | NH | 2 | 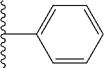 [SMILES: *c1ccccc1] |
| 534 | OCH₃ | I | OCH₃ | CH₃ | 9 | NH | 1 | 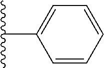 [SMILES: *c1ccccc1] |
| 535 | OCH₃ | I | OCH₃ | CH₃ | 1 | O | 9 | 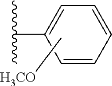 |
| 536 | OCH₃ | I | OCH₃ | CH₃ | 1 | O | 10 | 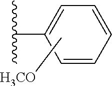 |
| 537 | OCH₃ | I | OCH₃ | CH₃ | 2 | O | 8 | 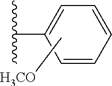 |
| 538 | OCH₃ | I | OCH₃ | CH₃ | 3 | O | 7 | 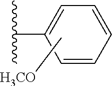 |
| 539 | OCH₃ | I | OCH₃ | CH₃ | 4 | O | 6 | 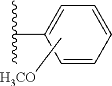 |
| 540 | OCH₃ | I | OCH₃ | CH₃ | 5 | O | 5 | 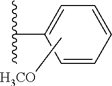 |
| 541 | OCH₃ | I | OCH₃ | CH₃ | 6 | O | 4 | 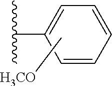 |
| 542 | OCH₃ | I | OCH₃ | CH₃ | 7 | O | 3 | 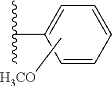 |
| 543 | OCH₃ | I | OCH₃ | CH₃ | 8 | O | 2 | 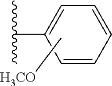 |
| 544 | OCH₃ | I | OCH₃ | CH₃ | 9 | O | 1 | 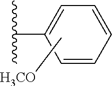 |
| 545 | OCH₃ | I | OCH₃ | CH₃ | 1 | S | 9 | 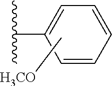 |
| 546 | OCH₃ | I | OCH₃ | CH₃ | 1 | S | 10 | 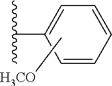 |
| 547 | OCH₃ | I | OCH₃ | CH₃ | 2 | S | 8 | 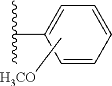 |
| 548 | OCH₃ | I | OCH₃ | CH₃ | 3 | S | 7 | 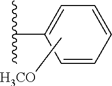 |
| 549 | OCH₃ | I | OCH₃ | CH₃ | 4 | S | 6 | 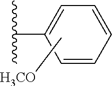 |
| 550 | OCH₃ | I | OCH₃ | CH₃ | 5 | S | 5 | 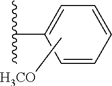 |
| 551 | OCH₃ | I | OCH₃ | CH₃ | 6 | S | 4 | 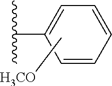 |
| 552 | OCH₃ | I | OCH₃ | CH₃ | 7 | S | 3 | 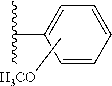 |
| 553 | OCH₃ | I | OCH₃ | CH₃ | 8 | S | 2 | 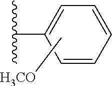 |
| 554 | OCH₃ | I | OCH₃ | CH₃ | 9 | S | 1 | 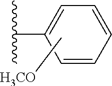 |
| 555 | OCH₃ | I | OCH₃ | CH₃ | 1 | NH | 9 | 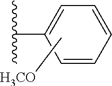 |
| 556 | OCH₃ | I | OCH₃ | CH₃ | 1 | NH | 10 | 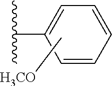 |
| 557 | OCH₃ | I | OCH₃ | CH₃ | 2 | NH | 8 | 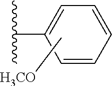 |
| 558 | OCH₃ | I | OCH₃ | CH₃ | 3 | NH | 7 | 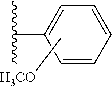 |
| 559 | OCH₃ | I | OCH₃ | CH₃ | 4 | NH | 6 | 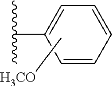 |
| 560 | OCH₃ | I | OCH₃ | CH₃ | 5 | NH | 5 | 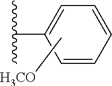 |
| 561 | OCH₃ | I | OCH₃ | CH₃ | 6 | NH | 4 | 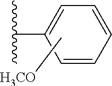 |
| 562 | OCH₃ | I | OCH₃ | CH₃ | 7 | NH | 3 | 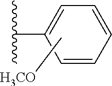 |
| 563 | OCH₃ | I | OCH₃ | CH₃ | 8 | NH | 2 | 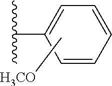 |
| 564 | OCH₃ | I | OCH₃ | CH₃ | 9 | NH | 1 | 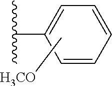 |
| 565 | OCH₃ | I | OCH₃ | CH₃ | 1 | O | 9 | 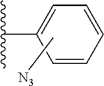 |
| 566 | OCH₃ | I | OCH₃ | CH₃ | 1 | O | 10 | 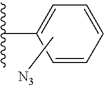 |
| 567 | OCH₃ | I | OCH₃ | CH₃ | 2 | O | 8 | 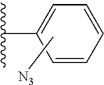 |
| 568 | OCH₃ | I | OCH₃ | CH₃ | 3 | O | 7 | 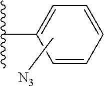 |
| 569 | OCH₃ | I | OCH₃ | CH₃ | 4 | O | 6 | 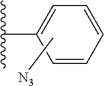 |
| 570 | OCH₃ | I | OCH₃ | CH₃ | 5 | O | 5 | 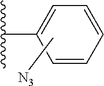 |
| 571 | OCH₃ | I | OCH₃ | CH₃ | 6 | O | 4 | 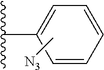 |
| 572 | OCH₃ | I | OCH₃ | CH₃ | 7 | O | 3 | 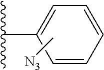 |
| 573 | OCH₃ | I | OCH₃ | CH₃ | 8 | O | 2 | 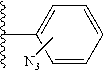 |
| 574 | OCH₃ | I | OCH₃ | CH₃ | 9 | O | 1 | 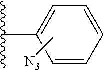 |
| 575 | OCH₃ | I | OCH₃ | CH₃ | 1 | S | 9 | 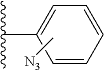 |
| 576 | OCH₃ | I | OCH₃ | CH₃ | 1 | S | 10 | 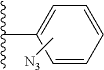 |
| 577 | OCH₃ | I | OCH₃ | CH₃ | 2 | S | 8 | 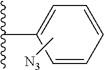 |
| 578 | OCH₃ | I | OCH₃ | CH₃ | 3 | S | 7 | 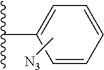 |
| 579 | OCH₃ | I | OCH₃ | CH₃ | 4 | S | 6 | 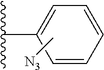 |
| 580 | OCH₃ | I | OCH₃ | CH₃ | 5 | S | 5 | 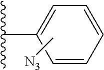 |
| 581 | OCH₃ | I | OCH₃ | CH₃ | 6 | S | 4 | 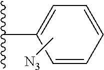 |
| 582 | OCH₃ | I | OCH₃ | CH₃ | 7 | S | 3 | 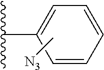 |
| 583 | OCH₃ | I | OCH₃ | CH₃ | 8 | S | 2 | 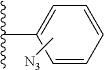 |
| 584 | OCH₃ | I | OCH₃ | CH₃ | 9 | S | 1 | 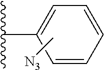 |
| 585 | OCH₃ | I | OCH₃ | CH₃ | 1 | NH | 9 | 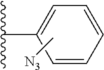 |
| 586 | OCH₃ | I | OCH₃ | CH₃ | 1 | NH | 10 | 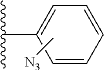 |
| 587 | OCH₃ | I | OCH₃ | CH₃ | 2 | NH | 8 | 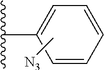 |
| 588 | OCH₃ | I | OCH₃ | CH₃ | 3 | NH | 7 | 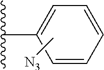 |
| 589 | OCH₃ | I | OCH₃ | CH₃ | 4 | NH | 6 | 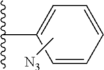 |
| 590 | OCH₃ | I | OCH₃ | CH₃ | 5 | NH | 5 | 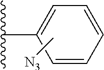 |
| 591 | OCH₃ | I | OCH₃ | CH₃ | 6 | NH | 4 | 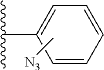 |
| 592 | OCH₃ | I | OCH₃ | CH₃ | 7 | NH | 3 | 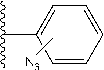 |
| 593 | OCH₃ | I | OCH₃ | CH₃ | 8 | NH | 2 | 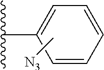 |
| 594 | OCH₃ | I | OCH₃ | CH₃ | 9 | NH | 1 | 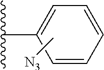 |
| 595 | OCH₃ | I | OCH₂CH₃ | CH₃ | 1 | O | 9 | 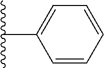 [SMILES: *c1ccccc1] |
| 596 | OCH₃ | I | OCH₂CH₃ | CH₃ | 2 | O | 8 | 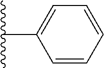 [SMILES: *c1ccccc1] |
| 597 | OCH₃ | I | OCH₂CH₃ | CH₃ | 3 | O | 7 | 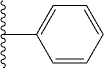 [SMILES: *c1ccccc1] |
| 598 | OCH₃ | I | OCH₂CH₃ | CH₃ | 4 | O | 6 | 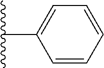 [SMILES: *c1ccccc1] |
| 599 | OCH₃ | I | OCH₂CH₃ | CH₃ | 5 | O | 5 | 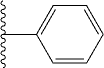 [SMILES: *c1ccccc1] |
| 600 | OCH₃ | I | OCH₂CH₃ | CH₃ | 6 | O | 4 | 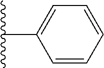 [SMILES: *c1ccccc1] |
| 601 | OCH₃ | I | OCH₂CH₃ | CH₃ | 7 | O | 3 | 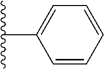 [SMILES: *c1ccccc1] |
| 602 | OCH₃ | I | OCH₂CH₃ | CH₃ | 8 | O | 2 | 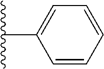 [SMILES: *c1ccccc1] |
| 603 | OCH₃ | I | OCH₂CH₃ | CH₃ | 9 | O | 1 | 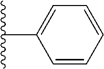 [SMILES: *c1ccccc1] |
| 604 | OCH₃ | I | OCH₂CH₂CH₃ | CH₃ | 1 | O | 9 | 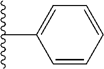 [SMILES: *c1ccccc1] |
| 605 | OCH₃ | I | OCH₂CH₂CH₃ | CH₃ | 2 | O | 8 | 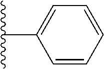 [SMILES: *c1ccccc1] |
| 606 | OCH₃ | I | OCH₂CH₂CH₃ | CH₃ | 3 | O | 7 | 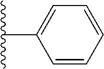 [SMILES: *c1ccccc1] |
| 607 | OCH₃ | I | OCH₂CH₂CH₃ | CH₃ | 4 | O | 6 | 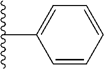 [SMILES: *c1ccccc1] |
| 608 | OCH₃ | I | OCH₂CH₂CH₃ | CH₃ | 5 | O | 5 | 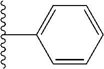 [SMILES: *c1ccccc1] |
| 609 | OCH₃ | I | OCH₂CH₂CH₃ | CH₃ | 6 | O | 4 | 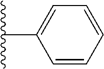 [SMILES: *c1ccccc1] |
| 610 | OCH₃ | I | OCH₂CH₂CH₃ | CH₃ | 7 | O | 3 | 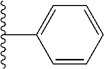 [SMILES: *c1ccccc1] |
| 611 | OCH₃ | I | OCH₂CH₂CH₃ | CH₃ | 8 | O | 2 | 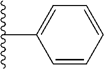 [SMILES: *c1ccccc1] |
| 612 | OCH₃ | I | OCH₂CH₂CH₃ | CH₃ | 9 | O | 1 | 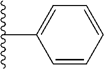 [SMILES: *c1ccccc1] |
| 613 | OCH₂CH₃ | I | OCH₃ | CH₃ | 1 | O | 9 | 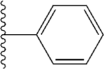 [SMILES: *c1ccccc1] |
| 614 | OCH₂CH₃ | I | OCH₃ | CH₃ | 2 | O | 8 | 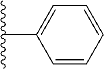 [SMILES: *c1ccccc1] |
| 615 | OCH₂CH₃ | I | OCH₃ | CH₃ | 3 | O | 7 | 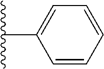 [SMILES: *c1ccccc1] |
| 616 | OCH₂CH₃ | I | OCH₃ | CH₃ | 4 | O | 6 | 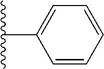 [SMILES: *c1ccccc1] |
| 617 | OCH₂CH₃ | I | OCH₃ | CH₃ | 5 | O | 5 | 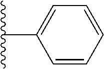 [SMILES: *c1ccccc1] |
| 618 | OCH₂CH₃ | I | OCH₃ | CH₃ | 6 | O | 4 | 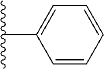 [SMILES: *c1ccccc1] |
| 619 | OCH₂CH₃ | I | OCH₃ | CH₃ | 7 | O | 3 | 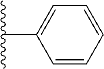 [SMILES: *c1ccccc1] |
| 620 | OCH₂CH₃ | I | OCH₃ | CH₃ | 8 | O | 2 | 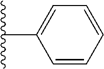 [SMILES: *c1ccccc1] |
| 621 | OCH₂CH₃ | I | OCH₃ | CH₃ | 9 | O | 1 | 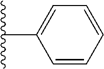 [SMILES: *c1ccccc1] |
| 622 | OCH₂CH₂CH₃ | I | OCH₃ | CH₃ | 1 | O | 9 | 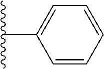 [SMILES: *c1ccccc1] |
| 623 | OCH₂CH₂CH₃ | I | OCH₃ | CH₃ | 2 | O | 8 | 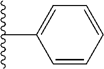 [SMILES: *c1ccccc1] |
| 624 | OCH₂CH₂CH₃ | I | OCH₃ | CH₃ | 3 | O | 7 | 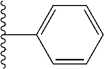 [SMILES: *c1ccccc1] |
| 625 | OCH₂CH₂CH₃ | I | OCH₃ | CH₃ | 4 | O | 6 | 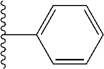 [SMILES: *c1ccccc1] |
| 626 | OCH₂CH₂CH₃ | I | OCH₃ | CH₃ | 5 | O | 5 | 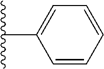 [SMILES: *c1ccccc1] |
| 627 | OCH₂CH₂CH₃ | I | OCH₃ | CH₃ | 6 | O | 4 | 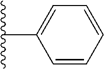 [SMILES: *c1ccccc1] |
| 628 | OCH₂CH₂CH₃ | I | OCH₃ | CH₃ | 7 | O | 3 | 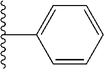 [SMILES: *c1ccccc1] |
| 629 | OCH₂CH₂CH₃ | I | OCH₃ | CH₃ | 8 | O | 2 | 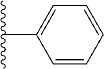 [SMILES: *c1ccccc1] |
| 630 | OCH₂CH₂CH₃ | I | OCH₃ | CH₃ | 9 | O | 1 | 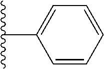 [SMILES: *c1ccccc1] |

In some embodiments, the compound is selected from the group consisting of:

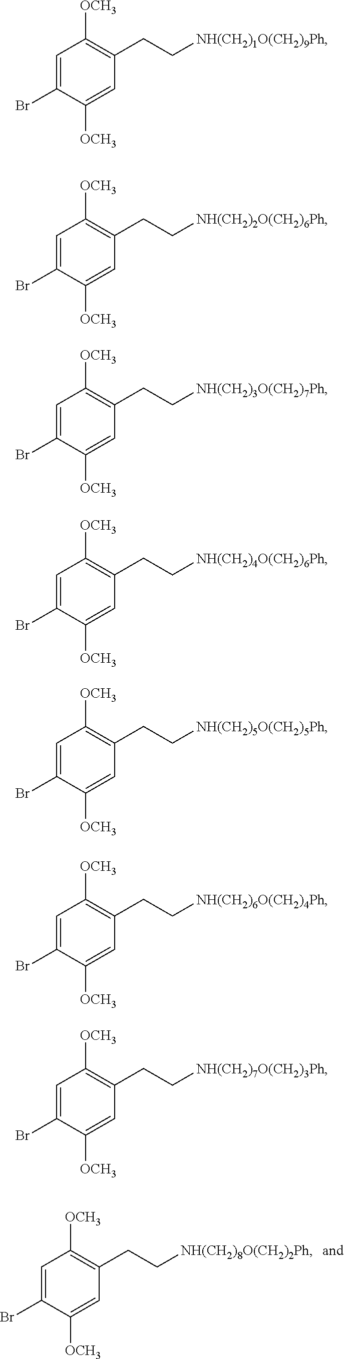

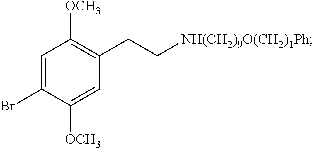

or a pharmaceutically acceptable salt thereof.

In some embodiments, the compound is

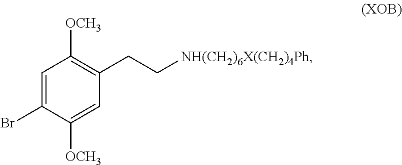

or a pharmaceutically acceptable salt thereof.

The individual compounds of the disclosed compositions will be understood to also encompass pharmaceutically acceptable salts of such compounds. The term “pharmaceutically acceptable salt” refers to salts prepared from pharmaceutically acceptable non-toxic acids or bases, and which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable. One of ordinary skill in the art can select from among a wide variety of available counterions those that are pharmaceutically acceptable. In specific applications, the selection of a given anion or cation for preparation of a salt may result in increased or decreased solubility of that salt. Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, 1-aspartate, 1-camsylate, 1-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like. (See Berge, et al., J. Pharm. Sci. 1997, 66, 1-19.)

Certain compounds disclosed herein contain one or more ionizable groups (groups from which a proton can be removed (e.g., —COOH) or added (e.g., amines) or which can be quaternized (e.g., amines)). All possible ionic forms of such molecules and salts thereof are included in the present disclosure.

A compound described herein can exist in solid or liquid form. In the solid state, the compound may exist in crystalline or noncrystalline form, or as a mixture thereof. The skilled artisan will appreciate that pharmaceutically acceptable solvates may be formed for crystalline or non-crystalline compounds. In crystalline solvates, solvent molecules are incorporated into the crystalline lattice during crystallization. Solvates may involve non-aqueous solvents such as, but not limited to, ethanol, isopropanol, DMSO, acetic acid, ethanolamine, or ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The subject matter described herein includes such solvates.

The skilled artisan will further appreciate that certain compounds described herein that exist in crystalline form, including the various solvates thereof, may exhibit polymorphism (i.e. the capacity to occur in different crystalline structures). These different crystalline forms are typically known as “polymorphs.” The subject matter disclosed herein includes such polymorphs. Polymorphs include the different crystal packing arrangements of the same elemental composition of a compound. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. Various factors such as the recrystallization solvent, rate of crystallization, and storage temperature may cause a single crystal form to dominate. In addition, one polymorph may spontaneously convert to another polymorph under certain conditions.

The compounds described herein may contain one or more asymmetric centers and give rise to enantiomers, diastereomers, and other stereoisomeric forms. Each chiral center may be defined, in terms of absolute stereochemistry, as (R)- or (S)-. The invention includes all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. Various methods are known in the art for preparing optically active forms and determining activity. Such methods include standard tests described herein and other similar tests which are well known in the art. Examples of methods that can be used to obtain optical isomers of the compounds according to the present disclosure include selective crystallization, enzymatic resolution, asymmetric synthesis (including asymmetric chemical synthesis and asymmetric enzymatic synthesis), kinetic resolution, and chiral chromatography (including chiral liquid chromatography, gas chromatography, and high-performance liquid chromatography). When the compounds described herein contain olefinic double bonds or other centers of geometric asymmetry, and unless specified otherwise, it is intended that the compounds include both E and Z geometric isomers. Likewise, tautomeric forms are included.

Also provided are compounds with at least one desired isotopic substitution of an atom, at an amount above the natural abundance of the isotope, i.e., isotopically enriched analogs of disclosed compounds. Isotopes are atoms having the same atomic number but different mass numbers, i.e., the same number of protons but a different number of neutrons. Examples of isotopes that can be incorporated into compounds described herein include isotopes of hydrogen, carbon, nitrogen, oxygen, and chlorine such as ²H, ³H, ¹¹C, ¹³C, ¹⁴C, ¹⁵N, ¹⁷O, ¹⁸O, and ³⁶Cl respectively. In one non-limiting embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F-labeled compound may be particularly desirable for PET or SPECT studies. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of this invention can generally be prepared by carrying out the procedures disclosed in the schemes or in the examples and preparations described below by substituting a readily available isotopically labeled reagent for a non-isotopically labeled reagent.

Also provided are prodrugs of disclosed compounds. A “prodrug” is a precursor of a biologically active pharmaceutical agent, which may undergo a chemical or a metabolic conversion to become the biologically active agent. A prodrug can be converted ex vivo to the biologically active pharmaceutical agent by chemical transformative processes. In vivo, a prodrug is converted to the biologically active pharmaceutical agent by the action of a metabolic process, an enzymatic process or a degradative process that removes the prodrug moiety to form the biologically active pharmaceutical agent. Typical examples of prodrugs include compounds with biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Commonly used functional groups include esters, carbonates, carbamates, amides, phosphates, and sulfonamides. These functional groups can be attached to the drug molecule via a linker that is designed to be cleaved under specific physiological conditions, such as enzymatic hydrolysis or pH-dependent cleavage. The choice of functional group depends on factors such as stability, ease of synthesis, enzymatic activity, and desired rate of prodrug conversion.

Generally, disclosed compounds are administered as part of a pharmaceutical composition or formulation, and are prepared for inclusion in such composition or formulation as isolated or purified compounds. The terms “isolated,” “purified,” or “substantially pure,” as used herein, refer to material that is substantially or essentially free from components that normally accompany the material when the material is synthesized, manufactured, or otherwise produced. An “isolated,” “purified,” or “substantially pure” preparation of a compound is accordingly defined as a preparation having a chromatographic purity (of the desired compound) of greater than 90%, more preferably greater than 95%, more preferably greater than 96%, more preferably greater than 97%, more preferably greater than 98%, more preferably greater than 99%, more preferably greater than 99.5%, and most preferably greater than 99.9%, as determined by area normalization of an HPLC profile or other similar detection method.

Preferably, a substantially pure compound of the disclosure is substantially free of any other active compounds which are not intended to be administered to a subject. In this context “substantially free” can be taken to mean that no active compound(s) other than the active compound intended to be administered to a subject are detectable by HPLC or other similar detection method, or are below a desired threshold of detection such as defined above.

### C. PHARMACEUTICAL COMPOSITIONS

In some aspects, provided herein are compositions, such as pharmaceutical compositions, comprising a disclosed compound, such as a compound of any disclosed Formulae or subformula thereof. “Pharmaceutical compositions” are compositions comprising disclosed compound(s) together in an amount (for example, in a unit dosage form) with a pharmaceutically acceptable carrier, diluent, or excipient. Some embodiments will not have a single carrier, diluent, or excipient alone, but will include multiple carriers, diluents, and/or excipients. Compositions can be prepared by standard pharmaceutical formulation techniques as disclosed in, e.g., Remington: The Science & Practice of Pharmacy (2020) 23th ed., Academic Press., Cambridge, Mass.; The Merck Index (1996) 12th ed., Merck Pub. Group, Whitehouse, N.J.; Pharm. Principles of Solid Dosage Forms (1993), Technomic Pub. Co., Inc., Lancaster, Pa.; and Ansel & Stoklosa, Pharm. Calculations (2001) 11th ed., Lippincott Williams & Wilkins, Baltimore, Md.; & Poznansky et al. Drug Delivery Systems (1980), R. L. Juliano, ed., Oxford, N.Y., pp. 253-315).

“Pharmaceutically acceptable” used in connection with an excipient, carrier, diluent, or other ingredient means the ingredient is generally safe and, within the scope of sound medical judgment, suitable for use in contact with cells of humans and animals without undue toxicity, irritation, allergic response, or complication, commensurate with a reasonable risk/benefit ratio.

In some embodiments, pharmaceutical compositions comprising a disclosed compound can be administered by a variety of routes including oral, mucosal (e.g., buccal, sublingual), rectal, transdermal, subcutaneous, intravenous, intramuscular, inhaled, and intranasal. In some embodiments, the compounds employed in the methods of this invention are effective as oral, mucosal (e.g., buccal, sublingual), rectal, transdermal, subcutaneous, intravenous, intramuscular, inhaled, and intranasal compositions. Such compositions are prepared in a manner well known in the pharmaceutical art and comprise at least one active compound. (See, e.g., Remington, 2020.)

The disclosed compositions are preferably formulated in a unit dosage form, each dosage containing a therapeutically effective amount of the active ingredients, for example in the dosage amounts disclosed below. The term “unit dosage form” refers to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose for a “booster” dose as described below), of the pharmaceutical composition administered.

Unit dosage forms include capsules, troches, cachets, lozenges, tablets, ampules and vials, which may include a composition in a freeze-dried or lyophilized state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis (including the mucosa) of a subject for an extended or brief period of time.

In some embodiments, the disclosed compositions are formulated in a pharmaceutically acceptable oral dosage form. Oral dosage forms include oral liquid dosage forms (such as tinctures, drops, emulsions, syrups, elixirs, suspensions, and solutions, and the like) and oral solid dosage forms. The disclosed pharmaceutical compositions also may be prepared as formulations suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions.

In some embodiments, a disclosed composition is formulated as an oral solid dosage form. Oral solid dosage forms may include but are not limited to, lozenges, troches, tablets, capsules, caplets, powders, pellets, multiparticulates, beads, spheres, and/or any combinations thereof. Oral solid dosage forms may be formulated as immediate release, controlled release, sustained release, extended release, or modified release formulations. Accordingly, in some embodiments, the disclosed oral solid dosage forms may be in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including a fast-melt tablet. Additionally, pharmaceutical formulations may be administered as a single capsule or in multiple capsule dosage form. In some embodiments, the pharmaceutical formulation is administered in two, three, four, or more capsules or tablets.

Oral solid dosage forms may contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Oral solid dosage forms also can comprise one or more pharmaceutically acceptable additives such as a compatible carrier, complexing agent, ionic dispersion modulator, disintegrating agent, surfactant, lubricant, colorant, moistening agent, plasticizer, stabilizer, penetration enhancer, wetting agent, anti-foaming agent, alone or in combination, as well as supplementary active compound(s).

Supplementary active compounds include preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of the formulation. Suitable preservatives are known in the art and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid.

In some embodiments, a disclosed composition is formulated as an oral liquid dosage form. Oral liquid dosage forms include tinctures, drops, emulsions, syrups, elixirs, suspensions, and solutions, and the like. These oral liquid dosage forms may be formulated with any pharmaceutically acceptable excipient known to those of skill in the art for the preparation of liquid dosage forms, and with solvents, diluents, carriers, excipients, and the like chosen as appropriate to the solubility and other properties of the active agents and other ingredients. Solvents may be, for example, water, glycerin, simple syrup, alcohol, medium chain triglycerides (MCT), and combinations thereof.

Liquid dosage forms for oral administration may be in the form of pharmaceutically acceptable emulsions, syrups, elixirs, suspensions, and solutions, which may contain an inactive diluent, such as water. Pharmaceutical formulations may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Liquid formulations also may be prepared as single dose or multi-dose beverages. Suspensions may include oils. Such oils include peanut oil, sesame oil, cottonseed oil, corn oil, and olive oil. Suitable oils also include carrier oils such as MCT and long chain triglyceride (LCT) oils. Suspension preparation may also contain esters of fatty acids such as ethyl oleate, isopropyl myristate, fatty acid glycerides, and acetylated fatty acid glycerides. Suspension formulations may include alcohols, (such as ethanol, isopropyl alcohol, hexadecyl alcohol), glycerol, and propylene glycol. Ethers, such as poly(ethylene glycol), petroleum hydrocarbons such as mineral oil and petrolatum, and water may also be used in suspension formulations. Suspension can thus include an aqueous liquid or a non-aqueous liquid, an oil-in-water liquid emulsion, or a water-in-oil emulsion.

In some embodiments, formulations are provided comprising the disclosed compositions and at least one dispersing agent or suspending agent for oral administration to a subject. The formulation may be a powder and/or granules for suspension, and upon admixture with water, a substantially uniform suspension is obtained. The aqueous dispersion can comprise amorphous and non-amorphous particles consisting of multiple effective particle sizes such that a drug is absorbed in a controlled manner over time.

Dosage forms for oral administration can be aqueous suspensions selected from the group including pharmaceutically acceptable aqueous oral dispersions, emulsions, solutions, and syrups. See, e.g., Singh et al., Encyclopedia of Pharm. Tech., 2nd Ed., 754-757 (2002). In addition to the disclosed compounds, the liquid dosage forms may comprise additives, such as one or more (a) disintegrating agents, (b) dispersing agents, (c) wetting agents, (d) preservatives, (e) viscosity enhancing agents, (f) sweetening agents, or (g) flavoring agents.

Disclosed compositions also may be prepared as formulations suitable for intramuscular, subcutaneous, intraperitoneal, or intravenous injection, comprising physiologically acceptable sterile aqueous or non-aqueous solutions, dispersions, suspensions or emulsions, liposomes, and sterile powders for reconstitution into sterile injectable solutions or dispersions.

In other embodiments, disclosed pharmaceutical compositions may be formulated into a topical dosage form. Topical dosage forms include transmucosal and transdermal formulations, such as aerosols, emulsions, sprays, ointments, salves, gels, pastes, lotions, liniments, oils, and creams. For such formulations, penetrants and carriers can be included in the pharmaceutical composition. Penetrants are known in the art, and include, for transmucosal administration, detergents, bile salts, and fusidic acid derivatives. For transdermal administration, carriers which may be used include Vaseline®, lanolin, PEG, alcohols, transdermal enhancers, and combinations thereof.

### D. PHARMACEUTICAL COMBINATIONS

It should be readily appreciated that the disclosed compositions are not limited to combinations of a single compound, or (when formulated as a pharmaceutical composition) limited to a single carrier, diluent, and/or excipient alone, but may also include combinations of multiple compounds (including additional active compounds), and/or multiple carriers, diluents, and excipients. Pharmaceutical compositions of this invention thus may comprise a compound of Formula (I) together with one or more other active agents (or their derivatives and analogs) in combination, together with one or more pharmaceutically-acceptable carriers, diluents, and/or excipients, and additionally with one or more other active compounds.

In some embodiments, a formulation of the invention will be prepared so as to increase an existing therapeutic effect, provide an additional therapeutic effect, increase a desired property such as stability or shelf-life, decrease an unwanted effect or property, alter a property in a desirable way (such as pharmacokinetics or pharmacodynamics), modulate a desired system or pathway (e.g., a neurotransmitter system), or provide synergistic effects.

“Therapeutic effects” that may be increased or added in embodiments of the invention include, but are not limited to, antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, empathogenic, psychedelic, sedative, and stimulant effects.

“Synergistic effects” should be understood to include increases in potency, bioactivity, bioaccessibility, bioavailability, or therapeutic effect, that are greater than the additive contributions of the components acting alone. Numerous methods known to those of skill in the art exist to determine whether there is synergy as to a particular effect, i.e., whether, when two or more components are mixed together, the effect is greater than the sum of the effects of the individual components applied alone, thereby producing “1+1>2.” Suitable methods include isobologram (or contour) analysis (Huang, Front Pharmacol., 2019; 10:1222), or the equation of Loewe additivity (Loewe & Muischnek, 1926, Arch. Exp. Pathol Pharmacol. 114:313-326). A synergistic effect also may be calculated using methods such as the Sigmoid-Emax equation (Holford & Scheiner, 1981, Clin. Pharmacokinet. 6:429-453) and the median-effect equation (Chou & Talalay, 1984, Adv. Enzyme Regul. 22:27-55). The corresponding graphs associated with the equations referred to above are the concentration-effect curve and combination index curve, respectively. Each equation referred to above may be applied to experimental data to generate a corresponding graph to aid in assessing the effects of the drug combination.

In some embodiments, a disclosed pharmaceutical composition comprises an additional active compound. In some embodiments, the additional active compound is selected from the group consisting of: amino acids, antioxidants, anti-inflammatory agents, analgesics, antineuropathic and antinociceptive agents, antimigraine agents, anxiolytics, antidepressants, antipsychotics, anti-PTSD agents, dissociatives, cannabinoids, immunostimulants, anti-cancer agents, antiemetics, orexigenics, antiulcer agents, antihistamines, antihypertensives, anticonvulsants, antiepileptics, bronchodilators, neuroprotectants, nootropics, empathogens, psychedelics, plasticity-inducing agents (e.g., psychoplastogens), monoamine oxidase inhibitors, tryptamines, terpenes, phenethylamines, sedatives, stimulants, serotonergic agents, and vitamins. In some embodiments, the additional active compound acts to increase a therapeutic effect, provide an additional therapeutic effect, decrease an unwanted effect, increase stability or shelf-life, improve bioavailability, induce synergy, increase plasticity (e.g., neural plasticity), or alter pharmacokinetics or pharmacodynamics. In some embodiments, the additional therapeutic effect is an antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, empathogenic, psychedelic, sedative, or stimulant effect.

In embodiments, an additional active compound is a tryptamine. As will be understood by those in the art, tryptamines are compounds having the general structure below, wherein R^{N1}, R^{N2}, R^{α}, R^{β}, R², R⁴, R⁵, R⁶, and R⁷ are as defined herein and as generally understood in the art:

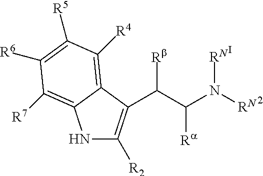

In some embodiments, R^{N1}, R^{N2}, R^{α}, R^{β}, R², R⁴, R⁵, R⁶, and R⁷ are each independently hydrogen, deuterium, halogen (F, Cl, Br, or I), OH, phosphoryloxy, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl. Additionally, any two of R^{N1}, R^{N2}, R^{α}, R^{β}, R², R⁴, R⁵, R⁶, and R7 and the intervening atoms can be taken together to form an optionally substituted cycloalkyl, optionally substituted cycloalkenyl, optionally substituted aryl, or optionally substituted heterocyclyl. In embodiments, the tryptamine is a quaternary salt, in which an additional R^{N3} is connected to the nitrogen to which R^{N1} and R^{N2} are bound; wherein R^{N3} is optionally substituted alkyl, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl.

In some embodiments, the additional active compound is a tryptamine selected from the group consisting of O-Phosphoryl-4-hydroxy-N,N-dimethyltryptamine (psilocybin), 6-allyl-N,N-diethyl-norlysergamide (AL-LAD), N,N-dibutyltryptamine (DBT), N,N-diethyltryptamine (DET), N,N-diisopropyltryptamine (DiPT), 5-methoxy-α-methyltryptamine (α,O-DMS), N,N-dimethyl-tryptamine (DMT), 2,α-dimethyltryptamine (2,α-DMT), α,N-dimethyltryptamine (α,N-DMT), N,N-dipropyltryptamine (DPT), N-ethyl-N-isopropyltryptamine (EiPT), α-ethyltryptamine (AET), 6,N,N-triethylnorlysergamide (ETH-LAD), 3,4-dihydro-7-methoxy-1-methylcarboline (Harmaline), 7-methoxy-1-methylcarboline (Harmine), N,N-dibutyl-4-hydroxytryptamine (4-HO-DBT), N,N-diethyl-4-hydroxytryptamine (4-HO-DET), N,N-diisopropyl-4-hydroxytryptamine (4-HO-DiPT), 4-hydroxy-N,N,N-trimethyltryptamine (4-HO-TMT), N,N-dimethyl-4-hydroxytryptamine (4-HO-DMT), N,N-dimethyl-5-hydroxytryptamine (5-HO-DMT, bufotenine), N,N-dipropyl-4-hydroxytryptamine (4-HO-DPT), N-ethyl-4-hydroxy-N-methyltryptamine (4-HO-MET), 4-hydroxy-N-isopropyl-N-methyltryptamine (4-HO-MiPT), 4-hydroxy-N-methyl-N-propyl-tryptamine (4-HO-MPT), 4-hydroxy-N,N-tetramethylenetryptamine (4-HO-pyr-T), 12-methoxyibogamine (Ibogaine), N-butyl-N-methyltryptamine (MBT), N,N-diisopropyl-4,5-methylenedioxytryptamine (4,5-MDO-DiPT), N,N-diisopropyl-5,6-methylenedioxytryptamine (5,6-MDO-DiPT), N,N-dimethyl-4,5-methylenedioxytryptamine (4,5-MDO-DMT), N,N-dimethyl-5,6-methylenedioxytryptamine (5,6-MDO-DMT), N-isopropyl-N-methyl-5,6-methylenedioxytryptamine (5,6-MDO-MiPT), N,N-diethyl-2-methyltryptamine (2-Me-DET), 2, N,N-trimethyltryptamine (2-Me-DMT), N-acetyl-5-methoxytryptamine (melatonin), N,N-diethyl-5-methoxytryptamine (5-MeO-DET), N,N-diisopropyl-5-methoxytryptamine (5-MeO-DiPT), N,N,diallyl-5-methoxytryptamine (5-MeO-DALT), 5-methoxy-N,N-dimethyltryptamine (5-MeO-DMT), N-isopropyl-4-methoxy-N-methyltryptamine (4-MeO-MiPT), N-isopropyl-5-methoxy-N-methyltryptamine (5-MeO-MiPT), 5,6-dimethoxy-N-isopropyl-N-methyltryptamine (5,6-MeO-MiPT), 5-methoxy-N-methyl-tryptamine (5-MeO-NMT), 5-methoxy-N,N-tetramethylenetryptamine (5-MeO-pyr-T), 6-methoxy-1-methyl-1,2,3,4-tetrahydrocarboline (6-MeO-THH), 5-methoxy-2,N,N-trimethyl-tryptamine (5-MeO-TMT), N,N-dimethyl-5-methylthiotryptamine (5-MeS-DMT), N-isopropyl-N-methyltryptamine (MiPT), α-methyltryptamine (α-MT), N-ethyltryptamine (NET), N-methyltryptamine (NMT), 6-propylnorlysergamide (PRO-LAD), N,N-tetramethylenetryptamine (pyr-T), tryptamine (T), 7-methoxy-1-methyl-1,2,3,4-tetrahydrocarboline (THH), or α,N-dimethyl-5-methoxytryptamine (α,N,O-TMS), or a pharmaceutically acceptable salt, hydrate, solvate, prodrug, stereoisomer, or tautomer thereof, or a combination thereof.

In some embodiments, an additional tryptamine will be a “complex tryptamine” or other indolamine and including such examples as iboga alkaloids such as ibogaine, and their analogs, metabolites, and derivatives, and beta-carbolines.

In some embodiments, the additional active compound is a phenethylamine. In some embodiments, and as will be understood by those in the art, a phenethylamine may be a phenylalkylamine having the structure of Formula (A), wherein R^{N1}, R^{N2}, R^{α}, R^{β}, and each of R²-R⁶ are as defined herein and as generally understood in the art.

In some embodiments, the additional active compound is a phenethylamine selected from the group of consisting α-ethyl-3,4,5-trimethoxy-phenethylamine (AEM), 4-allyloxy-3,5-dimethoxyphenethylamine (AL), 2,5-dimethoxy-4-methylthioamphetamine (ALEPH), 2,5-dimethoxy-4-ethylthioamphetamine (ALEPH-2), 2,5-dimethoxy-4-isopropylthioamphetamine (ALEPH-4), 2,5-dimethoxy-4-phenylthio-amphetamine (ALEPH-6), 2,5-dimethoxy-4-propylthioamphetamine (ALEPH-7), 2,5-dimethoxy-α-ethyl-4-methylphenethylamine (ARIADNE), 3,4-diethoxy-5-methoxyphenethylamine (ASB), 4-butoxy-3,5-dimethoxyphenethylamine (B), 2,5-dimethoxy-4,N-dimethylamphetamine (BEATRICE), 2,5-bismethylthio-4-methylamphetamine (BIS-TOM), 4-bromo-2,5,ß-trimethoxyphenethylamine (BOB), 2,5,ß-trimethoxy-4-methylphenethylamine (BOD), ß-methoxy-3,4-methylenedioxyphenethylamine (BOH), 2,5-dimethoxy-ß-hydroxy-4-methylphenethylamine (BOHD), 3,4,5,ß-tetramethoxyphenethylamine (BOM), 4-bromo-3,5-dimethoxyamphetamine (4-Br-3,5-DMA), 2-bromo-4,5-methylenedioxyamphetamine (2-Br-4,5-MDA), 3,4-methylenedioxy-N-ethylamphetamine (MDEA), 4-bromo-2,5-dimethoxyphenethylamine (2C-B), 4-benzyloxy-3,5-dimethoxyamphetamine (3C-BZ), 4-chloro-2,5-dimethoxyphenethylamine (2C-C), 2,5-dimethoxy-4-methyl-phenethylamine (2C-D), 2,5-dimethoxy-4-ethyl-phenethylamine (2C-E), 3,5-dimethoxy-4-ethoxyamphetamine (3C-E), 2,5-dimethoxy-4-fluorophenethylamine (2C-F), 2,5-dimethoxy-3,4-dimethylphenethylamine (2C-G), 2,5-dimethoxy-3,4-trimethylene-phenethylamine (2C-G-3), 2,5-dimethoxy-3,4-tetramethylenephenethylamine (2C-G-4), 3,4-norbornyl-2,5-dimethoxyphenethylamine (2C-G-5), 1,4-dimethoxynaphthyl-2-ethylamine (2C-G-N), 2,5-dimethoxyphenethylamine (2C-H), 4-iodo-2,5-dimethoxyphenethylamine (2C-I), 2,5-dimethoxy-4-nitro-phenethylamine (2C-N), 2,5-dimethoxy-4-isopropoxyphenethylamine (2C-O-4), 2,5-dimethoxy-4-propylphenethylamine (2C-P), 4-cyclopropylmethoxy-3,5-dimethoxyphenethylamine (CPM), 2,5-dimethoxy-4-methylselenophenethylamine (2C-SE), 2,5-dimethoxy-4-methylthiophenethylamine (2C-T), 2,5-dimethoxy-4-ethylthiophenethylamine (2C-T-2), 2,5-dimethoxy-4-isopropylthiophenethylamine (2C-T-4), 2,6-dimethoxy-4-isopropylthiophenethylamine (psi-2C-T-4), 2,5-dimethoxy-4-propylthiophenethylamine (2C-T-7), 4-cyclopropylmethylthio-2,5-dimethoxyphenethylamine (2C-T-8), 4-(t)-butylthio-2,5-dimethoxy-phenethylamine (2C-T-9), 2,5-dimethoxy-4-(2-methoxyethylthio) phenethylamine (2C-T-13), 4-cyclopropylthio-2,5-dimethoxyphenethylamine (2C-T-15), 4-(s)-butylthio-2,5-dimethoxyphenethylamine (2C-T-17), 2,5-dimethoxy-4-(2-fluoroethylthio) phenethylamine (2C-T-21), 3,5-dimethoxy-4-trideuteromethylphenethylamine (4-D), ß,ß-dideutero-3,4,5-trimethoxyphenethylamine (ß-D), 3,5-dimethoxy-4-methyl-phenethylamine (DESOXY), 2,4-dimethoxyamphetamine (2,4-DMA), 2,5-dimethoxyamphetamine (2,5-DMA), 3,4-dimethoxyamphetamine (3,4-DMA), 2-(2,5-dimethoxy-4-methylphenyl)cyclopropylamine (DMCPA), 3,4-dimethoxy-ß-hydroxyphenethylamine (DME), 2,5-dimethoxy-3,4-methylenedioxyamphetamine (DMMDA), 2,3-dimethoxy-4,5-methylenedioxyamphetamine (DMMDA-2), 3,4-dimethoxyphenethylamine (DMPEA), 4-amyl-2,5-dimethoxyamphetamine (DOAM), 4-bromo-2,5-dimethoxyamphetamine (DOB), 4-butyl-2,5-dimethoxyamphetamine (DOBU), 4-chloro-2,5-dimethoxyamphetamine (DOC), 2,5-dimethoxy-4-(2-fluoroethyl) amphetamine (DOEF), 2,5-dimethoxy-4-ethylamphetamine (DOET), 4-iodo-2,5-dimethoxyamphetamine (DOI), 2,5-dimethoxy-4-methylamphetamine (DOM (STP)), 2,6-dimethoxy-4-methylamphetamine (psi-DOM), 2,5-dimethoxy-4-nitroamphetamine (DON), 2,5-dimethoxy-4-propylamphetamine (DOPR), 3,5-dimethoxy-4-ethoxyphenethylamine (E), 2,4,5-triethoxyamphetamine (EEE), 2,4-diethoxy-5-methoxyamphetamine (EEM), 2,5-diethoxy-4-methoxyamphetamine (EME), 4,5-dimethoxy-2-ethoxyamphetamine (EMM), 2-ethylamino-1-(3,4-methylenedioxyphenyl) butane (ETHYL-J), 2-ethylamino-1-(3,4-methylenedioxyphenyl) pentane (ETHYL-K), 6-(2-aminopropyl)-5-methoxy-2-methyl-2,3-dihydrobenzofuran (F-2), 6-(2-aminopropyl)-2,2-dimethyl-5-methoxy-2,3-dihydrobenzofuran (F-22), N-hydroxy-N-methyl-3,4-methylenedioxyamphetamine (FLEA), 2,5-dimethoxy-3,4-(trimethylene) amphetamine (G-3), 2,5-dimethoxy-3,4-(tetramethylene) amphetamine (G-4), 3,6-dimethoxy-4-(2-aminopropyl)benzonorbornane (G-5), 2,5-dimethoxy-3,4-dimethylamphetamine (GANESHA), 1,4-dimethoxynaphthyl-2-isopropylamine (G-N), 2,5-dimethoxy-4-ethylthio-N-hydroxyphenethylamine (HOT-2), 2,5-dimethoxy-N-hydroxy-4-(n)-propylthiophenethylamine (HOT-7), 4-(s)-butylthio-2,5-dimethoxy-N-hydroxyphenethylamine (HOT-17), 2,5-dimethoxy-N,N-dimethyl-4-iodoamphetamine (IDNNA), 2,3,4-trimethoxy-phenethylamine (IM), 3,5-dimethoxy-4-isopropoxyphenethylamine (IP), 5-ethoxy-2-methoxy-4-methylamphetamine (IRIS), 2-amino-1-(3,4-methylenedioxyphenyl) butane (J, BDB), 3-methoxy-4,5-methylenedioxyphenethylamine (LOPHOPHINE), 3,4,5-trimethoxy-phenethylamine (M), 4-methoxyamphetamine (4-MA, PMA), 2,N-dimethyl-4,5-methylenedioxyamphetamine (MADAM-6), 3,5-dimethoxy-4-methallyloxyphenethylamine (MAL), 3,4-methylenedioxyamphetamine (MDA), N-allyl-3,4-methylenedioxyamphetamine (MDAL), N-butyl-3,4-methylenedioxyamphetamine (MDBU), N-benzyl-3,4-methylenedioxyamphetamine (MDBZ), N-cyclopropylmethyl-3,4-methylenedioxyamphetamine (MDCPM), N,N-dimethyl-3,4-methylenedioxyamphetamine (MDDM), N-ethyl-3,4-methylenedioxyamphetamine (MDE), N-(2-hydroxyethyl)-3,4-methylenedioxyamphetamine (MDHOET), N-isopropyl-3,4-methylenedioxyamphetamine (MDIP), N-methyl-3,4-methylenedioxyamphetamine (MDMA), 3,4-ethylenedioxy-N-methylamphetamine (MDMC), N-methoxy-3,4-methylenedioxyamphetamine (MDMEO), N-(2-methoxyethyl)-3,4-methylenedioxyamphetamine (MDMEOET), 3,4-methylenedioxy-α,α,N-trimethylphenethylamine (MDMP), N-hydroxy-3,4-methylenedioxyamphetamine (MDOH), 3,4-methylenedioxyphenethylamine (MDPEA), α,α-dimethyl-3,4-methylenedioxyphenethylamine (MDPH), 3,4-methylenedioxy-N-propargyl-amphetamine (MDPL), 3,4-methylenedioxy-N-propyl-amphetamine (MDPR), 3,4-dimethoxy-5-ethoxyphenethylamine (ME), 4,5-ethylenedioxy-3-methoxyamphetamine (MEDA), 4,5-diethoxy-2-methoxyamphetamine (MEE), 2,5-dimethoxy-4-ethoxyamphetamine (MEM), 4-ethoxy-3-methoxyphenethylamine (MEPEA), 5-bromo-2,4-dimethoxyamphetamine (META-DOB), 2,4-dimethoxy-5-methylthioamphetamine (META-DOT), 2,5-dimethoxy-N-methylamphetamine (METHYL-DMA), 4-bromo-2,5-dimethoxy-N-methylamphetamine (METHYL-DOB), 2-methylamino-1-(3,4-methylenedioxyphenyl) butane (METHYL-J, MBDB), 2-methylamino-1-(3,4-methylenedioxyphenyl) pentane (METHYL-K), 4-methoxy-N-methylamphetamine (METHYL-MA, PMMA), 2-methoxy-N-methyl-4,5-methylenedioxyamphetamine (METHYL-MMDA-2), 3-methoxy-4,5-methylenedioxyamphetamine (MMDA), 2-methoxy-4,5-methylenedioxyamphetamine (MMDA-2), 2-methoxy-3,4-methylenedioxyamphetamine (MMDA-3a), 4-methoxy-2,3-methylenedioxyamphetamine (MMDA-3b), 2,4-dimethoxy-5-ethoxyamphetamine (MME), 3,4-dimethoxy-5-(n)-propoxyphenethylamine (MP), 2,5-dimethoxy-4-(n)-propoxyamphetamine (MPM), 4,5-dimethoxy-2-methylthioamphetamine (ORTHO-DOT), 3,5-dimethoxy-4-propoxyphenethylamine (P), 3,5-dimethoxy-4-phenethyloxyphenethylamine (PE), phenethylamine (PEA), 3,5-dimethoxy-4-(2-propynyloxy) phenethylamine (PROPYNYL), 3,5-diethoxy-4-methoxyphenethylamine (SB), 2,3,4,5-tetra-methoxyamphetamine (TA), 4-ethoxy-3-ethylthio-5-methoxyphenethylamine (3-TASB), 3-ethoxy-4-ethylthio-5-methoxyphenethylamine (4-TASB), 3,4-diethoxy-5-methylthiophenethylamine (5-TASB), 4-(n)-butylthio-3,5-dimethoxyphenethylamine (TB), 4-ethoxy-5-methoxy-3-methylthiophenethylamine (3-TE), 3,5-dimethoxy-4-ethylthiophenethylamine (TE, 4-TE), 3,4-dimethoxy-2-methylthiophenethylamine (2-TIM), 2,4-dimethoxy-3-methylthiophenethylamine (3-TIM), 2,3-dimethoxy-4-methylthiophenethylamine (4-TIM), 3,4-dimethoxy-5-methylthiophenethylamine (3-TM), 3,5-dimethoxy-4-methylthiophenethylamine (4-TM), 3,4,5-trimethoxyamphetamine (TMA), 2,4,5-trimethoxyamphetamine (TMA-2), 2,3,4-trimethoxyamphetamine (TMA-3), 2,3,5-trimethoxyamphetamine (TMA-4), 2,3,6-trimethoxyamphetamine (TMA-5), 2,4,6-trimethoxyamphetamine (TMA-6), 4,5-dimethoxy-3-ethylthiophenethylamine (3-TME), 3-ethoxy-5-methoxy-4-methylthiophenethylamine (4-TME), 3-ethoxy-4-methoxy-5-methylthiophenethylamine (5-TME), 3,4-methylenedioxy-2-methylthioamphetamine (2T-MMDA-3a), 2-methoxy-4,5-methylene-thiooxyamphetamine (4T-MMDA-2), 2,4,5-trimethoxyphenethylamine (TMPEA), 4-ethyl-5-methoxy-2-methylthioamphetamine (2-TOET), 4-ethyl-2-methoxy-5-methylthioamphetamine (5-TOET), 5-methoxy-4-methyl-2-methylthioamphetamine (2-TOM), 2-methoxy-4-methyl-5-methylthioamphetamine (5-TOM), 2-methoxy-4-methyl-5-methyl-sulfinylamphetamine (TOMSO), 3,5-dimethoxy-4-propylthiophenethylamine (TP), 3,4,5-triethoxyphenethylamine (TRIS), 3-ethoxy-5-ethylthio-4-methoxyphenethylamine (3-TSB), 3,5-diethoxy-4-methylthiophenethylamine (4-TSB), 3,4-diethoxy-5-ethylthiophenethylamine (3-T-TRIS), 3,5-diethoxy-4-ethylthiophenethylamine (4-T-TRIS), (R)-2,5-dimethoxy-4-iodoamphetamine (R-DOI), or a pharmaceutically acceptable salt, hydrate, solvate, prodrug, stereoisomer, or tautomer thereof, or a combination thereof.

In some embodiments, the additional active compound is an ergoline. In embodiments, the additional active compound is an ergot alkaloid. In embodiments, the additional active compound is a lysergamide. As will be understood by those in the art, lysergamides are compounds having the general structure below, wherein R^{N1}, R^{N2}, R¹, R², R⁴, R⁶, R⁷, R⁸, R⁹, R¹², R¹³, and R¹⁴ are as defined herein and as generally understood in the art:

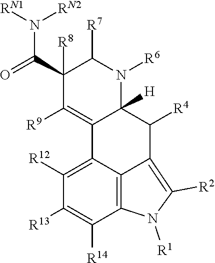

In some embodiments, R^{N1}, R^{N2}, R¹, R², R⁴, R⁶, R⁷, R⁸, R⁹, R¹², R¹³, and R¹⁴ are each independently hydrogen, deuterium, halogen, optionally substituted alkyl, optionally substituted alkenyl, optionally substituted alkynyl, optionally substituted cycloalkyl, optionally substituted cycloalkenyl, optionally substituted aryl, or optionally substituted heterocyclyl. Additionally, any two of R^{N1}, R^{N2}, R¹, R², R⁴, R⁶, R⁷, R⁸, R⁹, R¹², R¹³, and R¹⁴ and the intervening atoms can be taken together to form an optionally substituted optionally substituted cycloalkyl, optionally substituted cycloalkenyl, optionally substituted aryl, or optionally substituted heterocyclyl. In embodiments, the lysergamide is a quaternary salt, in which an additional R^{6A} is connected to the nitrogen to which R⁶ is bound; wherein R^{6A} is optionally substituted alkyl, optionally substituted alkoxy, optionally substituted alkyl, optionally substituted alkenyl, optionally substituted alkynyl, optionally substituted cycloalkyl, optionally substituted cycloalkenyl, optionally substituted aryl, or optionally substituted heterocyclyl.

In some embodiments, the additional active compound is a lysergamide selected from the group consisting of lysergic acid diethylamide (i.e., LSD, LSD-25, LAD, Delysid), 6-ethyl-6-nor-lysergic acid diethylamide (ETH-LAD), 6-propynyl-6-nor-lysergic acid diethylamide (PARGY-LAD), 6-allyl-6-nor-lysergic acid diethylamide (AL-LAD), 6-propyl-6-nor-lysergic acid diethylamide (PRO-LAD), 6-isopropyl-6-nor-lysergic acid diethylamide (IP-LAD), 6-cylopropyl-6-nor-lysergic acid diethylamide (CIP-LAD), 6-butyl-6-nor-lysergic acid diethylamide (BU-LAD), 6-(2-fluoroethyl)-6-nor-lysergic acid diethylamide (FLUOROETH-LAD), 1-acetyl-lysergic acid diethylamide (i.e., ALD, ALD-52, N-acetyl-LSD), 1-propionyl-lysergic acid diethylamide (1P-LSD), 1-butyryl-lysergic acid diethylamide (1B-LSD), 1-valeryl-lysergic acid diethylamide (1V-LSD), 1-(cyclopropyl-methanoyl)-lysergic acid diethylamide (1 cP-LSD), 1-(1,2-dimethylcyclobutane-1-carbonyl)-lysergic acid diethylamide (1D-LSD), 1-propionyl-6-allyl-6-nor-lysergic acid diethylamide (1P-AL-LAD), 1-(cyclopropylmethanoyl)-6-allyl-6-nor-lysergic acid diethylamide (1 cP-AL-LAD), 1-propionyl-6-ethyl-6-nor-lysergic acid diethylamide (1P-ETH-LAD), lysergic acid 2,4-dimethylazetidide (i.e., LA-SS-Az, LSZ), lysergic acid piperidide (LSD-Pip), and lysergic acid methylisopropyl amide (MIPLA).

Other tryptamines, phenethylamines, and lysergamides useful as additional active compounds for purposes of the invention and thus contemplated for inclusion therein will be as generally known in the art (see, e.g., Shulgin and Shulgin, PiHKAL: A Chemical Love Story, Transform Press (1991); Shulgin and Shulgin, TiHKAL: The Continuation, Transform Press (1997); Grob & Grigsby, Handbook of Medical Hallucinogens, 2021; Luethi & Liechti, Arch. Toxicol., 2020; 94, 1085-1133; Nichols, Pharmacological Reviews, 2016; 68 (2), 264-355; Glennon, Pharmacology Biochemistry and Behavior, 1999; 64, 251-256; each of which is incorporated by reference as if fully set forth herein).

### E. DOSE AND DOSAGE

In some embodiments, pharmaceutical compositions comprise a therapeutically effective amount or an effective amount of a disclosed compound, such as for administration to a subject. Administration of pharmaceutical compositions in a “therapeutically effective amount,” or an “effective amount” to a subject means administration of an amount of composition sufficient to achieve the desired effect. When an “effective amount” means an amount effective in treating the stated disorder or symptoms in a subject, “therapeutic effect” would be understood to mean the responses(s) in a mammal after treatment that are judged to be desirable and beneficial. Hence, depending on the mental health disorder to be treated, or improvement in mental health or functioning sought, and depending on the particular constituent(s) in the disclosed compositions under consideration, those responses shall differ, but would be readily understood by those of ordinary skill, through an understanding of the disclosure herein and the general knowledge of the art (e.g., by reference to the symptoms listed in the Diagnostic and Statistical Manual of Mental Disorders, 5th Edition (DSM-5) for the stated disorder).

In some embodiments, where a pharmaceutical composition includes a disclosed compound, it may be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, as well as amounts within these ranges.

In some embodiments, where a pharmaceutical composition includes a disclosed compound, it may be present in an amount so that a single dose is (in a milligram dosage amount calculated based on the kilogram weight of the patient) between about 0.01 mg/kg and 0.1 mg/kg, such as about 0.01 mg/kg, about 0.02 mg/kg, about 0.03 mg/kg, about 0.04 mg/kg, about 0.05 mg/kg, about 0.06 mg/kg, about 0.07 mg/kg about 0.08 mg/kg about 0.09 mg/kg, and about 0.1 mg/kg, as well as ranges between these values. In some embodiments, a single dose is between about 0.1 mg/kg and 1.0 mg/kg, such as about 0.1 mg/kg, about 0.2 mg/kg, about 0.3 mg/kg, about 0.4 mg/kg, about 0.5 mg/kg, about 0.6 mg/kg, about 0.7 mg/kg about 0.8 mg/kg about 0.9 mg/kg, and about 1.0 mg/kg, as well as ranges between these values.

In some embodiments, where a pharmaceutical composition includes a disclosed compound, it may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

In some embodiments, where a pharmaceutical composition includes a disclosed compound, it may be present in an amount so that a single dose is (whether or not such dose is present in a unit dosage form) between about 0.1 mg and 1.0 mg, such as about 0.1 mg, about 0.2 mg, about 0.3 mg, about 0.4 mg, about 0.5 mg, about 0.6 mg, about 0.7 mg, about 0.8 mg, about 0.9 mg, and about 1.0 mg, as well as ranges between these values. In embodiments, a single dose is between about 1 mg and 10 mg, such as about 1 mg, about 2 mg, about 3 mg, about 4 mg, about 5 mg, about 6 mg, about 7 mg, about 8 mg, about 9 mg, and about 10 mg, as well as ranges between these values. In some embodiments, a single dose is between about 10 mg and 100 mg.

In some embodiments, where a pharmaceutical composition includes an additional active compound, for instance where the additional active compound is a phenethylamine or another tryptamine, it may be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, as well as amounts within these ranges.

In some embodiments, where a pharmaceutical composition includes an additional active compound, for instance where the additional active compound is a phenethylamine or a tryptamine, it may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

It will be readily appreciated that dosages may vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender, and race of the subject, bioavailability, potential adverse systemic, regional or local side effects, the presence of other disorders or diseases in the subject, and other factors that will be appreciated by the skilled artisan (e.g., medical or familial history).

Dose amount, frequency or duration may be increased or reduced, as indicated by the clinical outcome desired, status of the pathology or symptom, any adverse side effects of the treatment or therapy, or concomitant medications. The skilled artisan with the teaching of this disclosure in hand will appreciate the factors that may influence the dosage, frequency, and timing required to provide an amount sufficient or effective for providing a therapeutic effect or benefit, and to do so depending on the type of therapeutic effect desired, as well as to avoid or minimize adverse effects.

It will be understood that, in some embodiments, the dose actually administered will be determined by a physician, in light of the relevant circumstances, including the disorder to be treated, the chosen route of administration, the actual composition or formulation administered, the age, weight, and response of the individual patient, and the severity of the patient's symptoms, and therefore any dosage ranges disclosed herein are not intended to limit the scope of the invention. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In some embodiments, especially where a formulation is prepared in single unit dosage form, such as a capsule, tablet, or lozenge, suggested dosage amounts shall be known by reference to the format of the preparation itself. In other embodiments, where a formulation is prepared in multiple dosage form, for instance liquid suspensions and topical preparations, suggested dosage amounts may be known by reference to the means of administration or by reference to the packaging and labeling, package insert(s), marketing materials, training materials, or other information and knowledge available to those of skill or the public.

Accordingly, another aspect of this disclosure provides pharmaceutical kits containing a pharmaceutical composition or formulation of the invention, suggested administration guidelines or prescribing information therefor, and a suitable container. Individual unit dosage forms can be included in multi-dose kits or containers. pharmaceutical formulations also can be packaged in single or multiple unit dosage forms for uniformity of dosage and ease of administration.

### F. KITS

Another aspect of this disclosure provides pharmaceutical kits containing a pharmaceutical composition or formulation of the invention, suggested administration guidelines or prescribing information therefor, and a suitable container. Individual unit dosage forms can be included in multi-dose kits or containers. pharmaceutical formulations also can be packaged in single or multiple unit dosage forms for uniformity of dosage and ease of administration.

Kits generally comprise suitable packaging. The kits may comprise one or more containers comprising any compound described herein. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

Preferably, information pertaining to dosing and proper administration (if needed) will be printed onto a multi-dose kit directly (e.g., on a blister pack or other interior packaging holding the compositions or formulations of the invention); however, kits of the invention can further contain package inserts and other printed instructions (e.g., on exterior packaging) for administering the disclosed compositions and for their appropriate therapeutic use.

### G. METHODS OF USE

In some aspects, provided herein are methods of using the disclosed compounds. In some embodiments, disclosed compounds are used to modulate neurotransmission. In embodiments, disclosed compounds are used to treat a condition, such as a disease or a disorder. In embodiments, disclosed compounds are used in the manufacture of a medicament for the therapeutic and/or the prophylactic treatment of a condition, such as a disease or a disorder. In embodiments, disclosed compounds are administered as part of therapy. In embodiments, disclosed compounds are administered along with psychotherapy, psychological support, or patient monitoring. In embodiments, disclosed compounds are administered in a therapeutically effective amount to a subject having a condition, such as a disease or a disorder. In embodiments, the condition is a mental health disorder. In embodiments, the condition is a neurodegenerative disorder. In embodiments, the condition is an inflammatory disorder. In embodiments, the condition is pain and/or inflammation. In embodiments, disclosed compounds are administered to a subject that is healthy.

As used herein, the terms “subject,” “user,” “patient,” and “individual” are used interchangeably, and refer to any mammal, including murines, simians, mammalian farm animals, mammalian sport animals, and mammalian pets, such as canines and felines, although preferably humans. Such terms will be understood to include one who has an indication for which a compound, composition, or method described herein may be efficacious, or who otherwise may benefit by the invention. In general, all of the compounds, compositions, and disclosed methods will be appreciated to work for all individuals, although individual variation is to be expected, and will be understood. The disclosed methods of treatment also can be modified to treat multiple patients at once, including couples or families. Hence, these terms will be understood to also mean two or more individuals.

In some embodiments, disclosed compounds or compositions thereof are orally, mucosally, rectally, subcutaneously, intravenously, intramuscularly, intranasally, by inhalation or transdermally administered to a subject. In embodiments, when administered through one or more such routes, the disclosed compounds and the disclosed compositions and formulations comprising them are useful in methods for treating a patient in need of such treatment.

a. Research Tools

In some embodiments, disclosed compounds are used as research tools, e.g., involved in determining the structure and function of a receptor in vitro, in vivo, or in silico. In embodiments, disclosed compounds may be used in receptor, ion channel, enzyme, and transporter binding studies. In embodiments, disclosed compounds may be used in mapping, and functional studies.

In embodiments, radiolabeled disclosed compounds may be used to identify binding sites. In embodiments, radiolabeled disclosed compounds may be used for tissue imaging.

In some embodiments, disclosed compounds may be used as research tools for 5-HT₂ receptors. In embodiments, disclosed compounds may be used as research tools for 5-HT_{2A} receptors. In embodiments, disclosed compounds may be used as research tools for 5-HT_{2B} receptors. In embodiments, disclosed compounds may be used as research tools for 5-HT_{2C} receptors. In embodiments, the research tool is a receptor probe, which may be used for determining downstream events of receptor-ligand interaction, e.g., calcium regulation, kinase, phosphatase and phospholipase activation, and lipid trafficking. In embodiments, the receptor is a recombinant receptor. In embodiments, the receptor is a wild-type receptor.

In some embodiments, disclosed compounds may be used as research tools, such as receptor probes, for 5-HT₂ receptors of mammalian origin. In embodiments, disclosed compounds may be used as research tools, such as receptor probes, for 5-HT₂ receptors of human (Homo sapiens) origin. In embodiments, disclosed compounds may be used as research tools, such as receptor probes, for 5-HT₂ receptors of non-human primate origin. Non-limiting examples of non-human primate 5-HT₂ receptors include chimpanzee (Pan troglodytes) and Rhesus macaque (Macaca mulatta) 5-HT₂ receptors.

In some embodiments, disclosed compounds may be used as research tools, such as receptor probes, for 5-HT₂ receptors of rodent origin. Non-limiting examples of rodent 5-HT₂ receptors include those of mouse (Mus musculus) and rat (Rattus norvegicus) origin. Common lab mouse strains include C57BL/6 and BALB/c, and common lab rat strains include Sprague-Dawley and Wistar. In embodiments, disclosed compounds may be used as research tools, such as receptor probes, for 5-HT₂ receptors of zebrafish origin. Non-limiting examples of zebrafish 5-HT₂ receptors include those of Danio spp., e.g., Danio rerio origin. In embodiments, disclosed compounds may be used as research tools, such as receptor probes, for 5-HT₂ receptors of nematode origin. In one non-limiting example, 5-HT₂ receptors of Caenorhabditis elegans may be probed with a disclosed compound. In embodiments, disclosed compounds may be used as research tools, such as receptor probes, for 5-HT₂ receptors of a dog, chicken, frog, or cow.

Sequences may be retrieved by consulting a nucleotide database, e.g., Genbank, or amino acid database, e.g., UniProtKB, as known to one of skill. As examples, sequences 1-45 in Table 5 are available on UniProtKB (www.uniprot.org) by reference to their accession number below; they are also disclosed in the priority document hereof, incorporated as if fully set forth herein.

TABLE 5
Exemplary Receptor Sequences

| No. | Accession Entry/Entry Name, Organism, Protein |
|---|---|
| 1 | P07550 ADRB2_HUMAN Beta-2 Homo sapiens adrenergic receptor |
| 2 | P18762 ADRB2_MOUSE Beta-2 Mus musculus adrenergic receptor |
| 3 | P10608 ADRB2_RAT Beta-2 Rattus norvegicus adrenergic receptor |
| 4 | A0A8I6A0X2_RAT Beta-2 Rattus norvegicus adrenergic receptor isoform |
| 5 | F7FBA1_RAT Beta-2 Rattus norvegicus adrenergic receptor isoform |
| 6 | H2QRR3_PANTR Beta-2 Pan troglodytes adrenergic receptor |
| 7 | Q28509_MACMU Beta-2 Macaca mulatta adrenergic receptor |
| 8 | P54833_CANLF Beta-2 Canis lupus familiaris adrenergic receptor |
| 9 | Q28044_BOVIN Beta-2 Bos taurus adrenergic receptor |
| 10 | A0A1D5P7A9_CHICK Beta-2 Gallus gallus domesticus adrenergic receptor |
| 11 | P28223 5HT_{2A—}HUMAN Homo sapiens 5-hydroxytryptamine receptor 2A |
| 12 | P28223-2 5HT_{2A—}HUMAN Homo sapiens 5-hydroxytryptamine receptor 2A isoform |
| 13 | P35363 5HT_{2A—}MOUSE Mus musculus 5-hydroxytryptamine receptor 2A |
| 14 | P14842 5HT_{2A—}RAT Rattus norvegicus 5-hydroxytryptamine receptor 2A |
| 15 | A0A2J8J9Q7_PANTR Pan troglodytes 5-hydroxytryptamine receptor 2A |
| 16 | P50128 5HT_{2A—}MACMU Macaca mulatta 5-hydroxytryptamine receptor 2A |
| 17 | 04665 5HT_{2A—}CANLF Canis lupus familiaris 5-hydroxytryptamine receptor 2A |
| 18 | Q75Z89 5HT_{2A—}BOVIN Bos taurus 5-hydroxytryptamine receptor 2A |
| 19 | P41595 5HT_{2B—}HUMAN Homo sapiens 5-hydroxytryptamine receptor 2B |
| 20 | Q02152 5HT_{2B—}MOUSE Mus musculus 5-hydroxytryptamine receptor 2B |
| 21 | G3UVT8 5HT_{2B—}MOUSE Mus musculus 5-hydroxytryptamine receptor 2B isoform |
| 22 | P30994 5HT_{2B—}RAT Rattus norvegicus 5-hydroxytryptamine receptor 2B |
| 23 | G3V8A0 5HT_{2B—}RAT Rattus norvegicus 5-hydroxytryptamine receptor 2B isoform |
| 24 | H2QJL0 5HT_{2B—}PANTR Pan troglodytes 5-hydroxytryptamine receptor 2B |
| 25 | F7H1F4 5HT_{2B—}MACMU Macaca mulatta 5-hydroxytryptamine receptor 2B |
| 26 | A0A8I3PC67_CANLF Canis lupus familiaris 5-hydroxytryptamine receptor 2B |
| 27 | F6Q178 5HT_{2B—}BOVIN Bos taurus 5-hydroxytryptamine receptor 2B |
| 28 | P28335 5HT_{2C—}HUMAN Homo sapiens 5-hydroxytryptamine receptor 2C |
| 29 | P28335-2 5HT_{2C—}HUMAN Homo sapiens 5-hydroxytryptamine receptor 2C isoform |
| 30 | P34968-1 5HT_{2C—}MOUSE Mus musculus 5-hydroxytryptamine receptor 2C precursor |
| 31 | S4R2T0 5HT_{2C—}MOUSE Mus musculus 5-hydroxytryptamine receptor 2C isoform |
| 32 | B1ATN4 5HT_{2C—}MOUSE Mus musculus 5-hydroxytryptamine receptor 2C isoform |
| 33 | P08909 5HT_{2C—}RAT Rattus norvegicus 5-hydroxytryptamine receptor 2C precursor |
| 34 | F1LPS1-1 5HT_{2C—}RAT Rattus norvegicus 5-hydroxytryptamine receptor 2C isoform |
| 35 | A0A0G2JY06-1_RAT Rattus norvegicus 5-hydroxytryptamine receptor 2C isoform |
| 36 | A0A8I6A845-1_RAT Rattus norvegicus 5-hydroxytryptamine receptor 2C isoform |
| 37 | Q5IS66 5HT_{2C—}PANTR Pan troglodytes 5-hydroxytryptamine receptor 2C |
| 38 | A0A2J8KYJ7-1_PANTR Pan troglodytes 5-hydroxytryptamine receptor 2C isoform |
| 39 | A0A2I3S8Z4_PANTR Pan troglodytes 5-hydroxytryptamine receptor 2C isoform |
| 40 | F7A578 5HT_{2C—}MACMU Macaca mulatta 5-hydroxytryptamine (5-HT) receptor 2C |
| 41 | Q60F97 5HT_{2C—}CANLF Canis lupus familiaris 5-HT receptor 2C precursor |
| 42 | A0A8I3NYQ9_CANLF Canis lupus familiaris 5-HT receptor 2C isoform |
| 43 | A0A8I3NNR2_CANLF Canis lupus familiaris 5-HT receptor 2C isoform |
| 44 | A0A8I3NXN8-1_CANLF Canis lupus familiaris 5-HT receptor 2C isoform |
| 45 | A0A8I3S1Q6-1_CANLF Canis lupus familiaris 5-HT receptor 2C isoform |

b. Serotonin Receptor Ligands

The 5-HT receptor system comprises 14 distinct receptors, which are grouped into seven receptor families (5-HT₁ to 5-HT₇). With the exception of the 5-HT₃ ligand-gated ion channel, all 5-HTR families are G-protein coupled receptors (GPCR) (Gothert, Pharmacol Rep., 2013; 65 (4): 771-86). In one representative example, the 5-HT_{2A} receptor has seven transmembrane helices and intracellular amphipathic helix H8, similar to other GPCRs. The receptor comprises a ligand binding site, termed an orthosteric site, in addition to an accessory site, a side-extended cavity that connects the orthosteric site to the plasma membrane. Herein, the side-extended cavity may be referred to herein as an “extended binding site” or an “exosite.”

In some embodiments, a disclosed compound binds to a serotonin (5-HT) receptor. In embodiments, the 5-HT receptor is a subfamily 1 (5-HT₁) receptor. In embodiments, the 5-HT receptor is a subfamily 2 (5-HT₂) receptor. In embodiments, the 5-HT₂ receptor is a subtype 2A, 2B, or 2C (5-HT_{2A}, 5-HT_{2B}, or 5-HT_{2C}) receptor. In embodiments, the 5-HT receptor is a subfamily 4 (5-HT₄) receptor. In embodiments, the receptor is a 5-HT receptor of subfamily 5 (5-HT₅) receptor. In embodiments, the receptor is a 5-HT receptor of subfamily 6 (5-HT₆) receptor. In embodiments, the 5-HT receptor is a subfamily 7 (5-HT₇) receptor. In embodiments, the receptor is of mammalian origin. In embodiments, the receptor is of human origin. In embodiments, the receptor is recombinant.

In some embodiments, a disclosed compound binds to a serotonin (5-HT) receptor at more than one site, such as an orthosteric site and an extended binding site. In embodiments, a disclosed compound binds to a 5-HT₁ receptor at one or more sites, such as an orthosteric site and an extended binding site. In embodiments, a disclosed compound binds to a 5-HT₂ receptor at one or more sites, such as an orthosteric site and an extended binding site. In embodiments, a disclosed compound binds to one or more 5-HT₂ receptor subtypes, such as 5-HT_{2A}, 5-HT_{2B}, and 5-HT_{2C} receptors, at one or more sites, such as an orthosteric site and an extended binding site. In embodiments, a disclosed compound binds to a 5-HT₄ receptor at one or more sites, such as an orthosteric site and an extended binding site. In embodiments, a disclosed compound binds to a 5-HT₅ receptor at one or more sites, such as an orthosteric site and an extended binding site. In embodiments, a disclosed compound binds to a 5-HT₆ receptor at one or more sites, such as an orthosteric site and an extended binding site. In embodiments, a disclosed compound binds to a 5-HT₇ receptor at one or more sites, such as an orthosteric site and an extended binding site. In embodiments, the receptor is of mammalian origin. In embodiments, the receptor is of human origin. In embodiments, the receptor is recombinant.

As determined in complex with the antagonist zotepine, the extended binding site spans transmembrane domains (TMDs) 4 and 5, and is surrounded by hydrophobic residues on TMD3, TMD4, TMD5, and extracellular loop (ECL) 2. A glycine residue at position 5.42×43 at the entrance of the side-extended cavity is essential for formation of the exosite. This glycine positioning is conserved only in the 5-HT₂ family of receptors (Kimura et al., Nat Struct Mol Biol., 2019; 26 (2): 121-128). An extended binding site has also been described for 5-HT_{1B} and 5-HT_{2B} receptors (McCorvy & Roth, Pharmacology & Therapeutics, 2015; 150:129-142).

Methods for determining binding to a 5-HT receptor are available to one of skill in the art, including, e.g., in vitro and in silico computational methods. For example, Kimura et al., describes constructing a 5-HT_{2A}R, crystallizing the receptor with an inverse agonist and an antagonist, and determining the binding using micro-crystallography and molecular docking software Glide (Schrodinger) (Kimura et al., Nature Structural & Molecular Biology, 2019; 26:121-128). Wacker et al. describes resolving the structure of LSD bound to an engineered 5-HT_{2B} receptor using X-ray crystallography. Given the homology between 5-HT_{2A} and 5-HT_{2B} receptors, the 5-HT_{2B} receptor was used as a model system for 5-HT_{2A} receptor (Wacker et al., Cell. 2017; 168 (3): 377-389.e12). X-ray crystallography has also been used to determine the structure of 5-HT_{2A}R complexed with LSD and inverse agonist methiothepin, whereas cryo-electron microscopy of prototypical hallucinogen 25CN—NBOH in complex with an engineered Gαq heterotrimer has been used to determine the active state of 5-HT_{2A}R (Kim et al., Cell. 2020 Sep. 17; 182 (6): 1574-1588.e19). Homology modeling based on the β (2)-adrenergic receptor and the G protein-bound opsin crystal structures have also been used to model the 5-HT_{2A}R (Isberg et al., J Chem Inf Model., 2011; 51 (2): 315-25).

Additionally, similar strategies that were used to elucidate the ADRB2 exosite may be applied to a 5-HT receptor, such as docking and mutagenic studies. For example, site-directed mutagenesis and evaluation of salmeterol-promoted cAMP accumulation, led to identification of amino acids in TMD4 (residues 149-174) as contributing to the β2 receptor exosite (Green et al., J Biol Chem., 1996; 271 (39): 24029-35). Direct photoaffinity labeling of the salmeterol binding site in the human β2 receptor using [¹²⁵I]iodoazidosalmeterol as well as x-ray diffraction analysis of salmeterol bound to ADRB2 revealed that TMD6 and TMD7 also contribute to the exosite (Masureel et al., Nat. Chem. Biol. 2018; 14 (11): 1059-1066; Rong et al., Biochemistry, 1999; 38 (35): 11278-11286). In the [¹²⁵I]iodoazidosalmeterol photolabeling study, the radioiodinated phenyl azide moiety of the photoaffinity probe, corresponding to the terminal phenyl ring in salmeterol, was specifically attached to tryptophan 313 in TMD7 (Rong et al., Biochemistry, 1999; 38 (35): 11278-11286). A third study, employing chimeric β_{1/2} receptors and alanine-substituted β₂ receptor mutants, also identified TMD7 as part of the exosite. The study identified tyrosine 308 and tyrosine 316 in TMD7 as sites of interaction with methylene groups near the protonated amine of salmeterol and the side-chain ether oxygen, respectively (Isogaya et al., Mol. Pharmacol. 1998; 54:616-622).

Both the 5-HT_{2A}R and ADRB2 receptors adopt various conformations in response to different ligands. This movement and consequential remodeling of the surrounding membrane influences known pharmacological activity of the ligands, such as full, partial or inverse activators of the receptor. For example, Shan et al. showed distinct 5-HT_{2A}R conformation in response to partial agonist LSD and inverse agonist ketanserin (Shan et al., PLOS Comput Biol. 2012; 8 (4): e1002473). Relating to ADRB2 ligand studies, the majority or receptor residues showed distinct patterns of reactivity, even between functionally similar ligands, and few receptor residues had reactivity patterns consistent with classical agonism (Kahsai et al., Nat Chem Biol. 2011; 7 (10): 692-700). Various conformational states of 5-HT receptors may result in distinct pharmacological outcomes, including those related to hallucinogenesis. See, e.g., Weinsten, AAPS J 7: E871-884. As such, in one aspect, the provided 5-HT receptor ligands may be useful to elucidate the structural basis and mechanisms for different states of 5-HT receptor activation, e.g., 5-HT_{2A}R, 5-HT_{2B}R, and 5-HT_{2C}R.

In some embodiments, a disclosed compound has increased binding affinity for a 5-HT receptor, relative to a comparator. In embodiments, a disclosed compound has decreased binding affinity for a 5-HT receptor, relative to a comparator. In embodiments, a disclosed compound has both increased binding affinity for a 5-HT receptor subtype and decreased binding affinity for another serotonin receptor subtype. In embodiments, the receptor is a 5-HT_{2A} receptor. In embodiments, the receptor is a 5-HT_{2B} receptor. In embodiments, the receptor is a 5-HT_{2C} receptor. In one example, a disclosed compound, such as an N-substituted 2C-B (e.g., XOB; see Examples 1 and 2), has increased affinity for a 5-HT₂ receptor relative to a comparator. In embodiments, the comparator is the corresponding unsubstituted phenylalkylamine. For example, in embodiments, the comparator for XOB is 2C-B. In other embodiments, the comparator is serotonin.

In some embodiments, a disclosed compound has increased selectivity or specificity for a 5-HT receptor relative to a comparator. In embodiments, a disclosed compound has relatively high selectivity at 5-HT₂ receptors, e.g., 5-HT_{2A}, 5-HT_{2B}, and 5-HT_{2C} receptors, relative to a comparator. In embodiments, the comparator is an unsubstituted phenylalkylamine, including, e.g., a compound having the same phenylalkylamine head group but lacking the side chain. In embodiments, a disclosed compound has fewer off-target effects, including, e.g., adverse effects.

Various strategies are available to determine binding affinity and specificity. Notably, the results of binding assays describe the strength of the interaction between a ligand and a target, not whether the ligand acts as an agonist or an antagonist. Radioligand binding experiments represent sensitive and quantitative techniques for determining binding affinity between a compound and a receptor. The general framework of a radioligand binding assay involves preparing a tissue that bears a target receptor and incubating with a radiolabeled ligand until equilibrium is reached. Then, equilibrium is broken, radiolabeled ligand bound to a receptor and free radioligand are separated, radioactivity is quantified, such as radioactivity bound to tissue, and results are analyzed, for example, with use of specialized computer software. Under identical conditions, non-specific binding is measured by incubating the tissue in the presence of an unlabeled ligand at a concentration that saturates target receptors. Specific binding is calculated by subtracting non-specific binding from total binding.

Use of radioligands may aid determination of binding affinity in a number of different experimental contexts, including kinetic experiments, wherein the time course of ligand association and dissociation is determined, competition binding assays, dissociation binding assays, saturation binding assays, and in quantitative autoradiography and image analyses (Maguire et al., Methods Mol Biol. 2012; 897:31-77).

In some embodiments, affinity can be evaluated by determining the inhibition constant of an N-aralkyloxyalkyl-substituted phenylalkylamine and a receptor, such as a 5-HT₂ receptor. An inhibition constant (Kᵢ) may be described as the concentration at which 50% of a radiolabeled ligand, such as an agonist, is displaced by the test ligand. Accordingly, as presented in the Cheng-Prusoff equation, an observed IC₅₀ can be used to determine Kᵢ, where Kᵢ=IC₅₀/(1+[R]/K_{d}), where the IC₅₀ is the concentration of the competitive inhibitor producing a 50% inhibition, R is the concentration of radioligand used in the competition binding assay, and K_{d} is the equilibrium dissociation constant of the radioligand in the assay.

A competition binding assay, otherwise referred to as a radioligand displacement assay, can be used to determine Kᵢ. Generally, the effect of a test ligand on the interactions between a radiolabeled ligand and a receptor preparation is assessed, e.g., the extent of radiolabeled ligand displacement is evaluated.

In some examples, displaced radioligands may be antagonists, for example, [³H]ketanserin for 5-HT_{2A} and [³H]mesulergine for 5-HT_{2C}. In some examples, displaced radioligands may be agonists, for example [³H]LSD for 5-HT_{2B}. However, consistency of test conditions is preferred for the purposes of making a comparison, as displacement of antagonists may reflect binding to both active and inactive receptor conformations, and displacement of agonists presumably reflects binding to an active conformation (Toro-Sazo et al., PLOS One, 2019; 14 (1): e0209804). Binding assays are further described in, e.g., Roth's National Institutes of Mental Health Psychoactive Drug Screening Program, Assay Protocol Book, Version III, 2018.

In some embodiments, a disclosed compound has a binding affinity for any one or more of 5-HT_{2A}, 5-HT_{2B}, and 5-HT_{2A}, that is less than 10 μM, less than 5 μM, less than 1 μM, less than 0.5 μM, or less than 0.1 μM. In embodiments, a disclosed compound has a binding affinity for any one or more of 5-HT_{2A}, 5-HT_{2B}, and 5-HT_{2A}, that is about 10 μM, 5 μM, 1 μM, 0.5 μM, or 0.1 μM. In embodiments, a disclosed compound has increased binding affinity for any one or more of 5-HT_{2A}, 5-HT_{2B}, and 5-HT_{2A}, relative to a comparator. In embodiments, a disclosed compound has decreased binding affinity for any one or more of 5-HT_{2A}, 5-HT_{2B}, and 5-HT_{2A}, relative to a comparator. In embodiments, the comparator is serotonin. In embodiments, the comparator is the corresponding unsubstituted phenylalkylamine. In embodiments, the comparator for XOB is 2C-B. For example, XOB has a binding affinity for 5-HT_{2A} of 2.6 μM (see Example 2), and no measurable affinity at 5-HT_{1A}. In comparison, in vitro studies using [¹²⁵I]-DOI competition binding in heterologous cells expressing human or rat 5-HT_{2A} receptors report a high affinity for 2C-B with a K_{I} of 0.88 nM and 0.66 nM, respectively (McLean et al., J Med Chem., 2006; 49 (19): 5794-5803). Furthermore, serotonin exhibits moderate K values of 330 nM, 470 nM, and 120 nM at 5-HT_{1A}, 5-HT_{2A}, and 5-HT_{2C} receptors, respectively. In embodiments, the binding affinity of a disclosed compound for any one or more of 5-HT_{2A}, 5-HT_{2B}, and 5-HT_{2A}, is increased by about 2-fold, 5-fold, 10-fold, 20-fold, 30-fold, 50-fold, 70-fold, 80-fold, 90-fold, 100-fold, 200-fold, 500-fold, 1000-fold or at least 1000-fold relative to a comparator. In embodiments, the binding affinity of a disclosed compound for any one or more of 5-HT_{2A}, 5-HT_{2B}, and 5-HT_{2A}, is decreased by about 2-fold, 5-fold, 10-fold, 20-fold, 30-fold, 50-fold, 70-fold, 80-fold, 90-fold, 100-fold, 200-fold, 500-fold, 1000-fold or at least 1000-fold relative to a comparator.

In some embodiments, a disclosed compound has increased selectivity for the 5-HT_{2A} receptor over another serotonin receptor (e.g., the 5-HT_{2B} receptor and/or the 5-HT_{2C} receptor) relative to a comparator. In embodiments, a disclosed compound has increased selectivity for the 5-HT_{2A} receptor over the 5-HT_{2B} receptor relative to a comparator. In embodiments, a disclosed compound has increased selectivity for the 5-HT_{2A} receptor over the 5-HT_{2C} receptor relative to a comparator. In embodiments, a disclosed compound has increased selectivity for the 5-HT_{2A} receptor over both the 5-HT_{2B} and 5-HT_{2C} receptors, relative to a comparator.

In some embodiments, selectivity is defined by the ratio of the half-maximal effective concentration (EC₅₀) of a disclosed compound for the 5-HT_{2A} receptor as compared to another receptor (e.g., a serotonin receptor, such as the 5-HT_{2B} receptor, or the 5-HT_{2C} receptor). For example, if a hypothetical compound had a 5-HT_{2A} EC₅₀ of 0.2 μM and a 5-HT_{2A} EC₅₀ of 1.0 μM, the compound could be said to have a 5-fold selectivity for the 5-HT_{2A} receptor over the 5-HT_{2B} receptor. In embodiments, a disclosed compound has about a 2-fold, 5-fold, 10-fold, 20-fold, 30-fold, 50-fold, 70-fold, 80-fold, 90-fold, 100-fold, 200-fold, or at least 200-fold selectivity for the 5-HT_{2A} receptor over the 5-HT_{2B} receptor. In embodiments, a disclosed compound has about a 2-fold, 5-fold, 10-fold, 20-fold, 30-fold, 50-fold, 70-fold, 80-fold, 90-fold, 100-fold, 200-fold, or at least 200-fold selectivity for the 5-HT_{2A} receptor over the 5-HT_{2C} receptor.

In some embodiments, a disclosed compound has an increased association rate at a serotonin receptor, such as a 5-HT₂ receptor (e.g., 5-HT_{2A}, -HT_{2B}, and 5-HT_{2A}) relative to a comparator. In some embodiments, a disclosed compound has a decreased association rate at a serotonin receptor, such as a 5-HT₂ receptor (e.g., 5-HT_{2A}, -HT_{2B}, and 5-HT_{2A}) relative to a comparator. In embodiments, the comparator is serotonin. In embodiments, the comparator is the corresponding unsubstituted phenylalkylamine. In embodiments, the comparator for XOB is 2C-B. In embodiments, the association rate of a disclosed compound at any one or more of 5-HT_{2A}, 5-HT_{2B}, and 5-HT_{2A}, is increased by about 2-fold, 5-fold, 10-fold, 20-fold, 30-fold, 50-fold, 70-fold, 80-fold, 90-fold, 100-fold, 200-fold, or at least 200-fold. In embodiments, the association rate of a disclosed compound at any one or more of 5-HT_{2A}, -HT_{2B}, and 5-HT_{2A}, is decreased by about 2-fold, 5-fold, 10-fold, 20-fold, 30-fold, 50-fold, 70-fold, 80-fold, 90-fold, 100-fold, 200-fold, or at least 200-fold. For example, in embodiments, the association rate of XOB at 5-HT_{2A} is about 24-fold slower than the association rate for serotonin (see Example 2).

c. Modulating Neurotransmission

In some embodiments, disclosed compounds modulate neurotransmission in a subject, such as following administration of a therapeutically effective amount to said subject. In embodiments, modulating neurotransmission by administering a disclosed compound to a subject treats a disease or disorder in the subject.

In some embodiments, modulating neurotransmission comprises modulating serotonergic neurotransmission. In some embodiments, disclosed compounds can modulate the activity of 5-HT receptors (5-HTRs). 5-HTRs are G-protein coupled receptors (GPCRs) that act through Gαi, Gαq/11, or Gas pathways and affect various signaling mechanisms throughout the body. Modulation of such receptors produces both distinct and overlapping pharmacological effects (Zięba et al., Int J Mol Sci., 2022; 23 (1): 10).

Each of the three 5-HT₂ receptor subtypes are G-protein-linked single protein molecules of similar size and homology, comprising between 458-471 amino acids. The pharmacology of three subtypes of the 5-HT₂ receptor, 5-HT_{2A}, 5-HT_{2B}, and 5-HT_{2C}, have been characterized, and functional activity, such as agonism and antagonism, may be determined according to certain events in resultant signal transduction cascades. See, e.g., Pithadia & Jain, J Clin Med Res. 2009; 1 (2): 72-80 and Raote et al., “Serotonin Receptors in Neurobiology,” Chapter 6, Boca Raton (FL): CRC Press/Taylor & Francis; 2007.

5-HT_{2A} activation leads to activation of GPCR subunit Gαq/11 and effector enzyme phospholipase C (PLC), which promotes release and accumulation of inositol triphosphate (IP3), diacylglycerol (DAG), and PKC (Singh et al., Int'l J Neuropsychopharmacol, 2009; 12 (5): 651-665). In one example, the released inositol phosphate (IP) can be used as an indicator of 5-HT₂ receptor signaling activity. See, e.g., 5-HT_{2A}, 5-HT_{2B} and 5-HT_{2C} Receptors: Inositol monophosphate (IP-1) formation described in Eshleman et al., Biochem Pharmacol, 2018; 158:27-34. Additionally, accumulation of IP3 causes a release of calcium, which may be monitored by loading cells with Ca²⁺ sensitive fluorescent dyes, applying a test ligand, and measuring spectral shifts that result from the dye binding to released Ca²⁺. 5-HT₂ receptor functional assay methods are described in, e.g., Klein et al., ACS Pharmacol Transl Sci. 2021; 4 (2): 533-542.

IP3 accumulation itself, e.g., accumulation of total radiolabeled IP, such as inositol mono-phosphate, inositol bis-phosphate, and inositol tris-phosphate, may also be used to measure receptor activation and desensitization, including a temporal aspect. In one example, a decrease in basal levels of IP3 provides a measure of antagonism (Raote et al., “Serotonin Receptors in Neurobiology,” Chapter 6, Boca Raton (FL): CRC Press/Taylor & Francis; 2007).

In some embodiments, disclosed compounds can modulate the activity of a 5-HT receptor, including any of activating, inhibiting, partially activating, and partially inhibiting the activity of the receptor. In embodiments, the disclosed compounds are 5-HT receptor ligands that bind to, activate, block, inhibit, or otherwise influence, e.g., via allosteric modulation, activity at a 5-HT receptor. In embodiments, a disclosed compound is a 5-HT₂ receptor ligand, such a ligand for one or more of a 5-HT_{2A} receptor, 5-HT_{2B} receptor, and 5-HT_{2C} receptor.

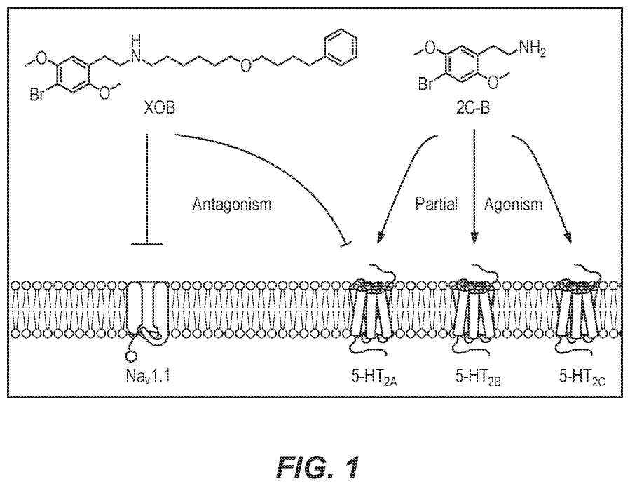

In some embodiments, a disclosed compound agonizes 5-HT₂ receptors. In embodiments, a disclosed compound antagonizes 5-HT₂ receptors. In embodiments, a disclosed compound partially agonizes 5-HT₂ receptors. In embodiments, a disclosed compound partially antagonizes 5-HT₂ receptors. (See, e.g., Example 2; FIG. 1) In embodiments, the 5-HT₂ receptor is a 5-HT_{2A} receptor. In embodiments, the 5-HT₂ receptor is a 5-HT_{2B} receptor. In embodiments, the 5-HT₂ receptor is a 5-HT_{2C} receptor.

In some embodiments, a disclosed compound has an in vitro EC₅₀ (agonist mode) for any one or more of 5-HT_{2A}, 5-HT_{2B}, and 5-HT_{2A}, that is less than 10 μM, less than 5 M, less than 1 μM, less than 0.5 μM, or less than 0.1 μM. In embodiments, a disclosed compound has an in vitro EC₅₀ (agonist mode) for any one or more of 5-HT_{2A}, -HT_{2B}, and 5-HT_{2A}, that is about 10 μM, 5 μM, 1 μM, 0.5 μM, or 0.1 μM. In embodiments, a disclosed compound has an in vitro EC₅₀ (antagonist mode) for any one or more of 5-HT_{2A}, 5-HT_{2B}, and 5-HT_{2A}, that is less than 10 μM, less than 5 μM, less than 1 μM, less than 0.5 μM, or less than 0.1 μM. In embodiments, a disclosed compound has an in vitro EC₅₀ (antagonist mode) for any one or more of 5-HT_{2A}, 5-HT_{2B}, and 5-HT_{2A}, that is about 10 μM, 5 μM, 1 μM, 0.5 μM, or 0.1 μM. For example, XOB antagonizes 5-HT_{2A} with an EC₅₀ of 1.3 μM.

In some embodiments, modulating neurotransmission comprises modulating voltage-gated ion channel activity. In embodiments, a disclosed compound modulates the activity of one or more of a voltage-gated calcium ion (Ca²⁺) channel, a voltage-gated chloride ion (Cl⁻) channel, a voltage-gated potassium ion (K⁺) channel, and a voltage-gated sodium ion (Na⁺) channel (VGSC).

In some embodiments, a disclosed compound has a binding affinity for VGSC that is less than 10 μM, less than 5 μM, less than 1 μM, less than 0.5 μM, or less than 0.1 μM. In embodiments, a disclosed compound has a binding affinity for VGSC that is about 10 μM, 5 μM, 1 μM, 0.5 μM, or 0.1 μM. In embodiments, a disclosed compound has increased binding affinity for VGSC relative to a comparator. In embodiments, the comparator is serotonin. In embodiments, the comparator is the corresponding unsubstituted phenylalkylamine. In embodiments, the comparator for XOB is 2C-B. For example, XOB has a binding affinity for 5-HT_{2A} of 2.6 μM (see Example 2). In comparison, 2C-B is not known to bind VGSC. In embodiments, the binding affinity of a disclosed compound for VGSC is increased by about 2-fold, 5-fold, 10-fold, 20-fold, 30-fold, 50-fold, 70-fold, 80-fold, 90-fold, 100-fold, 200-fold, 500-fold, 1000-fold or at least 1000-fold relative to a comparator.

In some embodiments, a disclosed compound is a VGSC inhibitor. In embodiments, a disclosed compound has an in vitro IC₅₀ for VGSC that is less than 10 μM, less than 5 μM, less than 1 μM, less than 0.5 μM, or less than 0.1 μM. In embodiments, a disclosed compound has an in vitro IC₅₀ for VGSC that is about 10 μM, 5 μM, 1 μM, 0.5 μM, or 0.1 μM. For example, XOB is a VGSC inhibitor with an IC₅₀ of 4.29 μM (see Example 2).

b. Treatment

In some embodiments, disclosed compounds are used to treat a medical condition, such as a disease or disorder. In embodiments, disclosed compounds are used in the manufacture of a medicament to treat a condition, such as a disease or disorder. Also provided are methods of administering disclosed compounds to a subject having a condition, such as a disease or disorder, thereby treating said condition.

In some embodiments, disclosed compounds and pharmaceutical compositions thereof are used to treat serotonin-mediated disorders. In embodiments, disclosed compounds, when administered to a subject in a pharmacologically effective amount, provide beneficial therapeutic effects for the treatment of serotonin-mediated disorders. Serotonin-mediated disorders include, e.g., mental health disorders, neurodegenerative diseases, pain syndromes, headaches, such as migraines, and inflammation.

In some embodiments, disclosed compounds or pharmaceutical compositions comprising the disclosed compounds are administered to a subject by one or more routes of administration, including, e.g., oral, mucosal, rectal, subcutaneous, intravenous, intramuscular, intranasal, inhaled, ocular, intraocular, topical, and transdermal routes. When administered through one or more of such routes, the disclosed compound(s) and the disclosed compositions and formulations comprising them are useful in methods for treating a patient in need of such treatment.

In embodiments, the invention provides methods of treating and/or preventing a condition in a mammal, the method comprising administering to the mammal a therapeutically effective amount of a disclosed compound or pharmaceutical composition. In some embodiments, “treating” or “treatment” refers to treating a disease or disorder in a mammal, and preferably in a human, and includes causing a desired biological or pharmacological effect, such as: (a) preventing a disorder from occurring in a subject who may be predisposed to the disorder but has not yet been diagnosed with it; (b) inhibiting a disorder, i.e. arresting its development; (c) relieving a disorder, i.e., causing regression thereof; (d) protecting from or relieving a symptom or pathology caused by or related to a disorder; (e) reducing, decreasing, inhibiting, ameliorating, or preventing the onset, severity, duration, progression, frequency or probability of one or more symptoms or pathologies associated with a disorder; and (f) preventing or inhibiting of a worsening or progression of symptoms or pathologies associated with a disorder or comorbid with a disorder. Other such measurements, benefits, and surrogate or clinical endpoints, alone or in combination, will be understood to one of ordinary skill based on the teachings herein and the knowledge in the art.

As used herein, “an effective amount,” “a pharmacologically effective amount,” or “a therapeutically effective amount” refers to an amount of an active agent that is non-toxic and sufficient to provide the desired therapeutic effect with performance at a reasonable benefit/risk ratio attending any medical treatment. The effective amount will vary depending upon the subject and the disease condition being treated or health benefit sought, the weight and age of the subject, the severity of the disease condition or degree of health benefit sought, the manner of administration, and the like, all of which can readily be determined by one of skill in the art.

As used herein, “therapeutic effect” or “therapeutic efficacy” means the responses(s) in a mammal, and preferably a human, after treatment that are judged to be desirable and beneficial. Hence, depending on the disorder to be treated, or improvement in mental health or functioning sought, and depending on the particular constituent(s) in the formulations of the invention under consideration, those responses shall differ, but would be readily understood by those of skill.

Measures of therapeutic effect includes any outcome measure, endpoint, effect measure, or measure of effect within clinical or medical practice or research which is used to assess the effect, both positive and negative, of an intervention or treatment, whether patient-reported (e.g., questionnaires), based on other patient data (e.g., patient monitoring), gathered through laboratory tests such as blood work, urine samples, etc., through medical examination by a doctor or other medical professional, or by digital tools or means, e.g., electronic tools such as online tools, smartphones, wireless devices, biosensors, or health apps.

In some embodiments, measures of therapeutic effect will include an assessment. “Assessment” refers to any means or method used with a patient, whether before, during, after, or unrelated in time to a specific treatment protocol, to measure, estimate, or evaluate a nature, ability, symptom, disorder, or other characteristic of the patient, whether qualitatively or quantitatively, and whether performed by the therapist or other clinician (e.g., an interview), by the patient his or herself (e.g., a self-reported questionnaire), by a third-party or by a computer, including a medical device (e.g., as such as defined by the FDA or other regulatory body) or other device (e.g., a medical sensor or biosensor, a watch or fitness tracker, or a “wearable”), and whether graded by a human decision-maker or an artificial intelligence, machine learning, or computer algorithm. An assessment may be computer-assisted, and other computer-assisted assessments may be performed besides the assessments above. The term “computer-assisted” in “computer-assisted assessment” means an assessment comprising the use of electronic tools such as online tools, smartphones, wireless devices, or health apps (in some such examples, also known as “digital phenotyping”). Computer-assisted assessment will include the use of an electronic psychiatric notes system, where relevant clinical information will be recorded for the duration of the therapy by a therapist interacting face-to-face with a patient, and will also include the use of computer systems where the therapist and patient interact virtually (either synchronously or asynchronously), as well as where a patient only interacts with a computer (“computer” broadly meaning any electronic tool suitable for such purposes, including desktop, laptop, and notebook computers; tablets, smartphones, and other mobile devices; watches, fitness trackers, and personal electronic devices; and the like). One or more other aspects of a psychosocial, behavioral, or drug-assisted therapy also may be “computer-assisted,” wherein one or more steps of such therapy involve the use of a computer in addition to or as a replacement for some work which would otherwise be performed by a therapist.

i. Mental, Behavioral, or Neurodevelopmental Disorders

In some embodiments, disclosed compounds are used to treat a mental, behavioral, or neurodevelopmental disorder. In some embodiments, disclosed compounds are administered, such as in a therapeutically effective amount, to a subject having a mental, behavioral, or neurodevelopmental disorder, thereby treating said mental, behavioral, or neurodevelopmental disorder. In some methods herein, the disclosed compositions, when administered in a therapeutically effective amount, provide beneficial therapeutic effects for the treatment of a mental, behavioral, or neurodevelopmental disorder.

The ICD-11, which is incorporated by reference herein in its entirety, defines “mental, behavioral, or neurodevelopmental disorders” as syndromes characterized by clinically significant disturbance in an individual's cognition, emotional regulation, or behavior that reflects a dysfunction in the psychological, biological, or developmental processes that underlie mental and behavioral functioning. Such disorders include, but are not limited to, neurodevelopmental disorders, schizophrenia or other primary psychotic disorders, catatonia, mood disorders, anxiety or fear-related disorders, obsessive-compulsive or related disorders, disorders specifically associated with stress, dissociative disorders, feeding (or eating) disorders, elimination disorders, disorders of bodily distress or bodily experience, disorders due to substance use or addictive behaviors, impulse control disorders, disruptive behavior or dissocial disorders, personality disorders (and related traits), paraphilic disorders, factitious disorders, neurocognitive disorders, mental or behavioral disorders associated with pregnancy, childbirth or the puerperium, sleep-wake disorders, sexual dysfunctions, and gender incongruence.

A mental, behavioral, or neurodevelopmental disorder where otherwise undefined, will be understood to refer to the disorder as defined in the ICD-11. Within the category of mental, behavioral, or neurodevelopmental disorders, the term mental disorder (or “mental health disorder”) generally refers to a disease condition that involves negative changes in emotion, mood, thinking, and/or behavior. In general, mental health disorders are characterized by clinically significant disturbances in an individual's cognition, emotion, behavior, or a combination thereof, resulting in impaired functioning, distress, or increased risk of suffering. Although the terms “mental disorder” and “mental health disorder,” as well as terms that define specific diseases and disorders, generally shall refer to the criteria in the ICD-11, or a patient with a diagnosis based thereon, it will be appreciated that disclosed methods are equally applicable to patients having an equivalent underlying disorder, whether that disorder is diagnosed based on the criteria in ICD-11, ICD-10, DSM-5, or DSM-IV (each of which is incorporated by reference herein in its entirety) whether the diagnosis is based on other clinically acceptable criteria, or whether the patient has not yet had a formal clinical diagnosis.

In some embodiments, disclosed compounds are used to treat a mental health disorder. In some embodiments, disclosed compounds are administered, such as in a therapeutically effective amount, to a subject having a mental health disorder, thereby treating said mental health disorder. In some methods herein, the disclosed compositions, when administered in a therapeutically effective amount, provide beneficial therapeutic effects for the treatment of a mental health disorder. In some embodiments, the compounds and compositions of the invention are used to reduce the symptoms of a mental health disorder. The symptoms of the mental health disorder to be treated shall be able to be determined by one of skill in the art, by reference to the general understanding of the art regarding that disorder.

In some embodiments, measures of therapeutic efficacy include reports by a subject or an observer. In some embodiments, measures of therapeutic efficacy include responses to a questionnaire. Non-limiting representative examples of applicable measures of symptom improvement include the Generalized Anxiety Disorder Scale-7 (GAD-7), Montgomery-Asberg Depression Rating Scale (MADRS), Global Assessment of Functioning (GAF) Scale, Clinical Global Impression (CGI), Substance Abuse Questionnaire (SAQ), Mini International Neuropsychiatric Interview 5 (MINI 5), Columbia Suicide Severity Rating Scale (C-SSRS), Patient Health Questionnaire (PHQ-9), Pittsburgh Sleep Quality Index (PSQI), Interpersonal Reactivity Index (IRI), Short Form (36) Health Survey (SF-36), Self-Compassion Scale (SCS), Trauma History Questionnaire (THQ), Beck Depression Index (BDI), and related subject- or observer-reported measures.

In some embodiments, a disclosed compound is used to treat schizophrenia (or another primary psychotic disorder, as defined in the DSM-IV, DSM-5, ICD-10, or ICD-11). Such disorders may be characterized by significant impairments in reality and alterations in behavior manifest in positive symptoms like persistent delusions, persistent hallucinations, disorganized thinking and speech, grossly disorganized behavior, as well as experience of negative symptoms such as blunted or flat affect and avolition and psychomotor disturbances. Serotonin receptors, and the 5-HT_{2A} receptor in particular, have been targets for antipsychotic therapeutics for decades (Schmidt et al., Life Sci. 1995; 56 (25): 2209-2222), and recent research supports the use of 5-HT_{2A} antagonists for the treatment of negative symptoms in patients with schizophrenia (Romeo et al., Psychiatry Res. 2023; 321:115104). In some embodiments, a disclosed compound is used to treat schizophrenia, schizoaffective disorder, schizotypal disorder, acute and transient psychotic disorder, delusional disorder, or a substance-induced psychotic disorder. In some embodiments, measurements of therapeutic efficacy in treating schizophrenia or a related psychotic disorder include the Clinical Global Impression scale (CGI), the Brief Psychiatric Rating Scale (BPRS), the Positive and Negative Syndrome Scale (PANSS), the Scale for the Assessment of Negative Symptoms (SANS), the Scale for the Assessment of Positive Symptoms (SAPS), the 16-item Negative Symptoms Assessment (NSA-16), the Schedule for Deficit Syndrome (SDS), the Clinical Assessment Interview for Negative Symptoms (CAINS), and the Brief Negative Symptoms Scale (BNSS).

In some embodiments, a disclosed compound is used to treat a mood disorder. As defined in the ICD-11, mood disorders are categorized according to the specific type(s) of mood episodes, and their pattern over time, with the primary types of mood episodes being Depressive episodes, Manic episodes, Mixed episodes, and Hypomanic episodes. Antagonism of the 5-HT_{2A} receptor is a common mechanism of numerous FDA-approved antipsychotic medications used in the treatment of mood disorders (Casey et al., Biochem. Pharmacol. 2022; 200:115028). In some embodiments, the mood disorder is a bipolar or related disorder (e.g., bipolar type I disorder, bipolar type II disorder, cyclothymic disorder) a depressive disorder (e.g., single-episode depressive disorder, recurrent depressive disorder, dysthymic disorder, mixed depressive and anxiety disorder), or a substance-induced mood disorder. In some embodiments, measurements of therapeutic efficacy in treating a mood disorder (e.g., bipolar disorder) include the General Behavior Inventory (GBI), Mood Disorder Questionnaire (MDQ), Young Mania Rating Scale, Bech-Rafaelsen Mania Rating Scale, Altman Self-Rating Mania Scale, and the Self-Report Manic Inventory.

ii. Neurodegenerative Disorders

In some embodiments, disclosed compounds and compositions thereof are used to treat neurodegenerative conditions. In embodiments, a therapeutically effective amount of a disclosed compound, or a pharmaceutical composition thereof, is administered to a subject in need thereof to treat a neurodegenerative condition. In embodiments, administration of a therapeutically effective amount of a disclosed compound slows or prevents the progression of neurodegeneration. In embodiments, administration of a therapeutically effective amount of a disclosed compound reduces the incidence or severity of at least one symptom of a neurodegenerative condition.

Neurodegeneration may be assessed, e.g., by measuring markers of neuronal loss, such as cerebrospinal fluid markers, e.g., visinin-like protein 1 (VILIP-1), tau, and p-tau181 (Tarawneh et al., Neurol. 2015; 72 (6): 656-665). Cognitive decline may also be used as a measure of neurodegeneration. Methods for assessing cognitive decline, e.g., comprehensive neuropsychological testing, are known to one of skill in the art. Exemplary cognitive evaluations include Mini-Mental State Examination (MMSE) and Montreal Cognitive Assessment (MoCA). See, e.g., Toh et al., Transl Neurodegener. 2014; 3:15. Cognitive decline and the progression of disease state may also be assessed using a condition-specific measure, e.g., the Unified Huntington's Disease Rating Scale (UHDRS).

Neurodegenerative conditions, such as diseases or disorders include, e.g., dementia, Alzheimer's disease, Huntington's disease, multiple sclerosis, and Parkinson's disease. A feature of neurodegenerative conditions is neuronal cell death, which, among other aspects, has been implicated in the promotion of inflammation. See, e.g., Chan et al., Annu Rev Immunol. 2015; 33:79-106 and Chi et al., Int J Mol Sci. 2018; 19 (10): 3082. Neurodegenerative diseases can be classified according to primary clinical features, e.g., dementia, parkinsonism, or motor neuron disease, anatomic distribution of neurodegeneration, e.g., frontotemporal degenerations, extrapyramidal disorders, or spinocerebellar degenerations, or principal molecular abnormality (Dugger & Dickson, Cold Spring Harb Perspect Biol. 2017; 9 (7): a028035).

iii. Pain and Inflammation

In some embodiments, disclosed compounds and compositions thereof are used to treat a pain disorder and/or inflammation. In embodiments, a therapeutically effective amount of a disclosed compound, or a pharmaceutical composition thereof, is administered to a subject in need thereof to treat a pain disorder and/or inflammation. In embodiments, administration of a therapeutically effective amount of a disclosed compound reduces the incidence or severity of at least one symptom of a pain disorder and/or an inflammatory disorder.

In some embodiments, a pain disorder treated by the disclosed compounds is a chronic pain disorder. Chronic pain disorders include, e.g., central pain, complex regional pain syndrome, phantom pain, such as phantom limb pain, neuropathic pain, fibromyalgia, arthritis, spinal stenosis, temporomandibular joint syndrome, bowel disease, pain related to surgery, and pain related to a disease or disorder, e.g., pain related to cancer.

In some embodiments, disclosed compounds and compositions thereof are used to treat headaches. In embodiments, a therapeutically effective amount of a disclosed compound, or a composition thereof, is administered to a subject in need thereof to treat headaches. Headaches include, e.g., tension headaches, migraine headaches, and cluster headaches.

In some embodiments, disclosed compounds and compositions thereof are used to reduce inflammation, for example, systemic inflammation. In embodiments, disclosed compounds and compositions thereof are used to treat inflammatory diseases. In embodiments, a therapeutically effective amount of a disclosed compound, or a pharmaceutical composition thereof, is administered to a subject in need thereof to treat an inflammatory disease. Inflammatory diseases include, e.g., Alzheimer's disease, ankylosing spondylitis, arthritis (osteoarthritis, rheumatoid arthritis (RA), psoriatic arthritis), asthma, atherosclerosis, Crohn's disease, colitis, dermatitis, diverticulitis, fibromyalgia, hepatitis, irritable bowel syndrome (IBS), systemic lupus erythematosus (SLE), nephritis, Parkinson's disease, and ulcerative colitis.

The International Association for the Study of Pain (IASP) defines pain as “an unpleasant sensory and emotional experience associated with actual or potential tissue damage, or described in terms of such damage.” Although the mechanism for serotonin modulators, such as 5-HT_{2A} agonists and 5-HT_{2A} antagonists, to ameliorate pain remains unclear, the synaptic plasticity associated with such compounds may alter pathologic changes in neural connections seen in chronic pain states, potentially resulting in a reduced pain intensity and duration (Castellanos et al., Reg Anesth Pain Med. 2020; 45 (7): 486-494). Additionally, 5-HT_{2A}R activation has been shown to promote anti-inflammatory effects, e.g., a reduction of TNF-α-induced inflammation. See, e.g., Pelletier & Siegel, Mol Interv., 2009; 9 (6): 299-301, Flanagan et al., Sci Rep. 2019; 9 (1): 13444, Nichols et al., Clin Pharmacol Ther. 2017; 101 (2): 209-219; Int Rev Psychiatry. 2018; 30 (4): 363-375, Okamoto et al., Neuroscience. 2005; 130 (2): 465-74.

Pain, such as chronic pain, and improvements thereof, such as a reduction of symptoms, may be measured according to known methods, e.g., by subject reporting, pain diaries, pain scales, applicable questionnaires (assessments of chronic pain and its impact on physical, emotional and social functions), ecological momentary assessments and computerized versions thereof. See, e.g., Salaffi et al., Best Practice & Research Clinical Rheumatology, 2015; 29 (1): 164-186 and Hawker et al., Arthritis Care Res (Hoboken). 2011; 63 Suppl 11: S240-52. Exemplary questionnaires include the Visual Analog Scale for Pain (VAS Pain), Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), and Measure of Intermittent and Constant Osteoarthritis Pain (ICOAP), Migraine Diagnosis Questionnaire, the Migraine-Screen Questionnaire (MS-Q), the Fibromyalgia Survey Questionnaire (FSQ).

A reduction in inflammation, such as chronic systemic inflammation, may be measured according to various methods available to one of skill. Inflammatory biomarkers may be detected from biological specimens, for example, a subject's blood, such as plasma or serum, or saliva. In one example, inflammation may be detected by measuring high-sensitivity C-reactive protein (CRP) and white blood cell count from a blood test. CRP may also be detected in a saliva sample. Salivary CRP is not synthesized locally in the mouth and may reflect more systemic levels of inflammation compared to other inflammatory biomarkers, such as cytokines (Szabo & Slavish, Psychoneuroendocrinology. 202; 124:105069). Additionally clinical pathology data, e.g., hematology data on erythrocyte parameters, platelet count, total number of leukocytes, and leukocyte differentials and morphology, coagulation data on clotting times and fibrinogen, and clinical chemistry data on total protein, albumin and globulin, liver enzymes, renal parameters, electrolytes, and bilirubin can provide an initial indication of the presence and potentially the location of inflammation, in the absence of specific data on immune tissues. See, e.g., Germolec et al., Methods Mol Biol. 2018; 1803:57-79 and Luo et al., Clin Lab. 2019 1; 65 (3).

iv. Ion Channel-Mediated Conditions

In some embodiments, disclosed compounds and compositions thereof are used to treat an ion channel-mediated condition. An “ion channel-mediated condition” refers to a disease or disorder that is related to dysfunction of an ion channel, such as a voltage-gated ion channel or a ligand-gated ion channel. In embodiments, a therapeutically effective amount of a disclosed compound, or a pharmaceutical composition thereof, is administered to a subject in need thereof to treat an ion channel-mediated condition. In embodiments, the ion channel-mediated condition is one or more of a calcium ion (Ca²⁺) channel, a chloride ion (Cl⁻) channel, a potassium ion (K⁺) channel, and a sodium ion (Na⁺) channel.

In some embodiments, a disclosed compound modulates the activity of one or more of a calcium ion (Ca²⁺) channel, a chloride ion (Cl⁻) channel, a potassium ion (K⁺) channel, and a sodium ion (Na⁺) channel. In embodiments, the ion channel is voltage-gated. In embodiments, the ion channel is ligand-gated. In embodiments, a disclosed compound modulates the activity of one or more of a voltage-gated calcium ion (Ca²⁺) channel, a voltage-gated chloride ion (Cl⁻) channel, a voltage-gated potassium ion (K⁺) channel, and a voltage-gated sodium ion (Na⁺) channel. In embodiments, modulating the activity of an ion channel comprises blocking, such as inhibiting or decreasing the activity of, said ion channel. In embodiments, modulating the activity of an ion channel comprises activating said ion channel.

In some embodiments, disclosed compounds or compositions thereof are used to treat a seizure disorder, such as epilepsy. In embodiments, a therapeutically effective amount of a disclosed compound, or a pharmaceutical composition thereof, is administered to a subject in need thereof to treat a seizure disorder. In embodiments, administration of a therapeutically effective amount of a disclosed compound reduces the incidence of seizures and/or the severity of a seizure disorder. In embodiments, the seizure disorder is epileptic seizure disorder. In embodiments, the seizure is a focal seizure. In embodiments, the seizure is a generalized seizure. In embodiments, administration of a disclosed compound to a subject results in a reduction in severity of epilepsy comorbidities, for example, anxiety and depression, and/or an increase in quality of life, for example, as evaluated using the Quality-of-Life questionnaire in Epilepsy (QOLIE-31).

A seizure is a burst of uncontrolled electrical activity between neurons that causes temporary abnormalities in muscle tone or movements, behaviors, sensations, or states of awareness (Types of Seizures, Johns Hopkins Med., accessed Jun. 24, 2022). Due to their roles in maintaining cellular ionic and electrical homeostasis, ion channels are crucial components of neuronal functioning. Certain subunits are expressed exclusively in the brain, and ion channel dysfunction may lead to epilepsy, which is characterized by recurring seizures (Armijo et al., Curr Pharm Des. 2005; 11 (15): 1975-2003; Graves, QJM: An Int'l J Med., 2006; 99 (4): 201-217). Various antiepileptic treatment options decrease membrane excitability by interacting with neurotransmitter receptors or ion channels, e.g., phenytoin and carbamazepine inhibit sodium channel activation, thereby decreasing high-frequency repetitive firing of associated action potentials (Macdonald & Kelly, Epilepsia. 1995; 36 Suppl 2: S2-12). Non-limiting examples of comorbidities of epilepsy, which may benefit from the disclosed compounds, include depression, anxiety, and migraines (Keezer et al., Lancet Neurol. 2016; 15 (1): 106-15).

Determining whether a disclosed compound modulates the activity of an ion channel may be accomplished by monitoring the electrophysiological activity of cells. For example, the patch-clamp technique allows for measurements of currents through ion channels in a cell membrane. See, e.g., Lenkey et al., PLOS One. 2010; 5 (12): e15568, Liu et al., Assay Drug Dev Technol. 201; 9 (6): 628-34, and Dolzer, Methods Mol Biol. 2021; 2188:21-49. Patch-clamp experiments can be performed on cultured cells, acutely dissociated cells, or on acute vibratome slices. Additionally, fluorescence resonance energy transfer (FRET) technology using membrane potential-sensitive dyes can provide a measurement of voltage-gated sodium channel activity in stably transfected cell lines (Felix et al., Assay Drug Dev Technol. 2004 June; 2 (3): 260-8).

v. Mental Functioning

In some embodiments, the invention provides methods of improving mental health and/or functioning, such as cognitive functioning. Improvements in mental health and functioning may include one or more of a reduction of neuroticism or psychological defensiveness, an increase in creativity or openness to experience, an increase in decision-making ability, an increase in feelings of wellness or satisfaction, or an increase in ability to fall or stay asleep. Additionally, improvements in mental health and functioning may include improvements in or a return to baseline in processing speed, learning and memory, autobiographical memory, shifting, and IQ. Measurements of such will be readily understood and appreciated according to ordinary skill. See, e.g., cognitive functioning aspects reviewed by Ahern & Semskova, Neuropsychology. 2017; 31 (1): 52-72. Exemplary measures of improvements of mental health and/or functioning include the Global Assessment of Functioning (GAF) scale, the Sleep Quality Scale (SQS) and other measures of sleep quality (see, e.g., Fabbri et al., Int J Environ Res Public Health. 2021; 18 (3): 1082, and the Social Functioning Scale (SFS) (see, e.g., Chan et al., Psychiatry Res. 2019; 276:45-55). In some embodiments, the invention provides methods of improving mental health and/or functioning, such as cognitive functioning, in healthy people, such as “healthy normals,” and the invention will thus include in some embodiments the “betterment of the well.”

vi. Administration in Conjunction with Psychotherapy

In some embodiments, a disclosed compound, or a composition thereof, is administered together with psychotherapy, such as psychosocial or behavioral therapy, including any of (or adapted from any of) cognitive behavioral therapy (e.g., as described in Arch. Gen. Psychiatry, 1999; 56:493-502), interpersonal therapy (e.g., as described in Psychol Addict Behav 2009; 23 (1): 168-174), contingency management based therapy (e.g., as described in Psychol Addict Behav 2009; 23 (1): 168-174; in J. Consul. Clin. Psychol., 2005; 73 (2): 354-59; or in Case Reports in Psychiatry, Vol. 2012, Article ID 731638), motivational interviewing based therapy (e.g., as described in J. Consul. Clin. Psychol., 2001; 69 (5): 858-62), or meditation based therapy, such as transcendental meditation based therapy (e.g., as described in J. Consul. Clin. Psychol. 2000; 68 (3): 515-52).

In some embodiments, disclosed compounds or compositions thereof are administered in conjunction with psychotherapy. “Psychotherapy” may refer to “psychedelic-assisted psychotherapy.” Psychedelic-assisted psychotherapy, broadly, includes a range of related approaches that involve at least one session where the patient ingests a psychedelic and is monitored, supported, or otherwise engaged by one or more trained mental health professionals while under the effects of the psychedelic (see, e.g., Schenberg, Front. Pharmacology, 2018; 9 (733). Protocols have been developed for the standardization of procedures which emphasize a high degree of care (see, e.g., Johnson et al., J. Psychopharmacol., 2008; 22, 603-620), such as the therapeutic approach used by MAPS to treat patients with PTSD using MDMA (see, e.g., A Manual for MDMA-Assisted Psychotherapy in the Treatment of Posttraumatic Stress Disorder (2015), published by the Multidisciplinary Association for Psychedelic Studies (MAPS) and available at: http://www.maps.org/research-archive/mdma/MDMA-Assisted-Psychotherapy-Treatment-Manual-Version7-19Aug15-FINAL.pdf).

In some embodiments, the psychotherapy conducted with a compound or composition of the invention is conducted in widely spaced sessions, typically with two administrations of a compound of the invention per session (a first dose, and a “booster” dose, although in some embodiments, only a single dose). These sessions can be as frequently as weekly but are more often approximately monthly or less frequently. In most cases, a small number of sessions, on the order of one to three, is needed for a patient to experience significant clinical progress, as indicated, for example, by a reduction in the symptoms of the mental health disorder being treated. In some embodiments, psychotherapy comprises multiple sessions, during some of which a compound of the invention is administered (“psychedelic-assisted psychotherapy,” as above, and also “drug-assisted psychotherapy”); in others, the patient participates in psychosocial or behavioral therapy without concomitant administration of a drug, or without administration of a compound of the invention.

In some embodiments, a compound or composition of the invention is administered together with standardized psychological treatment or support, which refers to any accepted modality of standard psychotherapy or counseling sessions, whether once a week, twice a week, or as needed; whether in person or virtual (e.g., over telemedicine or by means of a web program or mobile app); and whether with a human therapist or a virtual or AI “therapist.” As used herein, “therapist” refers to a person who treats a patient using the compositions and methods of the invention, whether that person is a psychiatrist, clinical psychologist, clinical therapist, registered therapist, psychotherapist, or other trained clinician, counselor, facilitator, or guide, although it will be understood that certain requirements will be appropriate to certain aspects of the drug-assisted therapy (e.g., prescribing, dispensing, or administering a drug, offering psychotherapeutic support). In some embodiments, a “person” may also include an AI.

In some embodiments, a patient will participate in a treatment protocol or a method of the invention, or be administered a composition of the invention as part of such a method, if the patient meets certain specified inclusion criteria, does not meet certain specified exclusion criteria, does not meet any specified withdrawal criteria during the course of treatment, and otherwise satisfies the requirements of the embodiment of the invention as claimed.

Preferably, where the pharmaceutical compositions of the invention are administered, such administration occurs without or with reduced risk of side effects that would require physician supervision, and therefore allow for treatment at home or otherwise outside of a clinic and without the need for such supervision, and/or additionally without the requirement of adjunctive psychotherapy (although it also may be provided in certain embodiments herein).

In some embodiments, the compounds and compositions of the invention may be administered in conjunction with or as an adjunct to psychotherapy. In other embodiments, psychotherapy is neither necessitated nor desired, or no specific type of psychotherapy is necessitated or desired, however any of the disclosed methods can be used in combination with one or more psychotherapy sessions. The flexibility to participate in specific therapies, as well as to choose between any such therapies (or to decide to forgo any specific therapy), while still receiving clinically significant therapeutic effects, is among the advantages of the invention.

In some embodiments, a patient will participate in one or more therapeutically beneficial activities, where such participation follows or is in conjunction with the administration of the provided compound or composition, including breathing exercises, meditation and concentration practices, focusing on an object or mantra, listening to music, physical exercise, yoga, stretching or bodywork, journaling, grounding techniques, positive self-talk, or engaging with a pet or animal, and it should be understood that such participation can occur with or without the participation or guidance of a therapist.

### H. EXAMPLES

The following examples are included for illustrative purposes only and are not intended to limit the scope of the invention.

### General Methods and Procedures

Drugs and Reagents: All drug recording solutions used in electrophysiology experiments were prepared on the day of recording. XOB was dissolved in 100% ethanol (EtOH). Tetrodotoxin (TTX) citrate was obtained from Alomone Labs (Jerusalem, Israel). Reagents and solvents were obtained from Sigma-Aldrich (St. Louis, MO) or other stated commercial sources.

Statistical Analysis: All data analysis of the recorded INa was performed using the software packages Clampfit v10.4 (Molecular Devices), Microsoft Excel, Graph Pad Prism v9.0 (San Diego, CA). Active and passive neuronal properties of brain slice recordings were analyzed using custom MATLAB (MathWorks) software. Results are presented as mean±S.E unless indicated otherwise.

### Example 1: Synthesis of XOB

The test compound N-(4-bromo-2,5-dimethoxyphenethyl)-6-(4-phenylbutoxy)hexan-1-amine (XOB) was synthesized according to Scheme 1.

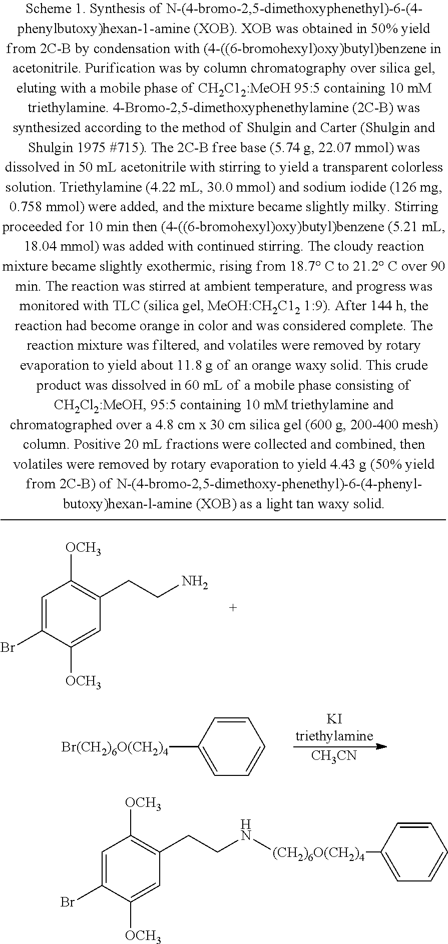

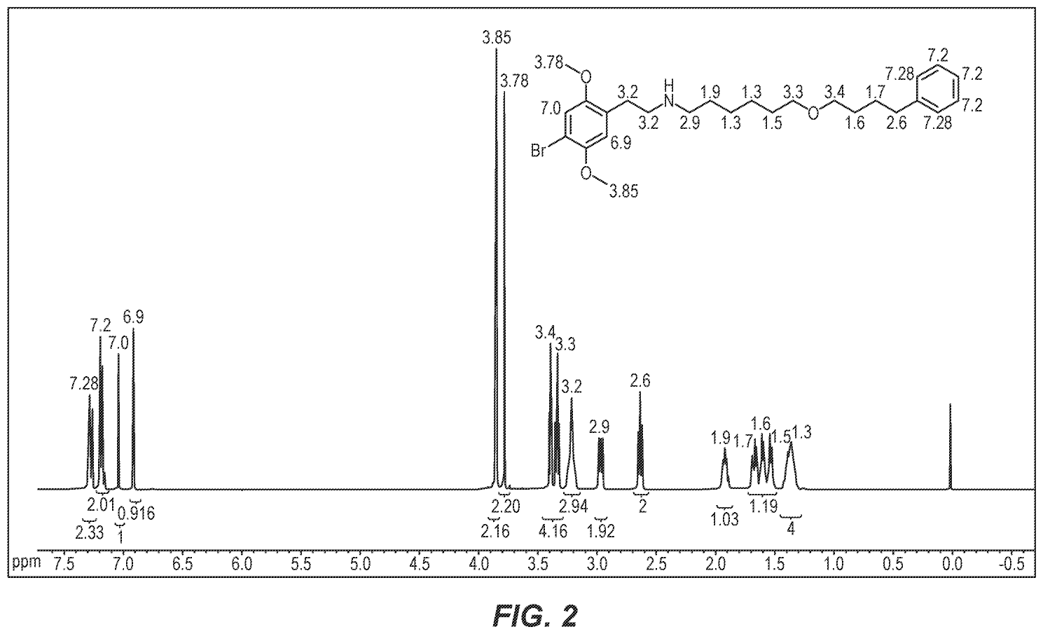

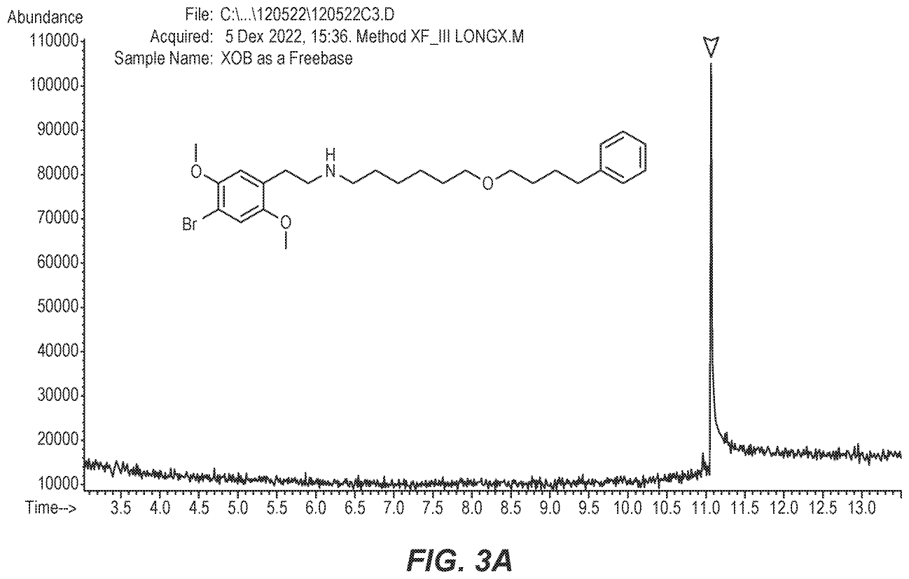

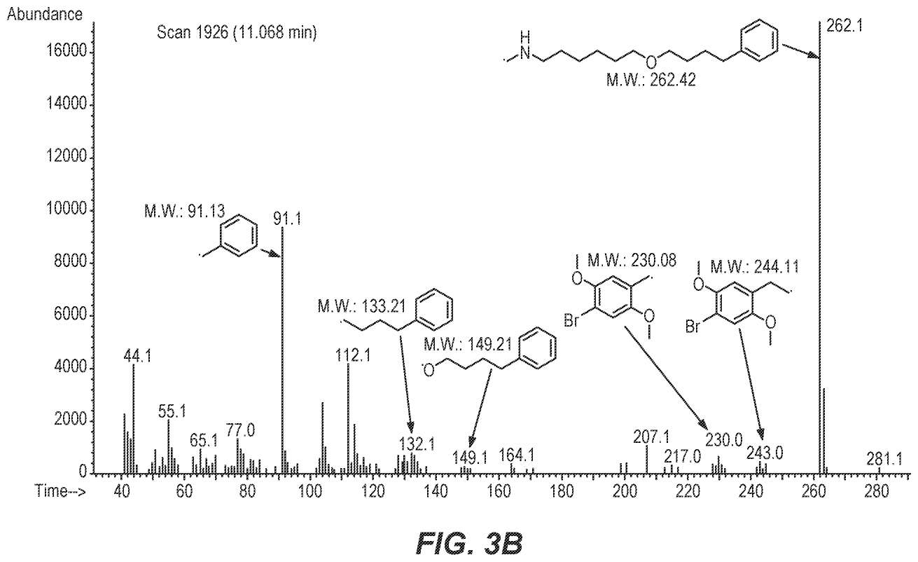

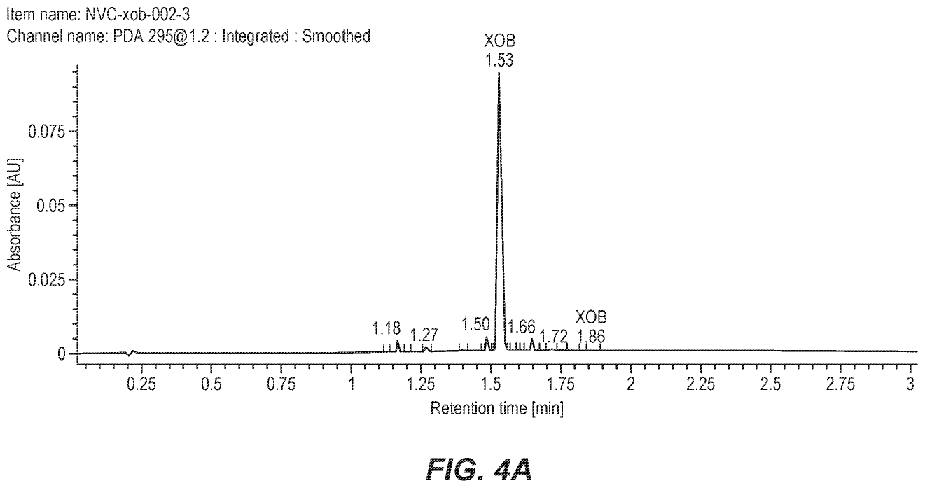

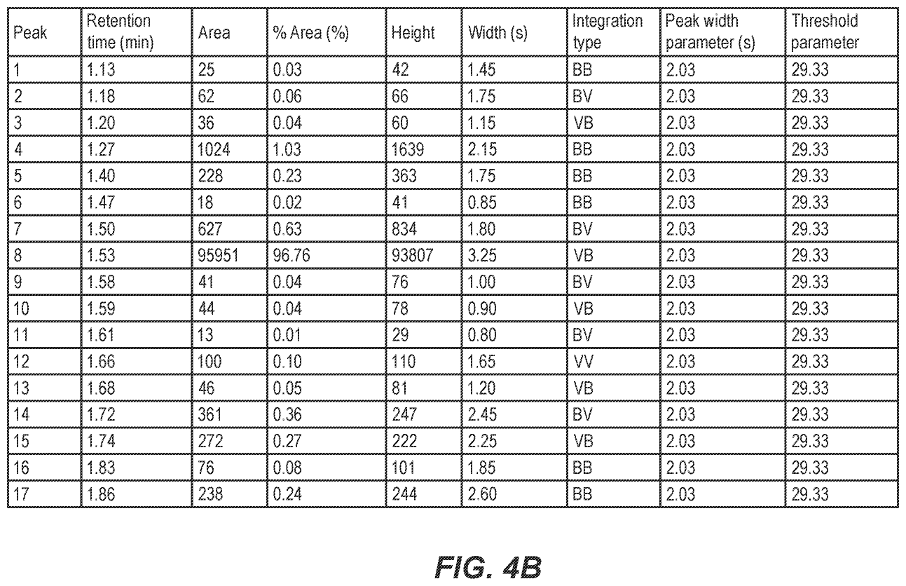

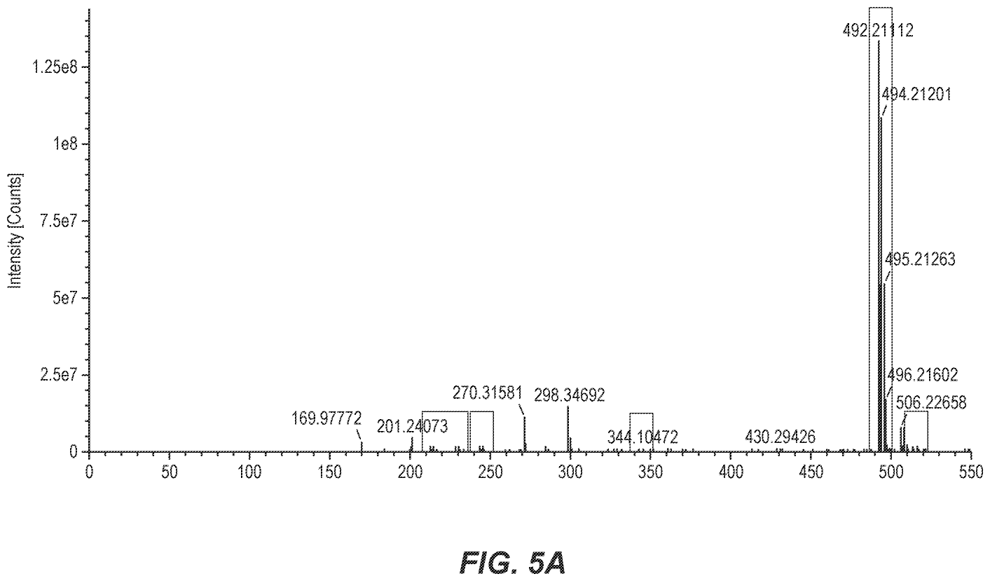

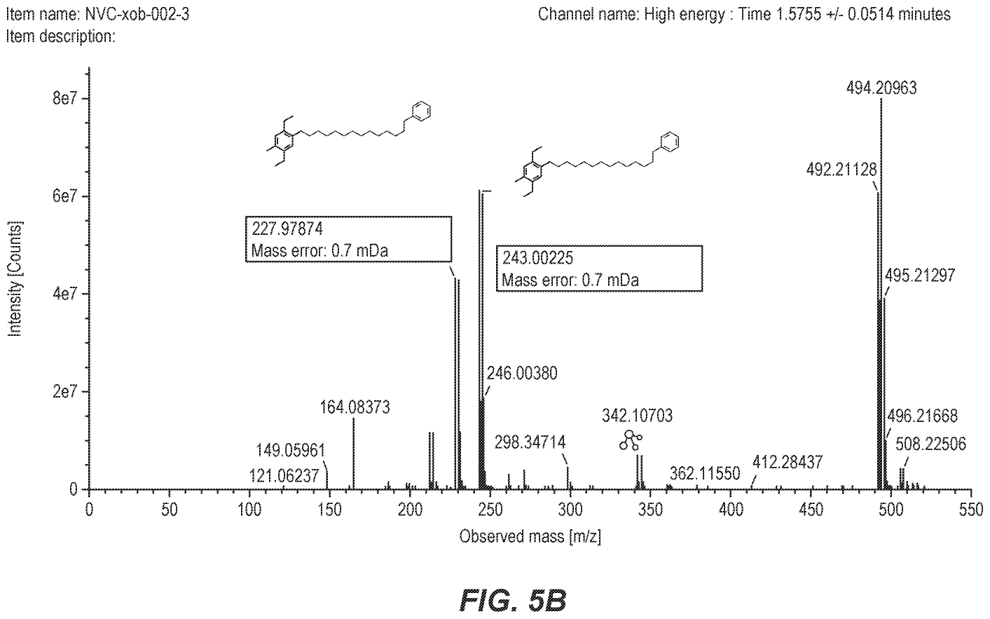

Analysis by ¹H NMR (FIG. 2), GC/MS (FIG. 3), and LC/MS/MS (FIGS. 4A, 4B, and 5) confirmed the predicted structure.

NMR: Proton NMR spectra were acquired in CDCl₃ containing 0.1% tetramethylsilane (TMS) using a 500 MHz Bruker Avance NEO spectrometer equipped with an iProbe. A 2D COSY spectrum was acquired to unambiguously assign resonances. ¹H NMR (500 MHZ, CDCl₃) δ 1.38 (4H, m, J=6.5 Hz), δ 1.55 (2H, p, J=7 Hz), δ 1.61 (2H, m, J=2.5 Hz), δ 1.68 (2H, m, J=2.5 Hz), δ 1.93 (2H, p, J=7 Hz), δ 2.64 (2H, t, J=7 Hz), δ 2.97 (2H, p, J=7 Hz), δ 3.22 (4H, m, J=5.5 Hz), δ 3.34 (2H, t, J=6.5 Hz), δ 3.40 (2H, t, J=6.5 Hz), δ 3.78 (3H, s), δ 3.85 (3H, s), δ 6.91 (1H, s), δ 7.04 (1H, s), δ 7.18 (3H, d, J=7 Hz), δ 7.28 (2H, t, J=7 Hz)

GC-MS: Samples were dissolved in ethyl acetate for GC-MS analysis using an Agilent 6890 gas chromatograph with a 7673 autosampler/injector and an Agilent 5973 mass-selective detector (MSD) system. GC-MS parameters: inlet was operated in splitless mode, injector temperature, 250C; injection volume, 1 μL; column, Agilent 19091A-105 HPULTRA1, 50 m×0.20 mm×0.33 μm film thickness; oven temperature, 50° C. for 0.5 min, ramp to 95° C. (10° C./min), hold for 2 min, ramp to 260° C. (20° C./min), hold for 4.75 min; carrier gas, hydrogen; pressure, 10 psi; flow rate, 29.6 mL/min; MSD transfer line heater temperature, 280° C.; MS scan range, m/z 40-500; total run time, 20 min.

LC MS-MS: Liquid chromatography high-resolution mass spectrometry (LC-HRMS) was performed employing a Waters Acquity I-Class UPLC system equipped with a Waters HSS T3 column (2.5 μm particle size, dimensions 2.1 mm×30 mm). The system operated in gradient mode utilizing mobile phases of water with 0.1% formic acid and acetonitrile with 0.1% formic acid at a flow rate of 0.6 mL/min. The sample for analysis was prepared by diluting in a mixture of acetonitrile and water (1:1 v/v), yielding a final concentration of 1 mg/mL. An aliquot of 0.1 μL from this prepared solution was subsequently injected for analysis. The evaluation of chemical purity was achieved by the integration of chromatographic peaks at a wavelength of 269 nm, using a photodiode array (PDA) detector. The acquisition of high-resolution mass spectra was accomplished in-line with PDA detector, using a Waters Xevo G2-XS quadrupole time-of-flight (QTof) mass spectrometer operating in ESI-positive mode. For both low and high collision energy mass spectra, data were recorded using a Waters MSe experiment and processed using the Unifi software (version 1.8).

### Example 2: Pharmacology of XOB

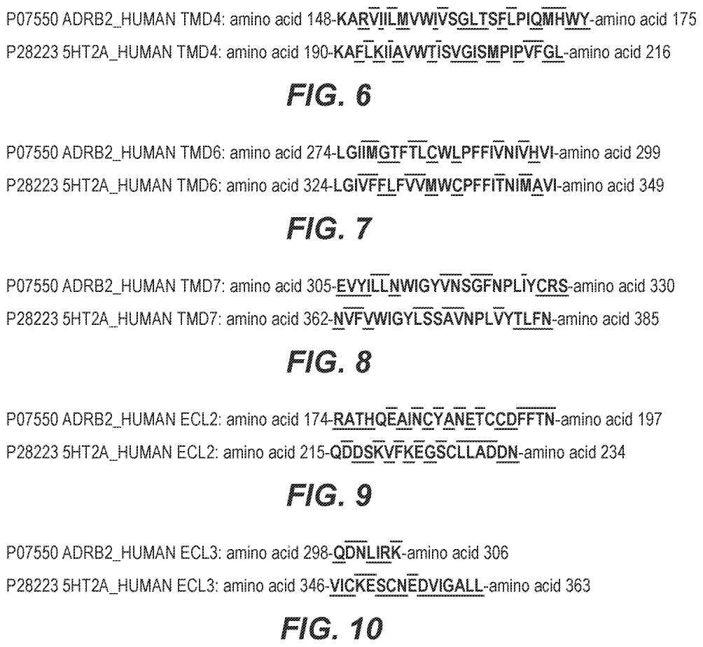

Purpose: Based on the amino acid sequence alignment between the human β₂ receptor and the human 5-HT_{2A} receptor (see, e.g., FIGS. 6-10), it was hypothesized that the 5-HT_{2A} receptor, like the β₂ receptor, contains an exosite that might engage an extended N-linked side-chain of a modified 5-HT_{2A} agonist. To test this, N-(4-bromo-2,5-dimethoxyphenethyl)-6-(4-phenylbutoxy)hexan-1-amine (XOB) was synthesized as a probe for the putative 5-HT_{2A} exosite. Binding and functional activity of XOB was tested at 5-HT_{1A}, 5-HT_{2A}, 5-HT_{2B}, and 5-HT_{2C} receptors. Additionally, in early studies, it was observed by chance that XOB produced a profound and long-lasting local anesthetic effect upon contact with mucous membranes. Thus, to test the hypothesis that XOB also affects electrical signal transduction, we evaluated its acute effects on sodium current (I_{Na}) generated by human voltage-gated sodium channel (VGSC) Naᵥ1.1 a subunits co-expressed with Naᵥβ1 subunits in human embryonic kidney (HEK) cells. In addition, we used patch clamp electrophysiology to test the acute effects of XOB on neuronal excitability in mouse cortical brain slices.

Methods: Measuring target engagement in transiently expressed HiBiT-5-HT receptors: We sought to compare the binding characteristics of XOB and the endogenous ligand serotonin at several 5-HT receptors using a NanoBRET Target Engagement assay. This assay quantifies dynamic interactions between ligands and their cognate HiBiT-tagged GPCRs on the surface of living cells through competitive binding with fluorescent tracers (Boursier et al., J Biol Chem., 2020; 295 (15): 5124-5135). For this evaluation, we used DNA constructs encoding for either 5-HT_{1A}, 5-HT_{2A}, or 5-HT_{2C} receptors genetically fused to an N-terminal HiBiT tag, which can generate bright luminescence upon high affinity complementation with LgBiT, an impermeable 18 kDa subunit derived from NanoLuc (Killoran et al., Molecules., 2021; 26 (10): 2857).

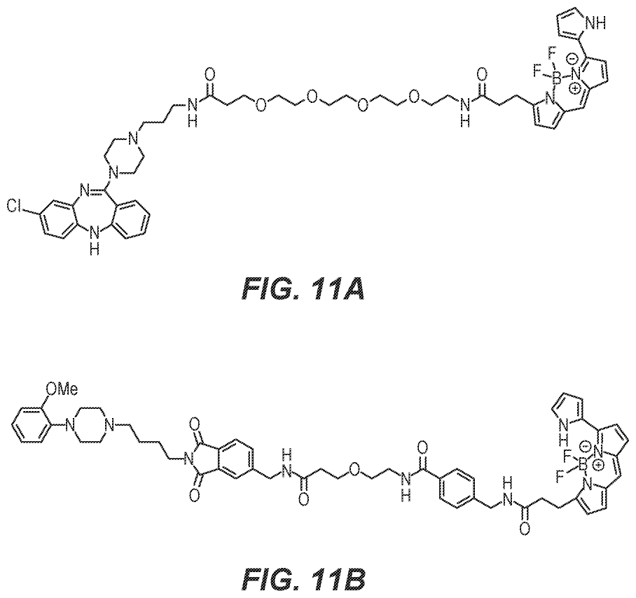

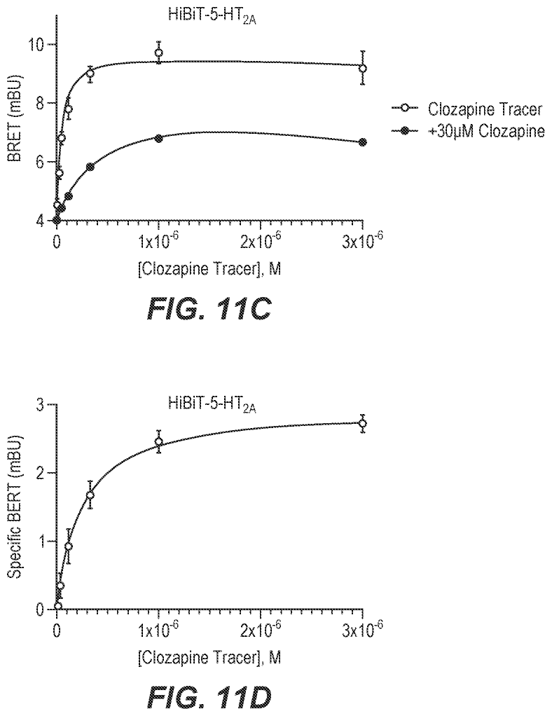

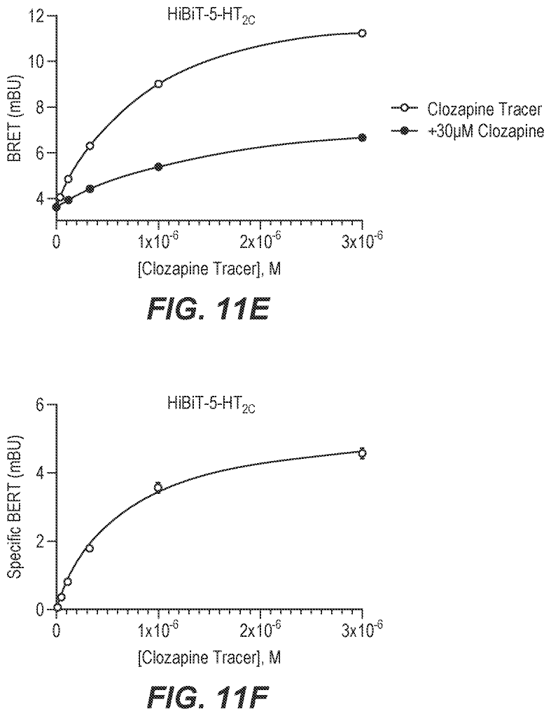

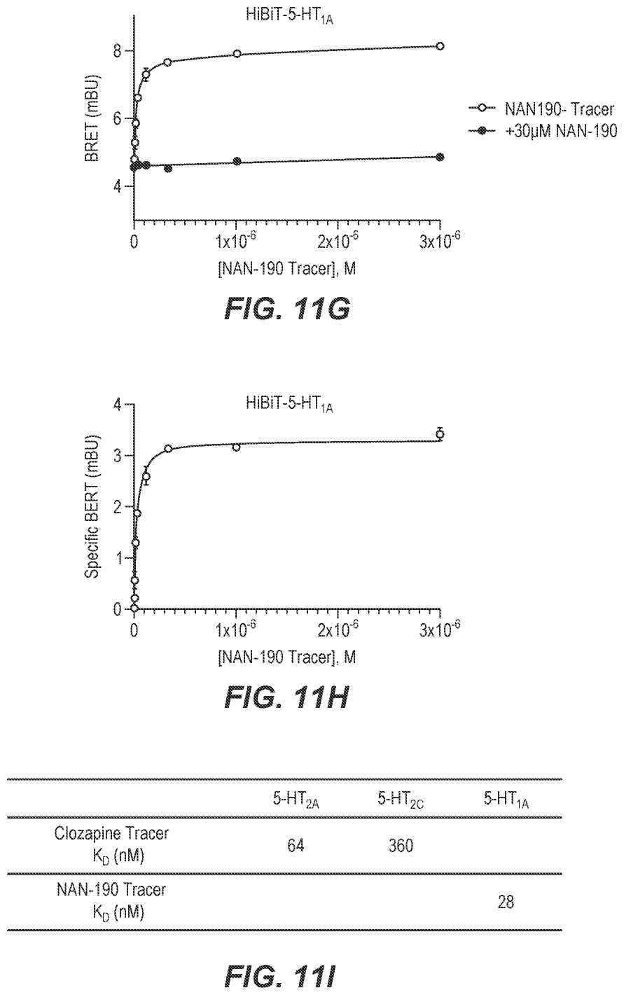

First, for each receptor, we determined the binding constant for one of two fluorescent tracers comprising a NanoBRET-590 fluorophore conjugated to either clozapine or NAN-190 (Killoran et al., Molecules, 2021; 26 (10): 2857). To this end, HEK293 cells transiently expressing a HiBiT-tagged receptor were treated with increasing concentrations of a fluorescent tracer in the absences or presence of excess unmodified ligand (FIGS. 11A-11I). Binding constants (K_{D}) derived from these saturation binding analyses revealed affinities of 64 nM and 360 nM for the clozapine tracer at 5-HT_{2A} and 5-HT_{2C} receptors, respectively, and affinity of 28 nM for the NAN-190 tracer at 5-HT_{1A} receptors.

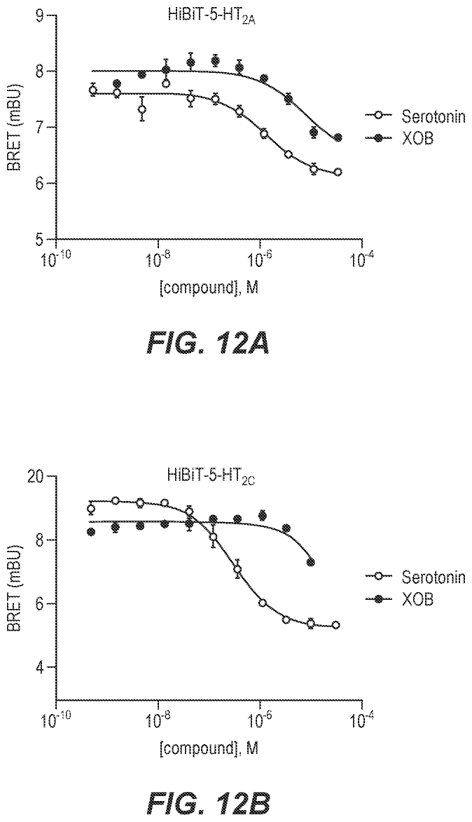

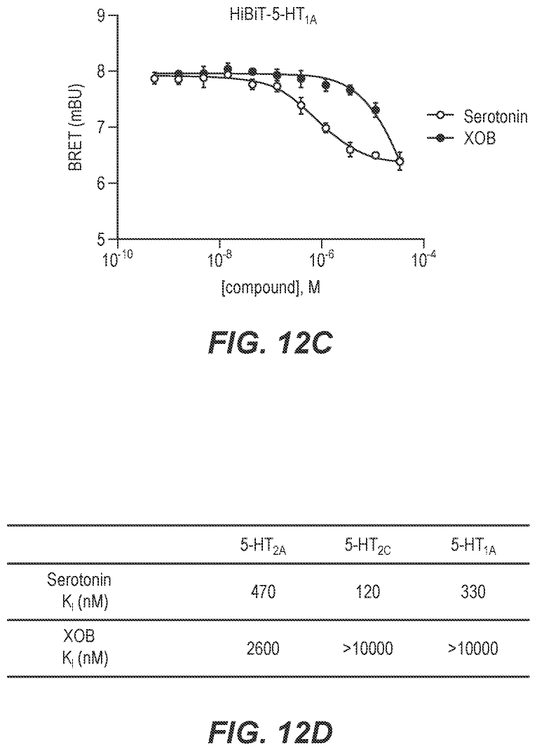

Having the binding constants for the fluorescent tracers in hand, we further evaluated the binding affinities for unmodified XOB and serotonin at these receptors. HEK293 cells transiently expressing the HiBiT-tagged receptors were tested for competitive displacement of a fixed EC₈₀ concentration of a fluorescent tracer by increasing concentrations (1 pM-30 μM) of unmodified XOB or serotonin (FIGS. 12A-12D). Binding constants calculated according to the Cheng-Prusoff equation (Cheng and Prusoff, Biochem Pharmacol., 1973; 22 (23): 3099-3108).

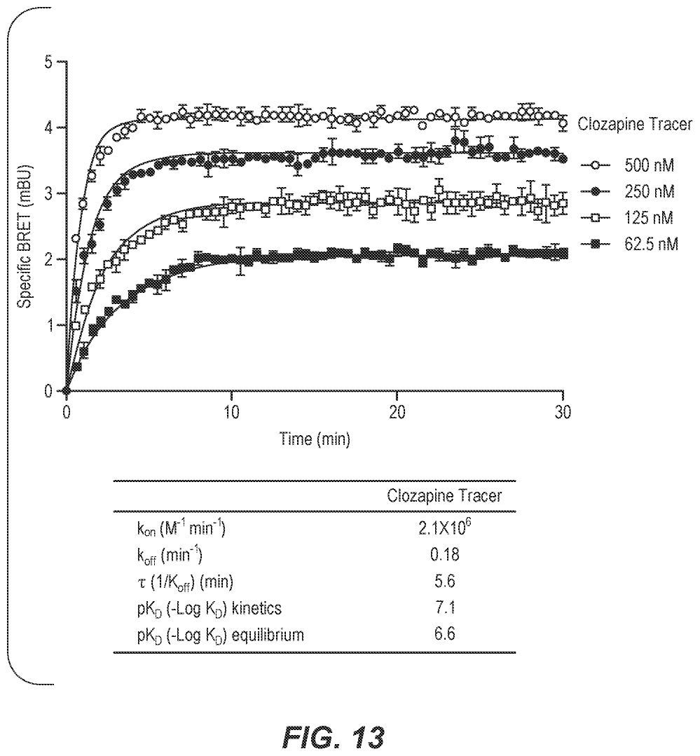

We opted to further compare XOB and serotonin for their binding kinetics at 5-HT_{2A} receptors, which may be more relevant for predicting potency. First, we determined the kinetic constants for the clozapine tracer. To this end, HEK293 cells transiently expressing HiBiT-5-HT_{2A} receptors were treated with varying concentrations of clozapine tracer in the presence and absence of excess unmodified clozapine and binding was monitored over 30 minutes (FIG. 13). The binding affinity derived from this kinetic analysis matched the one derived from saturation binding experiments and revealed moderate association and fast dissociation rates. We further monitored over 30 minutes the binding of serotonin and XOB to HiBiT-5-HT_{2A} receptors in the presence of fixed EC₈₀ concentration of clozapine Tracer. Kinetic-derived binding affinities were generally comparable with affinities derived from equilibrium analyses but at the same time revealed that the weak potency of XOB for 5-HT_{2A} receptors is likely underlined by its slow association rate, which is two orders of magnitude slower than the association rate for serotonin.

Cell lines: Human Embryonic Kidney (HEK) 293 cells stably expressing human VGSC a subunit Naᵥ1.1 (GenBank accession number NP_008851.3) were a gift from Dr. Massimo Mantegazza. Human VGSC β₁ subunit GenBank accession number NP_001028.1) cDNA was transfected into this line to generate a second, stable line. Cells were cultured in Dulbecco's Modified Eagle Medium containing (4.5 g/L D-glucose, L-glutamine, 110 mg/L sodium pyruvate, 200 μg/mL G418, 100 U/mL penicillin/streptomycin). All cells were maintained in an incubator at 37° C. with 5% CO₂.

Animals: Wildtype C57BL/6J mice were obtained from the Jackson Labs. Male and female pups, age postnatal day (P) 17-23, were used for the electrophysiological experiments. Animals were housed in the Unit for Laboratory Animal Medicine at the University of Michigan. All procedures were performed in accordance with the NIH and approved by the University of Michigan IACUC.

Manual Sodium Current Recording: Sodium current (I_{Na}) was measured at room temperature using the manual whole-cell patch clamp technique with previously described electrophysiological methods (Chen et al., J Biol Chem., 2012; 287 (46): 39061-39069). Cells were plated on 12 mm diameter clear glass poly-D-lysine coated coverslips (Neuvitro) and used for electrophysiological recordings within 48 hours after plating. Cells were identified with an AIR upright confocal microscope (Nikon). Micropipettes were obtained from 1.5 mm outer diameter capillary glass tubing (Harvard Apparatus) using a P-97 horizontal puller (Sutter Instrument Co.). Micropipettes were then polished using a MF-830 micro forge (Narishige) to obtain a resistance between 2.0 to 5.0 M (2. The intracellular solution contained the following (in mM): 1 NaCl, 125 N-methyl-D-glucamine, 2 MgCl₂, 10 EGTA, 40 HEPES, 5 phosphocreatine-tris, 2 Mg-ATP, 0.2 Na₂-GTP, 0.1 leupeptin, 270-275 mOsm, pH 7.2 with H₂SO₄. Extracellular solution contained the following (in mM): 120 NaCl, 1 BaCl₂, 2 MgCl₂, 0.2 CdCl₂, 1 CaCl₂), 20 sucrose, 10 glucose, 10 HEPES, 10 tetraethylammonium chloride, 300-305 mOsm, pH 7.35 with NaOH. Signals were amplified using a Multiclamp 700B amplifier (Molecular Devices). Data were acquired with a Digidata 1440A interface (Molecular Devices) and analyzed using pClamp10 offline. Pipette and whole-cell capacitance were fully compensated, and series resistance was predicted and compensated at 50%. Signals were low pass-filtered at 10 kHz, and data were sampled at 20 kHz. Residual linear capacity and leak currents were eliminated using online P/4 subtraction. Gravity-powered perfusion of XOB was used in all manual patch clamp experiments with a flow rate of 2-3 mL/min. All manual whole-cell I_{Na} recorded under XOB conditions was conducted after 3.9 minutes of perfusion unless indicated otherwise.

Automated Sodium Current Recording (SyncroPatch); Automated whole cell sodium current recordings were conducted on the SyncroPatch 384, housed in the University of Michigan Center for Chemical Genomics, according to Nanion's standard procedures. Single-hole medium resistance chips were used. Immediately prior to recording, HEK 293 cells were dissociated with TryplE, quantified with an automated cell counter (Nano EnTek) and resuspended in extracellular solution at a concentration of 800 thousand cells per mL. Extracellular solution contained the following (in mM): 140 NaCl, 4 KCl, 2 CaCl₂, 1 MgCl₂, 5 glucose, 10 HEPES, 298 mOsm. pH 7.4 with NaOH. Intracellular solution contained the following (in mM): 110 KF, 10 NaCl, 10 KCl, 10 EGTA, 10 HEPES, 285 mOsm, pH 7.2 with KOH. Currents were digitized at 20 kHz and lowpass filtered at 5 kHz. Series resistance was automatically compensated. Currents were leak subtracted using the leak correction method implemented in PatchControl384 with 1 or 2 square pulses stepping from the holding potential of −80 mV to −60 mV for 10 ms. The combined I-V protocol used to examine activation and inactivation was sampled at 10 kHz and lowpass filtered at 5 kHz. To determine the INa amplitude and the voltage dependence of activation, I_{Na} was evoked from a holding potential of −80 mV to −120 mV for 500 ms followed by a pre-step to −10 mV for 50 ms. The membrane was hyperpolarized back to −120 mV for 500 ms before a 500 ms test pulse ranging from −120 mV to +30 mV (in 5 mV increments). Immediately following the test pulse, the voltage-dependence of inactivation was determined by stepping to −10 mV for 50 ms from the same voltages as described for the voltage dependence of activation. SyncroPatch quality control criteria for the I-V protocol were set to capacitance <35 pF, peak I_{Na} more negative than −200 pA, series resistance between 1 and 35 MΩ, and seal resistance>200 MΩ2. Peak I_{Na} was normalized to cell capacitance to obtain current density, used to plot I-V curves and calculate conductance with the following equation: g=I÷(V−Vᵣₑᵥ).

where g is conductance, I is current, V is the test potential, and Vᵣₑᵥ is the measured reversal potential. Peak currents were normalized to the maximum peak I_{Na} amplitude. The V_{1/2} of activation represents the voltage of the membrane at which half-maximal peak I_{Na} amplitude occurred. Normalized voltage-dependence of activation and inactivation curves were fit with the following Boltzmann equation:

$$\frac{1}{1 + {e}^{\frac{V - {V}_{1 / 2}}{k}}} .$$

where V_{1/2} is the membrane potential in the midpoint of the curve, and k is the slope factor. The peak I_{Na} protocol was sampled at 20 kHz and low pass filtered at 5 kHz. The membrane was brought from the holding potential of −80 mV to −120 mV for 500 ms, then stepped to −10 mV for 500 ms to elicit peak I_{Na} before returning to −80 mV. The peak I_{Na} protocol was implemented as a repeating sweep running at 0.1 Hz throughout drug perfusion to monitor compound effects over 42 sweeps. Normalized peak I_{Na} inhibition was calculated using the following equation: Peak I_{Na} Denisty Inhibition=1−(I_{Drug}÷I_{Reference}).

Where I_{Reference} is the average peak I_{Na} density elicited from the first 10 sweeps under perfusion of the Nanion standard external (reference) solution, and I_{Drug} is the average peak I_{Na} density elicited during the last 10 sweeps after 3.5 minutes of vehicle, TTX or XOB perfusion. The quality control settings for the peak I_{Na} protocol were the same as the I-V protocol except the cell capacitance was <30 pF. To obtain the XOB concentration response curve in FIG. 2A, the normalized peak I_{Na} density inhibition data was fit with nonlinear regression using the four-parameter variable slope inhibitor vs response equation in GraphPad Prism. The data point for 1 pM XOB was set equivalent to the vehicle response and 1 mM XOB was set equivalent to the response of 1 μM TTX.

Brain Slice Preparation: Acute brain slices were prepared as described previously (Hull et al., Ann Clin Transl Neur., 2020; 7 (11): 2137-2149). Briefly, mice were anesthetized with isoflurane anesthesia and decapitated. Brains were carefully removed from the skull and placed in ice-cold carbogen-aerated slice solution containing (in mM): 110 sucrose, 62.5 NaCl, 2.5 KCl, 6 MgCl₂, 1.25 KH₂PO₄, 26 NaHCO₃, 0.5 CaCl₂), 20 D-glucose, pH 7.35-7.40. Brains were blocked and slices were cut using a vibrating microtome (Electron Microscopy Sciences) in 250 μm thick coronal sections from prefrontal cortex. Slices were incubated in an aerated holding chamber containing slice solution for 30 minutes at room temperature and then incubated in 1:1 slice: artificial cerebrospinal solution (ACSF) for 30 minutes at 35° C. ACSF contained in mM (125 NaCl; 2.5 KCl; 1 MgCl₂; 1.25 KH₂PO₄; 26 NaHCO₃; 2 CaCl₂); and 20 D-glucose (pH 7.35-7.40). Slices were then aerated in a holding chamber containing 100% ACSF for at least 30 min at room temperature before recording.

Action Potential Recording and Analysis: Individual brain slices were placed in an RC-26 recording chamber (Warner Instruments) and superfused with aerated ACSF at a flowrate of 2-3 mL/min equipped with an inline heater (Warner Instruments) to maintain the recording temperature at 33-35° C. Layer 5 pyramidal neurons were identified based on their large soma size, shape, and location using an AIR upright confocal microscope (Nikon) equipped with IR-DIC optics and a 40× water immersion objective. Only vertically oriented pyramidal cells were selected for recording. Recording electrodes had a resistance of 4-8 MΩ with solutions containing (in mM): 140 K-Gluconate, 4 NaCl, 0.5 CaCl₂, 10 HEPES, 5 EGTA, 5 phosphocreatine, 2 Mg-ATP, and 0.4 GTP (pH 7.2-7.3 with KOH). The junction potential was calculated to be 14.3 mV using the P-clamp junction potential calculator and all values presented in the study are uncorrected. Following break in at −94.3 mV in voltage clamp mode, the resting membrane potential was defined as the membrane potential in current clamp <10 s after initial break in for baseline or immediately after XOB perfusion. Repetitive action potential firing was elicited in whole-cell current clamp from the resting membrane potential in 1 s long current injections in 10 pA steps from −20 pA to +400 pA. There was a 1 s long 0 pA current injection between each sweep. Data were acquired at 20 kHz and were filtered at 10 kHz. Cells with an access resistance measured in voltage clamp >20 MΩ or RMP more depolarized than −64.3 mV were not used. Access resistance and pipette capacitance were compensated using bridge balance. Whole cell capacitance was measured using P-clamp whole cell capacitance compensation in voltage clamp with 10 mV depolarizing steps from −94.3 mV. Automated action potential (AP) quantification and analysis was performed using custom MATLAB (MathWorks) software. APs were defined as the voltage crossing 0 mV after a dV/dt>10 mV/ms, defined here as the AP threshold. Input resistance was calculated using Ohm's law with a −10 pA current injection from the resting membrane potential after 250 ms.

### 5-HT Receptor Binding Assays:

NanoBRET-based ligand binding assays: HEK293 cells expressing a HiBiT-tagged 5-HT receptor were treated with a serial dilution of a fluorescent tracer in the presence or absence of 30 μM competing unmodified ligand. Plates were mixed briefly and incubated for 90 minutes at room temperature. To measure BRET, cells were treated with a 2× detection solution comprising 100-fold dilution of LgBiT (Promega) and 50-fold dilution of furimazine Live Cell Substrate (Promega) in Opti-MEM. Plates were mixed for 10 minutes to allow HiBiT/LgBiT complementation. Filtered luminescence was then measured using GloMax Discover Microplate Reader (Promega) equipped with a 450 nm (8-nm bandpass) filter (donor) and a 600-nm long pass filter (acceptor). BRET was calculated by dividing the acceptor >600 nm light output by the donor 450 nm emission. Values were background corrected by subtracting the BRET values from samples treated with excess unmodified ligand.

For competitive displacement experiments, cells were treated with serial dilution of unmodified ligand in the presence of a fixed EC₈₀ concentration of a fluorescent tracer. Plates were mixed briefly and incubated for 90 minutes at room temperature. Cells were then treated with a 2× detection solution and BRET measurements were performed as described above. Affinity values (K_{I}) were calculated from the observed IC₅₀ values according to the Cheng-Prusoff equation (Cheng and Prusoff, Biochem Pharmacol., 1973; 22 (23): 3099-3108).

Kinetic measurements of ligand binding to HiBiT-tagged 5-HT receptors: For binding kinetics of clozapine tracer, cells were treated with a 2× detection solution comprising 100-fold dilution of LgBiT (Promega) and 50-fold dilution of furimazine Live Cell Substrate (Promega) in Opti-MEM without phenol red. To determine specific binding, control wells were also treated with a final concentration of 30 μM clozapine. Plates were mixed for 15 minutes prior to the addition of serially diluted clozapine tracer. Following brief mixing kinetic reads were immediately collected on a GloMax Discover Microplate Reader (Promega).

For binding kinetics of an unmodified ligand, as described above, cells were first treated with a 2× detection solution and control wells were additionally treated with excess clozapine. Following 15 minutes incubation, cells were treated with serially diluted unmodified ligand and a fixed EC80 concentration of clozapine tracer. Following brief mixing, kinetic measurements were immediately collected on a GloMax Discover Microplate Reader (Promega).

Data analysis: GraphPad Prism software was used to derive saturation KD values from a “one site-binding” fitting and competition IC₅₀ values from a “log (inhibitor) vs. response-variable slope” fitting. The IC₅₀ values were then used to derive binding affinities for unmodified ligands (KI) according to the Cheng-Prusoff equation, where [L] is the concentration of the fluorescent ligand in the assay and KD is its affinity in a saturation binding experiment:

$${K}_{i} = \frac{{IC}_{50}}{1 + \frac{[ L ]}{{K}_{D}}}$$

Kinetic analysis for the fluorescent tracer was graphed using the association kinetics-two or more concentration of hot fit. Kinetic constants (kₒₙ and k_{off}) and binding constant (K_{D}) for the fluorescent tracer were determined from the resulting curves (Tummino et al., Biochemistry—US., 2008: 47 (20): 5481-5492). Kinetic analyses for unmodified compounds were graphed using the kinetics of competitive binding fit (Motulsky-Mahan model for kinetics of competitive binding) (Motulsky and Mahan, Mol Pharmacol., 1984; 25 (1): 1-9). Binding affinity and other kinetic constants were determined from the resulting curves.

5-HT Receptor Functional Assay: A stably expressing 5-HT₂ receptor Flp-In 293 T-Rex Tetracycline inducible system (Invitrogen, mycoplasma-free) was used for calcium flux assays, as described and utilized previously (Klein et al., ACS Pharmacol Transl Sci., 2021; 4 (2): 533-542). Cell lines were maintained in DMEM containing 10% FBS, 10 μg/mL Blasticidin (Invivogen), and 100 μg/mL Hygromycin B (GoldBio). Day before the assay, receptor expression was induced with tetracycline (2 μL/mL) and seeded into 384-well poly-L-lysine-coated black plates at a density of 7,500 cells/well in DMEM containing 1% dialyzed FBS. On the day of the assay, the cells were incubated with Fluo-4 Direct dye (Invitrogen, 20 μl/well) for 1 h at 37° C., which was reconstituted in drug buffer (20 mM HEPES-buffered HBSS, pH 7.4) containing 2.5 mM probenecid. After dye load, cells were allowed to equilibrate to room temperature for 15 minutes, and then placed in a FLIPRTETRA fluorescence imaging plate reader (Molecular Devices). Drug dilutions were prepared at 5× final concentration in drug buffer (20 mM HEPES-buffered HBSS, pH 7.4) supplemented with 0.3% BSA fatty-acid free and 0.03% ascorbic acid. Drug dilutions were aliquoted into 384-well plastic plates and placed in the FLIPRTETRA for drug stimulation. Fluorescence reads were programmed to record baseline fluorescence for 10 s (1 read/s), and afterward 5 μl of drug per well was added and read for a total of 2 min (1 read/s). Fluorescence in each well was normalized to the average of the first 10 reads for baseline fluorescence, and then maximum-fold peak increase was calculated. Peak was plotted as a function of drug concentration, and data were normalized to percent 5-HT stimulation. For antagonist mode, plates were challenged with 3.2 nM 5-HT to measure calcium flux blockade response. Data were plotted and non-linear regression was performed using “log (agonist) vs. response” in GraphPad Prism 9 to yield Emax and EC₅₀ parameter estimates.

Results: Binding affinities for XOB and serotonin at HiBiT-tagged receptors transiently expressed in HEK293 cells revealed moderate K values for serotonin of 330 nM, 470 nM, and 120 nM at 5-HT_{1A}, 5-HT_{2A}, and 5-HT_{2C} receptors, respectively, in general agreement with reported values. Binding affinities for XOB were significantly lower with K_{I}=2.6 μM at 5-HT_{2A} receptors and K_{I}>10 μM at 5-HT_{1A} and 5-HT_{2C} receptors (FIGS. 12A-12D).

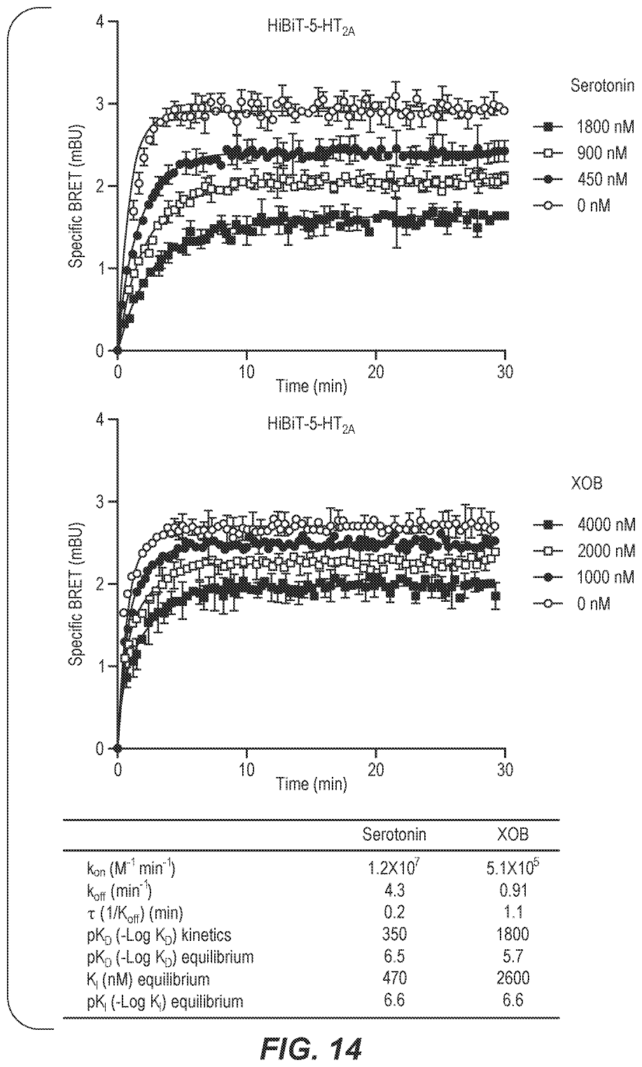

The binding affinity derived from the kinetic analysis matched the affinity derived from saturation binding experiments and revealed moderate association and fast dissociation rates. We monitored the binding of serotonin and XOB to HiBiT-5-HT_{2A} in the presence of fixed EC₈₀ concentration of the clozapine tracer over 30 minutes (FIG. 14). Kinetic-derived binding affinities were generally comparable with affinities derived from equilibrium analyses but at the same time revealed that the weak potency of XOB at 5-HT_{2A} is likely explained by its slow association rate, which is about 24-fold slower than the association rate for serotonin.

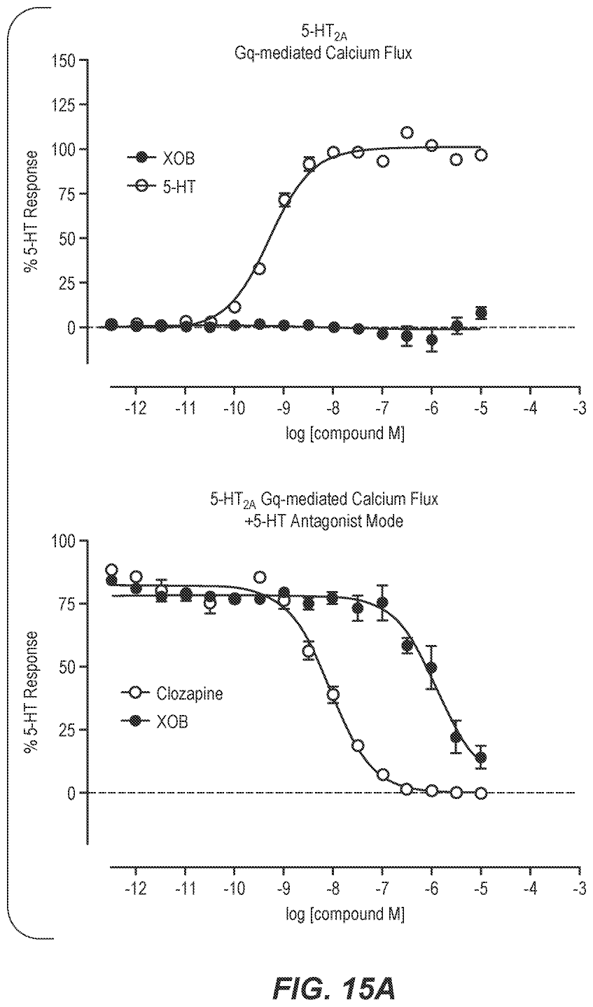

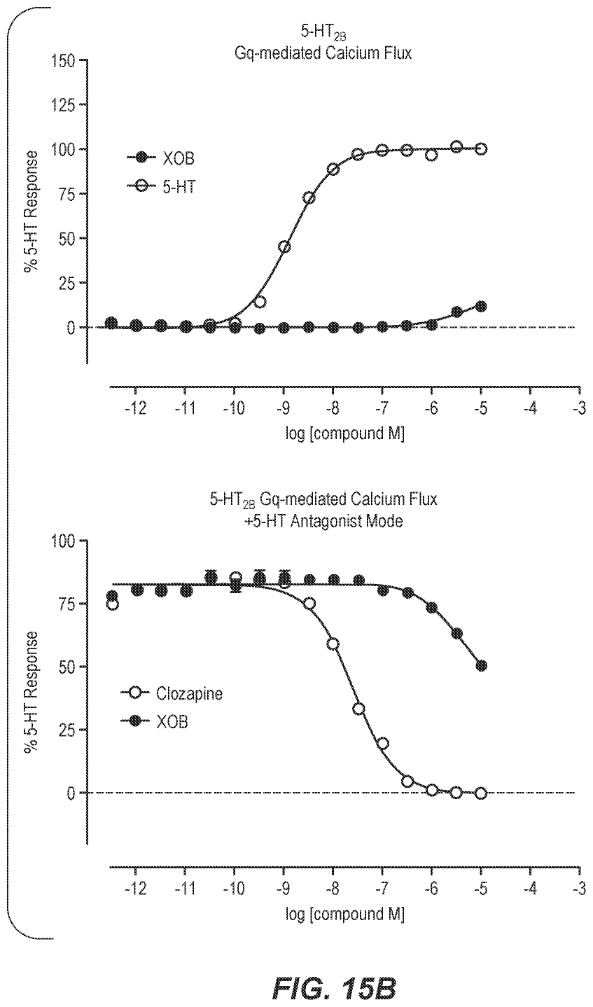

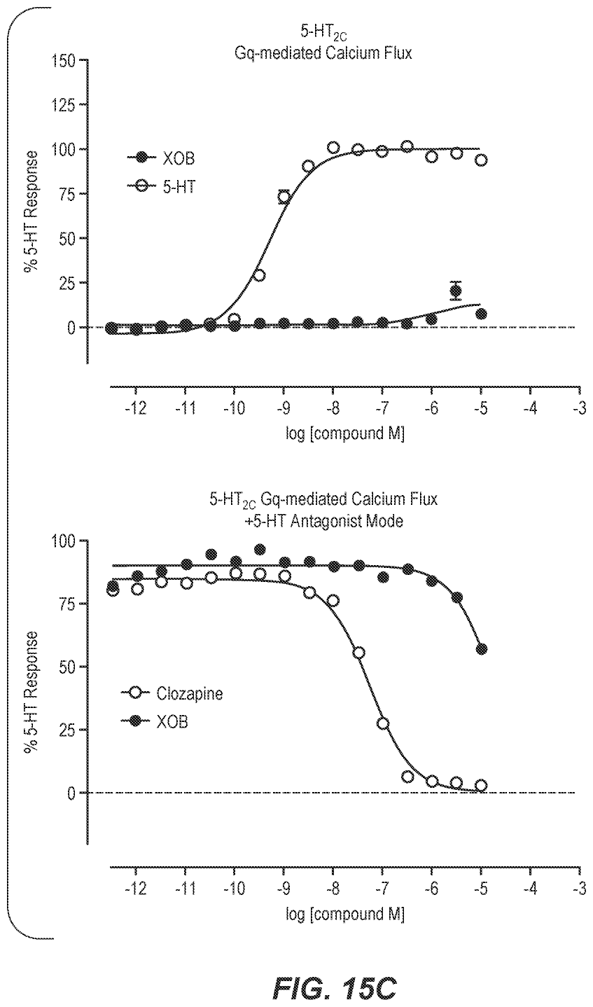

To measure functional activity at 5-HT₂ receptors, functional assays were performed measuring Gq-mediated calcium flux activity at human 5-HT_{2A}, -HT_{2B}, and 5-HT_{2C} receptors stably expressed in a T-Rex tetracycline-inducible cell line (FIGS. 15A-15C). XOB showed little to no measurable agonist activity at any of the 5-HT₂ receptors up to 10 μM concentrations. However, when XOB was tested in competition with 5-HT in antagonist mode, XOB exhibited low micromolar antagonism at 5-HT_{2A} (IC₅₀=1.3 μM), which was similar to the measured affinity (K_{I}=2-3 μM) value obtained using NanoBRET. XOB was less potent, however, at blocking 5-HT-mediated calcium flux responses at 5-HT_{2B} and 5-HT_{2C} receptors, producing <50% inhibition at concentrations up to 10 μM, thus showing 5-HT_{2A} antagonist selectivity over the closely related 5-HT₂ receptors.

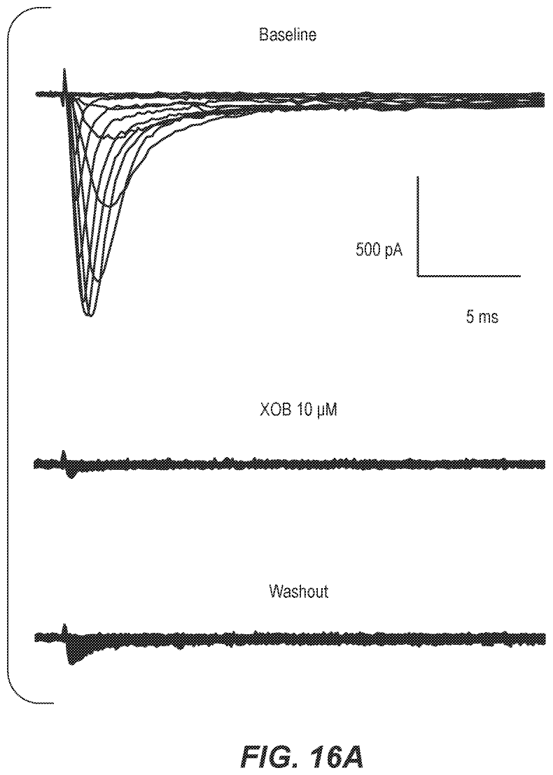

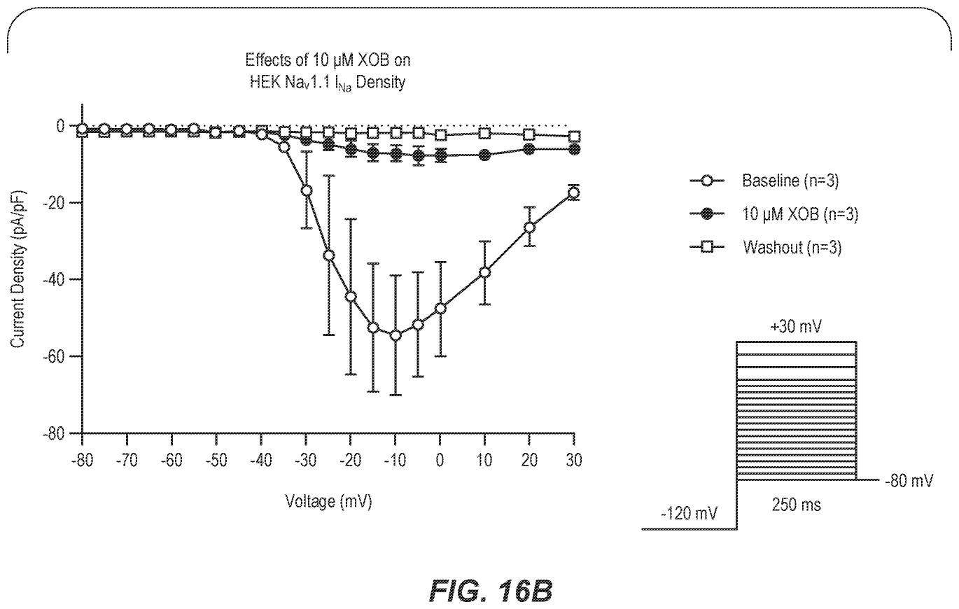

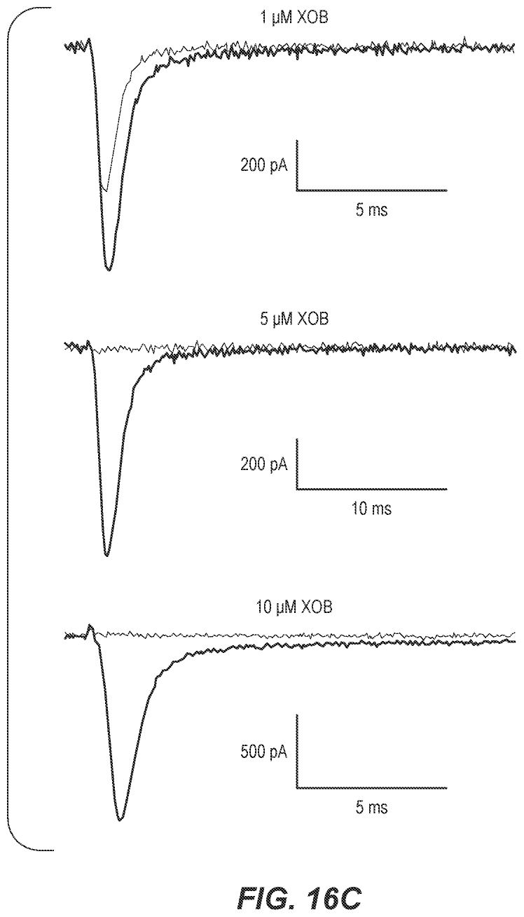

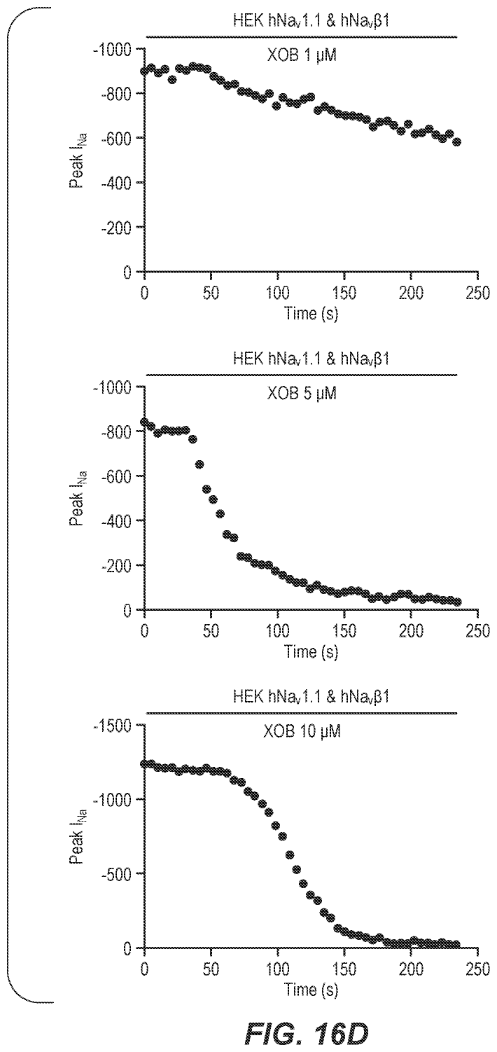

XOB inhibition of Nav1.1-generated sodium current by manual and automated patch clamp: The investigation into modulation of VGSC function by XOB began with an initial analysis of 10 μM XOB on I_{Na} mediated by human Naᵥ1.1 stably expressed in HEK cells using manual whole-cell patch clamp (FIG. 16A). In three cells, the acute perfusion of 10 μM XOB reduced the mean Naᵥ1.1 peak I_{Na} density at 0 mV by 94.1±1.31% compared to baseline. The acute inhibition of Naᵥ1.1 by XOB was prolonged, with only 17.5±3.64% of the original baseline I_{Na} density at 0 mV recovered after 10 minutes of washing out XOB. The inhibitory effect of 10 μM XOB on the current-voltage (I-V) relationship of Naᵥ1.1 is shown in (FIG. 16B). We next examined if the coexpression of human Naᵥβ1 subunits, which are integral components of VGSC structure in native cells (O'Malley, Annu Rev Physiol., 2015; 77:481-504), altered the acute inhibitory effect of XOB at concentrations of 10 μM, 5 μM, and 1 μM (FIG. 16C). After 3.9 minutes of perfusion, 10 μM and 5 μM XOB led to a 98.6% and 95.7% reduction in Naᵥ1.1 peak I_{Na}, respectively. 1 μM XOB reduced Naᵥ1.1 peak I_{Na} by 35.3% (Table 6). A diary plot showing the perfusion time course of acute I_{Na} inhibition in cells expressing hNaᵥ1.1+hb1 at 10, 5, and 1 μM XOB is shown in (FIG. 16D).

TABLE 6
Diary plot of peak I_{Na} evoked using a voltage step
from −120 mV to 0 mV for 250 ms every 5.21 s.

% Initial Peak I_{Na}

| Time (s) | 10 μM XOB | 5 μM XOB | 1 μM XOB |
|---|---|---|---|
| 0 | 100 | 100 | 100 |
| 31.2 | 97.1 | 95.4 | 100.6 |
| 62.4 | 94.8 | 40.0 | 93.0 |
| 93.6 | 73.7 | 23.7 | 88.8 |
| 124.8 | 28.5 | 11.2 | 87.0 |
| 150.8 | 8.7 | 9.3 | 77.7 |
| 182 | 2.9 | 5.4 | 75.1 |
| 213.2 | 2.5 | 6.4 | 70.9 |
| 234 | 1.4 | 4.3 | 64.7 |

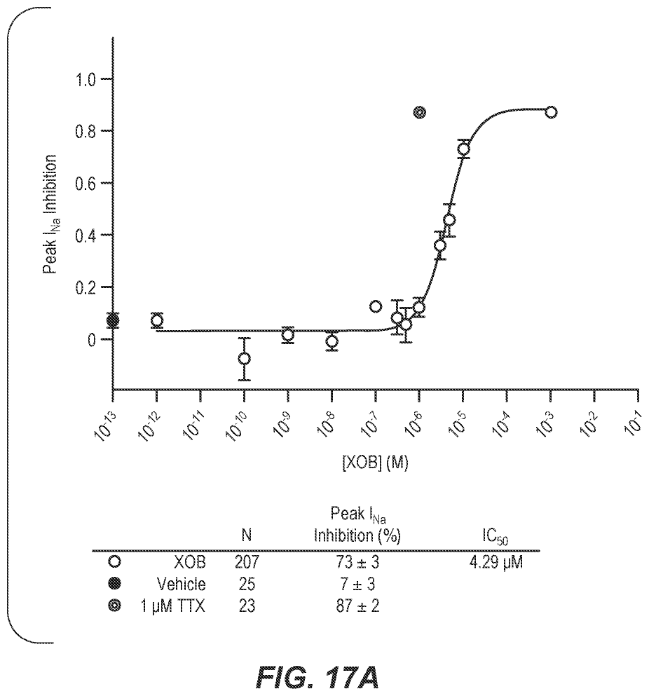

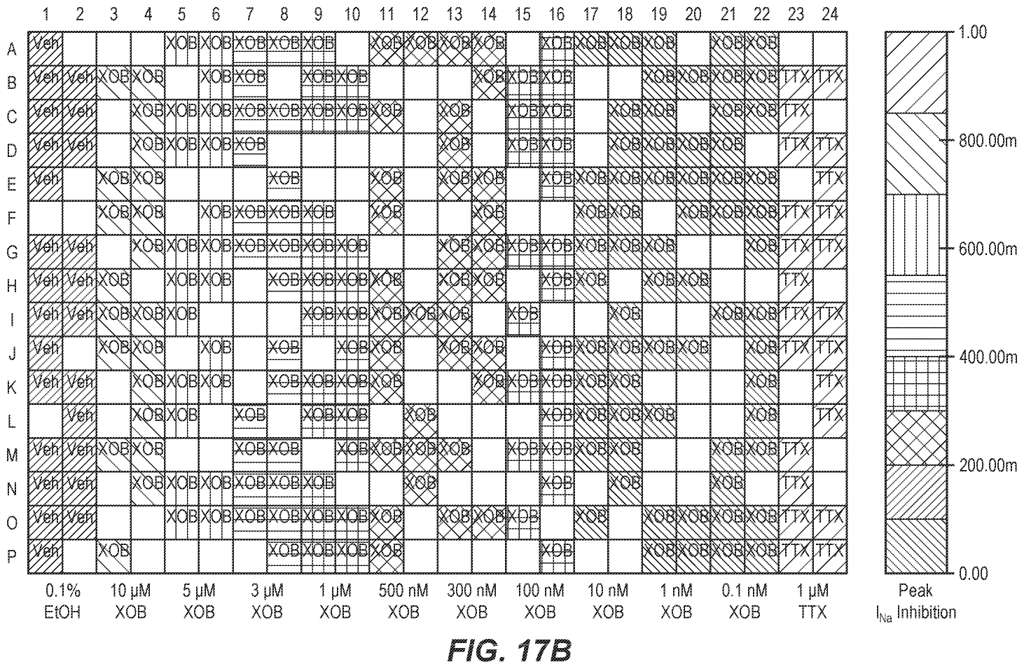

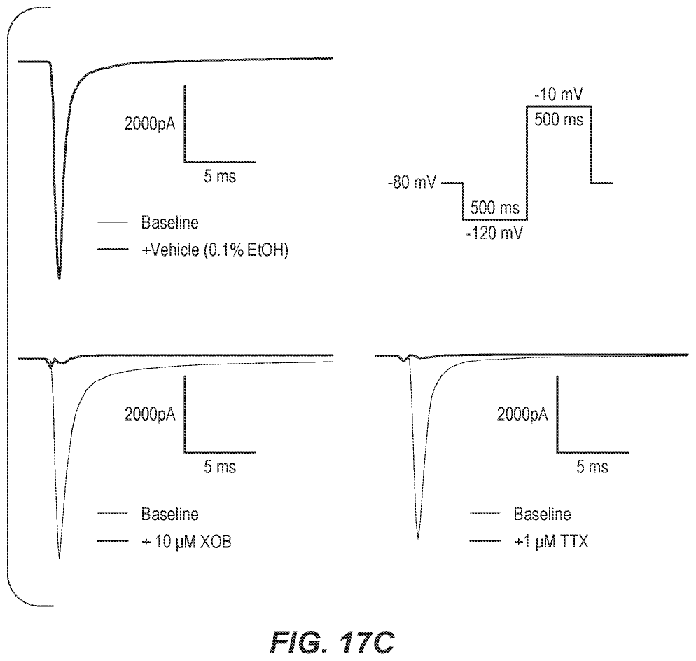

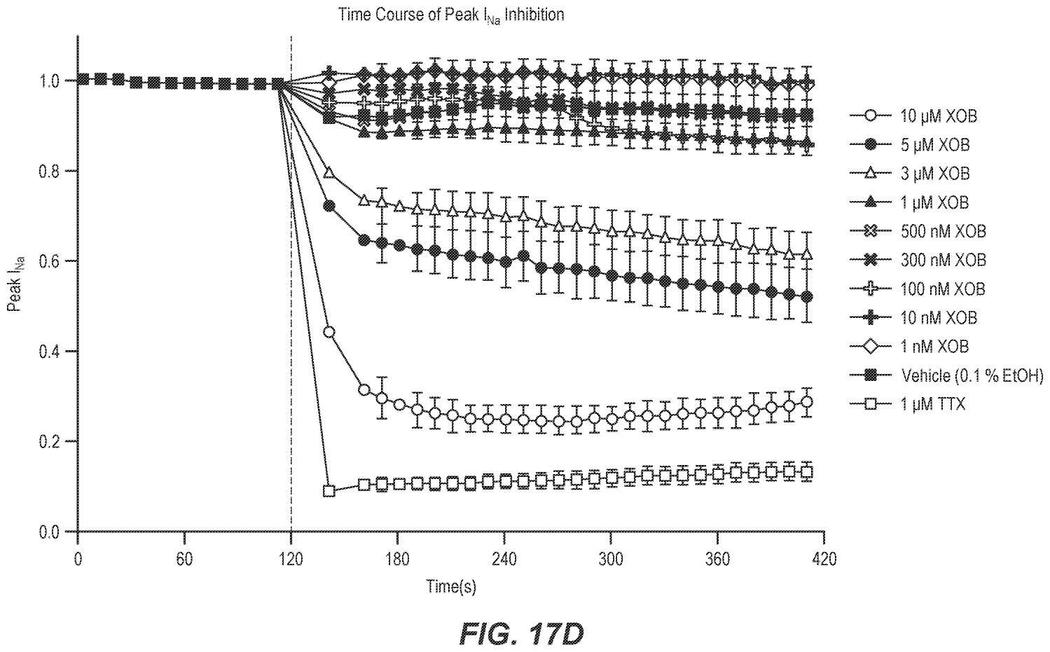

To efficiently characterize the concentration-response relationship for XOB-mediated inhibition of I_{Na}, we used high-throughput automated patch clamp technology (Nanion SyncroPatch 384) to record from HEK cells expressing hNaᵥ1.1+ hb1. The concentration-response curve of XOB on peak I_{Na} generated an IC₅₀ value of 4.29 μM, with a hill slope of 1.45 (FIG. 17A; Table 7). At 10 μM, XOB reduced baseline peak I_{Na} by 73±3%. This level of peak I_{Na} inhibition was similar to that of 1 μM TTX, which reduced peak I_{Na} by 87±2% from baseline in this assay. A heatmap showing the relative levels of Naᵥ1.1 peak I_{Na} inhibition by vehicle (0.1% EtOH), 1 μM TTX, or increasing concentrations of XOB is shown in (FIG. 17B). Representative peak I_{Na} traces using a protocol stepping from −120 mV to −10 mV in the presence of vehicle, 10 μM XOB, or 1 μM TTX are shown in (FIG. 17C). The mean peak I_{Na} value for each concentration group of cells at all recorded peak I_{Na} sweeps under baseline and drug conditions is shown in (FIG. 17D).

TABLE 7
XOB concentration response curve parameters.

XOB

| | Mean | 95% CI |
|---|---|---|
| Bottom | 0.02 | (−0.01 to 0.06) |
| Top | 0.88 | (0.79 to 0.97) |
| IC₅₀ | 4.29 μM | (3.24 μM to 5.55 μM) |
| Hill Slope | 1.45 | (0.99 to 2.19) |

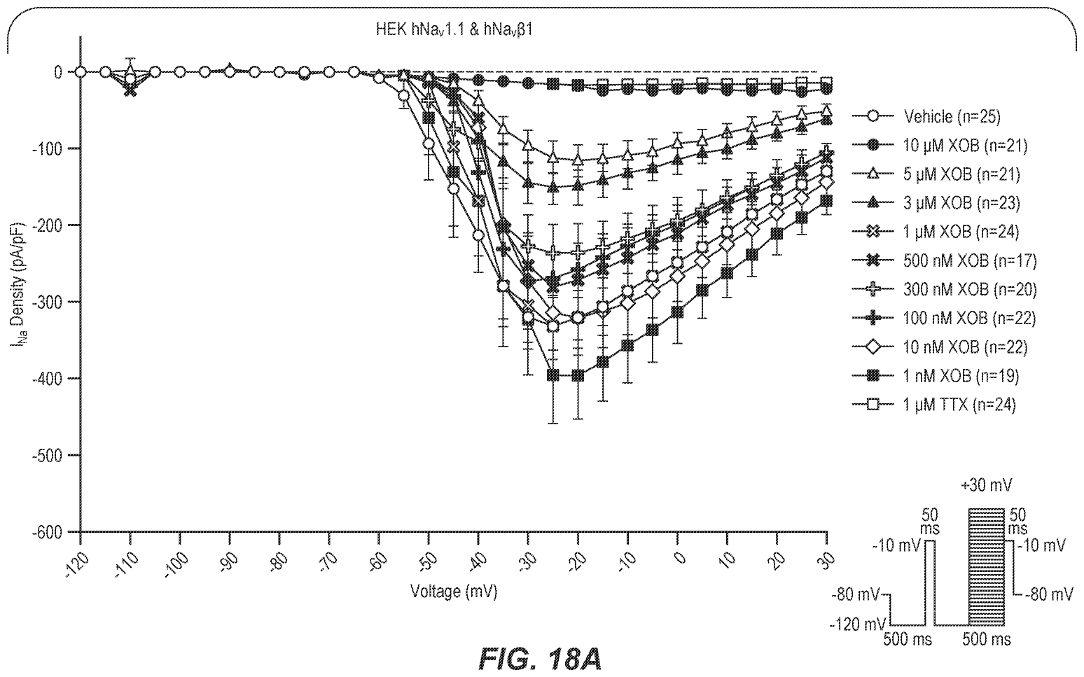

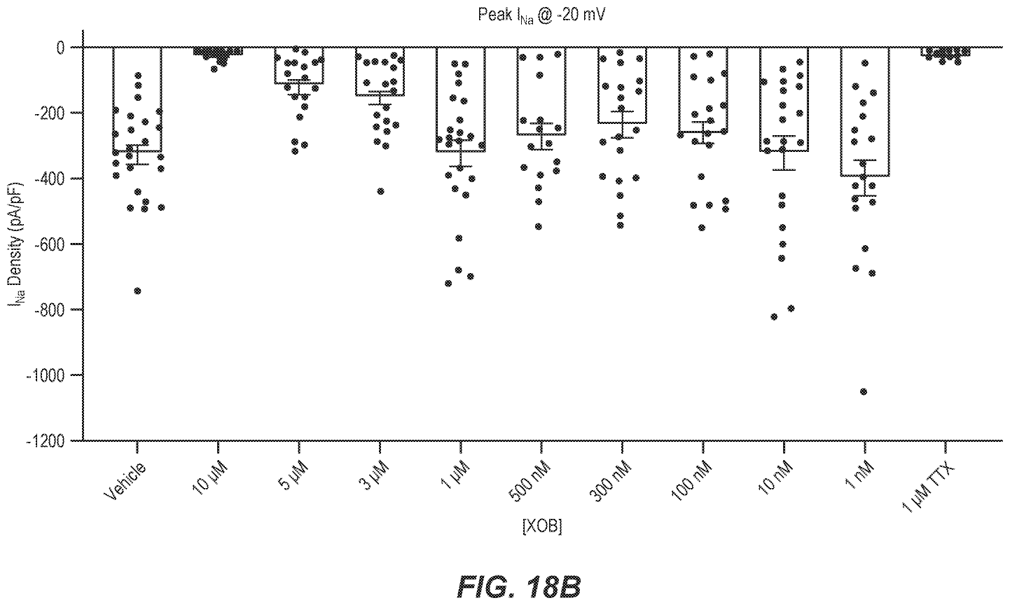

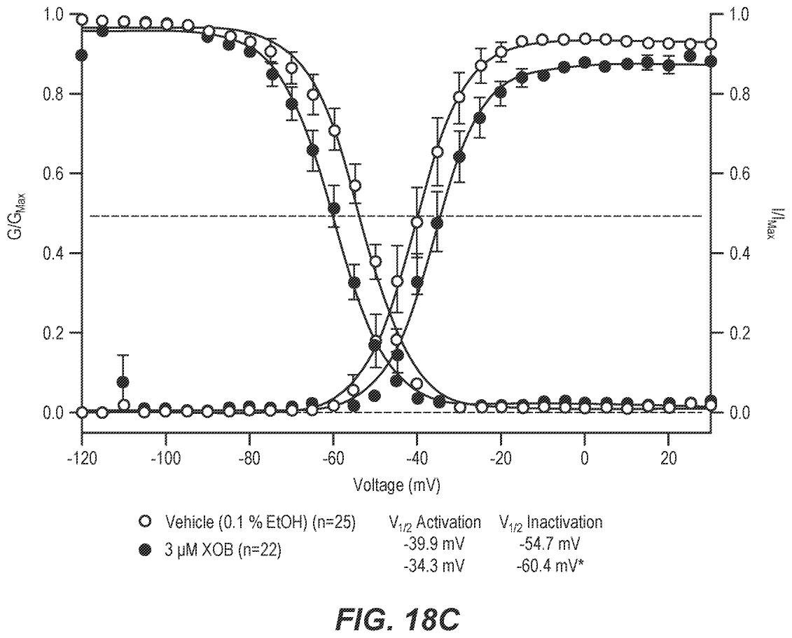

Probing further into the mechanism of XOB's reduction in peak I_{Na}, we ran protocols to elicit I-V relationships to test whether XOB modulates the voltage-dependent properties of Naᵥ1.1+b1 I_{Na} in a concentration-dependent manner. I-V curves in the presence of vehicle, 1 μM TTX, or increasing concentrations of XOB are shown in (FIG. 18A, and Table 8). The corresponding peak I_{Na} density at the −20 mV step of the I-V curve is shown in (FIG. 18B). As expected from the manual patch clamp experiments, cell groups perfused with 3, 5, or 10 μM XOB all showed significant inhibition of peak I_{Na} density at −20 mV compared to vehicle (3 μM: P=0.0019; 5 μM: P=0.0002; 10 μM: P<0.0001; one-way ANOVA, see Table 10). Comparable levels of peak I_{Na} density were observed in cells groups perfused with 1 μM TTX (−15.84±2.16 pA/pF) and 10 μM XOB (−17.46±3.1 pA/pF). When analyzing the voltage-dependence of sodium conductance, no effect was observed. However, the perfusion of 3 μM XOB did induce a significant-5.7 mV shift in the voltage-dependence of I_{Na} availability (P-value: 0.023; unpaired t-test, see FIG. 18C and Table 9). This hyperpolarizing shift in the voltage-dependence of inactivation is a likely contributor to the inhibitory effect of XOB on Naᵥ1.1.

TABLE 8
Concentration-dependent XOB inhibition of peak I_{Na} in HEK cells
stably co-expressing human Naᵥ1.1 and Naᵥβ1 elicited with
peak protocol using automated patch clamp (SyncroPatch).

| | | Peak I_{Na} | Normalized | | Valid |
|---|---|---|---|---|---|
| | | Density | Peak I_{Na} | | Wells |
| Compound | | (pA/pF) | Inhibition | n | (%) |
| 10 μM | XOB | −85.4 ± 12.9 | 0.73 ± 0.03 | 20 | 62 |
| 5 μM | XOB | −171.3 ± 22.2 | 0.46 ± 0.06 | 21 | 65 |
| 3 μM | XOB | −187.1 ± 23.3 | 0.36 ± 0.05 | 22 | 68 |
| 1 μM | XOB | −319.1 ± 34.5 | 0.12 ± 0.03 | 23 | 71 |
| 500 nM | XOB | −284.6 ± 35.2 | 0.05 ± 0.07 | 16 | 50 |
| 300 nM | XOB | −259.2 ± 38.0 | 0.08 ± 0.06 | 19 | 59 |
| 100 nM | XOB | −276.0 ± 31.0 | 0.13 ± 0.02 | 21 | 65 |
| 10 nM | XOB | −311.1 ± 41.7 | −0.011 ± 0.03 | 22 | 68 |
| 1 nM | XOB | −386.1 ± 56.6 | 0.015 ± 0.03 | 19 | 59 |
| 0.1 nM | XOB | −429.6 ± 50.7 | −0.077 ± 0.08 | 24 | 75 |
| Vehicle | | −321.8 ± 26.5 | 0.07 ± 0.03 | 25 | 78 |
| 1 μM | TTX | −56.3 ± 9.4 | 0.870 ± 0.02 | 23 | 71 |

TABLE 9
Voltage-dependent properties of conductance (G/G_{Max})
and voltage-dependence of availability (I/I_{Max}) between
vehicle (black) and 3 μM XOB (orange) conditions.

| | Vehicle | 3 μM XOB |
|---|---|---|
| | (n = 25) | (n = 22) |
| Capacitance (pF) | 11.06 ± 0.9 | 11.17 ± 1.0 |
| Activation V_{1/2} (mV) | −39.97 ± 2.1 | −34.32 ± 1.8 |
| Activation Slope Factor (mV⁻¹) | 6.16 ± 0.9 | 6.18 ± 1.1 |
| Inactivation V_{1/2} (mV) | −54.76 ± 1.8 | −60.46 ± 1.5* |
| Inactivation Slope Factor (mV⁻¹) | −6.35 ± 0.6 | −6.41 ± 0.7 |

*P-value: 0.023; unpaired t-test

### Acute Effects of 10 μM XOB on Mouse Prefrontal Cortex Layer V Pyramidal Neurons

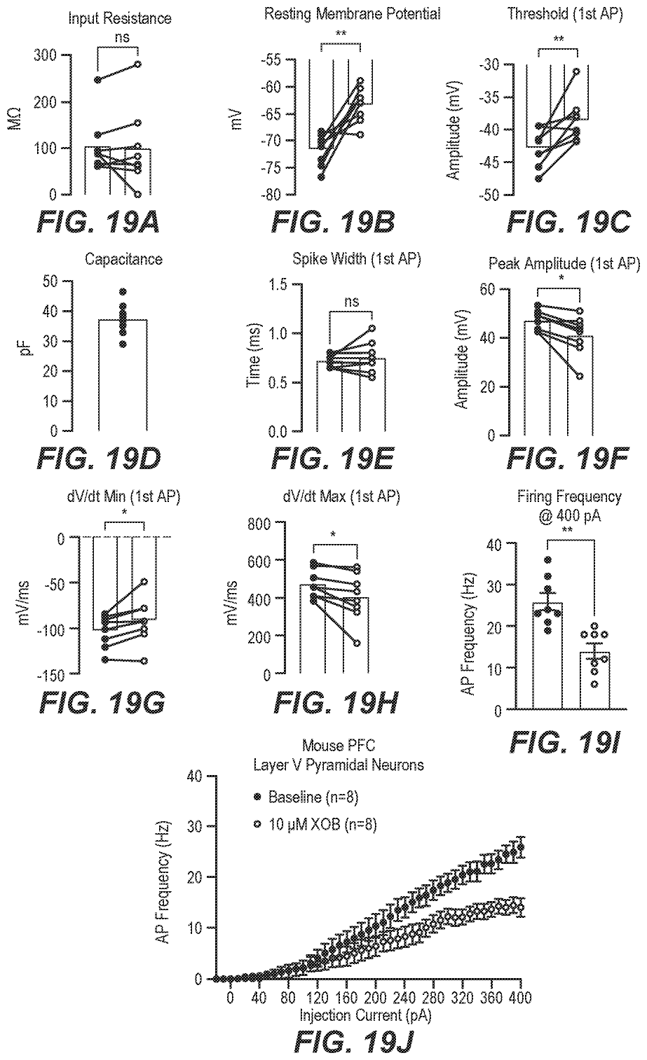

After characterizing the effects of XOB across a range of concentrations on Naᵥ1.1+b1 function in HEK cells, we asked if this observed inhibition of a major neuronal sodium channel subtype in a heterologous system translates to an in vivo model. Thus, we recorded passive and active properties of layer V pyramidal neurons in slices of wildtype mouse prefrontal cortex (PFC) (FIG. 19). Acute perfusion of 10 μM XOB led to ˜12 Hz reduction in maximum firing frequency of PFC layer V pyramidal neurons at 400 pA current injections (baseline=24.88±2.07 Hz; 10 μM XOB=14±1.82 Hz). 10 μM XOB induced a +8.45±1.15 mV depolarization of the resting membrane potential (baseline=−71.7±1.04 mV vs. 10 μM XOB=−63.25±1.15 mV) and significantly reduced the first AP peak amplitude (baseline=47.4±1.42 mV vs. 10 μM XOB=41.21±2.92 mV), maximum dV/dt (baseline=471.73±27.12 mV/ms vs. 10 μM XOB=402.68±46.20 mV/ms), minimum dV/dt (baseline=−103±6.28 mV/ms vs. 10 μM XOB=−91.32±8.95 mV/ms), and threshold of the first AP (baseline=−42.84±1.02 mV vs. 10 μM XOB=−38.43±1.23 mV). No significant changes in input resistance (baseline=104.52±21.84 MΩ vs. 10 μM XOB=100.08±30.18 MΩ2) or spike width (baseline=0.72±0.02 ms vs. 10 μM XOB=0.76±0.06 ms) were observed after perfusion of 10 μM XOB. (See FIGS. 19A-19J and Table 11.)

TABLE 10
Peak I_{Na} density obtained from I-V curve elicited
using a voltage step from −120 mV to −20 mV
using automated patch clamp (SyncroPatch).

| | | | |
|---|---|---|---|
| | Peak I_{Na} Density | | |
| Compound | @ −20 mV (pA/pF) | n | P Value |

| | | | | |
|---|---|---|---|---|
| 10 μM | XOB | −17.46 ± 3.1 | 21 | <0.001* |
| 5 μM | XOB | −115.6 ± 20.4 | 21 | 0.0002* |
| 3 μM | XOB | −149.4 ± 22.4 | 23 | 0.0019* |
| 1 μM | XOB | −320.5 ± 40.0 | 24 | ns |
| 500 nM | XOB | −269.2 ± 38.8 | 17 | ns |
| 300 nM | XOB | −235.1 ± 37.9 | 20 | ns |
| 100 nM | XOB | −258.3 ± 34.1 | 22 | ns |
| 10 nM | XOB | −319.1 ± 50.5 | 22 | ns |
| 1 nM | XOB | −395.9 ± 56.0 | 19 | ns |

| | | | |
|---|---|---|---|
| Vehicle | −320.6 ± 29.3 | 25 | — |

| | | | | |
|---|---|---|---|---|
| 1 μM | TTX | −15.84 ± 2.16 | 24 | <0.0001* |

TABLE 11
Effects of 10 μM XOB on wildtype mouse prefrontal cortex Layer
V pyramidal neuron membrane properties and action potential firing.

| | Baseline | 10 μM XOB |
|---|---|---|
| | (n = 8) | (n = 8) |
| Capacitance (pF) | 37.51 ± 1.88 | |
| Resting Vₘ (mV) | −71.7 ± 1.04 | −63.25 ± 1.15** |
| Input Resistance (MΩ) | 104.52 ± 21.84 | 100.08 ± 30.18 |
| Max Firing Rate @ 300 pA (Hz) | 18.88 ± 1.86 | 12.25 ± 1.53 |
| Max Firing Rate @ 400 pA (Hz) | 25.88 ± 2.07 | 14 ± 1.82** |
| Spike Width (ms) | 0.72 ± 0.02 | 0.76 ± 0.06 |
| First AP Peak Amplitude (mV) | 47.40 ± 1.42 | 41.21 ± 2.29* |
| First AP Max dv/dt (mV/ms) | 471.73 ± 27.12 | 402.68± |
| First AP Min dv/dt (mV/ms) | −103.00 ± 6.28 | −91.32 ± 8.95* |
| First AP Threshold (mV) | −42.84 ± 1.02 | −38.43 ± 1.23** |

Discussion: Tryptamine-class psychedelic compounds such as the psilocybin and DMT have reemerged as promising therapeutic candidates for treating numerous psychiatric conditions including depression, substance use, and anxiety-related disorders (Vollenweider and Preller, Nat Rev Neurosci, 2020; 21 (11): 611-624; D'Souza et al., Neuropsychopharmacol., 2022; 47 (10): 1854-1862).

Phenylalkylamine psychedelics such as 2C-B also possess clinically desirable properties such as oral activity at 12-24 mg, low sympathomimetic effects, and medium duration of action 4-8 h (Shulgin and Carter, Psychopharmacol Commun., 1975; 1 (1): 93-98; Papaseit et al., Front Pharmacol., 2018; 9:206) and there exist preliminary indications of efficacy in psychiatric conditions (González et al., Biomed Res Int., 2015; 2015). In vitro studies using [¹²⁵I]-2,5-dimethoxy-4-iodoamphetamine ([125I]-DOI) competition binding in heterologous cells expressing human or rat 5-HT_{2A} receptors report a high affinity for 2C-B with a K_{I} of 0.88 nM and 0.66 nM, respectively (McLean et al., J Med Chem., 2006; 49 (19): 5794-5803). Functionally, 2C-B shows high potency for 5-HT_{2A} (EC₅₀=2.1 nM), 5-HT_{2B} (EC₅₀=57 nM), and 5-HT_{2C} (EC₅₀=43 nM) receptors measured via in vitro Ca²⁺ mobilization assays (Luethi et al., Neuropharmacol., 2018; 134:141-148). Showing high affinity and potency at the 5-HT_{2A} receptor, 2C-B has been shown to act as a partial agonist with widely variable amounts of efficacy in PLC-, PLA₂-, and G_{q}-coupled Ca²⁺-mediated assays (McLean et al., J Med Chem., 2006; 49 (19): 5794-5803; Moya et al., J Pharmacol Exp Ther., 2007; 321 (3): 1054-1061; Rickli et al., Neuropharmacol., 2015; 99:546-553; Luethi et al., Neuropharmacol., 2018; 134:141-148). Intriguingly, 2C-B has been reported to act as an antagonist in 5-HT_{2A}-mediated inward current recordings from Xenopus oocytes transiently expressing 5-HT_{2A} receptors (Acuña-Castillo et al., Br J Pharmacol., 2002; 136 (4): 510-519; Villalobos et al., Br J Pharmacol., 2004; 21 (11): 611-624). The response generated in the oocyte model likely represents chloride current produced by Ca²⁺ transients coupled to PLC through activation of the transiently overexpressed 5-HT_{2A} receptors, raising the question of its physiological relevance to the action of 2C-B and related phenylalkylamines in mammalian neurons.

In this work, we sought to explore the chemical space of 2C-B binding at serotonin receptors by synthesizing the extended side-chain compound XOB as a probe for a putative exosite, analogous to the adrenergic β₂ receptor exosite (McCorvy et al., Nat Struct Mol Biol., 2018; 25 (9): 787-796; Kim et al. Cell, 2020; 182 (6): 1574-1588). We hypothesized that the aralkyloxyalkyl side-chain would enhance affinity and slow the kinetics of XOB binding at 5-HT_{2A} receptor when compared to the parent 2C-B molecule. Although XOB did display a much slower association rate, it showed a reduced affinity for the 5-HT_{2A} receptor compared to 2C-B and very poor binding for 5-HT_{2B}, 5-HT_{2C}, and 5-HT_{1A} receptors. Functionally, XOB shows a degree of selective antagonism at the 5-HT_{2A} receptor (FIGS. 15A-15C).

Rather than strengthening the interaction of XOB with the hypothetical 5-HT_{2A} receptor exosite, it is possible that the aralkyloxyalkyl side-chain extension on XOB increased non-specific interactions with the lipid bilayer or another membrane protein. The unexpected discovery of VGSC inhibition by XOB supports off-target engagement of another membrane protein by XOB. At low micromolar concentrations, XOB significantly decreased peak I_{Na} density and hyperpolarized the voltage-dependence of I_{Na} fast inactivation (FIGS. 17A-17D). Initial experiments using manual patch clamp showed that XOB inhibited I_{Na} generated by hNaᵥ1.1+hb1 in HEK cells. The lack of I_{Na} restoration after XOB wash out aligns with its slow association rate observed in our 5-HT receptor binding kinetic analysis and the molecule's lipophilic properties. To better understand the range of XOB's inhibition of I_{Na}, we used high-throughput patch clamp technology. We observed a concentration-dependent inhibition of Naᵥ1.1+b1-generated I_{Na} by XOB with an IC₅₀ of 4.29 μM, which is comparable to the affinity (K_{I}=2.6 μM) and potency (IC₅₀=1.3 μM) of XOB at 5-HT_{2A} receptors. In addition to its blockade of peak I_{Na}, XOB impacted the voltage-dependent gating of Naᵥ1.1. While XOB had no significant effects on the voltage-dependence of activation, 3 μM XOB caused a significant hyperpolarizing shift in the voltage-dependence of I_{Na} fast-inactivation. This shift in the voltage-dependence of availability provides mechanistic insight into XOB's inhibitory action on VGSCs. In the presence of XOB, more VGSCs in the brain would be inactivated, or unavailable, at hyperpolarized membrane potentials. This shift would be expected to reduce window current and elevate AP threshold, ultimately diminishing the intrinsic excitability of neurons in the presence of XOB.

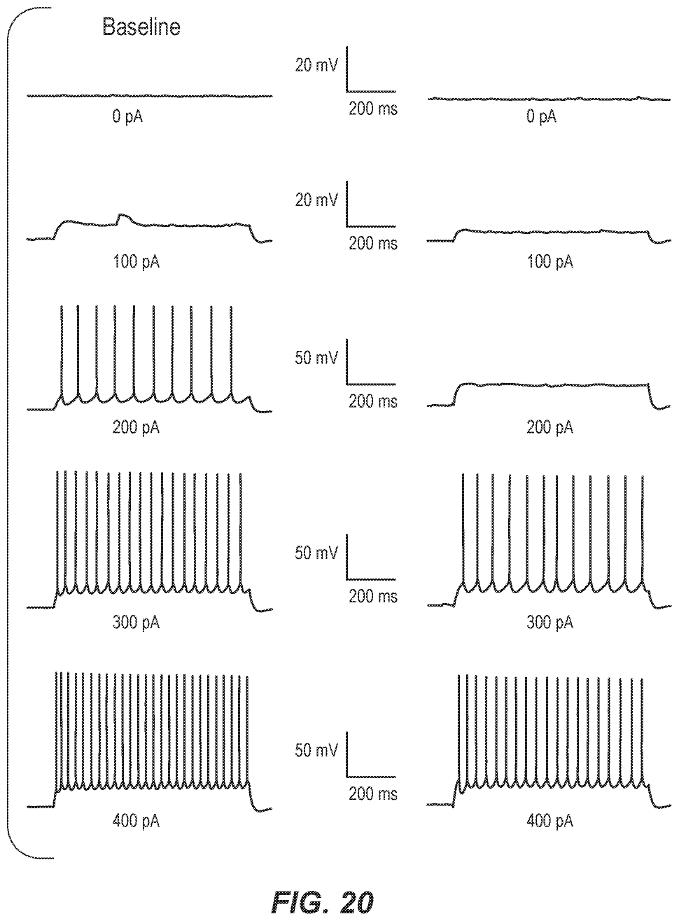

Indeed, the acute perfusion of 10 μM XOB significantly reduced maximum AP firing frequency, AP peak amplitude, rates of AP depolarization and repolarization (dV/dt), and depolarized the resting membrane potential and AP threshold of mouse PFC layer V pyramidal neurons in brain slices (FIGS. 19A-19J, FIG. 20). A particularly predominant expression of 5-HT_{2A} receptors is found in cortical layer V pyramidal neurons, where they are enriched in apical dendritic compartments (Willins et al., Synapse, 1997; 27 (1): 79-82; Jakab and Goldman-Rakic, Proc Natl Acad Sci USA, 1998; 95 (2): 735-740; Weber and Andrade, Front Neurosci, 2010; 4:36). 5-HT_{2A} receptor activation on layer V pyramidal neurons has generally been considered excitatory (making the cell more likely to spike), presumably via Gag coupled pathways, while 5-HT_{1A} receptors are thought to inhibit pyramidal neurons via activation of G protein-coupled inwardly-rectifying K⁺ channels (GIRK) channels (Araneda and Andrade, Neuroscience, 1991; 40 (2): 399-412; Andrade, Ann NY Acad Sci., 1998; 861 (1): 190-203; Andrade, Neuropharmacol., 2011: 61 (3): 382-386). With dual antagonism for 5-HT_{2A} receptors and VGSCs, we hypothesized that XOB would have depressing effects on the excitability of PFC layer V pyramidal neurons. We attribute the significant reduction in PFC layer V pyramidal neuron AP firing frequency, peak amplitude, as well as rates of depolarization and repolarization by XOB to its inhibition of VGSCs. Based on the proposed excitatory effects of 5-HT_{2A} agonists, we expected that the 5-HT_{2A} antagonism of XOB would result either in no change, or a hyperpolarization of the resting membrane potential. The significant depolarization in the resting membrane potential challenges this expectation and what would be anticipated of a classical VGSC blocker, raising the possibility that XOB modulates other ion conductances that contribute to the resting membrane potential. The lack of significant change in input resistance raises the possibility that other ion channels may be opening in response to XOB that are masking the expected change in input resistance caused by VGSC inhibition. Although no significant changes in spike half-width that would indicate modulation of other voltage-dependent conductances were observed after XOB perfusion, it is also plausible that XOB's VGSC inhibition had a negligible effect on membrane potential and input resistance when measured at rest, where a majority of VGSCs in the neuron would exist in a non-conducting state.

Future analysis of the acute effects of XOB in layer V pyramidal neurons from 5-HT_{2A}-knockout mice would be helpful in separating the contributions that 5-HT_{2A} and VGSC antagonism have on XOB's depression of excitability. It is important to note the mice used in this study were juvenile (ca. 3 weeks postnatal), and consideration of neurodevelopmental factors, including the age-dependence of 5-HT receptor expression in the rodent cortex, should be accounted for when interpreting the effects of XOB of neuronal excitability (Béïque et al., J Neurosci., 2004; 24 (20): 4807-4817). Additionally, neurons recorded from the medial PFC were not further subdivided into prelimbic, infralimbic, and anterior cingulate cortex. Therefore, region-specific differences in the effects of XOB on neurons of the mPFC may have been obscured in the present study. Another limitation of these physiological data involves the 5-HT_{2A} receptor amino acid sequence variation between human and rodent species. Although highly conserved, regions of divergence between humans and rodents may impact the functional interactions of ligand binding to the 5-HT_{2A} receptor (Dougherty and Aloyo, Psychopharmacol (Berl), 2011; 215 (3): 581-593). Use of a humanized 5-HT_{2A} receptor transgenic rodent model may be more physiologically relevant in future slice physiology studies.

Because XOB showed negligible binding affinity at 5-HT_{1A} receptors, and selectivity for 5-HT_{2A} over 5-HT_{2B} and 5-HT_{2C} receptors, we attribute the serotonergic component of its effects on layer V pyramidal neuron intrinsic excitability to 5-HT_{2A} antagonism. In addition to intrinsic modulation, 5-HT_{2A} receptors can impact the excitability of neurons through circuit-level synaptic effects, which were not measured in our current clamp experiments with XOB. It is well established that 5-HT_{2A} agonists increase the frequency of excitatory postsynaptic potentials and currents, especially asynchronous (nonelectrically evoked) release in PFC layer V pyramidal neurons (Aghajanian and Marek, Neuropharmacol., 1997; 36 (4-5): 589-599; Aghajanian and Marek, Brain Res., 1999; 825 (1-2): 161-171; Marek and Aghajanian, Eur J Pharmacol., 1999; 367 (2-3): 197-206; Andrade, Ann NY Acad Sci., 1998; 861 (1): 190-203; Andrade, Neuropharmacol., 2011: 61 (3): 382-386). In the presence of a psychedelic 5-HT_{2A} agonist such as DOI, we would expect XOB to significantly reduce the amplitude and frequency of EPSCs in layer V pyramidal neurons. 5-HT_{2A} receptors are also expressed on cortical GABAergic interneurons, (Willins et al., Synapse, 1997; 27 (1): 79-82; Jakab and Goldman-Rakic, Proc Natl Acad Sci USA, 1998; 95 (2): 735-740; Santana et al., Cereb Cortex., 2004; 14 (10): 1100-1109). As Naᵥ1.1 is thought to be predominantly localized to the axon initial segment of parvalbumin-positive (PV+) GABAergic fast-spiking interneurons in the cortex, we would expect XOB to reduce the excitability of cortical PV+ interneurons (Ogiwara et al., J Neurosci., 2007; 27 (22): 5903-5914). Future evaluation of the effects of XOB on GABAergic interneurons, as well as other isolated VGSC subtypes will be informative.

Clinically efficacious drugs used in the treatment of schizophrenia and bipolar disorder display significant antagonist (or inverse agonist) activity at 5-HT_{2A} receptors (e.g., risperidone, clozapine) and VGSCs (e.g., carbamazepine, lamotrigine), respectively, and these targets are thought to be important contributors in their therapeutic action. Pharmacologically, atypical antipsychotic drugs have been linked to a dual antagonism at D₂ receptors and 5-HT_{2A} receptors (Stahl, Antipsychotics and mood stabilizers: Stahl's essential psychopharmacology. Cambridge University Press. 2008). Furthermore, 5-HT_{2A} antagonism is hypothesized to improve negative symptoms and reduce the adverse extrapyramidal symptoms often observed with antipsychotic treatment (Meltzer, Neuropsychopharmacol, 1999; 25 (1): 1-9). Targeted sequencing of large patient populations has implicated VGSCs in the pathophysiology of schizophrenia (Rees et al., Biol Psychiatry., 2019; 85 (7): 554-562). Moreover, adjunctive lamotrigine therapy has shown efficacy in the treatment of schizophrenia, possibly through modulation of cortical excitability and glutamate transmission (Large et al., Psychopharmacol., 2005; 181:415-436). Together, these findings highlight the strong translational value that XOB has as a novel class displaying actions at 5-HT_{2A} receptors and VGSCs.

In summary, we report the discovery of a new structural class of substituted phenylalkylamines that antagonize both 5-HT_{2A} receptors and VGSCs, two key molecular actions for psychiatric therapeutics. As a therapeutic lead, XOB offers advancements for the development of more targeted psychiatric therapeutics with reduced adverse effects. As a tool, XOB has the potential to advance our understanding of the complex contributions of 5-HT_{2A} receptors and VGSCs in many neuropsychiatric disorders. While further characterization and structural optimization may be beneficial, XOB represents a good starting point for further exploration in chemical neuroscience and biological psychiatry.

### Example 4: Synthesis of Disclosed N-Substituted Phenylalkylamine Compounds

Additional N-substituted phenylalkylamines, e.g., compounds of Formula (I) or (II), can be synthesized according to the general reaction sequence shown in Scheme 2.

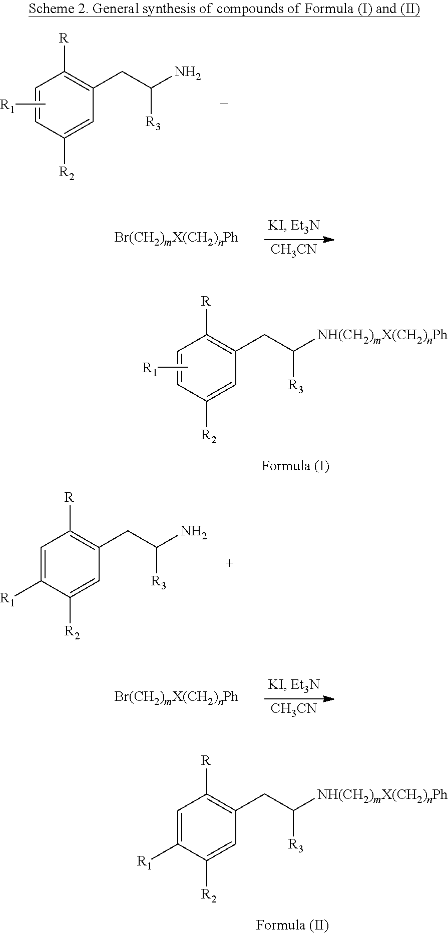

Briefly, a suitable phenylalkylamine precursor is reacted with a side chain precursor (e.g., Br(CH₂)ₘX(CH₂)ₙPh as shown above) under condensation conditions to produce a compound of Formula (I) or (II), by nucleophilic substitution of the leaving group (in this exemplary case, bromide) by the phenylalkylamine amine.

In this example, potassium iodide is used to accelerate the nucleophilic substitution reaction. Addition of an inorganic iodide salt (e.g., potassium iodide, sodium iodide) to improve the efficiency of nucleophilic substitution reactions is a well-known technique. However, potassium iodide is not necessary for the reaction to proceed. The person of skill in the art can determine whether potassium iodide should be added, and if so, how much should be used.

In this example, triethylamine is used as an exemplary base. However, as will be appreciated by the person of skill, other bases may be used. For example, diisopropylethylamine (DIPEA) and pyridine are common organic bases. Likewise, although acetonitrile is depicted as an exemplary solvent, substitution of acetonitrile for another suitable solvent can be performed according to the knowledge of a person of ordinary skill.

The synthesis of any other necessary starting materials or reagents will be readily apparent to the skilled artisan in view of this disclosure along with general references well known in the art. (See, e.g., Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995; Glennon et al. 1986. J. Med. Chem., 29 (2), 194-199; Nichols et al. 1991. J. Med. Chem., 34 (1), 276-281; Kedrowski et al. 2007. Organic Letters, 9 (17), 3205-3207; Heravi & Zadsirjan. 2016. Current Organic Synthesis, 13 (6), 780-833; Keri et al. 2017. European J. Med. Chem., 138, 1002-1033; Pérez-Silanes et al. 2001. J. Heterocyclic Chem, 38 (5), 1025-1030), and EP1937626 (U.S. Pat. No. 8,648,214), which describes synthesis of salmeterol and analogs thereof, all of which may be used to synthesize the compounds of the invention, in combination with the disclosure herein.)

### Example 5: Synthesis of N-Substituted Phenylalkylamine Analogs with Variant Side Chains

Purpose: To synthesize and physically characterize novel N-substituted analogs of phenylalkylamines with isomeric side chains.

Methods: To further probe the extended binding site of 5-HT_{2A}, 5-HT_{2B}, and 5-HT_{2C} receptors, the position of the heteroatom “hinge” in the analog series will be moved along the methylene units to create isomeric side chain variants. For example, for a 10-carbon side chain, “1 and 9”, “2 and 8”, “3 and 7”, “4 and 6”, “5 and 5”, “7 and 3”, “8 and 2”, or “9 and 1” variants, as well as a “no oxygen” compound containing only methylene units will be synthesized and compared (see Table 1). Such compounds are synthesized according to Examples 1 and 4, and accompanying discussion above, where the exemplary “6 and 4” side chain is replaced with “1 and 9”, “2 and 8”, “3 and 7”, “4 and 6”, “5 and 5”, “7 and 3”, “8 and 2”, or “9 and 1” variants, as well as a “no oxygen” side chain containing only methylene units. A series of N-substituted phenylalkylamines in accordance with Formula (I) or (II) will also be synthesized. In other examples, phenylalkylamine 5-HT_{2A} receptor ligands such as 2C-I, 2C-E, 2C-T, mescaline, and others known to one of skill in the art may be modified according to the described methods.

Results: Specific reaction conditions and isolation procedures will be determined empirically for each target compound. New compounds will be physically characterized by thermal analysis, proton and carbon nuclear magnetic resonance spectroscopy (¹H NMR, ¹³C NMR), GC-MS, LC-MS, X-ray diffraction, and elemental analysis. The described syntheses, which are straightforward and utilize chemistry known to one of skill in the art, are expected to yield novel N-substituted phenethylamine compounds. Such compounds may exhibit improved properties, such as enhanced binding affinity, specificity, or duration of action.

### Example 6: Assessing In Vitro Pharmacological Activity of Disclosed Compounds

Purpose: A receptor screen is performed to characterize the binding profiles and functional activity of disclosed compounds at various receptors, channels, and transporters. The results will facilitate comparisons to unsubstituted phenylalkylamines and other psychedelics. Additionally, the data will be used to support or modify the primary exosite hypothesis, and structure activity analyses of the data will aid in the design of new compounds.

Methods: Disclosed compounds are synthesized and submitted to the Psychoactive Drug Screening Program (PDSP) sponsored by the National Institute of Mental Health. The PDSP screen includes evaluations at 45 receptor and transporter binding sites. The majority of screenings are performed with cloned human receptors, with some exceptions. See, e.g., PDSP screening and results for DALT and derivatives thereof (Cozzi & Daley, Bioorganic & Medicinal Chemistry Letters, 2016; 26 (3): 959-964; Klein et al., Neuropharmacology, 2018; 142:231-239).

Briefly, test compounds are dissolved in DMSO and are tested at 10 mM in competition assays against radioactive probe compounds. Sites exhibiting >50% inhibition at 10 mM are tested in secondary assays at the identified receptor or transporter using 12 concentrations of the test compound, measured in triplicate, to generate competition binding isotherms. Kᵢ values are obtained from nonlinear regression of these binding isotherms from best-fit IC₅₀ values using the Cheng-Prusoff equation (Cheng & Prusoff, Biochem Pharmacol., 1973; 22 (23): 3099-108). For purposes of data analysis and comparison, Kᵢ values are converted to pKᵢ (−log Kᵢ) values.

Binding assays are performed using the following radioligands: [³H]8-OH-DPAT (5-HT_{1A}), [³H]GR125743 (5-HT_{1B/1D}), [³H]5-HT (5-HT_{1E}), [³H]ketanserin (5-HT_{2A}), [³H]LSD (5-HT_{2A/2B/6/7}), [³H]mesulergine (5-HT_{2C}), [³H]citalopram (serotonin transporter), [³H]prazocin (α1A/1B/1D), [³H]rauwolscine (α2, 5-HT_{2A/2B}), [¹²⁵I]pindolol (b1), [³H]CGP12177 (b2, b3), [³H]nisoxetine (norepinephrine transporter), [³H]SCH23390 (D1, D5), [³H]N-methylspiperone (D2/3/4), [³H]WIN35428 (dopamine transporter), [³H]DAMGO (μ-opioid), [³H]DADLE (δ-opioid), [³H]U69593 (K-opioid), [³H]muscimol (GABAA), [³H]funitrazepam (central benzodiazepine), [³H]PK11195 (peripheral benzodiazepine), [³H]pyrilamine (H1), [³H]tiotidine (H2), [³H]α-methylhistamine (H3), [³H]histamine (H4), [³H]QNB (M1e5), [³H](b)-pentazocine (s1), and [³H]DTG (s2).

Experimental protocols are available from the NIMH PDSP website, e.g., “Assay Protocol Book, Version III, March 2018, Bryan L. Roth, MD, PhD.” Briefly, both primary and secondary radioligand binding assays are carried out in appropriate binding buffers. The hot ligand concentration is comparable to the K_{d}. Total binding and nonspecific binding are determined in the absence and presence of 10 μM of the appropriate reference compound, respectively. Plates are incubated at room temperature and in the dark for 90 min. Reactions are stopped by vacuum filtration onto 0.3% polyethyleneimine (PEI) soaked 96-well filter mats using a 96-well Filtermate harvester, followed by three washes with cold wash buffers. Scintillation cocktail is then melted onto the microwave-dried filters on a hot plate and radioactivity is counted in a Microbeta counter.

Results: Disclosed compounds are anticipated to show affinity for the 5-HT_{2A} receptor based on the head groups of the psychedelic lead compounds. However, the binding affinity and efficacy is expected to be altered if the side chain interacts with the 5-HT_{2A} receptor exosite in a manner as would be understood to be analogous to the binding of XOB, when appreciated in view of the teachings herein.

### Example 7: Metabolic Stability

Purpose: To determine the metabolic stability of disclosed compounds. Metabolic stability assays measure the intrinsic clearance (CLᵢₙₜ) of a compound, providing data that can be used to calculate other key pharmacokinetic parameters such as bioavailability and half-life (t_{1/2}).

Methods: A high-throughput assay is used to determine metabolic stability of disclosed compounds and undeuterated analogs thereof in various matrices, including human liver microsomes, using LCMS analysis to quantify the percent compound remaining after incubation. Briefly, the disclosed compound is mixed with liver microsomes and activated. Following this incubation, acetonitrile is added to terminate the reaction. Then, the samples are centrifuged and the supernatant is dried. The residue is reconstituted and analyzed using liquid chromatography-mass spectrometry. Pharmacokinetic parameters are calculated using a noncompartmental model. The half-life (t_{1/2}) is estimated from the slope of the initial linear range of the logarithmic curve of compound remaining (%) versus time, assuming first order kinetics.

Results & Significance: Disclosed compounds may have altered clearance and half-life relative to a comparator, such as a corresponding unsubstituted phenethylamine. Such features provide advantages, such as an increased or reduced duration of action, that facilitate use in the treatment applications described herein.

### Example 8: In Vitro Metabolic Profiling

Purpose: To determine whether the disclosed compounds are metabolized and to identify metabolites thereof.

Methods: An in vitro study is conducted to evaluate metabolism and metabolites of disclosed compounds in human liver microsomes, such as S9 hepatocytes. Briefly, disclosed compounds are incubated with human liver microsomes and/or various recombinant enzymes to determine metabolism and formation of metabolites. Following incubation, the supernatant is analyzed directly by ultra-high performance liquid chromatography-mass spectrometry.

Phase I and/or Phase II metabolites are identified using mass spectrometry (MS). The % compound remaining and half-life of the disclosed compound (parent compound) are determined. MS data, such as extracted ion chromatograms, show parent and major metabolites. Metabolic transformation for each observed metabolite is elucidated, and metabolite masses, peak areas, and retention times are determined. Metabolic profiling may also be conducted according to the methods described in Muller & Rentsch, Anal Bioanal Chem, 2012; 402:2141-2151 and Pedersen et al., Drug Metab Dispos, 2013; 41:1247-1255.

Results & Significance: Compounds that undergo metabolism in vivo may produce pharmacologically active or chemically reactive metabolites that produce unexpected effects or potential toxicities. The FDA Guidance for Industry on Safety Testing of Drug Metabolites highlights the relevance of in vitro metabolite profiling early in drug development, as metabolites which are unique to or disproportionate in humans may require additional toxicological studies.

### Example 9: In Vitro CYP Enzyme Inhibition

Purpose: To assess the interactions between disclosed compounds and cytochrome P450 (CYP450) enzymes. Such interactions will provide insight into metabolism-mediated drug-drug interactions, which can occur when a compound affects the pharmacokinetics, such as the absorption, distribution, metabolism, and excretion, of simultaneously administered drugs by altering the activities of drug metabolizing enzymes and/or drug transporters.

Methods: An in vitro study is conducted to assess the inhibitory effect of the disclosed compound on recombinant human CYP450 isoenzymes. Recombinant human CYP450 isoenzymes are used to metabolize pro-fluorescent probe substrates to fluorescent products. Inhibition of human P450 isoforms is measured by reduced fluorescence following treatment with the disclosed compound at various concentrations.

Briefly, a disclosed compound is incubated in different concentrations in a mix containing buffer, enzymes, and substrate. Then, fluorescence is measured using a plate reader and percentage inhibition may be extrapolated out from the readings. Alternatively, the inhibitory effects of the disclosed compound on CYP enzymes may be assessed using high-performance liquid chromatography. Inhibition is evaluated using the Michaelis-Menten method. CYP enzyme inhibition may be conducted according to the methods described in Lin et al., J Pharm Sci. 2007 September; 96 (9): 2485-95 and Wójcikowski et al., Pharmacol Rep. 2020 June; 72 (3): 612-621.

Results & Significance: Metabolizing enzymes in the liver, such as CYP450 enzymes, are responsible for the majority of drug metabolism that occurs in the body. Six CYP450 class enzymes metabolize 90 percent of drugs, and two of the most significant metabolizers are CYP3A4 and CYP2D6 (Lynch & Price, Am Fam Physician. 2007; 76 (3): 391-6). Compounds can interact with such enzymes by inhibiting their enzymatic activity (CYP inhibition) or by inducing their gene expression (CYP induction).

### Example 10: In Vitro Evaluation of Membrane Permeability and Interactions with P-Glycoprotein (P-Gp) in MDCKII MDR1 Cells

Purpose: To assess the permeability and transport liability of disclosed compounds. Permeability is assessed using MDCK (Madin-Darby canine kidney) cells, and the effects of P-glycoprotein (P-gp) are evaluated to determine drug transport.

Methods: A bidirectional permeability study (apical to basolateral [AB] and basolateral to apical [BA]) is conducted to evaluate the apparent permeability of the disclosed compound. Additionally, an evaluation to determine if the disclosed compound acts as a P-gp substrate in MDCKII-MDR1 and mock MDCKII cell lines is performed.

Briefly, the disclosed compound and reference compounds are evaluated in two directions in the absence and presence of a P-gp inhibitor. The MDCKII and MDCKII-MDR1 cells are incubated in a transport buffer on both apical [A] and basolateral [B] sides. Then, the disclosed compound is added to each side of the cells and incubated. The rate of transport of the disclosed compound is determined in the absence or presence of a P-gp inhibitor. Following incubation, where the disclosed compound will permeate the cells in both AB and BA directions, the permeability of the cells is measured using a LC MS/MS system. The efflux ratio of the disclosed compound is calculated to determine if it is a P-gp substrate.

Results & Significance: This screening provides insight into the movement of the disclosed compound in a biological system. Compounds are classified as follows (Cambridge MedChem Consulting, ADME, 2019):

| Papp (nm/s) | Classification |
|---|---|
| >150 | High Permeability |
| 50-150 | Medium Permeability |
| <50 | Low Permeability |

Mass balance as a percentage (%) is calculated using the following equation:

$$\% Recovery = 100 \times ( C D ( t ) + C R ( t ) ) / {C}_{0}$$

Where CD (t) is the measured concentration in the donor well at time t (expressed as IS ratio), CR (t) is the measured concentration in the receiver well at time t (expressed as IS ratio), Co is the initial concentration in the donor solution (expressed as IS ratio).

The percentage of cell integrity is calculated using the following equation:

$$\% Integrity = 100 \times [ 1 - RFUbasolateral / RFUapical ]$$

LY RFU values are normalized by background mean values. A test item is considered to be a P-gp substrate when the efflux ratio in the absence of the inhibitor is >2 and if the ratio is significantly reduced in the presence of a P-gp inhibitor.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents.

1. A compound of Formula (II): 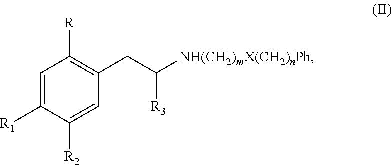
wherein:
m and n are each independently an integer from 1 to 13, provided that the sum of m+n is from 6 to 14;
X is O, S, or NH;
R and R2 are each independently C1-C8 alkoxy, wherein each C1-C8 alkoxy is optionally substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate;
R1 is selected from the group consisting of hydrogen, halogen, C1-C8 alkyl, C1-C8 alkoxy, C1-C8 thioalkyl, C2-C8 alkenyl, C2-C8 alkynyl, C3-C8 cycloalkyl, C3-C8 cycloalkenyl, aryl or heterocyclyl, each of which is optionally substituted by halogen, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate;
R3 is hydrogen or C1-C8 alkyl; and
Ph is phenyl optionally substituted by halogen, azido, alkyl, alkyl ester, hydroxy, alkoxy, carboxy, formyl, aryl, aryloxy, heterocyclyl, amino, alkylamino, arylamido, alkylamido, thiol, thioalkyl, thioaryl, alkylsulfonyl, alkylcarbamoyl, arylcarbamoyl, nitro, cyano, or nitrate;
or a pharmaceutically acceptable salt thereof.

2. The compound of claim 1, or a pharmaceutically acceptable salt thereof, wherein R and R2 are both —OCH₃.

3. The compound of claim 1, or a pharmaceutically acceptable salt thereof, wherein R3 is hydrogen, —CH₃, or —CH₂CH₃.

4. The compound of claim 1, or a pharmaceutically acceptable salt thereof, wherein R1 is F, Cl, Br, or I.

5. The compound of claim 4, or a pharmaceutically acceptable salt thereof, wherein R1 is Br.

6. The compound of claim 1, or a pharmaceutically acceptable salt thereof, wherein R1 is C1-C8 alkyl.

7. The compound of claim 6, or a pharmaceutically acceptable salt thereof, wherein R1 is —CH₃ or —CH₂CH₃.

8. The compound of claim 1, or a pharmaceutically acceptable salt thereof, wherein R1 is C1-C8 thioalkyl.

9. The compound of claim 1, or a pharmaceutically acceptable salt thereof, wherein R1 is —SCH₃.

10. The compound of claim 1, or a pharmaceutically acceptable salt thereof, wherein R1 is unsubstituted phenyl.

11. The compound of claim 1, or a pharmaceutically acceptable salt thereof, wherein R1 is phenyl substituted by azido or C1-C8 alkoxy.

12. The compound of claim 1, or a pharmaceutically acceptable salt thereof, wherein X is O.

13. The compound of claim 1, or a pharmaceutically acceptable salt thereof, wherein the sum of m+n is from 8 to 12.

14. The compound of claim 1, or a pharmaceutically acceptable salt thereof, wherein the sum of m+n is from 9 to 11.

15. The compound of claim 1, or a pharmaceutically acceptable salt thereof, wherein the sum of m+n is 10.

16. The compound of claim 15, or a pharmaceutically acceptable salt thereof, wherein m is 6 and n is 4.

17. A compound selected from Table 4, or a pharmaceutically acceptable salt thereof.

18. The compound of claim 17, wherein the compound is 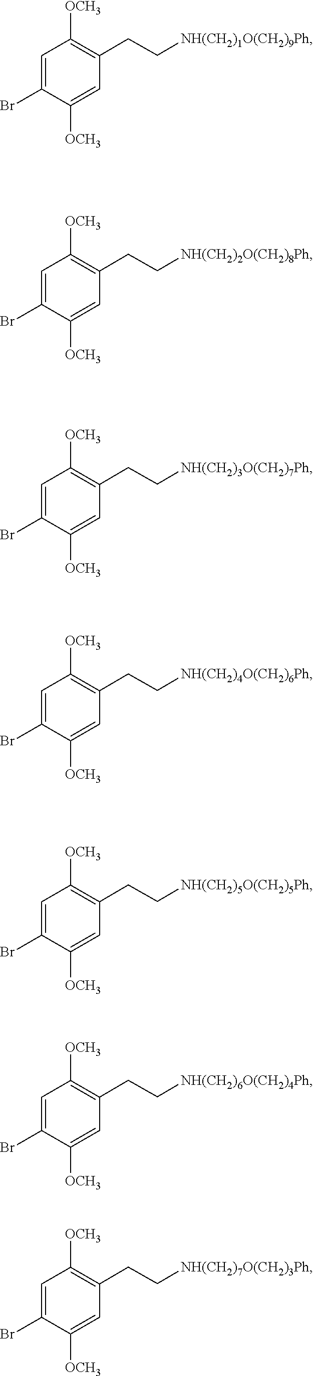 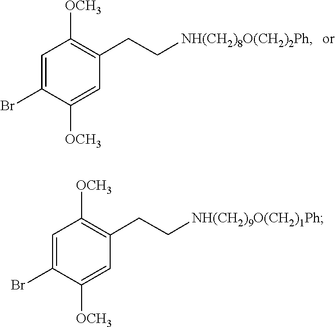
or a pharmaceutically acceptable salt thereof.

19. The compound of claim 18, wherein the compound is 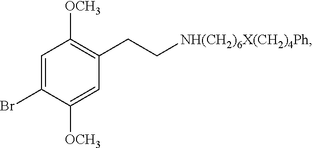
or a pharmaceutically acceptable salt thereof.

20. A pharmaceutical composition comprising a therapeutically effective amount of the compound of any one of claims 1-19, or a pharmaceutically acceptable salt thereof, and a pharmaceutically acceptable carrier, diluent, or excipient.

21. The pharmaceutical composition of claim 20, wherein the composition is suitable for oral, buccal, sublingual, intranasal, injectable, subcutaneous, intravenous, or transdermal administration.

22. The pharmaceutical composition of claim 21, wherein the composition is in unit dosage form.

23. The pharmaceutical composition of claim 22, wherein the unit dosage form comprises the compound, or a pharmaceutically acceptable salt thereof, in a total amount of between about 1 and about 500 mg, between about 2.5 and about 250 mg, or between about 5 and about 125 mg.

24. The pharmaceutical composition of claim 23, wherein the composition is an immediate release, controlled release, sustained release, extended release, or modified release formulation.

25. The pharmaceutical composition of claim 20, further comprising a therapeutically effective amount of an additional active compound, or a pharmaceutically acceptable salt thereof.

26. The pharmaceutical composition of claim 25, wherein the additional active compound is selected from the group consisting of amino acids, antioxidants, anti-inflammatory agents, analgesics, antineuropathic and antinociceptive agents, antimigraine agents, anxiolytics, antidepressants, antipsychotics, anti-PTSD agents, dissociatives, cannabinoids, immunostimulants, anti-cancer agents, antiemetics, orexigenics, antiulcer agents, antihistamines, antihypertensives, anticonvulsants, antiepileptics, bronchodilators, neuroprotectants, nootropics, empathogens, psychedelics, monoamine oxidase inhibitors, tryptamines, terpenes, phenethylamines, sedatives, stimulants, and vitamins; or a pharmaceutically acceptable salt thereof.

27. The pharmaceutical composition of claim 25, wherein the additional active compound, or a pharmaceutically acceptable salt thereof, acts to increase a therapeutic effect, provide an additional therapeutic effect, decrease an unwanted effect, increase stability or shelf-life, improve bioavailability, induce synergy, or alter pharmacokinetics or pharmacodynamics.

28. The pharmaceutical composition of claim 27, wherein the additional therapeutic effect is an antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, nootropic, empathogenic, psychedelic, sedative, or stimulant effect.

29. A compound of any one of claims 1-19, or a pharmaceutically acceptable salt thereof, for use in the treatment of a medical condition.

30. Use of the compound of any one of claims 1-19, or a pharmaceutically acceptable salt thereof, for the manufacture of a medicament for the treatment of a medical condition.

31. A method for modulating neurotransmission in a mammal, comprising administering to the mammal a therapeutically effective amount of the compound of any one of claims 1-19, or a pharmaceutically acceptable salt thereof.

32. A method of treating a medical condition in a mammal in need of such treatment, the method comprising administering the compound of any one of claims 1-19, or a pharmaceutically acceptable salt thereof.

33. The method of claim 32, wherein the medical condition is a disorder linked to dysregulation or inadequate functioning of neurotransmission.

34. The method of claim 33, wherein the disorder linked to dysregulation or inadequate functioning of neurotransmission is that of monoaminergic neurotransmission.

35. The method of claim 34, wherein the disorder linked to dysregulation or inadequate functioning of neurotransmission is that of serotonergic neurotransmission.

36. The method of claim 32, wherein the medical condition is a mental health disorder.

37. The method of claim 36, wherein the mental health disorder is selected from the group consisting of schizophrenia, schizoaffective disorder, schizotypal disorder, acute and transient psychotic disorder, delusional disorder, a substance-induced psychotic disorder, bipolar disorder, bipolar type I disorder, bipolar type II disorder, cyclothymic disorder, post-traumatic stress disorder (PTSD), adjustment disorder, affective disorder, depression, atypical depression, postpartum depression, catatonic depression, a depressive disorder due to a medical condition, premenstrual dysphoric disorder, seasonal affective disorder, dysthymia, anxiety, phobia disorders, binge disorders, body dysmorphic disorder, alcohol or drug abuse or dependence disorders, a substance use disorder, substance-induced mood disorder, a mood disorder related to another health condition, disruptive behavior disorders, eating disorders, impulse control disorders, obsessive compulsive disorder (OCD), attention deficit hyperactivity disorder (ADHD), personality disorders, attachment disorders, and dissociative disorders.

38. The method of claim 32, wherein the medical condition is a seizure disorder.

39. The method of claim 28, wherein the seizure disorder is epilepsy.

40. The method of claim 32, wherein the medical condition is a disorder linked to dysregulation or inadequate functioning of a voltage-gated ion channel.

41. The method of claim 40, wherein the voltage-gated ion channel is a voltage-gated sodium channel.

42. The method of claim 41, wherein the compound inhibits the activity of the voltage-gated sodium channel.

43. The method of any one of claims 31-42, wherein the mammal has a genetic variation associated with drug metabolism, including a genetic variation relating to CYP2D6 or CYP3A4 enzymes; or associated with a mental health disorder, trauma or stressor related disorder, depression, or anxiety, and including a genetic variation in mGluR5 or FKBP5; or relating to a membrane transporter, such as SERT, DAT, NET, or VMAT.

44. The method of any one of claims 31-43, wherein the mammal has altered epigenetic regulation of a gene the expression of which is associated with a mental health condition or susceptibility to a mental health treatment, such as the SIGMAR1 gene for the non-opioid sigma-1 receptor.

45. The method of any one of claims 31-44, wherein the mammal is a human.

46. A method of reducing the symptoms of a mental health disorder in a human, the method comprising identifying a human in need of said reducing, and administering to the human the compound of any one of claims 1-19, or a pharmaceutically acceptable salt thereof.

47. A method of reducing the symptoms of a mental health disorder in a human, the method comprising identifying a human in need of said reducing, and administering to the human the pharmaceutical composition of claim 20.

48. A method of reducing the symptoms of a mental health disorder in a human, the method comprising identifying a human in need of said reducing, and administering to the human the pharmaceutical composition of any one of claims 21-28.

49. A method of improving mental health or functioning in a human, the method comprising identifying a human in need of said improving, and administering to the human the compound of any one of claims 1-19, or a pharmaceutically acceptable salt thereof.

50. A method of improving mental health or functioning in a human, the method comprising identifying a human in need of said improving, and administering to the human the pharmaceutical composition of claim 20.

51. A method of improving mental health or functioning in a human, the method comprising identifying a human in need of said improving, and administering to the human the pharmaceutical composition of any one of claims 21-28.
